(19) United States
(12) Patent Application Publication
(10) Pub. No.: US 2024/0124419 A1
(43) Pub. Date: Apr. 18, 2024

(54) SMALL MOLECULE INHIBITORS OF GRP78 AND USES THEREOF

(71) Applicant: The Regents of the University of Michigan, Ann Arbor, MI (US)

(72) Inventors: Nouri Neamati, Ann Arbor, MI (US); Joyeeta Roy, Ann Arbor, MI (US); Soma Samanta, Ann Arbor, MI (US); Suhui Yang, Ann Arbor, MI (US); Ding Xue, Ann Arbor, MI (US)

(21) Appl. No.: 18/273,942

(22) PCT Filed: Feb. 2, 2022
(86) PCT No.: PCT/US2022/014893

§ 371 Date: Jul. 24, 2023

Related U.S. Application Data
(60) Provisional application No. 63/144,568, filed on Feb. 2, 2021.

(51) Int. Cl. C07D 401/14; A61K 45/06; A61P 35/00; C07D 215/28; C07D 401/06; C07D 405/06; C07D 405/14; C07D 417/14; C07D 487/08

(52) U.S. Cl. CPC C07D 401/14; A61K 45/06; A61P 35/00; C07D 215/28; C07D 401/06; C07D 405/06; C07D 405/14; C07D 417/14; C07D 487/08

(57) ABSTRACT

This invention is in the field of medicinal chemistry. In particular, the invention relates to a new class of small-molecules as defined within Formula I (as defined herein) which function as inhibitors of glucose-regulated protein 78 (GRP78) within cancer cells and/or immune cells, and which function as effective therapeutic agents for treating, ameliorating, and preventing various forms of cancer (e.g., pancreatic cancer), viral infections (e.g. SARS-CoV-2), and inflammatory diseases. In addition, this invention also relates to a new class of PROTACs having Formulas II, III and IV (as defined herein) which function as degraders of GRP78 within cancer and/or immune cells. Pharmaceutical compositions comprising said compounds of Formulas I, II, III, or IV are also within the scope of the present invention.

### CROSS-REFERENCE TO RELATED APPLICATIONS

This application claims the benefit of U.S. Provisional Application No. 63/144,568 filed Feb. 2, 2021, the contents of which is herein incorporated by reference in their entirety.

### FIELD OF THE INVENTION

This invention is in the field of medicinal chemistry. In particular, the invention relates to a new class of small-molecules as defined within Formula I (as defined herein) which function as inhibitors of glucose-regulated protein 78 (GRP78) within cancer cells and/or immune cells, and which function as effective therapeutic agents for treating, ameliorating, and preventing various forms of cancer (e.g., pancreatic cancer, leukemia, colon cancer, CNS cancers (e.g. glioblastoma), non-small lung cancer, melanoma, ovarian cancer, renal cancer, breast cancer, prostate cancer, esophageal cancer, cervical cancer and colorectal cancer), viral infections (e.g. SARS-CoV-2), and inflammatory diseases. In addition, this invention also relates to a new class of PROTACs having Formulas II, III and IV (as defined herein) which function as degraders of GRP78 within cancer and/or immune cells. Pharmaceutical compositions comprising said compounds of Formulas I, II, III, or IV are also within the scope of the present invention.

### INTRODUCTION

Cancer is the second most common cause of death in the United States. For example, pancreatic cancer remains one of the deadliest human diseases and options for effective systemic therapy are limited (1,2). Thus, there is an urgent need to develop new and more impactful therapies for this disease. Currently, gemcitabine-based regimens are considered the standard of care treatment for pancreatic cancer patients. Two front line regimens gemcitabine/nab-paclitaxel, and FOLFIRINOX have shown a survival benefit but at the expense of significant side effects (3). Moreover, lack of response and development of resistance to treatment limit the use of the front-line regimens. Thus, novel treatment options are needed to overcome drug resistance when used as a single agent or in combination with standard-of-care chemotherapy.

The endoplasmic reticulum (ER) is a multifunctional cellular organelle responsible for the proper folding of newly synthesized proteins, degradation of misfolded proteins, and maintenance of cellular homeostasis. Cancer cells are subject to intrinsic stress as they are highly proliferative and have a higher demand for protein synthesis and folding. Additionally, cancer cells are subject to extrinsic stress in the cancer microenvironment including hypoxia, low pH, and nutrient deprivation (4). Such conditions contribute to ER stress and impaired ER functions. As a result, cells activate the unfolded protein response (UPR) to mitigate the consequences of ER stress and to maintain cellular homeostasis. The UPR has dual functions; it can either mitigate the deleterious effect of ER stress or activate apoptosis (5,6). Cancer cells are known to direct the UPR to promote survival and growth. Thus, redirecting the UPR response to apoptosis in cancer cells is a promising approach for cancer therapy.

Glucose-regulated protein, 78 kDa (GRP78), also referred to as HSPA5/BiP, is a key molecular chaperone in the ER and also a master regulator of ER stress signaling (7). Under normal conditions, GRP78 associates with ER transmembrane receptors, protein kinase RNA-like endoplasmic reticulum kinase (PERK), inositol-requiring enzyme-1 (IRE1), and activating transcription factor 6 (ATF6) and maintains these sensors in an inactive state (8). Under stress, unfolded proteins accumulate in the ER resulting in GRP78 dissociation from the transmembrane receptors and causing activation of PERK, IRE1, and ATF6 (9). Activated PERK leads to phosphorylation of the α-subunit of eukaryotic initiation factor 2 (eIF2α) that in turn shuts off global mRNA translation reducing the protein load on the ER. This event protects cells from ER stress-related damage. However, prolonged ER stress leads to the activation of transcription factor 4 (ATF4) by the phosphorylated eIF2a resulting in the subsequent transcriptional upregulation of C/EBP homologous protein (CHOP) and growth arrest and DNA damage-inducible 34 (GADD34) (8). CHOP translocates to the nucleus and facilitates programmed cell death by upregulating its proapoptotic target genes. Activated IRE1 promotes the splicing of a retained intron from the mRNA encoding the transcription factor X box-binding protein 1 (XBP1) in the cytoplasm (10). The generated splicing variants, XBPIs, move to the nucleus and induce the transcription of genes coding for ER chaperones which protect the cells from the deleterious effects of ER stress (11). In response to ER stress, ATF6 dissociates from the ER membrane and moves to the Golgi apparatus, where its cytoplasmic domain undergoes proteolytic cleavage to form an active transcription factor. This active version of ATF6 translocates to the nucleus and promotes the transcription of several UPR genes encoding GRP78, GRP94, protein disulfide isomerase (PDI), and XBP1 allowing the cells to re-establish initial homeostasis (12). Thus, GRP78 regulates UPR by activating above mentioned ER transmembrane sensors and play important roles in regulating various cellular process required for tumorigenesis. Several murine cancer models confirm GRP78 requirement for tumorigenesis (13). Moreover, GRP78 interacts with and suppresses the activation of caspase-7 to prevent apoptosis (14), promoting cytoprotection and modulating chemosensitivity (15). Conversely, inhibition of GRP78 triggers UPR and causes caspase-4 mediated apoptosis (16). In cancer cells, ER stress inducers (such as thapsigargin and tunicamycin) cause UPR-mediated apoptosis (17). In mutant KRAS-driven pancreatic cancer in mice, GRP78 haploinsufficiency suppresses acinar-to-ductal metaplasia and oncogenic signaling (18). Thus, inhibition of GRP78 is an effective approach to disrupt ER homeostasis and suppress its anti-apoptotic properties. Furthermore, GRP78 induction in tumor, stromal, and dormant cancer cells, as an adaptive response to ER stress, promotes therapeutic resistance in cancer (19); therefore, inhibition of GRP78 overcomes resistance to multiple anti-cancer treatments (13). Moreover, increased GRP78 expression levels in patient tumor tissues correlate with poor survival in several cancers (20,21).

Accordingly, there is an urgent need for GRP78 inhibitors that are efficacious in suppressing tumor growth, including cancers related to GRP78 acivity, and overcome resistance.

The present invention addresses this need.

### SUMMARY

In recent times the whole world is traumatized by COVID-19 pandemic, challenged the global health care system, the scientific community is struggling to find medication to cure one of the most infectious and rapidly proliferating diseases. SARS-coronavirus 2 is causing the COVID-19 pandemic; SARS-CoV-2 instigates pulmonary and systemic inflammation leading to multi organ failure. The mechanism of entry of SARS-CoV-2 has been well documented (36); two strategies are employed to prevent the entry of virus are blockade of ACE2 (exopeptidase expressed on epithelial cells of the respiratory tract), and inhibition of TMPRSS2 (transmembrane protease serine 2). Reports suggest the SARS-CoV-2 spike protein (S glycoprotein) bind to its receptor human angiotensin-converting enzyme (hACE2) through its receptor-binding domain (RBD). Cathepsin L induces the fusion of SARS particles bound to ACE2 with the host cell (37). Cell-surface Glucose Regulated Protein 78 (CS-GRP78), also termed (HSPA5), has been reported to be a potential receptor of some viruses, including the novel SARS-CoV-2 (38). Ibrahim and coworkers predicted binding to be more favorable between regions III (C391-C525) and IV (C480-C488) of the spike protein model and GRP78; and the main driving force for GRP78 binding with the predicted binding affinity of −9.8 kcal/mol is region IV. They proposed milder human coronaviruses NL63, 229E, OC43, and HKU1 may serve vaccine against COVID-19 since they have similar HSPA5 recognition region on their spikes. Furthermore, Rangel and coworkers based on bioinformatics approaches also predicted, SARS-CoV2 could interact with GRP78 in mammalian species (cats, dogs, pigs, mice, and ferrets) although they do not play much role in host selectivity however can be targeted for control of virus replication (39). Koseler et al predicted SARS-CoV-2 infection leads to increased GRP78 concentrations (40). There are also evidences from other research groups that GRP78 has a potential role in SARS-CoV-2 entry (41, 42).

Recently, a large number of YUM70 analogues were synthesized containing novel features including better solubility, physicochemical, and pharmaceutical properties. Several of the newly synthesized compounds are significantly more potent than YUM70. In addition, a large number of PROTACs were recently synthesized using new GRP78 inhibitors as warhead. Such inhibitors and degraders were shown to be potent in a panel of cancer cell lines and quite synergistic with select FDA approved drugs.

Indeed, experiments conducted during the course of developing embodiments for the present invention resulted in the development of a series of novel hydroxyquinolines targeting GRP78. The analog, YUM70, showed significant efficacy in a pancreatic cancer xenograft model with no detectable toxicity to normal tissues. YUM70 treatment upregulates ER stress-related genes, induces apoptosis, and demonstrates synergy with the FDA approved drugs topotecan and vorinostat in killing pancreatic cancer cells.

Accordingly, the present invention relates to inhibitors of GRP78 having Formula I (as defined herein) within cancer cells and/or immune cells, and which function as effective therapeutic agents for treating, ameliorating, and preventing various forms of (e.g., pancreatic cancer, leukemia, colon cancer, CNS cancers (e.g. glioblastoma), non-small lung cancer, melanoma, ovarian cancer, renal cancer, breast cancer, prostate cancer, esophageal cancer, cervical cancer and colorectal cancer) viral infections (e.g. SARS-CoV-2), and inflammatory diseases. In addition, this invention also relates to a new class of PROTACs having Formula II, III and IV (as defined herein) which function as degraders of GRP78 within cancer and/or immune cells. Pharmaceutical compositions comprising said compounds of Formulas I, II, III, or IV are also within the scope of the present invention.

In a particular embodiment, compounds encompassed within Formula I are provided:

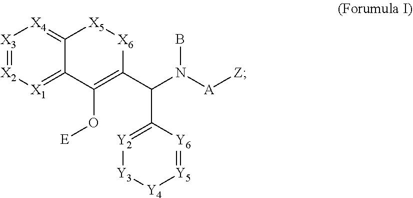

including pharmaceutically acceptable salts, solvates, and/or prodrugs thereof.

Formula I is not limited to a particular chemical moiety for X₁, X₂, X₃, X₄, X₅, X₆, Y₂, Y₃, Y₄, Y₅, Y₆, A, B, E and Z.

In some embodiments, the particular chemical moiety for X₁, X₂, X₃, X₄, X₅, X₆, Y₂, Y₃, Y₄, Y₅, Y₆, A, B, E and Z permits the resulting compound capable of one or more of
inhibiting GRP78 activity;
serving as an effective therapeutic agent for treating, ameliorating, and preventing various forms of cancer, viral infections, and inflammatory diseases;
inducing ER stress-mediated apoptosis in the tumor cells implanted in mice without major toxicity to normal tissues; and
inducing ER stress and triggers UPR by inhibiting GRP78.

In some embodiments, X₁ is either CH or N.

In some embodiments, X₂, X₃, X₄, X₅ and X₆ are each independently selected from CR¹ or N, with the proviso that at least three of them must be CR¹.

In some embodiments, A is selected from CO, SO, and SO₂.

In some embodiments, Y², Y³, Y⁴, Y⁵, Y⁶ are each independently selected from CH, CR² and N.

In some embodiments, Y⁵ is a bond, in which case one of Y³, Y⁴, or Y⁶ is NR², 0, or S, while the other two may be CR² or N.

In some embodiments, B, E are each independently selected from hydrogen and R³.

In some embodiments, Z is R³.

In some embodiments, R¹ is independently selected from the group consisting of H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano, CH₂F, CHF₂, CF₃, OCF₃, SOR⁷, SO₂R⁷, NO₂, COR⁴, C₁₋₆ alkyl-COR⁴, N(R⁷)C₂₋₆ alkyl-NR⁷R⁷, —N(R⁷)C₂₋₆ alkyl-R⁴, N(C₂₋₆ alkyl)₂-NR⁷, —O(CH₂)ₚR⁴, —S(CH₂)ₚR⁴, and —N(R⁷)C(═O)(CH₂)ₚR⁴, with a proviso that not more than three R¹ can be other than H.

In some embodiments, R² is independently selected from the group consisting of H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano,

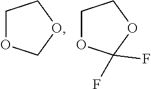

CF₃, OCF₃, SOR⁷, SO₂R⁷, NO₂, COR⁴, C₁₋₆ alkyl-COR⁴, N(R⁷)C₂₋₆ alkyl-NR⁷R⁷, N(C₂₋₆ alkyl)₂-NR⁷, CF₃, CO₂Et, CO₂H, R⁴, —N(R⁷)C₂₋₆ alkyl-R⁴, —O(CH₂)ₚR⁴, —S(CH₂)ₚR⁴, and —N(R⁷)C(═O)(CH₂)ₚR⁴, with a proviso that not more than two R² can be other than H.

In some embodiments, R³ is independently selected from the group consisting of C₁₋₆ alkyl, C₃₋₆ alkenyl, C₃₋₆ alkynyl, C₃₋₇ cycloalkyl, C₄₋₇ heterocycloalkyl, phenyl, naphthyl, 5-10 membered mono- or bicyclic heteroaryl, C₁₋₆ alkyl-C₃₋₇ cycloalkyl, C₁₋₆ alkyl-C₄₋₇ heterocycloalkyl, C₀₋₆ R⁴, and —N(R⁷)C₂₋₆ alkyl-R⁴.

In some embodiments, R⁴ is independently selected from the group consisting of OH, NR⁵R⁶, O(CH₂)_{q}NR⁵R⁶—, C₁₋₆ alkoxy, C₁₋₆ alkoxy-C₁₋₆ alkoxy, C₂₋₆ hydroxyalkoxy, cyclopropyl, oxetanyl, oxetanyloxy, oxetanylamino, oxolanyl, oxolanyloxy, oxolanylamino, oxanyl oxanyloxy, oxanylamino, oxepanyl, oxepanyloxy, oxepanylamino, azetidinyl, azetidinyloxy, azetidylamino, pyrrolidinyl, pyrolidinyloxy, pyrrolidinylamino, piperidinyl, piperidinyloxy, piperidinylamino, azepanyl, azepanyloxy, azepanylamino, dioxolanyl, dioxanyl, morpholino, thiomorpholino, thiomorpholino-S,S-dioxide, piperazino, dioxepanyl, dioxepanyloxy, dioxepanylamino, oxazepanyl, oxazepanyloxy, oxazepanylamino, diazepanyl, diazepanyloxy, and diazepanylamino, all of which may be optionally substituted with OH, OR⁷, oxo, halogen, R⁶, CH₂OR⁶, CH₂NR⁵R⁶ or CH₂CH₂CONR⁵R⁶.

In some embodiments, R⁵ and R⁶ are each independently selected from the group consisting of H, —CD₃, C₁₋₆ alkyl, C₃₋₆ alkenyl, C₃₋₆ alkynyl, C₃₋₈ cycloalkyl, —(C₁₋₃ alkyl)-(C₃₋₈ cycloalkyl), C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, and 5-11 membered heteroaryl; wherein R⁵ and R⁶ may be further independently substituted with up to three substituents selected from the group consisting of hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano, and halo; or alternatively, R⁵ and R⁶, taken together with the N atom to which they are both attached, may form a heterocycloalkyl ring of 4-7 members, containing up to one other heteroatom selected from O, S, or NR³, or a heterobicycloalkyl ring of 6-12 members which may be fused, bridged or spiro, and contain up to two other heteroatoms chosen from O, S(O)ₓ, or NR³.

In some embodiments, each R⁷ is independently selected from H, —CD₃, C₁₋₆ alkyl, C₃₋₆ cycloalkyl, phenyl, naphthyl, 5-10 membered mono- or bicyclo-heteroaryl, C₂₋₆ hydroxyalkyl, —SO₂-alkyl, NH—C₂₋₆ alkyl-NR⁵R⁶, C₁₋₆ alkoxy-C₁₋₆ alkyl, and C₂₋₆ alkyl-NR⁵R⁶; alternatively, two R⁷ taken together with the same N atom to which they are both attached, form a heterocyclic ring of 4-7 members, containing up to one other heteroatom selected from O, S, or NR³.

In some embodiments, p=0, 1, 2, 3, or 4.

In some embodiments, x=0, 1, or 2.

In some embodiments, compounds shown in Table I are contemplated for Formula I.

TABLE I

| SL NO | Structure | Name |
|---|---|---|
| G1 | 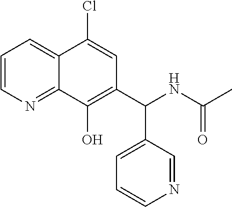 [SMILES: CC(=O)NC(c1cccnc1)c1cc(Cl)c2cccnc2c1O] | N-((5-chloro-8-hydroxyquinolin-7- G16yl)(pyridin-3-yl)methyl)acetamide |
| G2 | 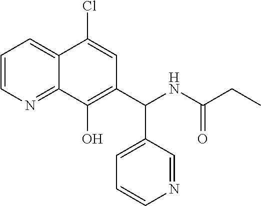 [SMILES: CCC(=O)NC(c1cccnc1)c1cc(Cl)c2cccnc2c1O] | N-((5-chloro-8-hydroxyquinolin-7- yl)(pyridin-3-yl)methyl)propionamide |
| G3 | 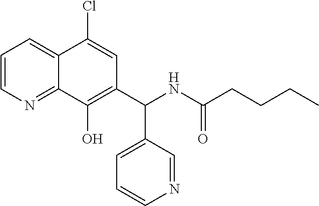 [SMILES: CCCCC(=O)NC(c1cccnc1)c1cc(Cl)c2cccnc2c1O] | N-((5-chloro-8-hydroxyquinolin-7- yl)(pyridin-3-yl)methyl)pentanamide |
| G4 | 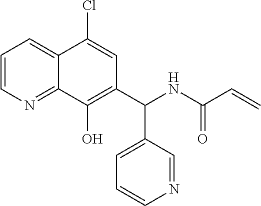 [SMILES: C=CC(=O)NC(c1cccnc1)c1cc(Cl)c2cccnc2c1O] | N-((5-chloro-8-hydroxyquinolin-7- yl)(pyridin-3-yl)methyl)acrylamide |
| G5 | 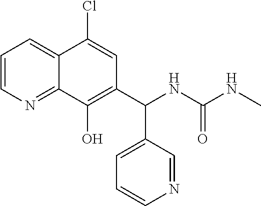 [SMILES: CNC(=O)NC(c1cccnc1)c1cc(Cl)c2cccnc2c1O] | 1-((5-chloro-8-hydroxyquinolin-7- yl)(pyridin-3-yl)methyl)-3-methylurea |
| G6 | 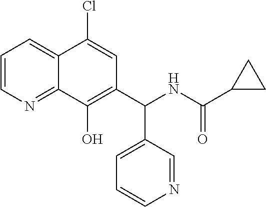 [SMILES: O=C(NC(c1cccnc1)c1cc(Cl)c2cccnc2c1O)C1CC1] | N-((5-chloro-8-hydroxyquinolin-7- yl)(pyridin-3- yl)methyl)cyclopropanecarboxamide |
| G7 | 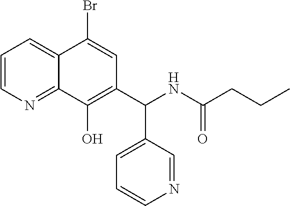 [SMILES: CCCC(=O)NC(c1cccnc1)c1cc(Br)c2cccnc2c1O] | N-((5-bromo-8-hydroxyquinolin-7- yl)(pyridin-3-yl)methyl)butyramide |
| G8 | 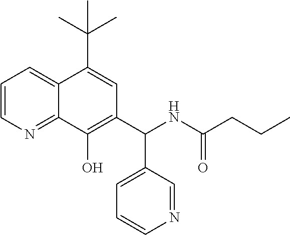 [SMILES: CCCC(=O)NC(c1cccnc1)c1cc(C(C)(C)C)c2cccnc2c1O] | N-((5-(tert-butyl)-8-hydroxyquinolin-7- yl)(pyridin-3-yl)methyl)butyramide |
| G9 | 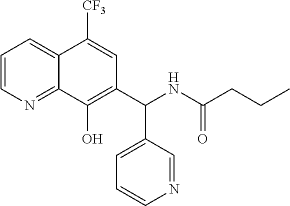 [SMILES: CCCC(=O)NC(c1cccnc1)c1cc(C(F)(F)F)c2cccnc2c1O] | N-((8-hydroxy-5- (trifluoromethyl)quinolin-7-yl)(pyridin- 3-yl)methyl)butyramide |
| G10 | 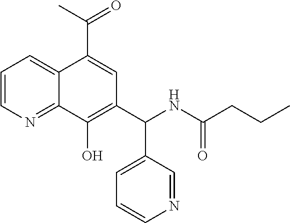 [SMILES: CCCC(=O)NC(c1cccnc1)c1cc(C(C)=O)c2cccnc2c1O] | N-((5-acetyl-8-hydroxyquinolin-7- yl)(pyridin-3-yl)methyl)butyramide |
| G11 | 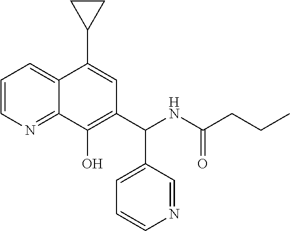 [SMILES: CCCC(=O)NC(c1cccnc1)c1cc(C2CC2)c2cccnc2c1O] | N-((5-cyclopropyl-8-hydroxyquinolin-7- yl)(pyridin-3-yl)methyl)butyramide |
| G12 | 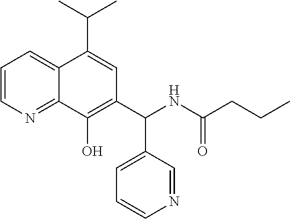 [SMILES: CCCC(=O)NC(c1cccnc1)c1cc(C(C)C)c2cccnc2c1O] | N-((8-hydroxy-5-isopropylquinolin-7- yl)(pyridin-3-yl)methyl)butyramide |
| G13 | 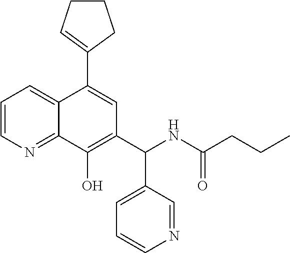 [SMILES: CCCC(=O)NC(c1cccnc1)c1cc(C2=CCCC2)c2cccnc2c1O] | N-((5-(cyclopent-1-en-1-yl)-8- hydroxyquinolin-7-yl)(pyridin-3- yl)methyl)butyramide |
| G14 | 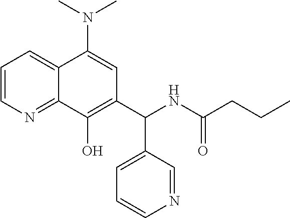 [SMILES: CCCC(=O)NC(c1cccnc1)c1cc(N(C)C)c2cccnc2c1O] | N-((5-(dimethylamino)-8- hydroxyquinolin-7-yl)(pyridin-3- yl)methyl)butyramide |
| G15 | 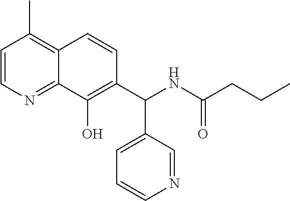 [SMILES: CCCC(=O)NC(c1cccnc1)c1ccc2c(C)ccnc2c1O] | N-((8-hydroxy-4-methylquinolin-7- yl)(pyridin-3-yl)methyl)butyramide |
| G16 | 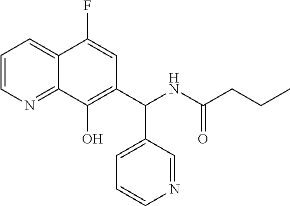 [SMILES: CCCC(=O)NC(c1cccnc1)c1cc(F)c2cccnc2c1O] | N-((5-fluoro-8-hydroxyquinolin-7- yl)(pyridin-3-yl)methyl)butyramide |
| G17 | 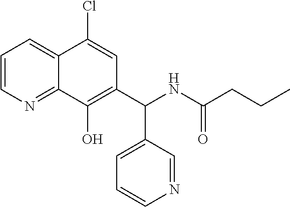 [SMILES: CCCC(=O)NC(c1cccnc1)c1cc(Cl)c2cccnc2c1O] | N-((5-chloro-8-hydroxyquinolin-7- yl)(pyridin-3-yl)methyl)butyramide |
| G18 | 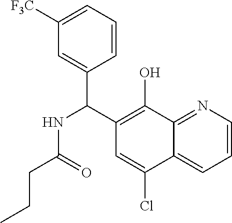 [SMILES: CCCC(=O)NC(c1cccc(C(F)(F)F)c1)c1cc(Cl)c2cccnc2c1O] | N-((5-chloro-8-hydroxyquinolin-7-yl)(3- (trifluoromethyl)phenyl)methyl)butyramide |
| G19 | 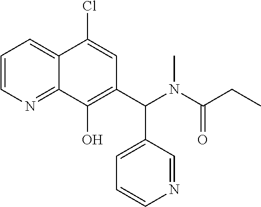 [SMILES: CCC(=O)N(C)C(c1cccnc1)c1cc(Cl)c2cccnc2c1O] | N-((5-chloro-8-hydroxyquinolin-7- yl)(pyridin-3-yl)methyl)-N- methylpropionamide |
| G20 | 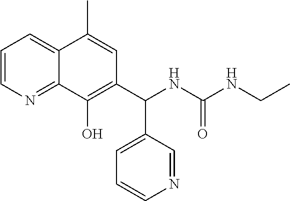 [SMILES: CCNC(=O)NC(c1cccnc1)c1cc(C)c2cccnc2c1O] | 1-ethyl-3-((8-hydroxy-5- methylquinolin-7-yl)(pyridin-3- yl)methyl)urea |
| G21 | 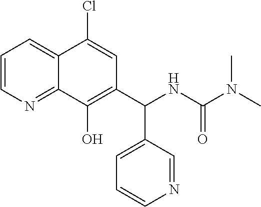 [SMILES: CN(C)C(=O)NC(c1cccnc1)c1cc(Cl)c2cccnc2c1O] | 3-((5-chloro-8-hydroxyquinolin-7- yl)(pyridin-3-yl)methyl)-1,1- dimethylurea |
| G22 | 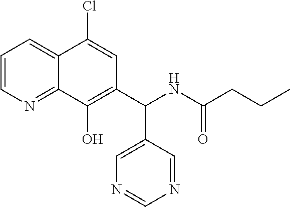 [SMILES: CCCC(=O)NC(c1cncnc1)c1cc(Cl)c2cccnc2c1O] | N-((5-chloro-8-hydroxyquinolin-7- yl)(pyrimidin-5-yl)methyl)butyramide |
| G23 | 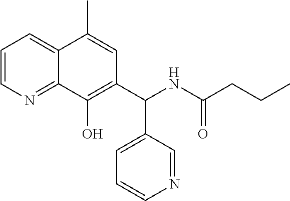 [SMILES: CCCC(=O)NC(c1cccnc1)c1cc(C)c2cccnc2c1O] | N-((8-hydroxy-5-methylquinolin-7- yl)(pyridin-3-yl)methyl)butyramide |
| G24 | 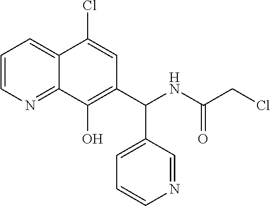 [SMILES: O=C(CCl)NC(c1cccnc1)c1cc(Cl)c2cccnc2c1O] | 2-chloro-N-((5-chloro-8- hydroxyquinolin-7-yl)(pyridin-3- yl)methyl)acetamide |
| G25 | 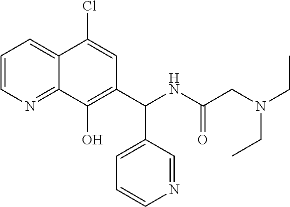 [SMILES: CCN(CC)CC(=O)NC(c1cccnc1)c1cc(Cl)c2cccnc2c1O] | N-((5-chloro-8-hydroxyquinolin-7- yl)(pyridin-3-yl)methyl)-2- (diethylamino)acetamide |
| G26 | 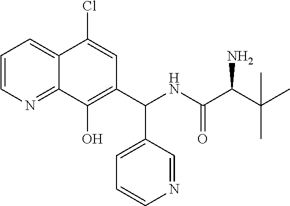 [SMILES: CC(C)(C)[C@H](N)C(=O)NC(c1cccnc1)c1cc(Cl)c2cccnc2c1O] | (2S)-2-amino-N-((5-chloro-8- hydroxyquinolin-7-yl)(pyridin-3- yl)methyl)-3,3-dimethylbutanamide |
| G27 | 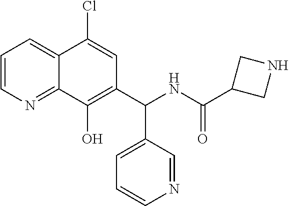 [SMILES: O=C(NC(c1cccnc1)c1cc(Cl)c2cccnc2c1O)C1CNC1] | N-((5-chloro-8-hydroxyquinolin-7- yl)(pyridin-3-yl)methyl)azetidine-3- carboxamide |
| G28 | 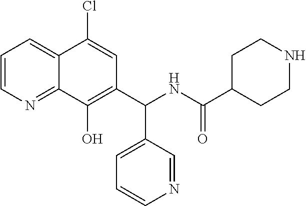 [SMILES: O=C(NC(c1cccnc1)c1cc(Cl)c2cccnc2c1O)C1CCNCC1] | N-((5-chloro-8-hydroxyquinolin-7- yl)(pyridin-3-yl)methyl)piperidine-4- carboxamide |
| G29 | 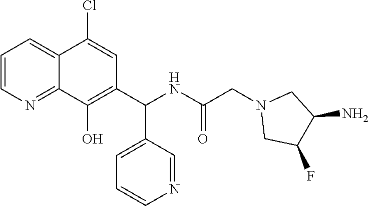 [SMILES: N[C@@H]1CN(CC(=O)NC(c2cccnc2)c2cc(Cl)c3cccnc3c2O)C[C@@H]1F] | 2-((3R,4S)-3-amino-4-fluoropyrrolidin- 1-yl)-N-((5-chloro-8-hydroxyquinolin-7- yl)(pyridin-3-yl)methyl)acetamide |
| G30 | 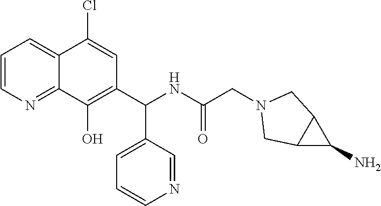 | 2-(6-amino-3-azabicyclo[3.1.0]hexan-3- yl)-N-((5-chloro-8-hydroxyquinolin-7- yl)(pyridin-3-yl)methyl)acetamide |
| G31 | 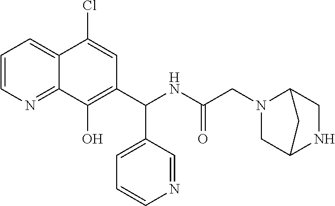 [SMILES: O=C(CN1CC2CC1CN2)NC(c1cccnc1)c1cc(Cl)c2cccnc2c1O] | 2-(2,5-diazabicyclo[2.2.1]heptan-2-yl)- N-((5-chloro-8-hydroxyquinolin-7- yl)(pyridin-3-yl)methyl)acetamide |
| G32 | 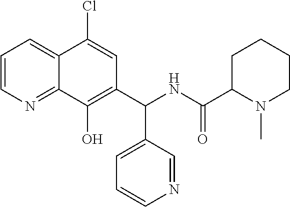 [SMILES: CN1CCCCC1C(=O)NC(c1cccnc1)c1cc(Cl)c2cccnc2c1O] | N-((5-chloro-8-hydroxyquinolin-7- yl)(pyridin-3-yl)methyl)-1- methylpiperidine-2-carboxamide |
| G33 | 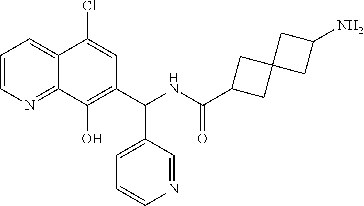 [SMILES: NC1CC2(C1)CC(C(=O)NC(c1cccnc1)c1cc(Cl)c3cccnc3c1O)C2] | 6-amino-N-((5-chloro-8- hydroxyquinolin-7-yl)(pyridin-3- yl)methyl)spiro[3.3]heptane-2- carboxamide |
| G34 | 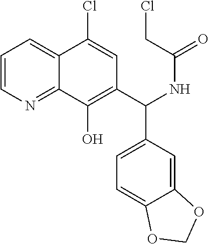 [SMILES: O=C(CCl)NC(c1ccc2c(c1)OCO2)c1cc(Cl)c2cccnc2c1O] | N-(benzo[d][1,3]dioxol-5-yl(5-chloro-8- hydroxyquinolin-7-yl)methyl)-2- chloroacetamide |
| G35 | 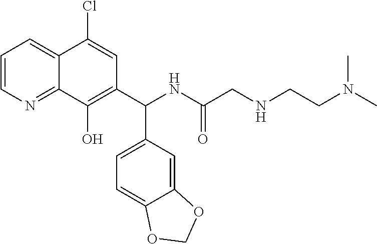 [SMILES: CN(C)CCNCC(=O)NC(c1ccc2c(c1)OCO2)c1cc(Cl)c2cccnc2c1O] | N-(benzo[d][1,3]dioxol-5-yl(5-chloro-8- hydroxyquinolin-7-yl)methyl)-2-((2- (dimethylamino)ethyl)amino)acetamide |
| G36 | 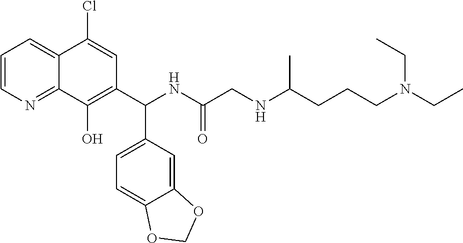 [SMILES: CCN(CC)CCCC(C)NCC(=O)NC(c1ccc2c(c1)OCO2)c1cc(Cl)c2cccnc2c1O] | N-(benzo[d][1,3]dioxol-5-yl(5-chloro-8- hydroxyquinolin-7-yl)methyl)-2-((5- (diethylamino)pentan-2- yl)amino)acetamide |
| G37 | 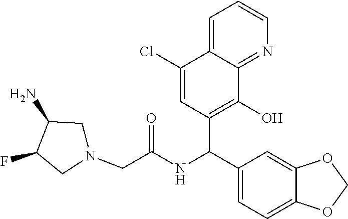 [SMILES: N[C@H]1CN(CC(=O)NC(c2ccc3c(c2)OCO3)c2cc(Cl)c3cccnc3c2O)C[C@H]1F] | 2-((3S,4R)-3-amino-4-fluoropyrrolidin- 1-yl)-N-(benzo[d][1,3]dioxol-5-yl(5- chloro-8-hydroxyquinolin-7- yl)methyl)acetamide |
| G38 | 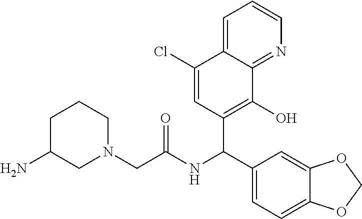 [SMILES: NC1CCCN(CC(=O)NC(c2ccc3c(c2)OCO3)c2cc(Cl)c3cccnc3c2O)C1] | 2-(3-aminopiperidin-1-yl)-N- (benzo[d][1,3]dioxol-5-yl(5-chloro-8- hydroxyquinolin-7-yl)methyl)acetamide |
| G39 | 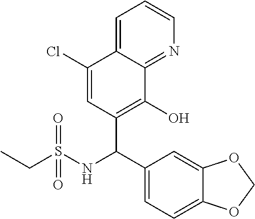 [SMILES: CCS(=O)(=O)NC(c1ccc2c(c1)OCO2)c1cc(Cl)c2cccnc2c1O] | N-(benzo[d][1,3]dioxol-5-yl(5-chloro-8- hydroxyquinolin-7- yl)methyl)ethanesulfonamide |
| G40 | 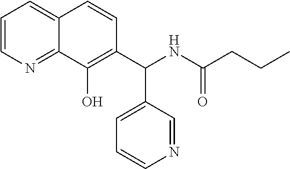 [SMILES: CCCC(=O)NC(c1cccnc1)c1ccc2cccnc2c1O] | N-((8-hydroxyquinolin-7-yl)(pyridin-3- yl)methyl)butyramide |
| G41 | 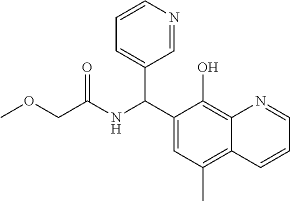 [SMILES: COCC(=O)NC(c1cccnc1)c1cc(C)c2cccnc2c1O] | N-((8-hydroxy-5-methylquinolin-7- yl)(pyridin-3-yl)methyl)-2- methoxyacetamide |
| G42 | 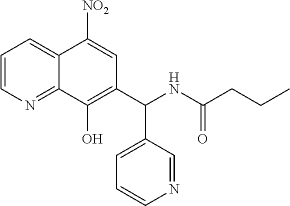 [SMILES: CCCC(=O)NC(c1cccnc1)c1cc([N+](=O)[O-])c2cccnc2c1O] | N-((8-hydroxy-5-nitroquinolin-7- yl)(pyridin-3-yl)methyl)butyramide |
| G43 | 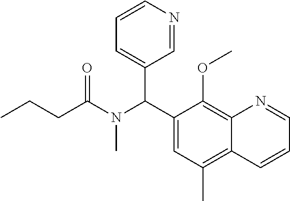 [SMILES: CCCC(=O)N(C)C(c1cccnc1)c1cc(C)c2cccnc2c1OC] | N-((8-methoxy-5-methylquinolin-7- yl)(pyridin-3-yl)methyl)-N- methylbutyramide |
| G44 | 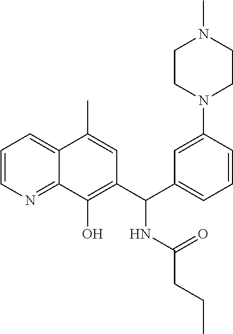 [SMILES: CCCC(=O)NC(c1cccc(N2CCN(C)CC2)c1)c1cc(C)c2cccnc2c1O] | N-((8-hydroxy-5-methylquinolin-7- yl)(3-(4-methylpiperazin-1- yl)phenyl)methyl)butyramide |
| G45 | 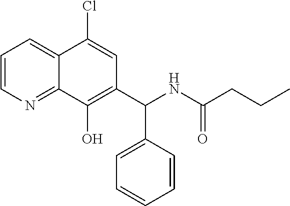 [SMILES: CCCC(=O)NC(c1ccccc1)c1cc(Cl)c2cccnc2c1O] | N-((5-chloro-8-hydroxyquinolin-7- yl)(phenyl)methyl)butyramide |
| G46 | 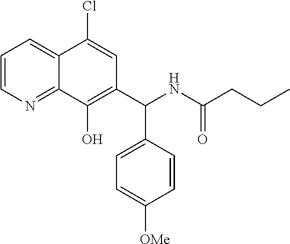 [SMILES: CCCC(=O)NC(c1ccc(OC)cc1)c1cc(Cl)c2cccnc2c1O] | N-((5-chloro-8-hydroxyquinolin-7-yl)(4- methoxyphenyl)methyl)butyramide |
| G47 | 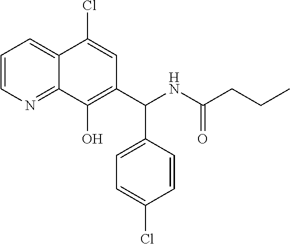 [SMILES: CCCC(=O)NC(c1ccc(Cl)cc1)c1cc(Cl)c2cccnc2c1O] | N-((5-chloro-8-hydroxyquinolin-7-yl)(4- chlorophenyl)methyl)butyramide |
| G48 | 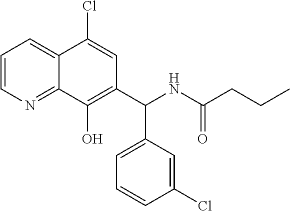 [SMILES: CCCC(=O)NC(c1cccc(Cl)c1)c1cc(Cl)c2cccnc2c1O] | N-((5-chloro-8-hydroxyquinolin-7-yl)(3- chlorophenyl)methyl)butyramide |
| G49 | 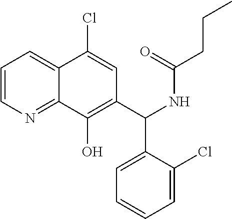 [SMILES: CCCC(=O)NC(c1ccccc1Cl)c1cc(Cl)c2cccnc2c1O] | N-((5-chloro-8-hydroxyquinolin-7-yl)(2- chlorophenyl)methyl)butyramide |
| G50 | 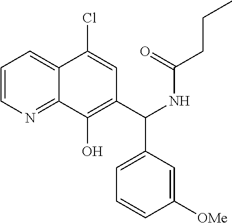 [SMILES: CCCC(=O)NC(c1cccc(OC)c1)c1cc(Cl)c2cccnc2c1O] | N-((5-chloro-8-hydroxyquinolin-7-yl)(3- methoxyphenyl)methyl)butyramide |
| G51 | 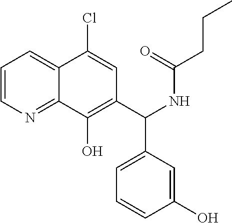 [SMILES: CCCC(=O)NC(c1cccc(O)c1)c1cc(Cl)c2cccnc2c1O] | N-((5-chloro-8-hydroxyquinolin-7-yl)(3- hydroxyphenyl)methyl)butyramide |
| G52 | 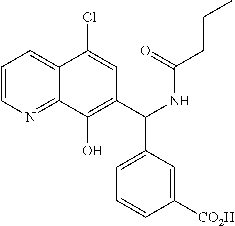 [SMILES: CCCC(=O)NC(c1cccc(C(=O)O)c1)c1cc(Cl)c2cccnc2c1O] | 3-(butyramido(5-chloro-8- hydroxyquinolin-7-yl)methyl)benzoic acid |
| G53 | 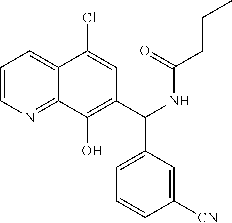 [SMILES: CCCC(=O)NC(c1cccc(C#N)c1)c1cc(Cl)c2cccnc2c1O] | N-((5-chloro-8-hydroxyquinolin-7-yl)(3- cyanophenyl)methyl)butyramide |
| G54 | 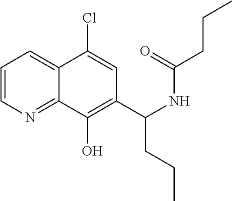 [SMILES: CCCC(=O)NC(CCC)c1cc(Cl)c2cccnc2c1O] | N-(1-(5-chloro-8-hydroxyquinolin-7- yl)butyl)butyramide |
| G55 | 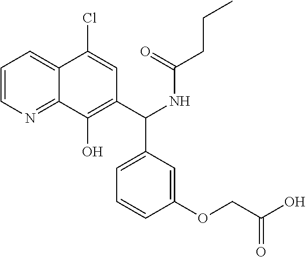 [SMILES: CCCC(=O)NC(c1cccc(OCC(=O)O)c1)c1cc(Cl)c2cccnc2c1O] | 2-(3-(butyramido(5-chloro-8- hydroxyquinolin-7- yl)methyl)phenoxy)acetic acid |
| G56 | 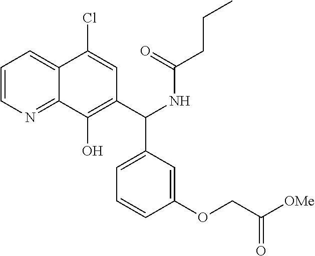 [SMILES: CCCC(=O)NC(c1cccc(OCC(=O)OC)c1)c1cc(Cl)c2cccnc2c1O] | Methyl 2-(3-(butyramido(5-chloro-8- hydroxyquinolin-7- yl)methyl)phenoxy)acetate |
| G57 | 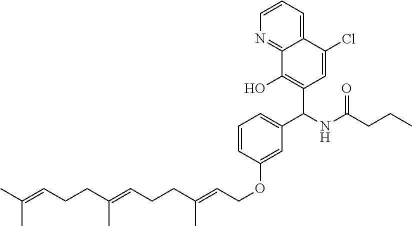 [SMILES: CCCC(=O)NC(c1cccc(OC/C=C(\C)CC/C=C(\C)CCC=C(C)C)c1)c1cc(Cl)c2cccnc2c1O] | N-((5-chloro-8-hydroxyquinolin-7-yl)(3- (((2E,6E)-3,7,11-trimethyldodeca- 2,6,10-trien-1- yl)oxy)phenyl)methyl)butyramide |
| G58 | 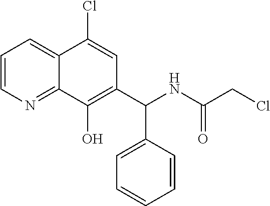 [SMILES: O=C(CCl)NC(c1ccccc1)c1cc(Cl)c2cccnc2c1O] | 2-chloro-N-((5-chloro-8- hydroxyquinolin-7- yl)(phenyl)methyl)acetamide |
| G59 | 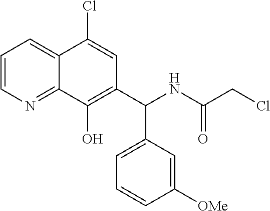 [SMILES: COc1cccc(C(NC(=O)CCl)c2cc(Cl)c3cccnc3c2O)c1] | 2-chloro-N-((5-chloro-8- hydroxyquinolin-7-yl)(3- methoxyphenyl)methyl)acetamide |
| G60 | 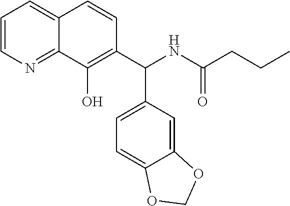 [SMILES: CCCC(=O)NC(c1ccc2c(c1)OCO2)c1ccc2cccnc2c1O] | N-(benzo[d][1,3]dioxol-5-yl(8- hydroxyquinolin-7- yl)methyl)butyramide |
| G61 | 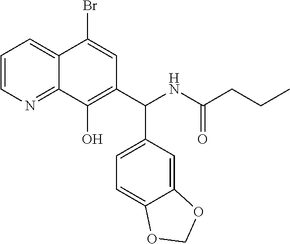 [SMILES: CCCC(=O)NC(c1ccc2c(c1)OCO2)c1cc(Br)c2cccnc2c1O] | N-(benzo[d][1,3]dioxol-5-yl(5-bromo-8- hydroxyquinolin-7- yl)methyl)butyramide |
| G62 | 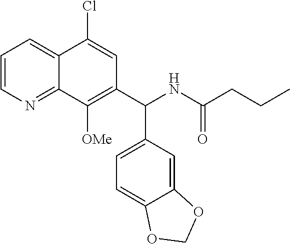 [SMILES: CCCC(=O)NC(c1ccc2c(c1)OCO2)c1cc(Cl)c2cccnc2c1OC] | N-(benzo[d][1,3]dioxol-5-yl(5-chloro-8- methoxyquinolin-7- yl)methyl)butyramide |
| G63 | 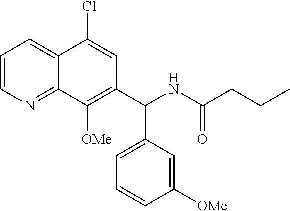 [SMILES: CCCC(=O)NC(c1cccc(OC)c1)c1cc(Cl)c2cccnc2c1OC] | N-((5-chloro-8-methoxyquinolin-7- yl)(3- methoxyphenyl)methyl)butyramide |
| G64 | 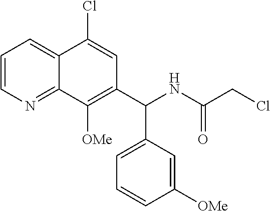 [SMILES: COc1cccc(C(NC(=O)CCl)c2cc(Cl)c3cccnc3c2OC)c1] | 2-chloro-N-((5-chloro-8- methoxyquinolin-7-yl)(3- methoxyphenyl)methyl)acetamide |
| G65 | 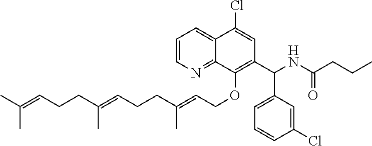 [SMILES: CCCC(=O)NC(c1cccc(Cl)c1)c1cc(Cl)c2cccnc2c1OC/C=C(\C)CC/C=C(\C)CCC=C(C)C] | N-((5-chloro-8-(((2E,6E)-3,7,11- trimethyldodeca-2,6,10-trien-1- yl)oxy)quinolin-7-yl)(3- chlorophenyl)methyl)butyramide |
| G66 | 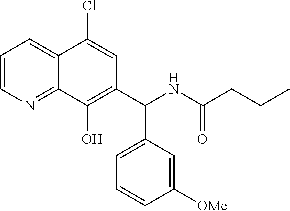 [SMILES: CCCC(=O)NC(c1cccc(OC)c1)c1cc(Cl)c2cccnc2c1O] | N-((5-chloro-8-hydroxyquinolin-7-yl)(3- methoxyphenyl)methyl)butyramide |
| G67 | 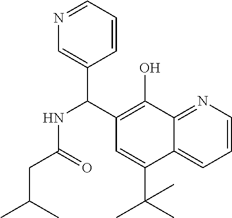 [SMILES: CC(C)CC(=O)NC(c1cccnc1)c1cc(C(C)(C)C)c2cccnc2c1O] | N-((5-(tert-butyl)-8-hydroxyquinolin-7- yl)(pyridin-3-yl)methyl)-3- methylbutanamide |
| G68 | 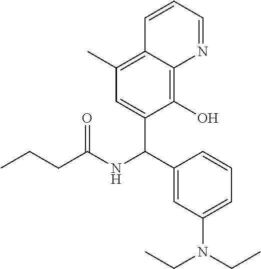 [SMILES: CCCC(=O)NC(c1cccc(N(CC)CC)c1)c1cc(C)c2cccnc2c1O] | N-((3-(diethylamino)phenyl)(8-hydroxy- 5-methylquinolin-7- yl)methyl)butyramide |
| G69 | 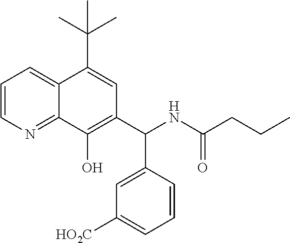 [SMILES: CCCC(=O)NC(c1cccc(C(=O)O)c1)c1cc(C(C)(C)C)c2cccnc2c1O] | 3-((5-(tert-butyl)-8-hydroxyquinolin-7- yl)(butyramido)methyl)benzoic acid |
| G70 | 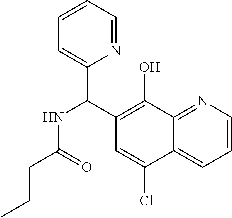 [SMILES: CCCC(=O)NC(c1ccccn1)c1cc(Cl)c2cccnc2c1O] | N-((5-chloro-8-hydroxyquinolin-7- yl)(pyridin-2-yl)methyl)butyramide |
| G71 | 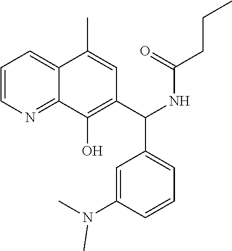 [SMILES: CCCC(=O)NC(c1cccc(N(C)C)c1)c1cc(C)c2cccnc2c1O] | N-((3-(dimethylamino)phenyl)(8- hydroxy-5-methylquinolin-7- yl)methyl)butyramide |
| G72 | 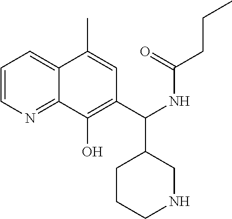 [SMILES: CCCC(=O)NC(c1cc(C)c2cccnc2c1O)C1CCCNC1] | N-((8-hydroxy-5-methylquinolin-7- yl)(piperidin-3-yl)methyl)butyramide |
| G73 | 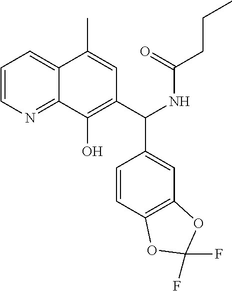 [SMILES: CCCC(=O)NC(c1ccc2c(c1)OC(F)(F)O2)c1cc(C)c2cccnc2c1O] | N-((2,2-difluorobenzo[d][1,3]dioxol-5- yl)(8-hydroxy-5-methylquinolin-7- yl)methyl)butyramide |
| G74 | 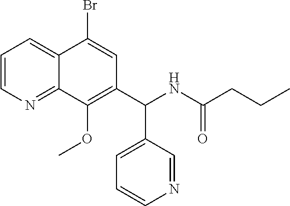 [SMILES: CCCC(=O)NC(c1cccnc1)c1cc(Br)c2cccnc2c1OC] | N-((5-bromo-8-methoxyquinolin-7- yl)(pyridin-3-yl)methyl)butyramide |
| G75 | 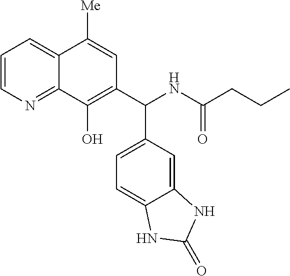 [SMILES: CCCC(=O)NC(c1ccc2[nH]c(=O)[nH]c2c1)c1cc(C)c2cccnc2c1O] | N-((8-hydroxy-5-methylquinolin-7- yl)(2-oxo-2,3-dihydro-1H- benzo[d]imidazol-5- yl)methyl)butyramide |
| G76 | 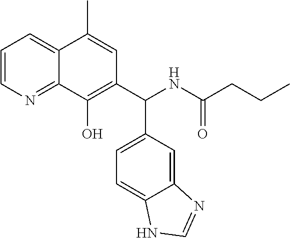 [SMILES: CCCC(=O)NC(c1ccc2[nH]cnc2c1)c1cc(C)c2cccnc2c1O] | N-((1H-benzo[d]imidazol-5-yl)(8- hydroxy-5-methylquinolin-7- yl)methyl)butyramide |
| G77 | 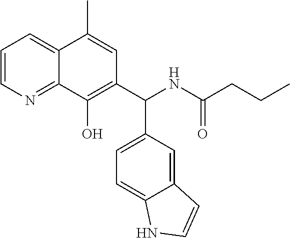 [SMILES: CCCC(=O)NC(c1ccc2[nH]ccc2c1)c1cc(C)c2cccnc2c1O] | N-((8-hydroxy-5-methylquinolin-7- yl)(1H-indol-5-yl)methyl)butyramide |
| G78 | 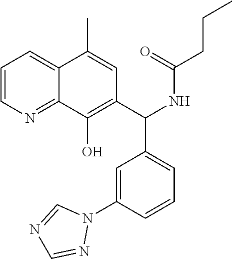 [SMILES: CCCC(=O)NC(c1cccc(-n2cncn2)c1)c1cc(C)c2cccnc2c1O] | N-((3-(1H-1,2,4-triazol-1-yl)phenyl)(8- hydroxy-5-methylquinolin-7- yl)methyl)butyramide |
| G79 | 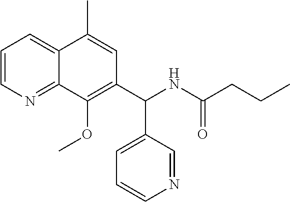 [SMILES: CCCC(=O)NC(c1cccnc1)c1cc(C)c2cccnc2c1OC] | N-((8-methoxy-5-methylquinolin-7- yl)(pyridin-3-yl)methyl)butyramide |
| G80 | 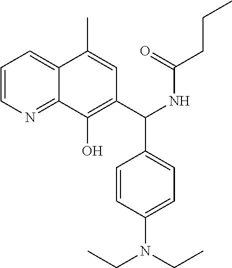 [SMILES: CCCC(=O)NC(c1ccc(N(CC)CC)cc1)c1cc(C)c2cccnc2c1O] | N-((4-(diethylamino)phenyl)(8-hydroxy- 5-methylquinolin-7- yl)methyl)butyramide |
| G81 | 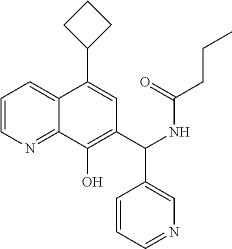 [SMILES: CCCC(=O)NC(c1cccnc1)c1cc(C2CCC2)c2cccnc2c1O] | N-((5-cyclobutyl-8-hydroxyquinolin-7- yl)(pyridin-3-yl)methyl)butyramide |
| G82 | 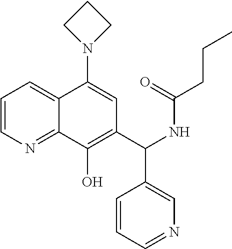 [SMILES: CCCC(=O)NC(c1cccnc1)c1cc(N2CCC2)c2cccnc2c1O] | N-((5-(azetidin-1-yl)-8- hydroxyquinolin-7-yl)(pyridin-3- yl)methyl)butyramide |
| G83 | 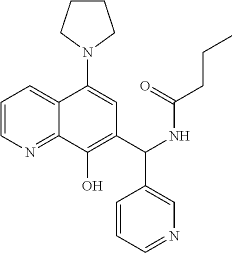 [SMILES: CCCC(=O)NC(c1cccnc1)c1cc(N2CCCC2)c2cccnc2c1O] | N-((8-hydroxy-5-(pyrrolidin-1- yl)quinolin-7-yl)(pyridin-3- yl)methyl)butyramide |
| G84 | 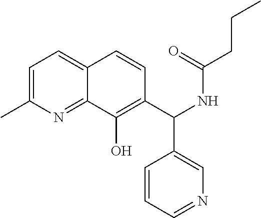 [SMILES: CCCC(=O)NC(c1cccnc1)c1ccc2ccc(C)nc2c1O] | N-((8-hydroxy-2-methylquinolin-7- yl)(pyridin-3-yl)methyl)butyramide |
| G85 | 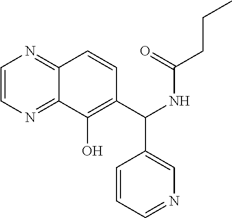 [SMILES: CCCC(=O)NC(c1cccnc1)c1ccc2nccnc2c1O] | N-((5-hydroxyquinoxalin-6-yl)(pyridin- 3-yl)methyl)butyramide |
| G86 | 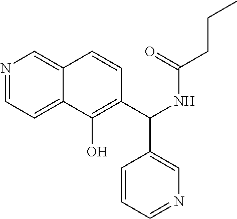 [SMILES: CCCC(=O)NC(c1cccnc1)c1ccc2cnccc2c1O] | N-((5-hydroxyisoquinolin-6-yl)(pyridin- 3-yl)methyl)butyramide |
| G87 | 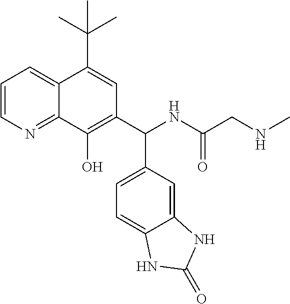 [SMILES: CNCC(=O)NC(c1ccc2[nH]c(=O)[nH]c2c1)c1cc(C(C)(C)C)c2cccnc2c1O] | N-((5-(tert-butyl)-8-hydroxyquinolin-7- yl)(2-oxo-2,3-dihydro-1H- benzo[d]imidazol-5-yl)methyl)-2- (methylamino)acetamide |
| G88 | 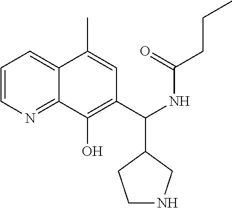 [SMILES: CCCC(=O)NC(c1cc(C)c2cccnc2c1O)C1CCNC1] | N-((8-hydroxy-5-methylquinolin-7- yl)(pyrrolidin-3-yl)methyl)butyramide |
| G89 | 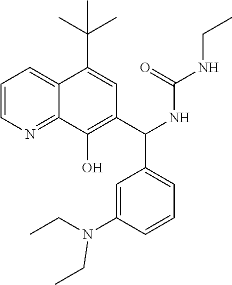 [SMILES: CCNC(=O)NC(c1cccc(N(CC)CC)c1)c1cc(C(C)(C)C)c2cccnc2c1O] | 1-((5-(tert-butyl)-8-hydroxyquinolin-7- yl)(3-(diethylamino)phenyl)methyl)-3- ethylurea |
| G90 | 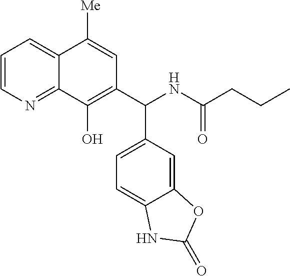 [SMILES: CCCC(=O)NC(c1ccc2[nH]c(=O)oc2c1)c1cc(C)c2cccnc2c1O] | N-((8-hydroxy-5-methylquinolin-7- yl)(2-oxo-2,3-dihydrobenzo[d]oxazol-6- yl)methyl)butyramide |
| G91 | 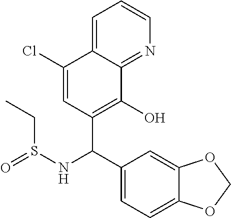 [SMILES: CCS(=O)NC(c1ccc2c(c1)OCO2)c1cc(Cl)c2cccnc2c1O] | N-(benzo[d][1,3]dioxol-5-yl(5-chloro-8- hydroxyquinolin-7- yl)methyl)ethanesulfinamide |
| G92 | 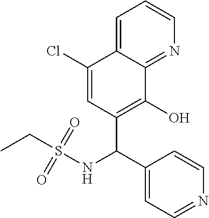 [SMILES: CCS(=O)(=O)NC(c1ccncc1)c1cc(Cl)c2cccnc2c1O] | N-((5-chloro-8-hydroxyquinolin-7- yl)(pyridin-4- yl)methyl)ethanesulfonamide |
| G93 | 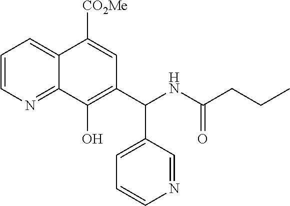 [SMILES: CCCC(=O)NC(c1cccnc1)c1cc(C(=O)OC)c2cccnc2c1O] | methyl 7-(butyramido(pyridin-3- yl)methyl)-8-hydroxyquinoline-5- carboxylate |
| G94 | 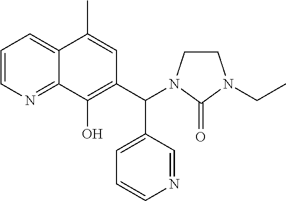 [SMILES: CCN1CCN(C(c2cccnc2)c2cc(C)c3cccnc3c2O)C1=O] | 1-ethyl-3-((8-hydroxy-5- methylquinolin-7-yl)(pyridin-3- yl)methyl)imidazolidin-2-one |
| G95 | 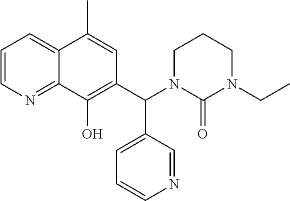 [SMILES: CCN1CCCN(C(c2cccnc2)c2cc(C)c3cccnc3c2O)C1=O] | 1-ethyl-3-((8-hydroxy-5- methylquinolin-7-yl)(pyridin-3- yl)methyl)tetrahydropyrimidin-2(1H)- one |
| G96 (YUM 70) | 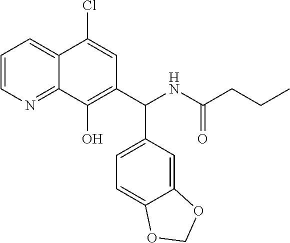 [SMILES: CCCC(=O)NC(c1ccc2c(c1)OCO2)c1cc(Cl)c2cccnc2c1O] | N-(benzo[d][1,3]dioxol-5-yl(5-chloro-8- hydroxyquinolin-7- yl)methyl)butyramide |
| G97 | 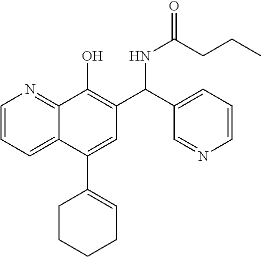 [SMILES: CCCC(=O)NC(c1cccnc1)c1cc(C2=CCCCC2)c2cccnc2c1O] | N-((5-(cyclohex-1-en-1-yl)-8- hydroxyquinolin-7-yl)(pyridin-3- yl)methyl)butyramide |
| G98 | 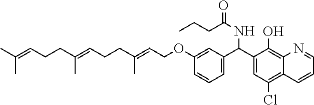 [SMILES: CCCC(=O)NC(c1cccc(OC/C=C(\C)CC/C=C(\C)CCC=C(C)C)c1)c1cc(Cl)c2cccnc2c1O] | N-((5-Chloro-8-hydroxyquinolin-7- yl)(3-(((2E,6E)-3,7,11-trimethyldodeca- 2,6,10-trien-1- yl)oxy)phenyl)methyl)butyramide |

In a particular embodiment, compounds encompassed within Formula II are provided:

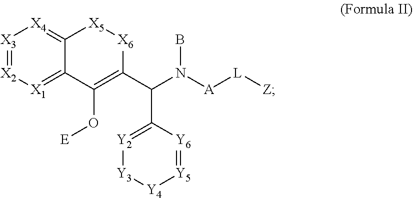

including pharmaceutically acceptable salts, solvates, and/or prodrugs thereof.

Formulas II is not limited to a particular chemical moiety for X₁, X₂, X₃, X₄, X₅, X₆, Y₂, Y₃, Y₄, Y₅, Y₆, A, B, E, L and Z. In some embodiments, the particular chemical moiety for X₁, X₂, X₃, X₄, X₅, X₆, Y₂, Y₃, Y₄, Y₅, Y₆, A, B, E, L and Z permits the resulting compound capable of one or more of:
inhibiting GRP78 activity;
serving as an effective therapeutic agent for treating, ameliorating, and preventing various forms of cancer, viral infections, and inflammatory diseases;
inducing ER stress-mediated apoptosis in the tumor cells implanted in mice without major toxicity to normal tissues; and
inducing ER stress and triggers UPR by inhibiting GRP78.

In some embodiments, X₁ is either CH or N.

In some embodiments, X₂, X₃, X₄, X₅ and X₆ are each independently selected from CR¹ and N, with the proviso that at least three of them must be CR¹.

In some embodiments, A is selected from the group consisting of CO, SO, and SO₂.

In some embodiments, Y², Y³, Y⁴, Y⁵, Y⁶ are each independently selected from the group consisting of CH, CR² and N.

In some embodiments, Y⁵ is a bond, in which case one of Y³, Y⁴, or Y⁶ is NR², 0, or S, while the other two may be CR² or N.

In some embodiments, B, E are each independently selected from H and R³.

In some embodiments, R¹ is independently selected from the group consisting of H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano, CF₃, OCF₃, SOR⁷, SO₂R⁷, NO₂, COR⁴, C₁₋₆ alkyl-COR⁴, N(R⁷)C₂₋₆ alkyl-NR⁷R⁷, —N(R⁷)C₂₋₆ alkyl-R⁴, N(C₂₋₆ alkyl)₂-NR⁷, —O(CH₂)ₚR⁴, —S(CH₂)ₚR⁴, and —N(R⁷)C(═O)(CH₂)ₚR⁴, with a proviso that not more than three R¹ can be other than H.

In some embodiments, R² is independently selected from the group consisting of H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano,

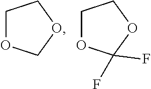

CF₃, OCF₃, SOR⁷, SO₂R⁷, NO₂, COR⁴, C₁₋₆ alkyl-COR⁴, N(R⁷)C₂₋₆ alkyl-NR⁷R⁷, N(C₂₋₆ alkyl)₂-NR⁷, CF₃, CO₂Et, CO₂H, —N(R⁷)C₂₋₆ alkyl-R⁴, —O(CH₂)ₚR⁴, —S(CH₂)ₚR⁴, and —N(R⁷)C(═O)(CH₂)ₚR⁴, with a proviso that not more than two R² can be other than H.

In some embodiments, R³ is independently selected from the group consisting of C₁₋₆ alkyl, C₃₋₆ alkenyl, C₃₋₆ alkynyl, C₃₋₇ cycloalkyl, C₄₋₇ heterocycloalkyl, phenyl, naphthyl, 5-10 membered mono- or bicyclic heteroaryl, C₁₋₆ alkyl-C₃₋₇ cycloalkyl, C₁₋₆ alkyl-C₄₋₇ heterocycloalkyl, and C₁₋₆ R⁴.

In some embodiments, R⁴ is independently selected from the group consisting of OH, NR⁵R⁶, O(CH₂)_{q}NR⁵R⁶, C₁₋₆ alkoxy, C₁₋₆ alkoxy-C₁₋₆ alkoxy, C₂₋₆ hydroxyalkoxy, cyclopropyl, oxetanyl, oxetanyloxy, oxetanylamino, oxolanyl, oxolanyloxy, oxolanylamino, oxanyl oxanyloxy, oxanylamino, oxepanyl, oxepanyloxy, oxepanylamino, azetidinyl, azetidinyloxy, azetidylamino, pyrrolidinyl, pyrolidinyloxy, pyrrolidinylamino, piperidinyl, piperidinyloxy, piperidinylamino, azepanyl, azepanyloxy, azepanylamino, dioxolanyl, dioxanyl, morpholino, thiomorpholino, thiomorpholino-S,S-dioxide, piperazino, dioxepanyl, dioxepanyloxy, dioxepanylamino, oxazepanyl, oxazepanyloxy, oxazepanylamino, diazepanyl, diazepanyloxy, and diazepanylamino, all of which may be optionally substituted with OH, OR⁷, oxo, halogen, R⁶, CH₂OR⁶, CH₂NR⁵R⁶ or CH₂CH₂CONR⁵R⁶.

In some embodiments, R⁵ and R⁶ are each independently selected from the group consisting of H, —CD₃, C₁₋₆ alkyl, C₃₋₆ alkenyl, C₃₋₆ alkynyl, C₃₋₈ cycloalkyl, —(C₁₋₃ alkyl)-(C₃₋₈ cycloalkyl), C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, and 5-11 membered heteroaryl; wherein R⁵ and R⁶ may be further independently substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo; or alternatively, R⁵ and R⁶, taken together with the N atom to which they are both attached, form a heterocycloalkyl ring of 4-7 members, containing up to one other heteroatom selected from O, S, or NR³, or a heterobicycloalkyl ring of 6-12 members which may be fused, bridged or spiro, and contain up to two other heteroatoms chosen from O, S(O)ₓ, or NR³.

In some embodiments, each R⁷ is independently selected from the group consisting of H, —CD₃, C₁₋₆ alkyl, C₃₋₆ cycloalkyl, phenyl, naphthyl, 5-10 membered mono- or bicyclo-heteroaryl, C₂₋₆ hydroxyalkyl, —SO₂-alkyl, NH—C₂₋₆ alkyl-NR⁵R⁶, C₁₋₆ alkoxy-C₁₋₆ alkyl, and C₂₋₆ alkyl-NR⁵R⁶; alternatively, two R⁷ taken together with the same N atom to which they are both attached, form a heterocyclic ring of 4-7 members, containing up to one other heteroatom selected from O, S, or NR³.

In some embodiments, p=0, 1, 2, 3, or 4.

In some embodiments, x=0, 1, or 2.

In some embodiments, L is a linker selected from a group consisting of —(CH₂)m-,—(CH₂CH₂O)n-,

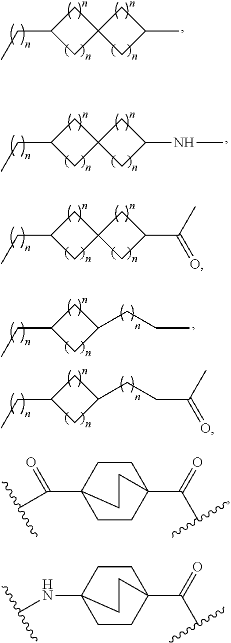

—(CH₂)ₘC≡C, —(CH₂CH₂O)ₙC≡C; wherein m=0, 1, 2, 3, 4, 5, 6, 7, 8, 9, or 10; and wherein n=0, 1, 2, 3, 4, 5, or 6.

In some embodiments, Z is a radical of an E3 ligase ligand selected from the group consisting of:

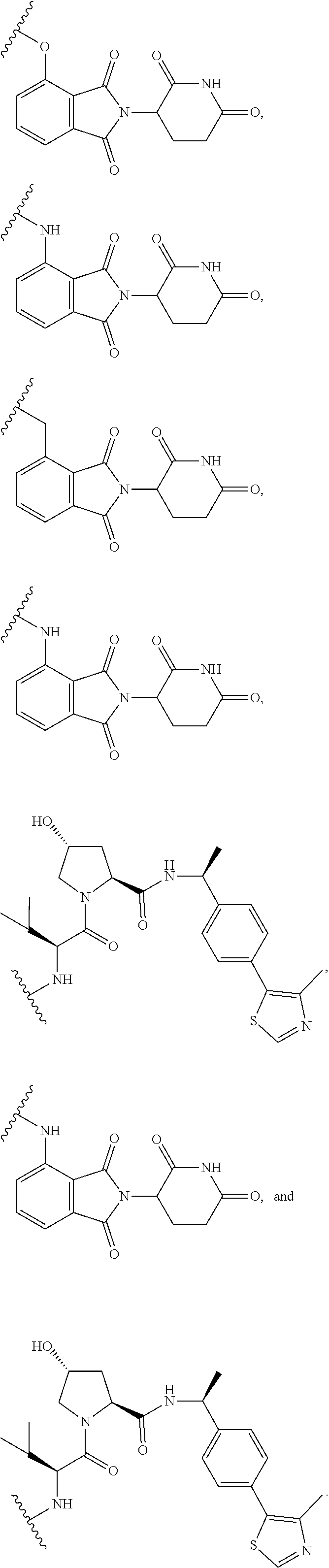

In a particular embodiment, compounds encompassed within Formula III are provided:

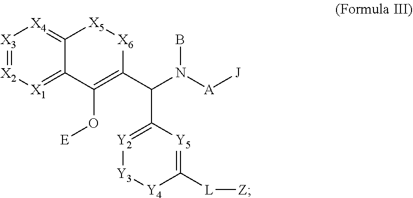

including pharmaceutically acceptable salts, solvates, and/or prodrugs thereof.

Formula III is not limited to a particular chemical moiety for X₁, X₂, X₃, X₄, X₅, X₆, Y₂, Y₃, Y₄, Y₅, A, B, E, J, L and Z. In some embodiments, the particular chemical moiety for X₁, X₂, X₃, X₄, X₅, X₆, Y₂, Y₃, Y₄, Y₅, A, B, E, J, L and Z permits the resulting compound capable of one or more of
inhibiting GRP78 activity;
serving as an effective therapeutic agent for treating, ameliorating, and preventing various forms of cancer, viral infections, and inflammatory diseases;
inducing ER stress-mediated apoptosis in the tumor cells implanted in mice without major toxicity to normal tissues; and
inducing ER stress and triggers UPR by inhibiting GRP78.

In some embodiments, X₁ is either CH or N.

In some embodiments, X₂, X₃, X₄, X₅ and X₆ are each independently selected from CR¹ and N, with the proviso that at least three of them must be CR¹.

In some embodiments, A is selected from CO, SO, and SO₂.

In some embodiments, Y₂, Y₃, Y₄, Y₅ are independently selected from CH, CR² and N.

In some embodiments, B, E and J are each independently selected from H and R³.

In some embodiments, R¹ is independently selected from the group consisting of H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano, CF₃, OCF₃, SOR⁷, SO₂R⁷, NO₂, COR⁴, C₁₋₆ alkyl-COR⁴, N(R⁷)C₂₋₆ alkyl-NR⁷R⁷, —N(R⁷)C₂₋₆ alkyl-R⁴, N(C₂₋₆ alkyl)₂-NR⁷, —O(CH₂)ₚR⁴, —S(CH₂)ₚR⁴, and —N(R⁷)C(═O)(CH₂)ₚR⁴, with a proviso that not more than three R¹ can be other than H.

In some embodiments, R² is independently selected from the group consisting of H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano, CF₃, OCF₃, SOR⁷, SO₂R⁷, NO₂, COR⁴, C₁₋₆ alkyl-COR⁴, N(R⁷)C₂₋₆ alkyl-NR⁷R⁷, N(C₂₋₆ alkyl)₂-NR⁷, CF₃, CO₂Et, CO₂H, —N(R⁷)C₂₋₆ alkyl-R⁴, —O(CH₂)ₚR⁴, —S(CH₂)ₚR⁴, and —N(R⁷)C(═O)(CH₂)ₚR⁴, with a proviso that not more than two R² can be other than H.

In some embodiments, R³ is independently selected from the group consisting of C₁₋₆ alkyl, C₃₋₆ alkenyl, C₃₋₆ alkynyl, C₃₋₇ cycloalkyl, C₄₋₇ heterocycloalkyl, phenyl, naphthyl, 5-10 membered mono- or bicyclic heteroaryl, C₁₋₆ alkyl-C₃₋₇ cycloalkyl, or C₁₋₆ alkyl-C₄₋₇ heterocycloalkyl, and C₁₋₆ R⁴.

In some embodiments, R⁴ is independently selected from the group consisting of OH, NR⁵R⁶, O(CH₂)_{q}NR⁵R⁶, C₁₋₆ alkoxy, C₁₋₆ alkoxy-C₁₋₆ alkoxy, C₂₋₆ hydroxyalkoxy, cyclopropyl, oxetanyl, oxetanyloxy, oxetanylamino, oxolanyl, oxolanyloxy, oxolanylamino, oxanyl oxanyloxy, oxanylamino, oxepanyl, oxepanyloxy, oxepanylamino, azetidinyl, azetidinyloxy, azetidylamino, pyrrolidinyl, pyrolidinyloxy, pyrrolidinylamino, piperidinyl, piperidinyloxy, piperidinylamino, azepanyl, azepanyloxy, azepanylamino, dioxolanyl, dioxanyl, morpholino, thiomorpholino, thiomorpholino-S,S-dioxide, piperazino, dioxepanyl, dioxepanyloxy, dioxepanylamino, oxazepanyl, oxazepanyloxy, oxazepanylamino, diazepanyl, diazepanyloxy, and diazepanylamino, all of which may be optionally substituted with OH, OR⁷, oxo, halogen, R⁶, CH₂OR⁶, CH₂NR⁵R⁶ or CH₂CH₂CONR⁵R⁶.

In some embodiments, R⁵ and R⁶ are each independently selected from the group consisting of H, —CD₃, C₁₋₆ alkyl, C₃₋₆ alkenyl, C₃₋₆ alkynyl, C₃₋₈ cycloalkyl, —(C₁₋₃ alkyl)-(C₃₋₈ cycloalkyl), C₃₋₈ cycloalkenyl, C₁-C6 acyl, 4-12 membered monocyclic or bicyclic heterocyclyl, 4-12 membered monocyclic or bicyclic heterocyclyl-C₁-C₆ alkyl-, C₆-C₁₂ aryl, and 5-11 membered heteroaryl; wherein R⁵ and R⁶ may be further independently substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo; or alternatively, R⁵ and R⁶, taken together with the N atom to which they are both attached, form a heterocycloalkyl ring of 4-7 members, containing up to one other heteroatom selected from O, S, or NR³, or a heterobicycloalkyl ring of 6-12 members which may be fused, bridged or spiro, and contain up to two other heteroatoms chosen from O, S(O)ₓ, or NR³.

In some embodiments, each R⁷ is independently selected from the group consisting of H, —CD₃, C₁₋₆ alkyl, C₃₋₆ cycloalkyl, phenyl, naphthyl, 5-10 membered mono- or bicyclo-heteroaryl, C₂₋₆ hydroxyalkyl, —SO₂-alkyl, NH—C₂₋₆ alkyl-NR⁵R⁶, C₁₋₆ alkoxy-C₁₋₆ alkyl, and C₂₋₆ alkyl-NR⁵R⁶; alternatively, two R⁷ taken together with the same N atom to which they are both attached, form a heterocyclic ring of 4-7 members, containing up to one other heteroatom selected from O, S, or NR³.

In some embodiments, p=0, 1, 2, 3, or 4.

In some embodiments, x=0, 1, or 2.

In some embodiments, L is a linker selected from a group consisting of —(CH₂)ₘ—, —NH(CH₂)ₘ—, —NH(CH₂CH₂O)ₙ—, —(CH₂CH₂O)ₙ—, —(CH₂)ₘCO—, —(CH₂CH₂O)ₙCO—,

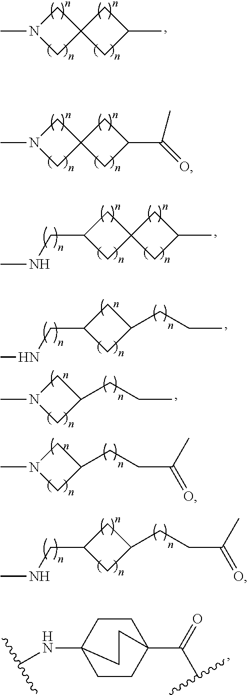

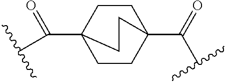 [SMILES: *C(=O)C12CCC(C(*)=O)(CC1)CC2]

—NH(CH₂)ₘC≡C, and —NH(CH₂CH₂O)ₙC≡C; wherein m=0, 1, 2, 3, 4, 5, 6, 7, 8, 9, or 10; and wherein n=0, 1, 2, 3, 4, 5, or 6.

In some embodiments, Z is a radical of an E3 ligase ligand selected from the group consisting of.

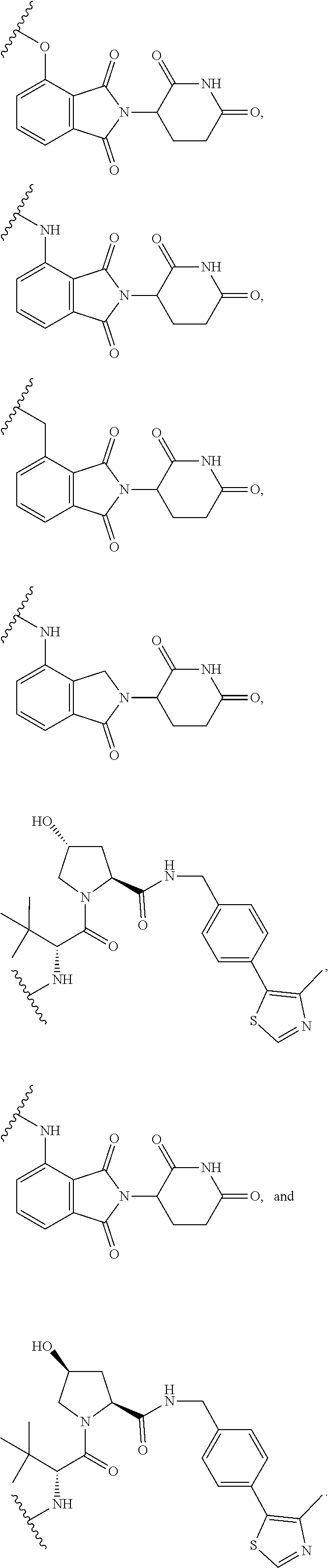

In a particular embodiment, compounds encompassed within Formula IV are provided:

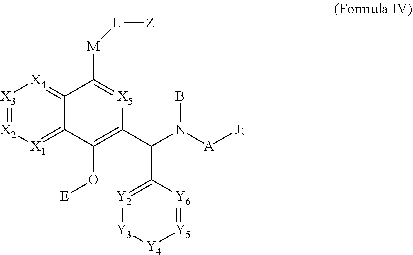

including pharmaceutically acceptable salts, solvates, and/or prodrugs thereof.

Formula IV is not limited to a particular chemical moiety for X₁, X₂, X₃, X₄, X₅, Y₂, Y₃, Y₄, Y₅, Y₆, A, B, E, M, J, L and Z. In some embodiments, the particular chemical moiety for X₁, X₂, X₃, X₄, X₅, Y₂, Y₃, Y₄, Y₅, Y₆, A, B, E, M, J, L and Z permits the resulting compound capable of one or more of:
inhibiting GRP78 activity;
serving as an effective therapeutic agent for treating, ameliorating, and preventing various forms of cancer, viral infections, and inflammatory diseases;
inducing ER stress-mediated apoptosis in the tumor cells implanted in mice without major toxicity to normal tissues; and
inducing ER stress and triggers UPR by inhibiting GRP78.

In some embodiments, X₁ is either CH or N.

In some embodiments, X₂, X₃, X₄, X₅ are independently selected from CR¹ and N, with the proviso that at least three of them must be CR¹.

In some embodiments, A is selected from the group consisting of CO, SO, and SO₂.

In some embodiments, Y₂, Y₃, Y₄, Y₅ are independently selected from the group consisting of CH, CR² or N.

In some embodiments, M is selected from the group consisting of NH and CO.

In some embodiments, B, E and J are each independently selected from the group consisting of H and R³.

In some embodiments, R¹ is independently selected from the group consisting of H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano, CF₃, OCF₃, SOR⁷, SO₂R⁷, NO₂, COR⁴, C₁₋₆ alkyl-COR⁴, N(R⁷)C₂₋₆ alkyl-NR⁷R⁷, —N(R⁷)C₂₋₆ alkyl-R⁴, N(C₂₋₆ alkyl)₂-NR⁷, —O(CH₂)ₚR⁴, —S(CH₂)ₚR⁴, and —N(R⁷)C(═O)(CH₂)ₚR⁴, with a proviso that not more than three R¹ can be other than H.

In some embodiments, R² is independently selected from the group consisting of H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano,

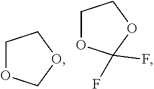

CF₃, OCF₃, SOR⁷, SO₂R⁷, NO₂, COR⁴, C₁₋₆ alkyl-COR⁴, N(R⁷)C₂₋₆ alkyl-NR⁷R⁷, N(C₂₋₆ alkyl)₂-NR⁷, CF₃, CO₂Et, CO₂H, —N(R⁷)C₂₋₆ alkyl-R⁴, —O(CH₂)ₚR⁴, —S(CH₂)ₚR⁴, and —N(R⁷)C(═O)(CH₂)ₚR⁴, with a proviso that not more than two R² can be other than H.

In some embodiments, R³ is independently selected from the group consisting of C₁₋₆ alkyl, C₃₋₆ alkenyl, C₃₋₆ alkynyl, C₃₋₇ cycloalkyl, C₄₋₇ heterocycloalkyl, phenyl, naphthyl, 5-10 membered mono- or bicyclic heteroaryl, C₁₋₆ alkyl-C₃₋₇ cycloalkyl, C₁₋₆ alkyl-C₄₋₇ heterocycloalkyl, and C₁₋₆ R⁴.

In some embodiments, R⁴ is independently selected from the group consisting of OH, NR⁵R⁶, O(CH₂)_{q}NR⁵R⁶, C₁₋₆ alkoxy, C₁₋₆ alkoxy-C₁₋₆ alkoxy, C₂₋₆ hydroxyalkoxy, cyclopropyl, oxetanyl, oxetanyloxy, oxetanylamino, oxolanyl, oxolanyloxy, oxolanylamino, oxanyl oxanyloxy, oxanylamino, oxepanyl, oxepanyloxy, oxepanylamino, azetidinyl, azetidinyloxy, azetidylamino, pyrrolidinyl, pyrolidinyloxy, pyrrolidinylamino, piperidinyl, piperidinyloxy, piperidinylamino, azepanyl, azepanyloxy, azepanylamino, dioxolanyl, dioxanyl, morpholino, thiomorpholino, thiomorpholino-S,S-dioxide, piperazino, dioxepanyl, dioxepanyloxy, dioxepanylamino, oxazepanyl, oxazepanyloxy, oxazepanylamino, diazepanyl, diazepanyloxy, and diazepanylamino, all of which may be optionally substituted with OH, OR⁷, oxo, halogen, R⁶, CH₂OR⁶, CH₂NR⁵R⁶ or CH₂CH₂CONR⁵R⁶.

In some embodiments, R⁵ and R⁶ are each independently selected from the group consisting of H, —CD₃, C₁₋₆ alkyl, C₃₋₆ alkenyl, C₃₋₆ alkynyl, C₃₋₈ cycloalkyl, —(C₁₋₃ alkyl)-(C₃₋₈ cycloalkyl), C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, and 5-11 membered heteroaryl; wherein R⁵ and R⁶ may be further independently substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo; or alternatively, R⁵ and R⁶, taken together with the N atom to which they are both attached, form a heterocycloalkyl ring of 4-7 members, containing up to one other heteroatom selected from O, S, or NR³, or a heterobicycloalkyl ring of 6-12 members which may be fused, bridged or spiro, and contain up to two other heteroatoms chosen from O, S(O)ₓ, or NR³.

In some embodiments, each R⁷ is independently selected from the group consisting of H, —CD₃, C₁₋₆ alkyl, C₃₋₆ cycloalkyl, phenyl, naphthyl, 5-10 membered mono- or bicyclo-heteroaryl, C₂₋₆ hydroxyalkyl, —SO₂-alkyl, NH—C₂₋₆ alkyl-NR⁵R⁶, C₁₋₆ alkoxy-C₁₋₆ alkyl, and C₂₋₆ alkyl-NR⁵R⁶; alternatively, two R⁷ taken together with the same N atom to which they are both attached, form a heterocyclic ring of 4-7 members, containing up to one other heteroatom selected from O, S, or NR³.

In some embodiments, p=0, 1, 2, 3, or 4.

In some embodiments, x=0, 1, or 2.

In some embodiments, L is a linker selected from a group consisting of —CO(CH₂)ₘ—, —NH(CH₂)ₘ—, —NH(CH₂CH₂O)ₙ—, —CO(CH₂CH₂O)ₙ—, —NH(CH₂)ₘCO—, —NH(CH₂CH₂O)ₙCO—,

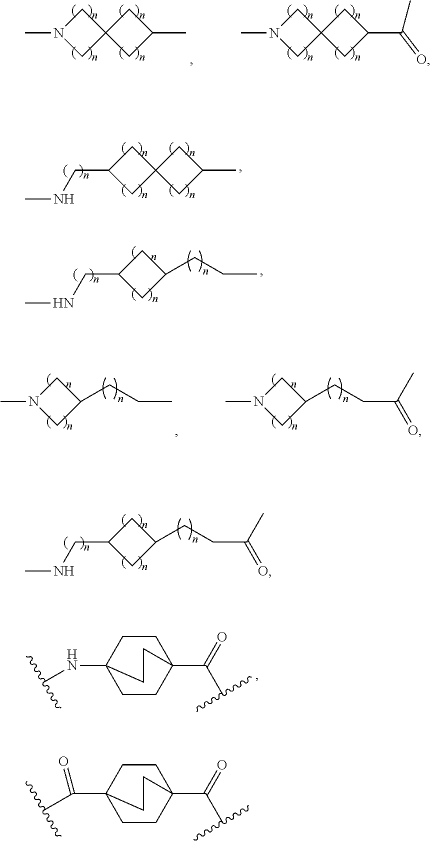

—NH(CH₂)ₘC≡C, —NH(CH₂CH₂O)ₙC≡C —CO(CH₂)ₘC≡C, and —CO(CH₂CH₂O)ₙC≡C; wherein m=0, 1, 2, 3, 4, 5, 6, 7, 8, 9, or 10, and wherein n=0, 1, 2, 3, 4, 5, or 6.

In some embodiments, Z is a radical of an E3 ligase ligand selected from the group consisting of:

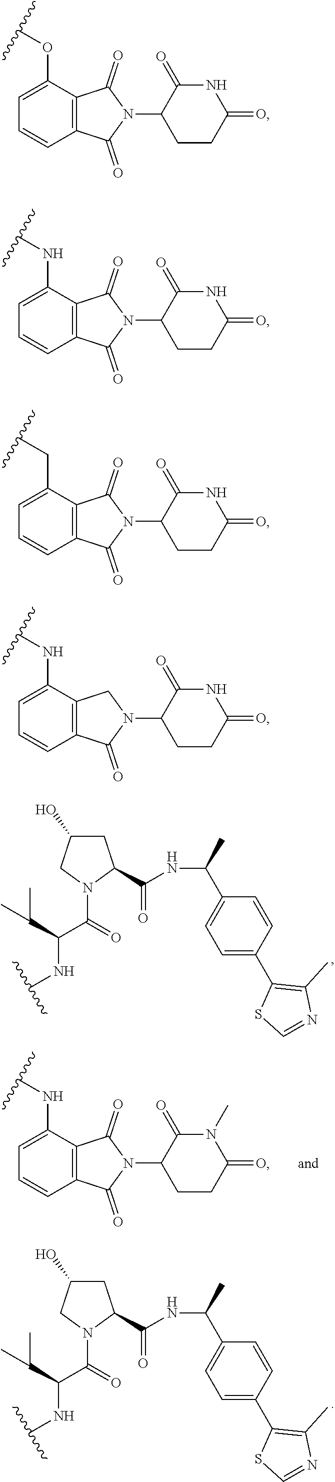

In some embodiments, compounds shown in Table II are contemplated for Formulas II, III and IV.

TABLE II

| SI No | Structure | Name |
|---|---|---|
| P1 | 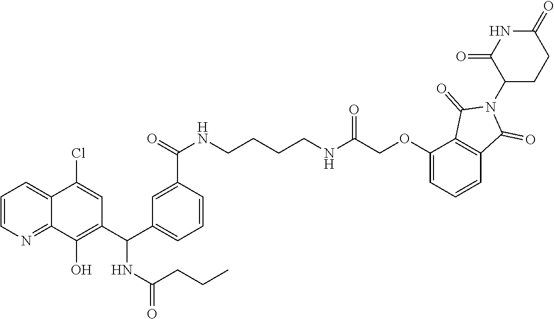 [SMILES: CCCC(=O)NC(c1cccc(C(=O)NCCCCNC(=O)COc2cccc3c2C(=O)N(C2CCC(=O)NC2=O)C3=O)c1)c1cc(Cl)c2cccnc2c1O] | 3-(butyramido(5- chloro-8-hydroxy- quinolin-7-yl)meth- yl)-N-(4-(2-((2-(2,6- dioxopiperidin-3- yl)-1,3-dioxoiso- indolin-4-yl)oxy)- acetamido)butyl)- benzamide |
| P2 | 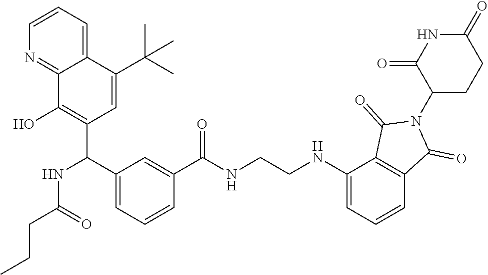 [SMILES: CCCC(=O)NC(c1cccc(C(=O)NCCNc2cccc3c2C(=O)N(C2CCC(=O)NC2=O)C3=O)c1)c1cc(C(C)(C)C)c2cccnc2c1O] | 3-((5-(tert-butyl)-8- hydroxyquinolin-7- yl)(butyramido)- methyl)-N-(2-((2- (2,6-dioxopiperidin- 3-yl)-1,3-dioxoiso- indolin-4-yl)amino)- ethyl)benzamide |
| P3 | 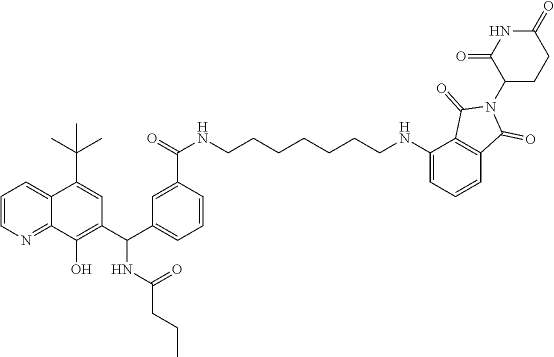 [SMILES: CCCC(=O)NC(c1cccc(C(=O)NCCCCCCCNc2cccc3c2C(=O)N(C2CCC(=O)NC2=O)C3=O)c1)c1cc(C(C)(C)C)c2cccnc2c1O] | 3-((5-(tert-butyl)-8- hydroxyquinolin-7- yl)(butyramido)- methyl)-N-(7-((2- (2,6-dioxopiperidin- 3-yl)-1,3-dioxoiso- indolin-4-yl)amino)- heptyl)benzamide |
| P4 | 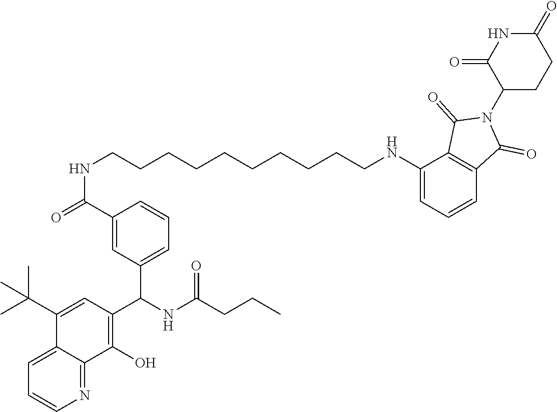 [SMILES: CCCC(=O)NC(c1cccc(C(=O)NCCCCCCCCCCNc2cccc3c2C(=O)N(C2CCC(=O)NC2=O)C3=O)c1)c1cc(C(C)(C)C)c2cccnc2c1O] | 3-((5-(tert-butyl)-8- hydroxyquinolin-7- yl)(butyramido)- methyl)-N-(10-((2- (2,6-dioxopiperidin- 3-yl)-1,3-dioxoiso- indolin-4-yl)amino)- decyl)benzamide |
| P5 | 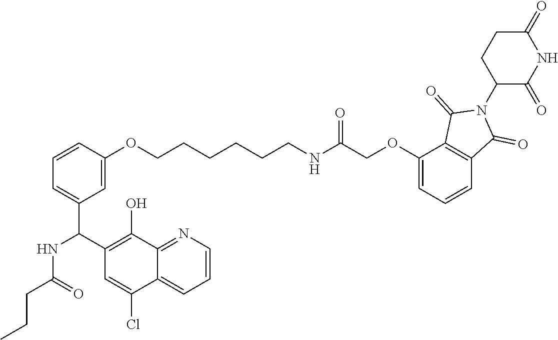 [SMILES: CCCC(=O)NC(c1cccc(OCCCCCCNC(=O)COc2cccc3c2C(=O)N(C2CCC(=O)NC2=O)C3=O)c1)c1cc(Cl)c2cccnc2c1O] | N-((5-chloro-8- hydroxyquinolin-7- yl)(3-((6-(2-((2- (2,6-dioxopiperidin- 3-yl)-1,3-dioxoiso- indolin-4-yl)oxy)- acetamido)hexyl)- oxy)phenyl)methyl)- butyramide |
| P6 | 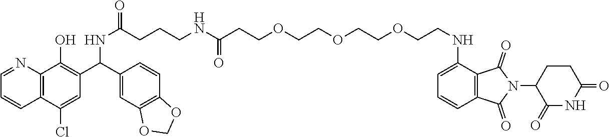 [SMILES: O=C(CCOCCOCCOCCNc1cccc2c1C(=O)N(C1CCC(=O)NC1=O)C2=O)NCCCC(=O)NC(c1ccc2c(c1)OCO2)c1cc(Cl)c2cccnc2c1O] | N-(benzo[d][1,3]- dioxol-5-yl(5- chloro-8-hydroxy- quinolin-7-yl)meth- yl)-4-(3-(2-(2-(2- ((2-(2,6-dioxopiper- idin-3-yl)-1,3-dioxo- isoindolin-4-yl)- amino)ethoxy)- |
| | | ethoxy)ethoxy)- |
| | | propanamido)butan- |
| | | amide |
| P7 | 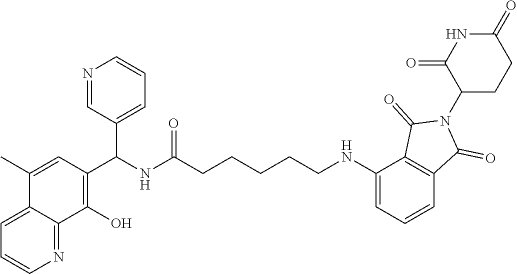 [SMILES: Cc1cc(C(NC(=O)CCCCCNc2cccc3c2C(=O)N(C2CCC(=O)NC2=O)C3=O)c2cccnc2)c(O)c2ncccc12] | 6-((2-(2,6-dioxo- piperidin-3-yl)-1,3- dioxoisoindolin-4- yl)amino)-N-((8- hydroxy-5-meth- ylquinolin-7-yl)- (pyridin-3-yl)meth- yl)hexanamide |
| P8 | 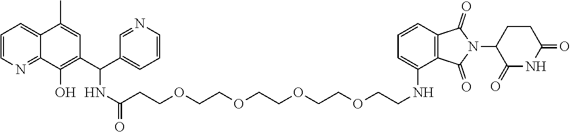 [SMILES: Cc1cc(C(NC(=O)CCOCCOCCOCCOCCNc2cccc3c2C(=O)N(C2CCC(=O)NC2=O)C3=O)c2cccnc2)c(O)c2ncccc12] | 1-((2-(2,6-dioxo- piperidin-3-yl)-1,3- dioxoisoindolin-4- yl)amino)-N-((8- hydroxy-5-methyl- quinolin-7-yl)(pyr- idin-3-yl)methyl)- 3,6,9,12-tetra-oxa- pentadecan-15-amide |
| P9 | 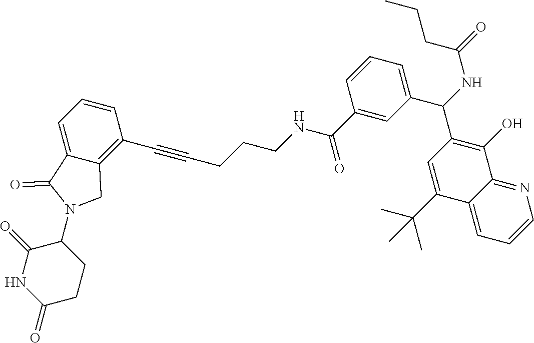 [SMILES: CCCC(=O)NC(c1cccc(C(=O)NCCCC#Cc2cccc3c2CN(C2CCC(=O)NC2=O)C3=O)c1)c1cc(C(C)(C)C)c2cccnc2c1O] | 3-((5-(tert-butyl)-8- hydroxyquinolin-7- yl)(butyramido)- methyl)-N-(5-(2- (2,6-dioxopiperidin- 3-yl)-1-oxoiso- indolin-4-yl)pent-4- yn-1-yl)benzamide |
| P10 | 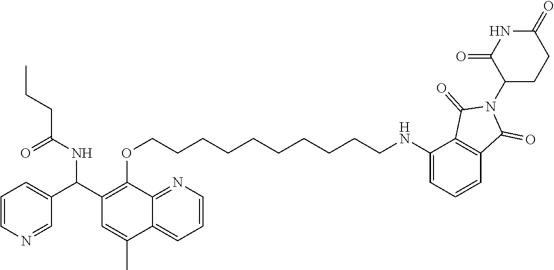 [SMILES: CCCC(=O)NC(c1cccnc1)c1cc(C)c2cccnc2c1OCCCCCCCCCCNc1cccc2c1C(=O)N(C1CCC(=O)NC1=O)C2=O] | N-((8-((10-((2-(2,6- dioxopiperidin-3- yl)-1,3-dioxoiso- indolin-4-yl)amino)- decyl)oxy)-5-meth- ylquinolin-7-yl)- (pyridin-3-yl)meth- yl)butyramide |
| P11 | 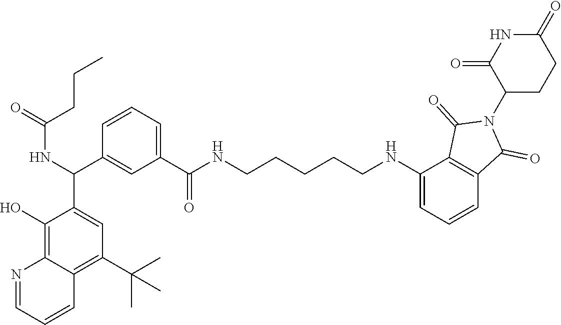 [SMILES: CCCC(=O)NC(c1cccc(C(=O)NCCCCCNc2cccc3c2C(=O)N(C2CCC(=O)NC2=O)C3=O)c1)c1cc(C(C)(C)C)c2cccnc2c1O] | 3-((5-(tert-butyl)-8- hydroxyquinolin-7- yl)(butyramido)- methyl)-N-(5-((2- (2,6-dioxopiperidin- 3-yl)-1,3-dioxoiso- indolin-4-yl)amino)- pentyl)benzamide |
| P12 | 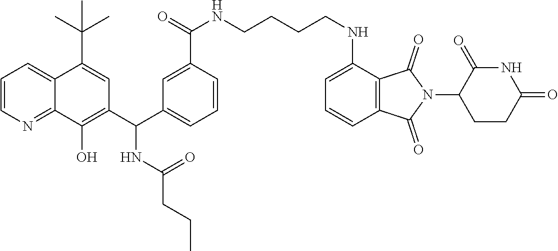 [SMILES: CCCC(=O)NC(c1cccc(C(=O)NCCCCNc2cccc3c2C(=O)N(C2CCC(=O)NC2=O)C3=O)c1)c1cc(C(C)(C)C)c2cccnc2c1O] | 3-((5-(tert-butyl)-8- hydroxyquinolin-7- yl)(butyramido)- methyl)-N-(4-((2- (2,6-dioxopiperidin- 3-yl)-1,3-dioxoiso- indolin-4-yl)amino)- butyl)benzamide |
| P13 | 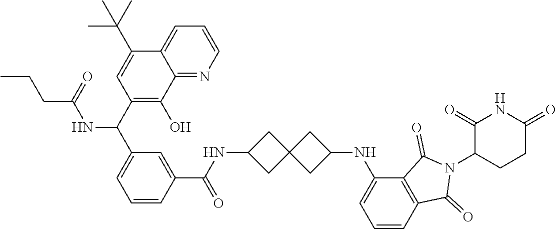 [SMILES: CCCC(=O)NC(c1cccc(C(=O)NC2CC3(C2)CC(Nc2cccc4c2C(=O)N(C2CCC(=O)NC2=O)C4=O)C3)c1)c1cc(C(C)(C)C)c2cccnc2c1O] | 3-((5-(tert-butyl)-8- hydroxyquinolin-7- yl)(butyramido)- methyl)-N-(6-((2- (2,6-dioxopiperidin- 3-yl)-1,3-dioxoiso- indolin-4-yl)amino)- spiro[3.3]heptan-2- yl)benzamide |
| P14 | 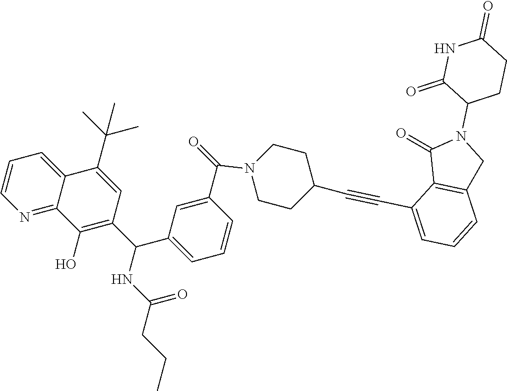 [SMILES: CCCC(=O)NC(c1cccc(C(=O)N2CCC(C#Cc3cccc4c3C(=O)N(C3CCC(=O)NC3=O)C4)CC2)c1)c1cc(C(C)(C)C)c2cccnc2c1O] | N-((5-(tert-butyl)-8- hydroxyquinolin-7- yl)(3-(4-((2-(2,6- dioxopiperidin-3- yl)-3-oxoisoindolin- 4-yl)ethynyl)piper- idine-1-carbonyl)- phenyl)methyl)- butyramide |
| P15 | 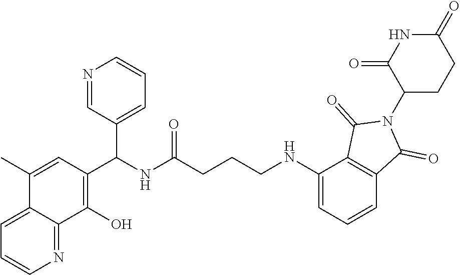 [SMILES: Cc1cc(C(NC(=O)CCCNc2cccc3c2C(=O)N(C2CCC(=O)NC2=O)C3=O)c2cccnc2)c(O)c2ncccc12] | 4-((2-(2,6-dioxo- piperidin-3-yl)-1,3- dioxoisoindolin-4- yl)amino)-N-((8- hydroxy-5-meth- quinolin-7-yl)- (pyridin-3-yl)meth- yl)butanamide |
| P16 | 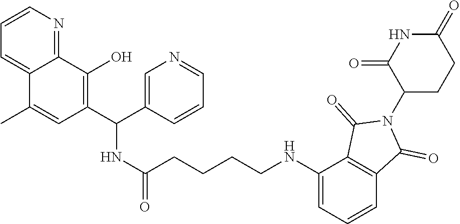 [SMILES: Cc1cc(C(NC(=O)CCCCNc2cccc3c2C(=O)N(C2CCC(=O)NC2=O)C3=O)c2cccnc2)c(O)c2ncccc12] | 5-((2-(2,6-dioxo- piperidin-3-yl)-1,3- dioxoisoindolin-4- yl)amino)-N-((8- hydroxy-5-methyl- quinolin-7-yl)- (pyridin-3-yl)meth- yl)pentanamide |
| P17 | 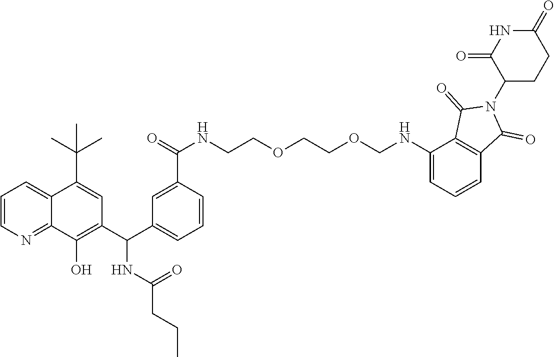 [SMILES: CCCC(=O)NC(c1cccc(C(=O)NCCOCCOCNc2cccc3c2C(=O)N(C2CCC(=O)NC2=O)C3=O)c1)c1cc(C(C)(C)C)c2cccnc2c1O] | 3-((5-(tert-butyl)-8- hydroxyquinolin-7- yl)(butyramido)- methyl)-N-(2-(2- (((2-(2,6-dioxopiper- idin-3-yl)-1,3- dioxoisoindolin-4- yl)amino)methoxy)- ethoxy)ethyl)benz- amide |
| P18 | 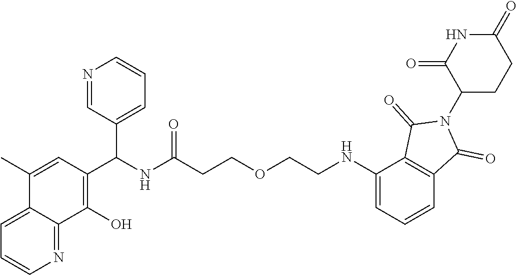 [SMILES: Cc1cc(C(NC(=O)CCOCCNc2cccc3c2C(=O)N(C2CCC(=O)NC2=O)C3=O)c2cccnc2)c(O)c2ncccc12] | 3-(2-((2-(2,6-dioxo- piperidin-3-yl)-1,3- dioxoisoindolin-4- yl)amino)ethoxy)- N-((8-hydroxy-5- methylquinolin-7- yl)(pyridin-3-yl)- methyl)propan- amide |
| P19 | 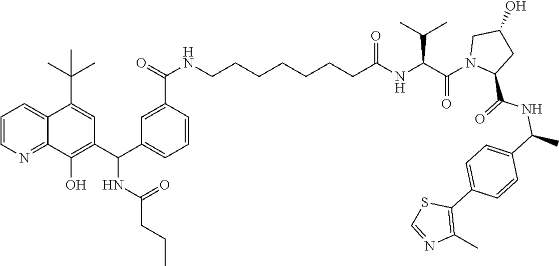 [SMILES: CCCC(=O)NC(c1cccc(C(=O)NCCCCCCCC(=O)N[C@H](C(=O)N2C[C@H](O)C[C@H]2C(=O)N[C@@H](C)c2ccc(-c3scnc3C)cc2)C(C)C)c1)c1cc(C(C)(C)C)c2cccnc2c1O] | (2S,4R)-1-((8-(3- ((5-(tert-butyl)-8- hydroxyquinolin-7- yl)(butyramido)- methyl)benzamido)- octanoyl)-L-valyl)- 4-hydroxy-N-((S)- 1-(4-(4-methylthia- zol-5-yl)phenyl)- ethyl)pyrrolidine- 2-carboxamide |
| P20 | 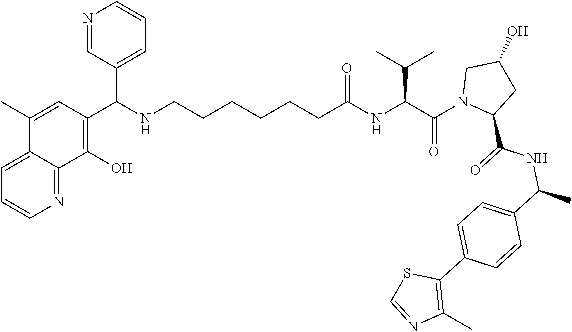 [SMILES: Cc1ncsc1-c1ccc([C@H](C)NC(=O)[C@@H]2C[C@@H](O)CN2C(=O)[C@@H](NC(=O)CCCCCCNC(c2cccnc2)c2cc(C)c3cccnc3c2O)C(C)C)cc1] | (2S,4R)-4-hydroxy- 1-((7-(((8-hydroxy- 5-methylquinolin-7- yl)(pyridin-3-yl)- methyl)amino)- heptanoyl)-L-valyl)- N-((S)-1-(4-(4- methylthiazol-5-yl)- phenyl)ethyl)pyrrol- idine-2-carboxamide |
| P21 | 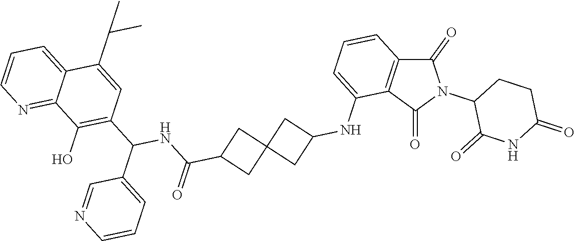 [SMILES: CC(C)c1cc(C(NC(=O)C2CC3(CC(Nc4cccc5c4C(=O)N(C4CCC(=O)NC4=O)C5=O)C3)C2)c2cccnc2)c(O)c2ncccc12] | N-((5-(tert-butyl)-8- hydroxyquinolin-7- yl)(pyridin-3-yl)- methyl)-6-((2-(2,6- dioxopiperidin-3- yl)-1,3-dioxoiso- indolin-4-yl)amino)- spiro[3.3]heptane-2- carboxamide |
| P22 | 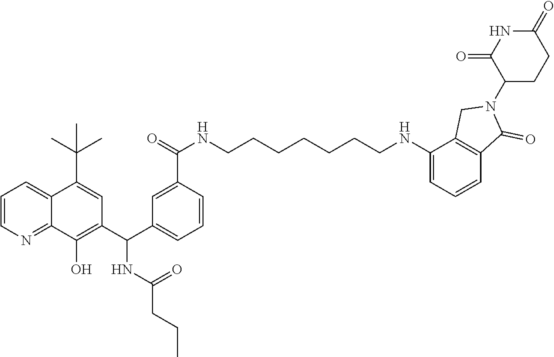 [SMILES: CCCC(=O)NC(c1cccc(C(=O)NCCCCCCCNc2cccc3c2CN(C2CCC(=O)NC2=O)C3=O)c1)c1cc(C(C)(C)C)c2cccnc2c1O] | 3-((5-(tert-butyl)-8- hydroxyquinolin-7- yl)(butyramido)- methyl)-N-(7-((2- (2,6-dioxopiperidin- 3-yl)-1-oxoiso- indolin-4-yl)amino)- heptyl)benzamide |
| P23 | 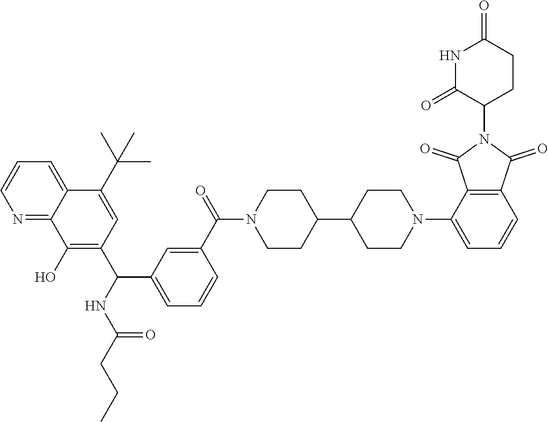 [SMILES: CCCC(=O)NC(c1cccc(C(=O)N2CCC(C3CCN(c4cccc5c4C(=O)N(C4CCC(=O)NC4=O)C5=O)CC3)CC2)c1)c1cc(C(C)(C)C)c2cccnc2c1O] | N-((5-(tert-butyl)-8- hydroxyquinolin-7- yl)(3-(1′-(2-(2,6- dioxopiperidin-3- yl)-1,3-dioxoiso- indolin-4-yl)-[4,4′- bipiperidine]-1- carbonyl)phenyl)- methyl)butyramide |
| P24 | 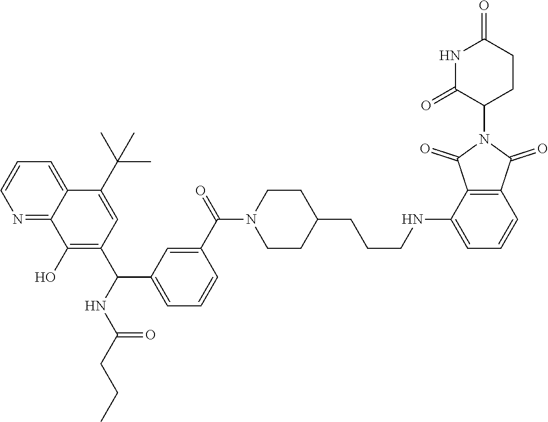 [SMILES: CCCC(=O)NC(c1cccc(C(=O)N2CCC(CCCNc3cccc4c3C(=O)N(C3CCC(=O)NC3=O)C4=O)CC2)c1)c1cc(C(C)(C)C)c2cccnc2c1O] | N-((5-(tert-butyl)-8- hydroxyquinolin-7- yl)(3-(4-(3-((2-(2,6- dioxopiperidin-3- yl)-1,3-dioxoiso- indolin-4-yl)amino)- propyl)piperidine-1- carbonyl)phenyl)- methyl)butyramide |
| P25 | 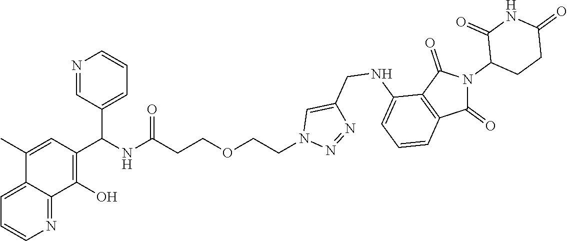 [SMILES: Cc1cc(C(NC(=O)CCOCCn2cc(CNc3cccc4c3C(=O)N(C3CCC(=O)NC3=O)C4=O)nn2)c2cccnc2)c(O)c2ncccc12] | 3-(2-(4-(((2-(2,6- dioxopiperidin-3- yl)-1,3-dioxoiso- indolin-4-yl)amino)- methyl)-1H-1,2,3- triazol-1-yl)ethoxy)- N-((8-hydroxy-5- methylquinolin-7- yl)(pyridin-3-yl)- methyl)propan- amide |
| P26 | 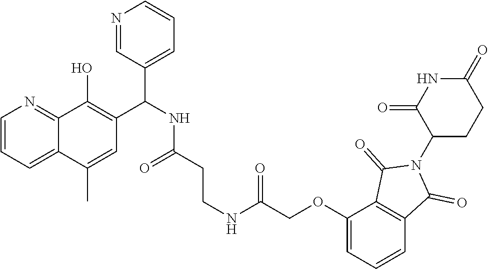 [SMILES: Cc1cc(C(NC(=O)CCNC(=O)COc2cccc3c2C(=O)N(C2CCC(=O)NC2=O)C3=O)c2cccnc2)c(O)c2ncccc12] | 3-(2-((2-(2,6-dioxo- piperidin-3-yl)-1,3- dioxoisoindolin-4- yl)oxy)acetamido)- N-((8-hydroxy-5- methylquinolin-7- yl)(pyridin-3-yl)- methyl)propan- amide |
| P27 | 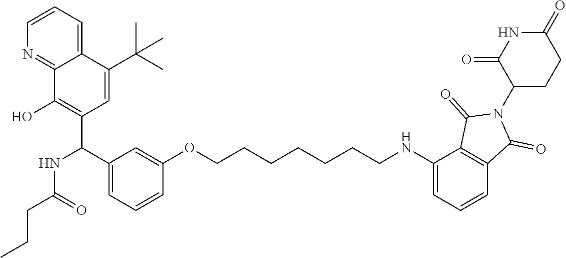 [SMILES: CCCC(=O)NC(c1cccc(OCCCCCCCNc2cccc3c2C(=O)N(C2CCC(=O)NC2=O)C3=O)c1)c1cc(C(C)(C)C)c2cccnc2c1O] | N-((5-(tert-butyl)-8- hydroxyquinolin-7- yl)(3-((7-((2-(2,6- dioxopiperidin-3- yl)-1,3-dioxoiso- indolin-4-yl)amino)- heptyl)oxy)phenyl)- methyl)butyramide |
| P28 | 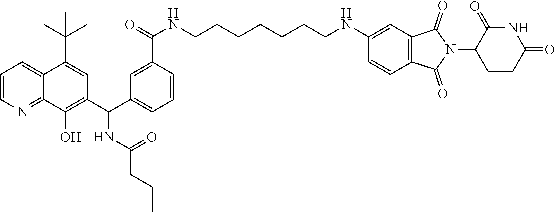 [SMILES: CCCC(=O)NC(c1cccc(C(=O)NCCCCCCCNc2ccc3c(c2)C(=O)N(C2CCC(=O)NC2=O)C3=O)c1)c1cc(C(C)(C)C)c2cccnc2c1O] | 3-((5-(tert-butyl)-8- hydroxyquinolin-7- yl)(butyramido)- methyl)-N-(7-((2- (2,6-dioxopiperidin- 3-yl)-1,3-dioxoiso- indolin-5-yl)amino)- heptyl)benzamide |
| P29 | 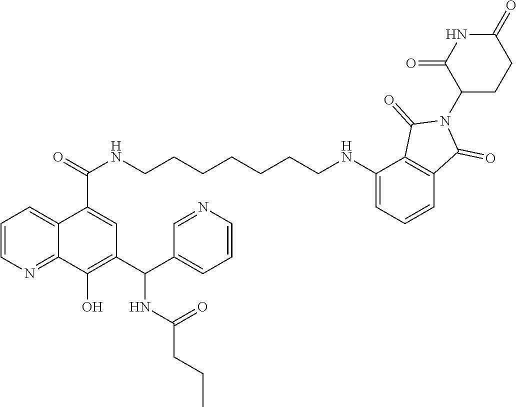 [SMILES: CCCC(=O)NC(c1cccnc1)c1cc(C(=O)NCCCCCCCNc2cccc3c2C(=O)N(C2CCC(=O)NC2=O)C3=O)c2cccnc2c1O] | 7-butyramido(pyr- idin-3-yl)methyl)-N- (7-((2-(2,6-dioxo- piperidin-3-yl)-1,3- dioxoisoindolin-4- yl)amino)heptyl)-8- hydroxyquinoline- 5-carboxamide |
| P30 | 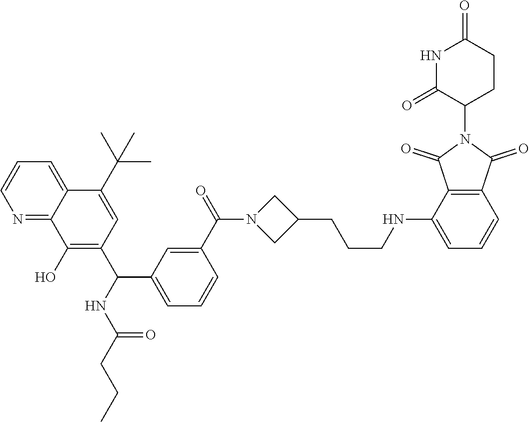 [SMILES: CCCC(=O)NC(c1cccc(C(=O)N2CC(CCCNc3cccc4c3C(=O)N(C3CCC(=O)NC3=O)C4=O)C2)c1)c1cc(C(C)(C)C)c2cccnc2c1O] | N-((5-(tert-butyl)-8- hydroxyquinolin-7- yl)(3-(3-(3-((2-(2,6- dioxopiperidin-3- yl)-1,3-dioxoiso- indolin-4-yl)amino)- propyl)azetidine-1- carbonyl)phenyl)- methyl)butyramide |
| P31 | 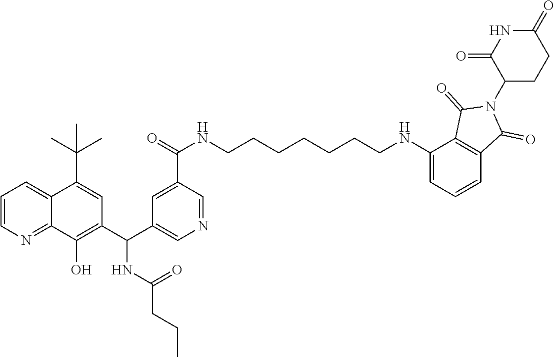 [SMILES: CCCC(=O)NC(c1cncc(C(=O)NCCCCCCCNc2cccc3c2C(=O)N(C2CCC(=O)NC2=O)C3=O)c1)c1cc(C(C)(C)C)c2cccnc2c1O] | 5-((5-(tert-butyl)-8- hydroxyquinolin-7- yl)(butyramido)- methyl)-N-(7-((2- (2,6-dioxopiperidin- 3-yl)-1,3-dioxoiso- indolin-4-yl)amino)- heptyl)nicotinamide |
| P32 | 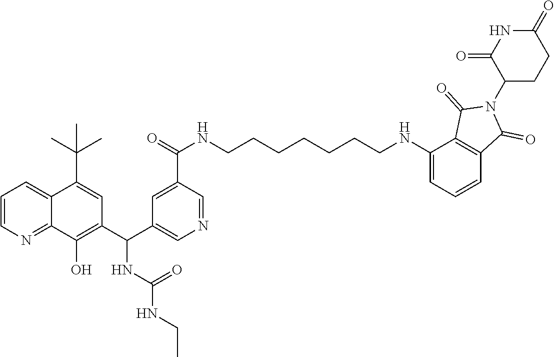 [SMILES: CCNC(=O)NC(c1cncc(C(=O)NCCCCCCCNc2cccc3c2C(=O)N(C2CCC(=O)NC2=O)C3=O)c1)c1cc(C(C)(C)C)c2cccnc2c1O] | 5-((5-(tert-butyl)-8- hydroxyquinolin-7- yl)(3-ethylureido)- methyl)-N-(7-((2- (2,6-dioxopiperidin- 3-yl)-1,3-dioxoiso- indolin-4-yl)amino)- heptyl)nicotinamide |
| P33 | 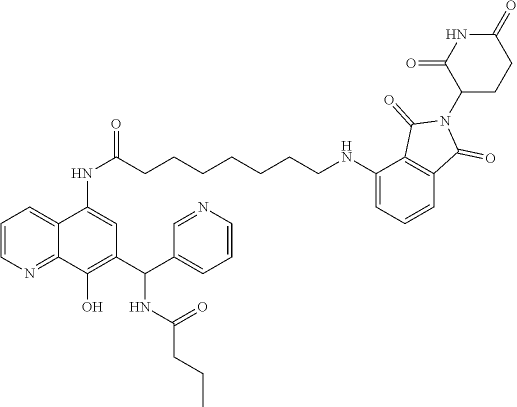 [SMILES: CCCC(=O)NC(c1cccnc1)c1cc(NC(=O)CCCCCCCNc2cccc3c2C(=O)N(C2CCC(=O)NC2=O)C3=O)c2cccnc2c1O] | N-(7-(butyramido- (pyridin-3-yl)meth- yl)-8-hydroxy- quinolin-5-yl)-8- ((2-(2,6-dioxopiper- idin-3-yl)-1,3-dioxo- isoindolin-4-yl)- amino)octanamide |
| P34 | 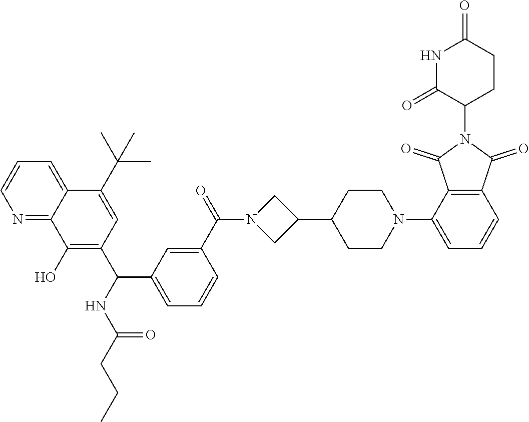 [SMILES: CCCC(=O)NC(c1cccc(C(=O)N2CC(C3CCN(c4cccc5c4C(=O)N(C4CCC(=O)NC4=O)C5=O)CC3)C2)c1)c1cc(C(C)(C)C)c2cccnc2c1O] | N-((5-(tert-butyl)-8- hydroxyquinolin-7- yl)(3-(3-(1-(2-(2,6- dioxopiperidin-3- yl)-1,3-dioxoiso- indolin-4-yl)piper- idin-4-yl)azetidine- 1-carbonyl)phen- yl)methyl)butyr- amide |
| P35 | 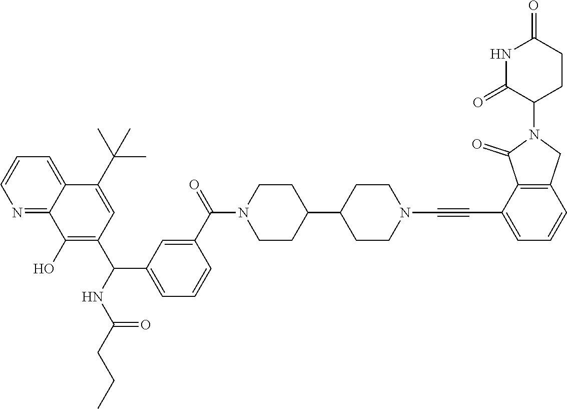 [SMILES: CCCC(=O)NC(c1cccc(C(=O)N2CCC(C3CCN(C#Cc4cccc5c4C(=O)N(C4CCC(=O)NC4=O)C5)CC3)CC2)c1)c1cc(C(C)(C)C)c2cccnc2c1O] | N-((5-(tert-butyl)-8- hydroxyquinolin-7- yl)(3-(4-((2-(2,6- dioxopiperidin-3- yl)-3-oxoisoindolin- 4-yl)ethynyl)-[1,4′- bipiperidine]-1′- carbonyl)phenyl)- methyl)butyramide |
| P36 | 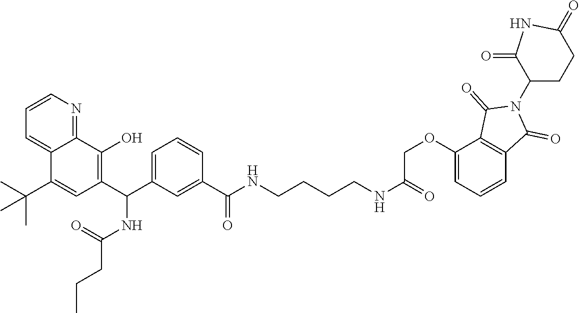 [SMILES: CCCC(=O)NC(c1cccc(C(=O)NCCCCNC(=O)COc2cccc3c2C(=O)N(C2CCC(=O)NC2=O)C3=O)c1)c1cc(C(C)(C)C)c2cccnc2c1O] | 3-((5-(tert-butyl)-8- hydroxyquinolin-7- yl)(butyramido)- methyl)-N-(4-(2-((2- (2,6-dioxopiperidin- 3-yl)-1,3-dioxoiso- indolin-4-yl)oxy)- acetamido)butyl)- benzamide |
| P37 | 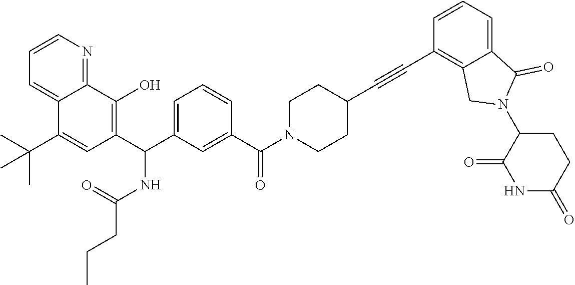 [SMILES: CCCC(=O)NC(c1cccc(C(=O)N2CCC(C#Cc3cccc4c3CN(C3CCC(=O)NC3=O)C4=O)CC2)c1)c1cc(C(C)(C)C)c2cccnc2c1O] | N-((5-(tert-butyl)-8- hydroxyquinolin-7- yl)(3-(4-((2-(2,6- dioxopiperidin-3- yl)-1-oxoisoindolin- 4-yl)ethynyl)piper- idine-1-carbonyl)- phenyl)methyl)- butyramide |
| P38 | 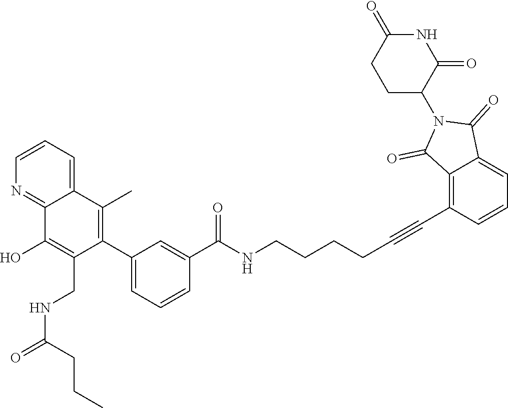 [SMILES: CCCC(=O)NCc1c(-c2cccc(C(=O)NCCCCC#Cc3cccc4c3C(=O)N(C3CCC(=O)NC3=O)C4=O)c2)c(C)c2cccnc2c1O] | 3-(butyramido(8- hydroxy-5-methyl- quinolin-7-yl)meth- yl)-N-(6-(2-(2,6- dioxopiperidin-3- yl)-1,3-dioxoiso- indolin-4-yl)hex-5- yn-1-yl)benzamide |
| P39 | 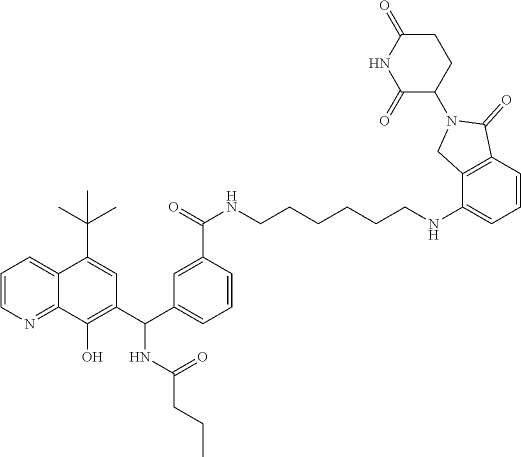 [SMILES: CCCC(=O)NC(c1cccc(C(=O)NCCCCCCNc2cccc3c2CN(C2CCC(=O)NC2=O)C3=O)c1)c1cc(C(C)(C)C)c2cccnc2c1O] | 3-((5-(tert-butyl)-8- hydroxyquinolin-7- yl)(butyramido)- methyl)-N-(6-((2- (2,6-dioxopiperidin- 3-yl)-1-oxoiso- indolin-4-yl)amino)- hexyl)benzamide |
| P40 | 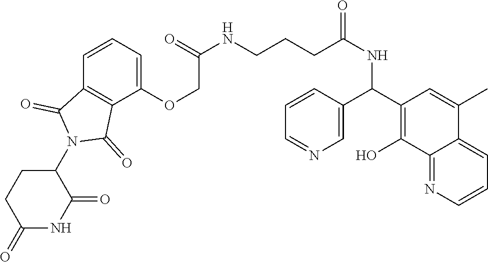 [SMILES: Cc1cc(C(NC(=O)CCCNC(=O)COc2cccc3c2C(=O)N(C2CCC(=O)NC2=O)C3=O)c2cccnc2)c(O)c2ncccc12] | 4-(2-((2-(2,6-dioxo- piperidin-3-yl)-1,3- dioxoisoindolin-4- yl)oxy)acetamido)- N-((8-hydroxy-5- methylquinolin-7- yl)(pyridin-3-yl)- methyl)butanamide |
| P41 | 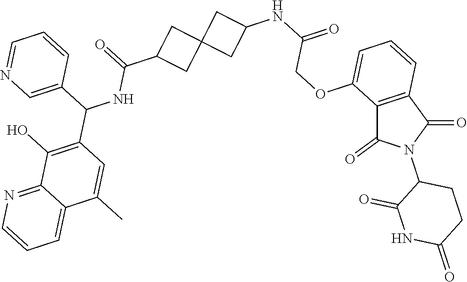 [SMILES: Cc1cc(C(NC(=O)C2CC3(CC(NC(=O)COc4cccc5c4C(=O)N(C4CCC(=O)NC4=O)C5=O)C3)C2)c2cccnc2)c(O)c2ncccc12] | 6-(2-((2-(2,6-dioxo- piperidin-3-yl)-1,3- dioxoisoindolin-4- yl)oxy)acetamido)- N-((8-hydroxy-5- methylquinolin-7- yl)(pyridin-3-yl)- methyl)spiro[3.3]- heptane-2-carbox- amide |
| P42 | 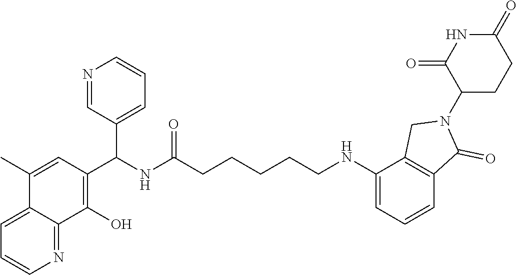 [SMILES: Cc1cc(C(NC(=O)CCCCCNc2cccc3c2CN(C2CCC(=O)NC2=O)C3=O)c2cccnc2)c(O)c2ncccc12] | 6-((2-(2,6-dioxo- piperidin-3-yl)-1- oxoisoindolin-4-yl)- amino)-N-((8- hydroxy-5-methyl- quinolin-7-yl)(pyr- idin-3-yl)methyl)- hexanamide |
| P43 | 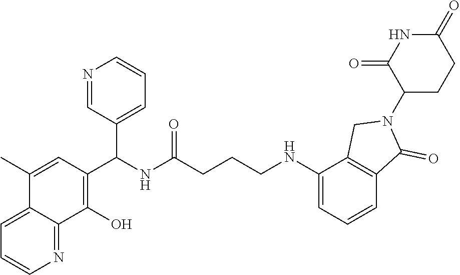 [SMILES: Cc1cc(C(NC(=O)CCCNc2cccc3c2CN(C2CCC(=O)NC2=O)C3=O)c2cccnc2)c(O)c2ncccc12] | 4-((2-(2,6-dioxo- piperidin-3-yl)-1- oxoisoindolin-4- yl)amino)-N-((8- hydroxy-5-methyl- quinolin-7-yl)(pyr- idin-3-yl)methyl)- butanamide |
| P44 | 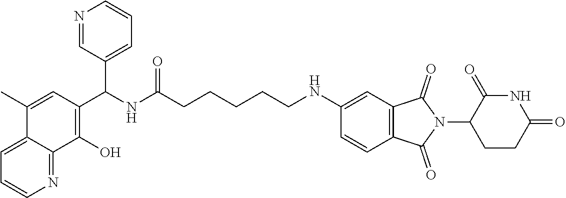 [SMILES: Cc1cc(C(NC(=O)CCCCCNc2ccc3c(c2)C(=O)N(C2CCC(=O)NC2=O)C3=O)c2cccnc2)c(O)c2ncccc12] | 6-((2-(2,6-dioxo- piperidin-3-yl)-1,3- dioxoisoindolin-5- yl)amino)-N-((8- hydroxy-5-methyl- quinolin-7-yl)(pyr- idin-3-yl)methyl)- hexanamide |
| P45 | 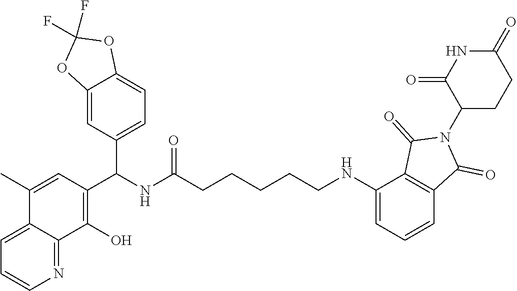 [SMILES: Cc1cc(C(NC(=O)CCCCCNc2cccc3c2C(=O)N(C2CCC(=O)NC2=O)C3=O)c2ccc3c(c2)OC(F)(F)O3)c(O)c2ncccc12] | N-((2,2-difluoro- benzo[d][1,3]dioxol- 5-yl)(8-hydroxy-5- methylquinolin-7- yl)methyl)-6-((2- (2,6-dioxopiperidin- 3-yl)-1,3-dioxoiso- indolin-4-yl)amino)- hexanamide |
| P46 | 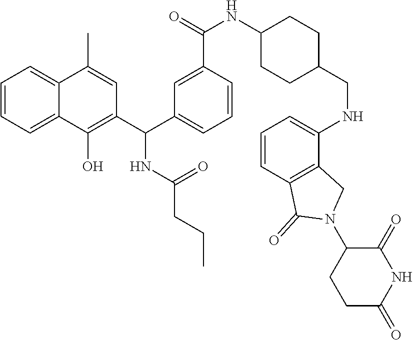 [SMILES: CCCC(=O)NC(c1cccc(C(=O)NC2CCC(CNc3cccc4c3CN(C3CCC(=O)NC3=O)C4=O)CC2)c1)c1cc(C)c2ccccc2c1O] | 3-(butyramido(8- hydroxy-5-methyl- quinolin-7-yl)meth- yl)-N-(4-(((2-(2,6- dioxopiperidin-3- yl)-1-oxoisoindolin- 4-yl)amino)meth- yl)cyclohexyl)benz- amide |
| P47 | 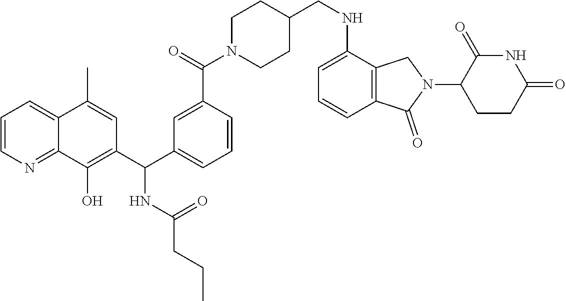 [SMILES: CCCC(=O)NC(c1cccc(C(=O)N2CCC(CNc3cccc4c3CN(C3CCC(=O)NC3=O)C4=O)CC2)c1)c1cc(C)c2cccnc2c1O] | N-((3-(4-(((2-(2,6- dioxopiperidin-3-yl)- 1-oxoisoindolin-4- yl)amino)methyl)- piperidine-1-carbon- yl)phenyl)(8- hydroxy-5-methyl- quinolin-7-yl)meth- yl)butyramide |
| P48 | 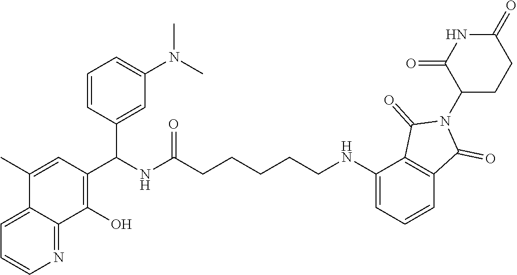 [SMILES: Cc1cc(C(NC(=O)CCCCCNc2cccc3c2C(=O)N(C2CCC(=O)NC2=O)C3=O)c2cccc(N(C)C)c2)c(O)c2ncccc12] | N-((3-(dimethyl- amino)phenyl)(8- hydroxy-5-methyl- quinolin-7-yl)meth- yl)-6-((2-(2,6- dioxopiperidin-3- yl)-1,3-dioxoiso- indolin-4-yl)amino)- hexanamide |
| P49 | 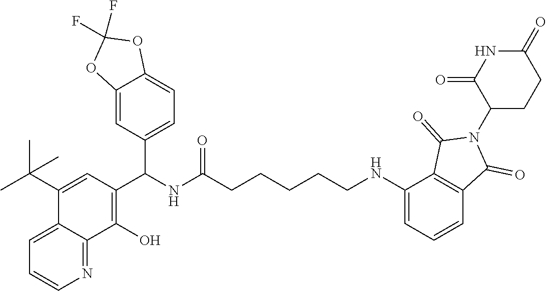 [SMILES: CC(C)(C)c1cc(C(NC(=O)CCCCCNc2cccc3c2C(=O)N(C2CCC(=O)NC2=O)C3=O)c2ccc3c(c2)OC(F)(F)O3)c(O)c2ncccc12] | N-((5-(tert-butyl)-8- hydroxyquinolin-7- yl)(2,2-difluoro- benzo[d][1,3]dioxol- 5-yl)methyl)-6-((2- (2,6-dioxopiperidin- 3-yl)-1,3-dioxoiso- indolin-4-yl)amino)- hexanamide |
| P50 | 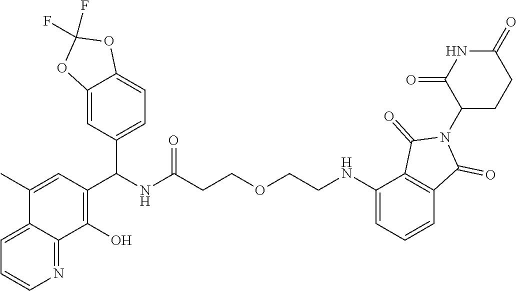 [SMILES: Cc1cc(C(NC(=O)CCOCCNc2cccc3c2C(=O)N(C2CCC(=O)NC2=O)C3=O)c2ccc3c(c2)OC(F)(F)O3)c(O)c2ncccc12] | N-((2,2-difluoro- benzo[d][1,3]dioxol- 5-yl)(8-hydroxy-5- methylquinolin-7- yl)methyl)-3-(2-((2- (2,6-dioxopiperidin- 3-yl)-1,3-dioxoiso- indolin-4-yl)amino)- ethoxy)propanamide |
| P51 | 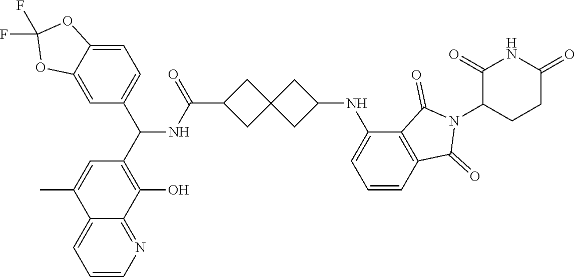 [SMILES: Cc1cc(C(NC(=O)C2CC3(CC(Nc4cccc5c4C(=O)N(C4CCC(=O)NC4=O)C5=O)C3)C2)c2ccc3c(c2)OC(F)(F)O3)c(O)c2ncccc12] | N-((2,2-difluoro- benzo[d][1,3]dioxol- 5-yl)(8-hydroxy-5- methylquinolin-7- yl)methyl)-6-((2- (2,6-dioxopiperidin- 3-yl)-1,3-dioxoiso- indolin-4-yl)amino)- spiro[3.3]heptane-2- carboxamide |
| P52 | 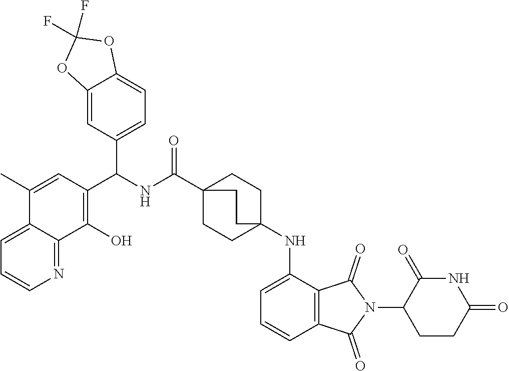 [SMILES: Cc1cc(C(NC(=O)C23CCC(Nc4cccc5c4C(=O)N(C4CCC(=O)NC4=O)C5=O)(CC2)CC3)c2ccc3c(c2)OC(F)(F)O3)c(O)c2ncccc12] | N-((2,2-difluoro- benzo[d][1,3]dioxol- 5-yl)(8-hydroxy-5- methylquinolin-7- yl)methyl)-4-((2- (2,6-dioxopiperidin- 3-yl)-1,3-dioxoiso- indolin-4-yl)amino)- bicyclo[2.2.2]octane- 1-carboxamide |
| P53 | 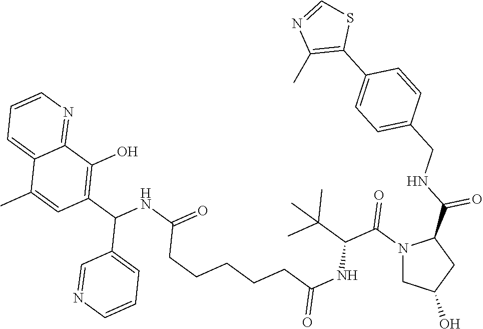 [SMILES: Cc1ncsc1-c1ccc(CNC(=O)[C@H]2C[C@H](O)CN2C(=O)[C@H](NC(=O)CCCCCC(=O)NC(c2cccnc2)c2cc(C)c3cccnc3c2O)C(C)(C)C)cc1] | N¹-((R)-1-((2R,4S)- 4-hydroxy-2-((4-(4- methylthiazol-5-yl)- benzyl)carbamoyl)- pyrrolidin-1-yl)-3,3- dimethyl-1-oxo- butan-2-yl)-N⁷-((8- hydroxy-5-methyl- quinolin-7-yl)(pyr- idin-3-yl)methyl)- heptanediamide |
| P54 | 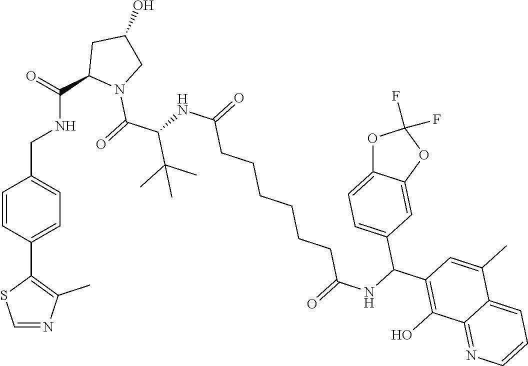 [SMILES: Cc1ncsc1-c1ccc(CNC(=O)[C@H]2C[C@H](O)CN2C(=O)[C@H](NC(=O)CCCCCCC(=O)NC(c2ccc3c(c2)OC(F)(F)O3)c2cc(C)c3cccnc3c2O)C(C)(C)C)cc1] | N¹-((2,2-difluoro- benzo[d][1,3]dioxol- 5-yl)(8-hydroxy-5- methylquinolin-7- yl)methyl)-N8-(2R- 1-((2R,4S)-4- hydroxy-2-((4-(4- methylthiazol-5-yl)- benzyl)carbamoyl)- pyrrolidine-1-yl)- 3,3-dimethyl-1-oxo- butan-2-yl)octane- diamide |
| P55 | 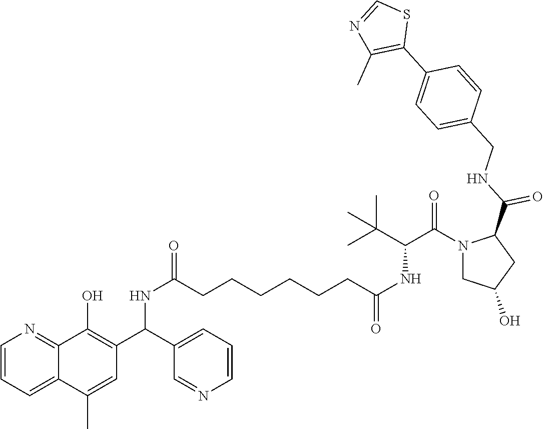 [SMILES: Cc1ncsc1-c1ccc(CNC(=O)[C@H]2C[C@H](O)CN2C(=O)[C@H](NC(=O)CCCCCCC(=O)NC(c2cccnc2)c2cc(C)c3cccnc3c2O)C(C)(C)C)cc1] | N¹-((R)-1-((2R,4S)- 4-hydroxy-2-((4-(4- methylthiazol-5-yl)- benzyl)carbamoyl)- pyrrolidin-1-yl)-3,3- dimethyl-1-oxo- butan-2-yl)-N8-((8- hydroxy-5-methyl- quinolin-7-yl)(pyr- idin-3-yl)methyl)- octanediamide |
| P56 | 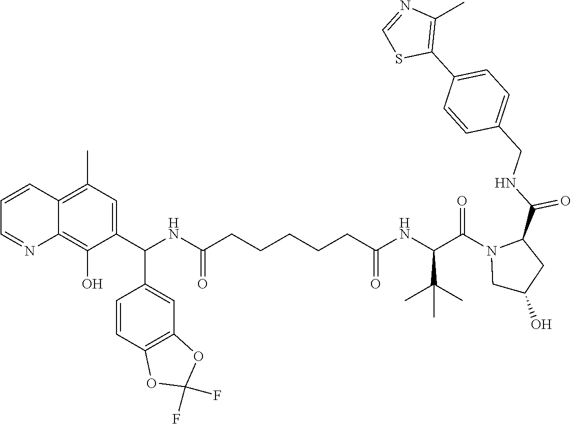 [SMILES: Cc1ncsc1-c1ccc(CNC(=O)[C@H]2C[C@H](O)CN2C(=O)[C@H](NC(=O)CCCCCC(=O)NC(c2ccc3c(c2)OC(F)(F)O3)c2cc(C)c3cccnc3c2O)C(C)(C)C)cc1] | N1-((2,2-difluoro- benzo[d][1,3]dioxol- 5-yl)(8-hydroxy-5- methylquinolin-7- yl)methyl)-N⁷-(R)-1- ((2R,4S)-4-hydroxy- 2-((4-(4-methyl- thiazol-5-yl)benzyl)- carbamoyl)pyrrol- idine-1-yl)-3,3- dimethyl-1-oxo- butan-2-yl)heptane- diamide |
| P57 | 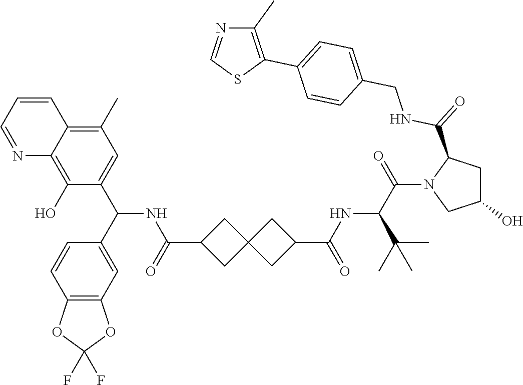 [SMILES: Cc1ncsc1-c1ccc(CNC(=O)[C@H]2C[C@H](O)CN2C(=O)[C@H](NC(=O)C2CC3(CC(C(=O)NC(c4ccc5c(c4)OC(F)(F)O5)c4cc(C)c5cccnc5c4O)C3)C2)C(C)(C)C)cc1] | N²-((2,2-difluoro- benzo[d][1,3]dioxol- 5-yl)(8-hydroxy-5- methylquinolin-7- yl)methyl)-N⁶-((R)- 1-((2R,4S)-4- hydroxy-2-((4-(4- methylthiazol-5-yl)- benzyl)carbamoyl)- pyrrolidin-1-yl)-3,3- dimethyl-1-oxo- butan-2-yl)spiro- [3.3]heptane-2,6- dicarboxamide |
| P58 | 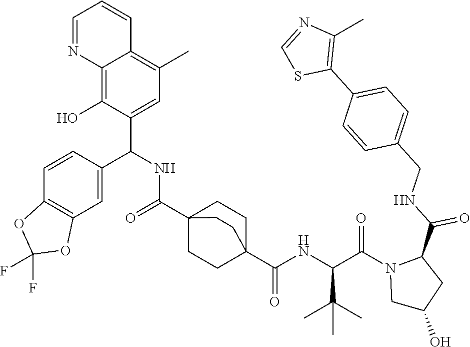 [SMILES: Cc1ncsc1-c1ccc(CNC(=O)[C@H]2C[C@H](O)CN2C(=O)[C@H](NC(=O)C23CCC(C(=O)NC(c4ccc5c(c4)OC(F)(F)O5)c4cc(C)c5cccnc5c4O)(CC2)CC3)C(C)(C)C)cc1] | N¹-((2,2-difluoro- benzo[d][1,3]dioxol- 5-yl)(8-hydroxy-5- methylquinolin-7- yl)methyl)-N⁴-((R)- 1-((2R,4S)-4- hydroxy-2-((4-(4- methylthiazol-5-yl)- benzyl)carbamoyl)- pyrrolidin-1-yl)-3,3- dimethyl-1-oxo- butan-2-yl)bicyclo- [2.2.2]octane-1,4- dicarboxamide |
| P59 | 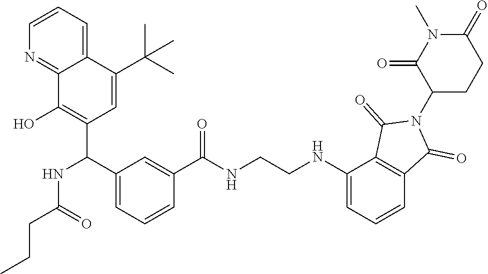 [SMILES: CCCC(=O)NC(c1cccc(C(=O)NCCNc2cccc3c2C(=O)N(C2CCC(=O)N(C)C2=O)C3=O)c1)c1cc(C(C)(C)C)c2cccnc2c1O] | 3-((5-(tert-butyl)-8- hydroxyquinolin-7- yl)(butyramido)- methyl)-N-(2-((2-(1- methyl-2,6-dioxo- piperidin-3-yl)-1,3- dioxoisoindolin-4- yl)amino)ethyl)- benzamide |
| P60 | 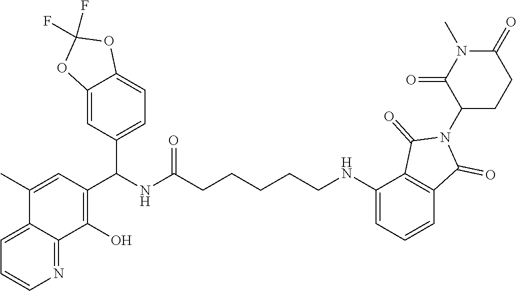 [SMILES: Cc1cc(C(NC(=O)CCCCCNc2cccc3c2C(=O)N(C2CCC(=O)N(C)C2=O)C3=O)c2ccc3c(c2)OC(F)(F)O3)c(O)c2ncccc12] | N-((2,2-difluoro- benzo[d][1,3]dioxol- 5-yl)(8-hydroxy-5- methylquinolin-7- yl)methyl)-6-((2-(1- methyl-2,6-dioxo- piperidin-3-yl)-1,3- dioxoisoindolin-4- yl)amino)hexan- amide |
| P61 | 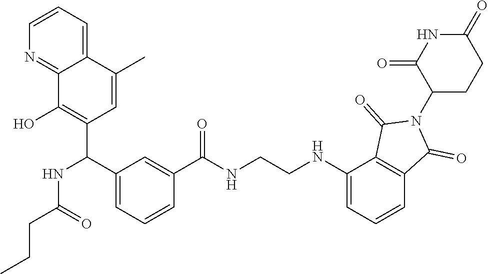 [SMILES: CCCC(=O)NC(c1cccc(C(=O)NCCNc2cccc3c2C(=O)N(C2CCC(=O)NC2=O)C3=O)c1)c1cc(C)c2cccnc2c1O] | 3-(butyramido(8- hydroxy-5-methyl- quinolin-7-yl)meth- yl)-N-(2-((2-(2,6- dioxopiperidin-3- yl)-1,3-dioxoiso- indolin-4-yl)amino)- ethyl)benzamide |
| P62 | 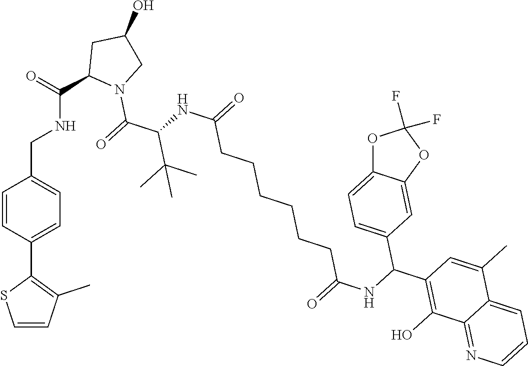 [SMILES: Cc1ccsc1-c1ccc(CNC(=O)[C@H]2C[C@@H](O)CN2C(=O)[C@H](NC(=O)CCCCCCC(=O)NC(c2ccc3c(c2)OC(F)(F)O3)c2cc(C)c3cccnc3c2O)C(C)(C)C)cc1] | N1-((2,2-difluoro- benzo[d][1,3]dioxol- 5-yl)(8-hydroxy-5- methylquinolin-7- yl)methyl)-N8-((R)- 1-((2R,4R)-4- hydroxy-2-((4-(4- methylthiazol-5-yl)- benzyl)carbamoyl)- pyrrolidin-1-yl)-3,3- dimethyl-1-oxo- butan-2-yl)octane- diamide |
| P63 | 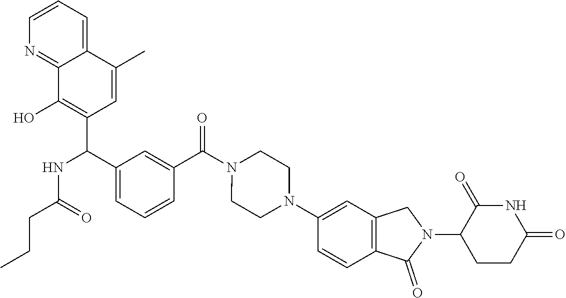 [SMILES: CCCC(=O)NC(c1cccc(C(=O)N2CCN(c3ccc4c(c3)CN(C3CCC(=O)NC3=O)C4=O)CC2)c1)c1cc(C)c2cccnc2c1O] | N-((3-(4-(2-(2,6- dioxopiperidin-3-yl)- 1-oxoisoindolin-5- yl)piperazine-1- carbonyl)phenyl)(8- hydroxy-5-methyl- quinolin-7-yl)meth- yl)butyramide |
| P64 | 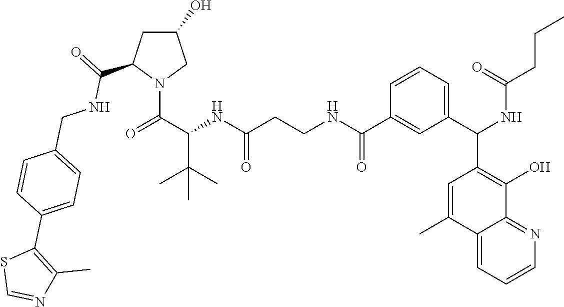 [SMILES: CCCC(=O)NC(c1cccc(C(=O)NCCC(=O)N[C@@H](C(=O)N2C[C@@H](O)C[C@@H]2C(=O)NCc2ccc(-c3scnc3C)cc2)C(C)(C)C)c1)c1cc(C)c2cccnc2c1O] | (2R,4S)-1-((2R)-2- (3-(3-(butyramido- (8-hydroxy-5-meth-ylquinolin-7-yl)- methyl)benzamido)- propanamido)-3,3- dimethylbutanoyl)- 4-hydroxy-N-(4-(4- methylthiazol-5-yl)- benzyl)pyrrolidine- 2-carboxamide |
| P65 | 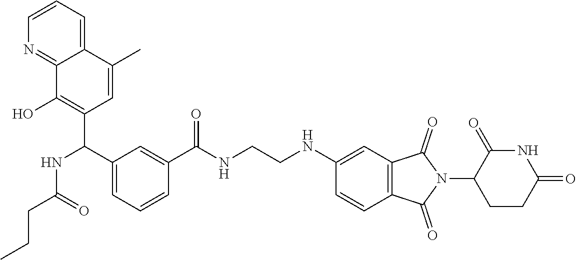 [SMILES: CCCC(=O)NC(c1cccc(C(=O)NCCNc2ccc3c(c2)C(=O)N(C2CCC(=O)NC2=O)C3=O)c1)c1cc(C)c2cccnc2c1O] | 3-(butyramido(8- hydroxy-5-methyl- quinolin-7-yl)meth- yl)-N-(2-((2-(2,6- dioxopiperidin-3-yl)- 1,3-dioxoisoindolin- 5-yl)amino)ethyl)- benzamide |
| P66 | 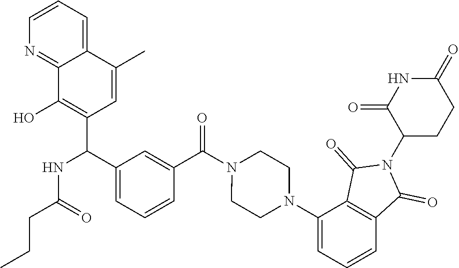 [SMILES: CCCC(=O)NC(c1cccc(C(=O)N2CCN(c3cccc4c3C(=O)N(C3CCC(=O)NC3=O)C4=O)CC2)c1)c1cc(C)c2cccnc2c1O] | N-((3-(4-(2-(2,6- dioxopiperidin-3- yl)-1,3-dioxoiso- indolin-4-yl)piper- azine-1-carbonyl)- phenyl)(8-hydroxy- 5-methylquinolin- 7-yl)methyl)butyr- amide |
| P67 | 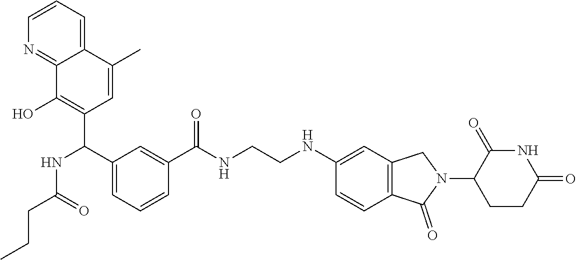 [SMILES: CCCC(=O)NC(c1cccc(C(=O)NCCNc2ccc3c(c2)CN(C2CCC(=O)NC2=O)C3=O)c1)c1cc(C)c2cccnc2c1O] | 3-(butyramido(8- hydroxy-5-methyl- quinolin-7-yl)meth- yl)-N-(2-((2-(2,6- dioxopiperidin-3- yl)-1-oxoisoindolin- 5-yl)amino)ethyl)- benzamide |
| P68 | 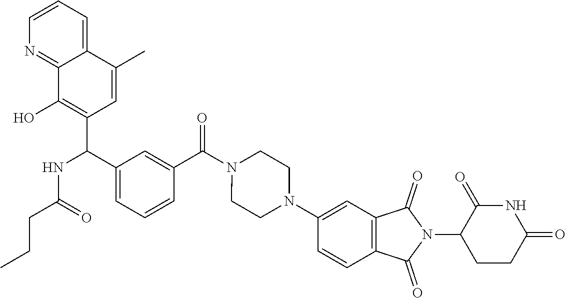 [SMILES: CCCC(=O)NC(c1cccc(C(=O)N2CCN(c3ccc4c(c3)C(=O)N(C3CCC(=O)NC3=O)C4=O)CC2)c1)c1cc(C)c2cccnc2c1O] | N-((3-(4-(2-(2,6- dioxopiperidin-3- yl)-1,3-dioxoiso- indolin-5-yl)piper- azine-1-carbonyl)- phenyl)(8-hydroxy- 5-methylquinolin- 7-yl)methyl)butyr- amide |

The invention further provides processes for preparing any of the compounds of the present invention.

The invention also provides the use of compounds to not only inhibit GRP78 activity but also signaling pathways dependent upon or related to GRP78. The invention also relates to the use of compounds for sensitizing cells to additional agent(s), such as agents known to be effective in the treatment of disorders related to GRP78 activity (e.g., cancer, viral infections, and inflammatory diseases).

The compounds of the invention are useful for the treatment, amelioration, or prevention of disorders associated with GRP78 activity (e.g., cancer, viral infections, and inflammatory diseases), such as those responsive to GRP78 activity inhibition. In certain embodiments, the compounds can be used to treat, ameliorate, or prevent cancer that is associated with GRP78 activity (e.g., pancreatic cancer, leukemia, colon cancer, CNS cancers (e.g. glioblastoma), non-small lung cancer, melanoma, ovarian cancer, renal cancer, breast cancer, prostate cancer, esophageal cancer, cervical cancer and colorectal cancer). In certain embodiments, the compounds can be used to treat, ameliorate, or prevent viral infections associated with GRP78 activity (e.g., SARS-CoV-2). In certain embodiments, the compounds can be used to treat, ameliorate, or prevent inflammatory diseases associated with GRP78 activity.

In certain embodiments, the present invention provides methods of treating, ameliorating, or preventing a disorder related to GRP78 activity in a patient comprising administering to said patient a therapeutically effective amount of the pharmaceutical composition comprising a compound recited in Tables I or II. In some embodiments, disorder related to GRP78 activity is a hyperproliferative condition and/or inflammatory condition. In some embodiments, the inflammatory condition is a chronic auto immune disorder and/or a viral infection such as SARS CoV-2. In some embodiments, the hyperproliferative condition is diabetes and/or cancer. In some embodiments, the cancer is one or more of leukemia, colon cancer, CNS cancers (e.g. glioblastoma), non-small lung cancer, melanoma, ovarian cancer, renal cancer, breast cancer, prostate cancer, esophageal cancer, cervical cancer and colorectal cancer. In some embodiments, patient is a human patient. In some embodiments, the method further comprises administering to said patient one or more agents for treating the disorder related to GRP78 activity. In some embodiments, the agents comprise topoisomerase I inhibitors and HDAC inhibitors. In some embodiments, the agents comprise anticancer agents, wherein said anticancer agent one or more of a chemotherapeutic agent, and radiation therapy. In some embodiments, administration of the compound results in induced ER stress-mediated apoptosis in the tumor cells implanted in mice without major toxicity to normal tissues. In some embodiments, administration of the compound induces ER stress and triggers UPR by inhibiting GRP78.

The invention also provides kits comprising a compound of the invention and instructions for administering the compound to an animal. The kits may optionally contain other therapeutic agents, e.g., agents useful in treating disorders related to GRP78 activity (e.g., cancer, viral infections, and inflammatory diseases).

### BRIEF DESCRIPTION OF THE DRAWINGS

FIG. 1: YUM70 is cytotoxic to pancreatic cancer cell lines. A. Chemical structure of YUM70. B. Cytotoxicity of YUM70 in a panel of pancreatic cancer cell lines and normal pancreatic tissue-derived cells (HPNE) measured by the MTT assay. IC₅₀ presented as mean±SD of three independent experiments performed in duplicate. C. YUM70 dose-dependently decreased PANC-1 and UM59 cell proliferation in 3D-culture systems. D. Quantification of cell viability of 3D spheroids was performed with CellTiter-Glo® 3D cell viability assay. Data are presented as mean±SD of three or more spheroids from three independent experiments. *p<0.01, **p<0.001, ***p<0.0001.

FIG. 2: YUM70 stabilized GRP78 full-length protein. Unfolding curves of GRP78 full-length protein at various concentrations of A. YUM70 B. VER and C. YUM117, in thermal shift assays. 1% DMSO was used as control. Lower panel: Apparent melting temperature (Tm) derived from thermal shift assay and the corresponding thermal shift were determined at various concentrations of A. YUM70 B. VER and C. YUM117. Data are presented as mean±SD. *p<0.01, **p<0.001, ***p<0.0001. Numbers in red are thermal shift at respective concentrations.

FIG. 3: GRP78 degradation by YUM-PROTAC. A. Chemical structure of DX2-145 (YUM70-PROTAC). B. Lysates of MIAPaCa-2 cells were immunoblotted with indicated antibodies. A representative experiment is shown. C. Degradation of GRP78 was quantified and presented as mean±SD from three independent experiments. D. Lysates of MIA PaCa-2 cells treated with DX2-145 in the presence or absence of MG132 (1 μM) pre-treatment (2 hr) were immunoblotted with indicated antibodies. E. Chemical structure of YUM513 (YUM401-PROTAC). F. Lysates of MIA PaCa-2 cells were immunoblotted with indicated antibodies. A representative experiment out of three independent experiments is shown. G. Degradation of GRP78 is quantified and presented as mean±SD of three independent experiments. The p-value was calculated against control using Student's t-test, *p<0.05, **p<0.01, ***p<0.001, H. MIA PaCa-2 cells were treated with YUM513 for the indicated times and lysate analyzed by SDS-PAGE-Coomassie staining. Two bands indicated by the red box were submitted separately to the UM Proteomics Core facility for proteomics analysis. I. One of the two proteins identified by mass spectrometry that showed over 2 fold degradation was confirmed as GRP78.

FIG. 4: YUM70 inhibits pancreatic tumor growth in vivo. A. Tumor growth curves of MIA PaCa-2 tumor-bearing nude mice treated with vehicle (n=5) or YUM70 (n=5). Data are shown as mean tumor volumes (error bars, SEM). A significant reduction in tumor volumes was observed upon YUM70 treatment (*p<0.05). B. Evaluation of mouse weights during the xenograft experiment. Error bars indicate mean±SEM. C. Ki67 immunohistochemistry staining in tumor sections. D. Percent of Ki67 positive cells were calculated as the fraction of Ki67 positive cells compared to the total number of cells in the field×100. (n=10; five fields of view from two tumors per group). Graphical data is presented as Mean±SD, ***p<0.0001. E. Lysate from two tumors per group was blotted for FAM129A, GRP78, CHOP, cleaved caspase 3. ‘M’ is the molecular weight marker. Normalized relative densities computed using ImageJ (NIH) are shown above. F. YUM70 did not show systemic toxicity in vivo. Representative micrographs of hematoxylin and eosin (H&E)-stained organ tissue sections. Images were taken with an Olympus IX83 inverted microscope at 20× magnification.

FIG. 5: Synergistic effect of YUM70 in combination with topotecan and vorinostat. MIA PaCa-2 cells were treated with YUM70 with or without topotecan (Topo) and vorinostat (SAHA), at stated concentrations and kept in culture until colonies were observed in DMSO treated control. A. A representative image is shown (one concentration). B and C. The number of colonies was quantified using Image Studio ver3.1 software from three independent experiments (more than one concentration). Graphical data is presented as mean±SD, *p<0.05, **p<0.01. The p-value of the combination was calculated and compared to YUM70 alone. D and E. The combined effect was calculated using CompuSyn software. CI<1 is defined as synergism. F. Combination regimen causes apoptosis in MIA PaCa-2 and PANC-1 cells. Top, cells in the bottom left quadrant of each panel (Annexin V-negative, PI-negative) are viable, whereas cells in the bottom right quadrant (Annexin V-positive, PI-negative) are in the early stage of apoptosis, and cells in the top right quadrant (Annexin V-positive, PI-positive) are in the late stage of apoptosis/necrosis. Bottom, the percentage of apoptotic cells is shown in a histogram. A representative image of three independent experiments is shown.

### DETAILED DESCRIPTION OF THE INVENTION

Experiments conducted during the course of developing embodiments for the present invention designed, synthesized and biologically evaluated compounds functioning as inhibitors GRP78 and their potential for use as therapeutics against disorders associated with GRP78 activity (e.g., cancer, viral infections, and inflammatory diseases). As such, the present invention addresses the need for effective therapies for disorders associated with GRP78 activity by providing potent and selective GRP78 inhibitors.

Indeed, experiments conducted during the course of developing embodiments for the present invention resulted in the development of a series of novel hydroxyquinolines targeting GRP78. The analog, YUM70, showed significant efficacy in a pancreatic cancer xenograft model with no detectable toxicity to normal tissues. YUM70 treatment upregulates ER stress-related genes, induces apoptosis, and demonstrates synergy with the FDA approved drugs topotecan and vorinostat in killing pancreatic cancer cells.

Accordingly, the present invention relates to inhibitors of GRP78 having Formula I (as defined herein) within cancer cells and/or immune cells, and which function as effective therapeutic agents for treating, ameliorating, and preventing various forms of (e.g., pancreatic cancer, leukemia, colon cancer, CNS cancers (e.g. glioblastoma), non-small lung cancer, melanoma, ovarian cancer, renal cancer, breast cancer, prostate cancer, esophageal cancer, cervical cancer and colorectal cancer), viral infections (e.g. SARS-CoV-2), and inflammatory diseases. In addition, this invention also relates to a new class of PROTACs having Formula II, III and IV (as defined herein) which function as degraders of GRP78 within cancer and/or immune cells. Pharmaceutical compositions comprising said compounds of Formulas I, II, III, or IV are also within the scope of the present invention.

In a particular embodiment, compounds encompassed within Formula I are provided:

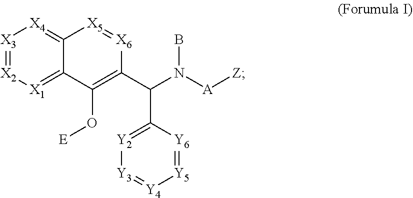

including pharmaceutically acceptable salts, solvates, and/or prodrugs thereof.

Formula I is not limited to a particular chemical moiety for X₁, X₂, X₃, X₄, X₅, X₆, Y₂, Y₃, Y₄, Y₅, Y₆, A, B, E and Z.

In some embodiments, the particular chemical moiety for X₁, X₂, X₃, X₄, X₅, X₆, Y₂, Y₃, Y₄, Y₅, Y₆, A, B, E and Z permits the resulting compound capable of one or more of.
inhibiting GRP78 activity;
serving as an effective therapeutic agent for treating, ameliorating, and preventing various forms of cancer, viral infections, and inflammatory diseases;
inducing ER stress-mediated apoptosis in the tumor cells implanted in mice without major toxicity to normal tissues; and
inducing ER stress and triggers UPR by inhibiting GRP78.

In some embodiments, X₁ is either CH or N.

In some embodiments, X₂, X₃, X₄, X₅ and X₆ are each independently selected from CR¹ or N, with the proviso that at least three of them must be CR¹.

In some embodiments, A is selected from CO, SO, and SO₂.

In some embodiments, Y², Y³, Y⁴, Y⁵, Y⁶ are each independently selected from CH, CR² and N.

In some embodiments, Y⁵ is a bond, in which case one of Y³, Y⁴, or Y⁶ is NR², 0, or S, while the other two may be CR² or N.

In some embodiments, B, E are each independently selected from hydrogen and R³.

In some embodiments, Z is R³.

In some embodiments, R¹ is independently selected from the group consisting of H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano, CH₂F, CHF₂, CF₃, OCF₃, SOR⁷, SO₂R⁷, NO₂, COR⁴, C₁₋₆ alkyl-COR⁴, N(R⁷)C₂₋₆ alkyl-NR⁷R⁷, —N(R⁷)C₂₋₆ alkyl-R⁴, N(C₂₋₆ alkyl)₂-NR⁷, —O(CH₂)ₚR⁴, —S(CH₂)ₚR⁴, and —N(R⁷)C(═O)(CH₂)ₚR⁴, with a proviso that not more than three R¹ can be other than H.

In some embodiments, R² is independently selected from the group consisting of H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano,

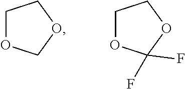 [SMILES: C1COCO1 ; FC1(F)OCCO1]

CF₃, OCF₃, SOR⁷, SO₂R⁷, NO₂, COR⁴, C₁₋₆ alkyl-COR⁴, N(R⁷)C₂₋₆ alkyl-NR⁷R⁷, N(C₂₋₆ alkyl)₂-NR⁷, CF₃, CO₂Et, CO₂H, R⁴, —N(R⁷)C₂₋₆ alkyl-R⁴, —O(CH₂)ₚR⁴, —S(CH₂)ₚR⁴, and —N(R⁷)C(═O)(CH₂)ₚR⁴, with a proviso that not more than two R² can be other than H.

In some embodiments, R³ is independently selected from the group consisting of C₁₋₆ alkyl, C₃₋₆ alkenyl, C₃₋₆ alkynyl, C₃₋₇ cycloalkyl, C₄₋₇ heterocycloalkyl, phenyl, naphthyl, 5-10 membered mono- or bicyclic heteroaryl, C₁₋₆ alkyl-C₃₋₇ cycloalkyl, C₁₋₆ alkyl-C₄₋₇ heterocycloalkyl, C₀₋₆ R⁴, and —N(R⁷)C₂₋₆ alkyl-R⁴.

In some embodiments, R⁴ is independently selected from the group consisting of OH, NR⁵R⁶, O(CH₂)_{q}NR⁵R⁶—, C₁₋₆ alkoxy, C₁₋₆ alkoxy-C₁₋₆ alkoxy, C₂₋₆ hydroxyalkoxy, cyclopropyl, oxetanyl, oxetanyloxy, oxetanylamino, oxolanyl, oxolanyloxy, oxolanylamino, oxanyl oxanyloxy, oxanylamino, oxepanyl, oxepanyloxy, oxepanylamino, azetidinyl, azetidinyloxy, azetidylamino, pyrrolidinyl, pyrolidinyloxy, pyrrolidinylamino, piperidinyl, piperidinyloxy, piperidinylamino, azepanyl, azepanyloxy, azepanylamino, dioxolanyl, dioxanyl, morpholino, thiomorpholino, thiomorpholino-S,S-dioxide, piperazino, dioxepanyl, dioxepanyloxy, dioxepanylamino, oxazepanyl, oxazepanyloxy, oxazepanylamino, diazepanyl, diazepanyloxy, and diazepanylamino, all of which may be optionally substituted with OH, OR⁷, oxo, halogen, R⁶, CH₂OR⁶, CH₂NR⁵R⁶ or CH₂CH₂CONR⁵R⁶.

In some embodiments, R⁵ and R⁶ are each independently selected from the group consisting of H, —CD₃, C₁₋₆ alkyl, C₃₋₆ alkenyl, C₃₋₆ alkynyl, C₃₋₈ cycloalkyl, —(C₁₋₃ alkyl)-(C₃₋₈ cycloalkyl), C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, and 5-11 membered heteroaryl; wherein R⁵ and R⁶ may be further independently substituted with up to three substituents selected from the group consisting of hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano, and halo; or alternatively, R⁵ and R⁶, taken together with the N atom to which they are both attached, may form a heterocycloalkyl ring of 4-7 members, containing up to one other heteroatom selected from O, S, or NR³, or a heterobicycloalkyl ring of 6-12 members which may be fused, bridged or spiro, and contain up to two other heteroatoms chosen from O, S(O)ₓ, or NR³.

In some embodiments, each R⁷ is independently selected from H, —CD₃, C₁₋₆ alkyl, C₃₋₆ cycloalkyl, phenyl, naphthyl, 5-10 membered mono- or bicyclo-heteroaryl, C₂₋₆ hydroxyalkyl, —SO₂-alkyl, NH—C₂₋₆ alkyl-NR⁵R⁶, C₁₋₆ alkoxy-C₁₋₆ alkyl, and C₂₋₆ alkyl-NR⁵R⁶; alternatively, two R⁷ taken together with the same N atom to which they are both attached, form a heterocyclic ring of 4-7 members, containing up to one other heteroatom selected from O, S, or NR³.

In some embodiments, p=0, 1, 2, 3, or 4.

In some embodiments, x=0, 1, or 2.

In some embodiments, compounds shown in Table I are contemplated for Formula I.

In a particular embodiment, compounds encompassed within Formula II are provided:

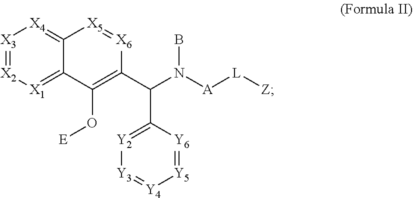

including pharmaceutically acceptable salts, solvates, and/or prodrugs thereof.

Formulas II is not limited to a particular chemical moiety for X₁, X₂, X₃, X₄, X₅, X₆, Y₂, Y₃, Y₄, Y₅, Y₆, A, B, E, L and Z. In some embodiments, the particular chemical moiety for X₁, X₂, X₃, X₄, X₅, X₆, Y₂, Y₃, Y₄, Y₅, Y₆, A, B, E, L and Z permits the resulting compound capable of one or more of:
inhibiting GRP78 activity;
serving as an effective therapeutic agent for treating, ameliorating, and preventing various forms of cancer, viral infections, and inflammatory diseases;
inducing ER stress-mediated apoptosis in the tumor cells implanted in mice without major toxicity to normal tissues; and
inducing ER stress and triggers UPR by inhibiting GRP78.

In some embodiments, X₁ is either CH or N.

In some embodiments, X₂, X₃, X₄, X₅ and X₆ are each independently selected from CR¹ and N, with the proviso that at least three of them must be CR¹.

In some embodiments, A is selected from the group consisting of CO, SO, and SO₂.

In some embodiments, Y², Y³, Y⁴, Y⁵, Y⁶ are each independently selected from the group consisting of CH, CR² and N.

In some embodiments, Y⁵ is a bond, in which case one of Y³, Y⁴, or Y⁶ is NR², 0, or S, while the other two may be CR² or N.

In some embodiments, B, E are each independently selected from H and R³.

In some embodiments, R¹ is independently selected from the group consisting of H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano, CF₃, OCF₃, SOR⁷, SO₂R⁷, NO₂, COR⁴, C₁₋₆ alkyl-COR⁴, N(R⁷)C₂₋₆ alkyl-NR⁷R⁷, —N(R⁷)C₂₋₆ alkyl-R⁴, N(C₂₋₆ alkyl)₂-NR⁷, —O(CH₂)ₚR⁴, —S(CH₂)ₚR⁴, and —N(R⁷)C(═O)(CH₂)ₚR⁴, with a proviso that not more than three R¹ can be other than H.

In some embodiments, R² is independently selected from the group consisting of H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano,

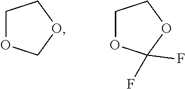 [SMILES: C1COCO1 ; FC1(F)OCCO1]

CF₃, OCF₃, SOR⁷, SO₂R⁷, NO₂, COR⁴, C₁₋₆ alkyl-COR⁴, N(R⁷)C₂₋₆ alkyl-NR⁷R⁷, N(C₂₋₆ alkyl)₂-NR⁷, CF₃, CO₂Et, CO₂H, —N(R⁷)C₂₋₆ alkyl-R⁴, —O(CH₂)ₚR⁴, —S(CH₂)ₚR⁴, and —N(R⁷)C(═O)(CH₂)ₚR⁴, with a proviso that not more than two R² can be other than H.

In some embodiments, R³ is independently selected from the group consisting of C₁₋₆ alkyl, C₃₋₆ alkenyl, C₃₋₆ alkynyl, C₃₋₇ cycloalkyl, C₄₋₇ heterocycloalkyl, phenyl, naphthyl, 5-10 membered mono- or bicyclic heteroaryl, C₁₋₆ alkyl-C₃₋₇ cycloalkyl, C₁₋₆ alkyl-C₄₋₇ heterocycloalkyl, and C₁₋₆ R⁴.

In some embodiments, R⁴ is independently selected from the group consisting of OH, NR⁵R⁶, O(CH₂)_{q}NR⁵R⁶, C₁₋₆ alkoxy, C₁₋₆ alkoxy-C₁₋₆ alkoxy, C₂₋₆ hydroxyalkoxy, cyclopropyl, oxetanyl, oxetanyloxy, oxetanylamino, oxolanyl, oxolanyloxy, oxolanylamino, oxanyl oxanyloxy, oxanylamino, oxepanyl, oxepanyloxy, oxepanylamino, azetidinyl, azetidinyloxy, azetidylamino, pyrrolidinyl, pyrolidinyloxy, pyrrolidinylamino, piperidinyl, piperidinyloxy, piperidinylamino, azepanyl, azepanyloxy, azepanylamino, dioxolanyl, dioxanyl, morpholino, thiomorpholino, thiomorpholino-S,S-dioxide, piperazino, dioxepanyl, dioxepanyloxy, dioxepanylamino, oxazepanyl, oxazepanyloxy, oxazepanylamino, diazepanyl, diazepanyloxy, and diazepanylamino, all of which may be optionally substituted with OH, OR⁷, oxo, halogen, R⁶, CH₂OR⁶, CH₂NR⁵R⁶ or CH₂CH₂CONR⁵R⁶.

In some embodiments, R⁵ and R⁶ are each independently selected from the group consisting of H, —CD₃, C₁₋₆ alkyl, C₃₋₆ alkenyl, C₃₋₆ alkynyl, C₃₋₈ cycloalkyl, —(C₁₋₃ alkyl)-(C₃-8 cycloalkyl), C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, and 5-11 membered heteroaryl; wherein R⁵ and R⁶ may be further independently substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo; or alternatively, R⁵ and R⁶, taken together with the N atom to which they are both attached, form a heterocycloalkyl ring of 4-7 members, containing up to one other heteroatom selected from O, S, or NR³, or a heterobicycloalkyl ring of 6-12 members which may be fused, bridged or spiro, and contain up to two other heteroatoms chosen from O, S(O)ₓ, or NR³.

In some embodiments, each R⁷ is independently selected from the group consisting of H, —CD₃, C₁₋₆ alkyl, C₃₋₆ cycloalkyl, phenyl, naphthyl, 5-10 membered mono- or bicyclo-heteroaryl, C₂₋₆ hydroxyalkyl, —SO₂-alkyl, NH—C₂₋₆ alkyl-NR⁵R⁶, C₁₋₆ alkoxy-C₁₋₆ alkyl, and C₂₋₆ alkyl-NR⁵R⁶; alternatively, two R⁷ taken together with the same N atom to which they are both attached, form a heterocyclic ring of 4-7 members, containing up to one other heteroatom selected from O, S, or NR³.

In some embodiments, p=0, 1, 2, 3, or 4.

In some embodiments, x=0, 1, or 2.

In some embodiments L is a linker selected from a group consisting of —(CH₂)m-, —(CH₂CH₂O)n-,

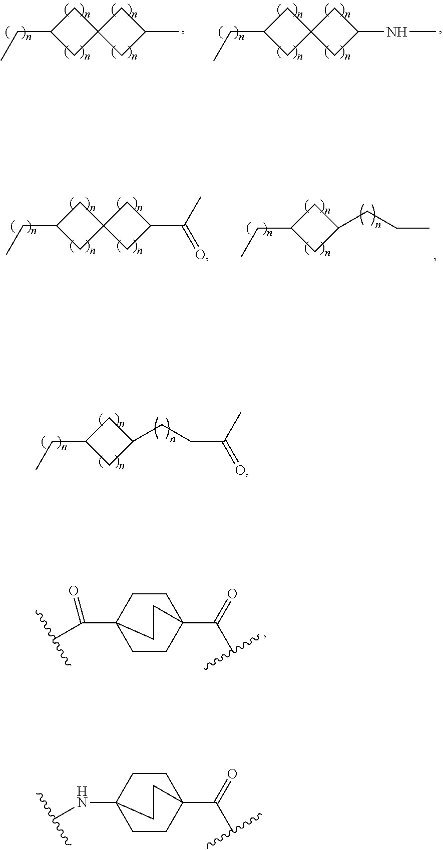

—(CH₂)ₘC≡C, —(CH₂CH₂O)ₙC≡C; wherein m=0, 1, 2, 3, 4, 5, 6, 7, 8, 9, or 10; and wherein n=0, 1, 2, 3, 4, 5, or 6.

In some embodiments, Z is a radical of an E3 ligase ligand selected from the group consisting of.

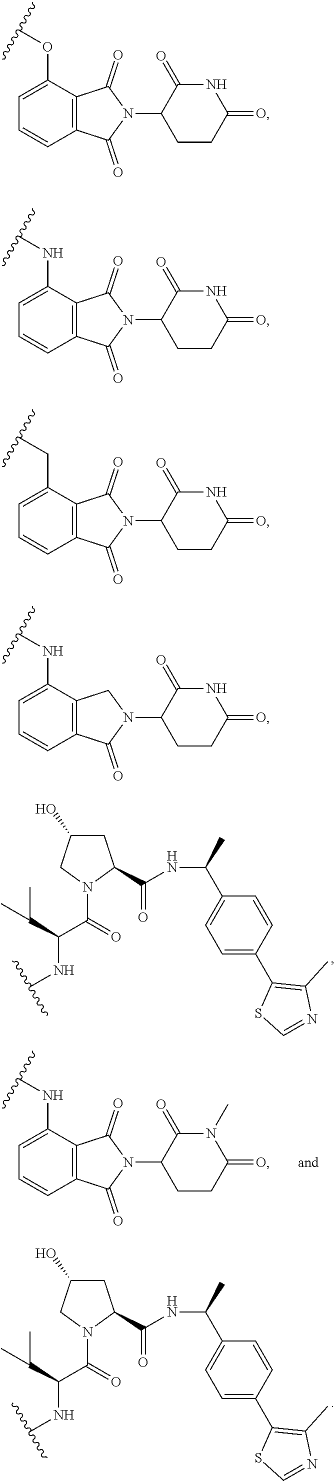

In a particular embodiment, compounds encompassed within Formula III are provided:

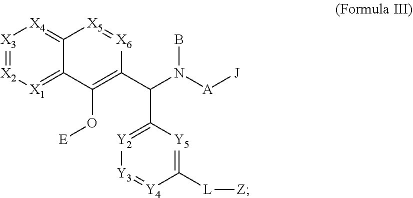

including pharmaceutically acceptable salts, solvates, and/or prodrugs thereof.

Formula III is not limited to a particular chemical moiety for X₁, X₂, X₃, X₄, X₅, X₆, Y₂, Y₃, Y₄, Y₅, A, B, E, J, L and Z. In some embodiments, the particular chemical moiety for X₁, X₂, X₃, X₄, X₅, X₆, Y₂, Y₃, Y₄, Y₅, A, B, E, J, L and Z permits the resulting compound capable of one or more of
inhibiting GRP78 activity;
serving as an effective therapeutic agent for treating, ameliorating, and preventing various forms of cancer, viral infections, and inflammatory diseases;
inducing ER stress-mediated apoptosis in the tumor cells implanted in mice without major toxicity to normal tissues; and
inducing ER stress and triggers UPR by inhibiting GRP78.

In some embodiments, X₁ is either CH or N.

In some embodiments, X₂, X₃, X₄, X₅ and X₆ are each independently selected from CR¹ and N, with the proviso that at least three of them must be CR¹.

In some embodiments, A is selected from CO, SO, and SO₂.

In some embodiments, Y₂, Y₃, Y₄, Y₅ are independently selected from CH, CR² and N.

In some embodiments, B, E and J are each independently selected from H and R³.

In some embodiments, R¹ is independently selected from the group consisting of H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano, CF₃, OCF₃, SOR⁷, SO₂R⁷, NO₂, COR⁴, C₁₋₆ alkyl-COR⁴, N(R⁷)C₂₋₆ alkyl-NR⁷R⁷, —N(R⁷)C₂₋₆ alkyl-R⁴, N(C₂₋₆ alkyl)₂-NR⁷, —O(CH₂)ₚR⁴, —S(CH₂)ₚR⁴, and —N(R⁷)C(═O)(CH₂)ₚR⁴, with a proviso that not more than three R¹ can be other than H.

In some embodiments, R² is independently selected from the group consisting of H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano, CF₃, OCF₃, SOR⁷, SO₂R⁷, NO₂, COR⁴, C₁₋₆ alkyl-COR⁴, N(R⁷)C₂₋₆ alkyl-NR⁷R⁷, N(C₂₋₆ alkyl)₂-NR⁷, CF₃, CO₂Et, CO₂H, —N(R⁷)C₂₋₆ alkyl-R⁴, —O(CH₂)ₚR⁴, —S(CH₂)ₚR⁴, and —N(R⁷)C(═O)(CH₂)ₚR⁴, with a proviso that not more than two R² can be other than H.

In some embodiments, R³ is independently selected from the group consisting of C₁₋₆ alkyl, C₃₋₆ alkenyl, C₃₋₆ alkynyl, C₃₋₇ cycloalkyl, C₄₋₇ heterocycloalkyl, phenyl, naphthyl, 5-10 membered mono- or bicyclic heteroaryl, C₁₋₆ alkyl-C₃₋₇ cycloalkyl, or C₁₋₆ alkyl-C₄₋₇ heterocycloalkyl, and C₁₋₆ R⁴.

In some embodiments, R⁴ is independently selected from the group consisting of OH, NR⁵R⁶, O(CH₂)_{q}NR⁵R⁶, C₁₋₆ alkoxy, C₁₋₆ alkoxy-C₁₋₆ alkoxy, C₂₋₆ hydroxyalkoxy, cyclopropyl, oxetanyl, oxetanyloxy, oxetanylamino, oxolanyl, oxolanyloxy, oxolanylamino, oxanyl oxanyloxy, oxanylamino, oxepanyl, oxepanyloxy, oxepanylamino, azetidinyl, azetidinyloxy, azetidylamino, pyrrolidinyl, pyrolidinyloxy, pyrrolidinylamino, piperidinyl, piperidinyloxy, piperidinylamino, azepanyl, azepanyloxy, azepanylamino, dioxolanyl, dioxanyl, morpholino, thiomorpholino, thiomorpholino-S,S-dioxide, piperazino, dioxepanyl, dioxepanyloxy, dioxepanylamino, oxazepanyl, oxazepanyloxy, oxazepanylamino, diazepanyl, diazepanyloxy, and diazepanylamino, all of which may be optionally substituted with OH, OR⁷, oxo, halogen, R⁶, CH₂OR⁶, CH₂NR⁵R⁶ or CH₂CH₂CONR⁵R⁶.

In some embodiments, R⁵ and R⁶ are each independently selected from the group consisting of H, —CD₃, C₁₋₆ alkyl, C₃₋₆ alkenyl, C₃₋₆ alkynyl, C₃₋₈ cycloalkyl, —(C₁₋₃ alkyl)-(C₃-8 cycloalkyl), C₃₋₈ cycloalkenyl, C₁-C6 acyl, 4-12 membered monocyclic or bicyclic heterocyclyl, 4-12 membered monocyclic or bicyclic heterocyclyl-C₁-C₆ alkyl-, C6-C12 aryl, and 5-11 membered heteroaryl; wherein R⁵ and R⁶ may be further independently substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo; or alternatively, R⁵ and R⁶, taken together with the N atom to which they are both attached, form a heterocycloalkyl ring of 4-7 members, containing up to one other heteroatom selected from O, S, or NR³, or a heterobicycloalkyl ring of 6-12 members which may be fused, bridged or spiro, and contain up to two other heteroatoms chosen from O, S(O)ₓ, or NR³.

In some embodiments, each R⁷ is independently selected from the group consisting of H, —CD₃, C₁₋₆ alkyl, C₃₋₆ cycloalkyl, phenyl, naphthyl, 5-10 membered mono- or bicyclo-heteroaryl, C₂₋₆ hydroxyalkyl, —SO₂-alkyl, NH—C₂₋₆ alkyl-NR⁵R⁶, C₁₋₆ alkoxy-C₁₋₆ alkyl, and C₂₋₆ alkyl-NR⁵R⁶; alternatively, two R⁷ taken together with the same N atom to which they are both attached, form a heterocyclic ring of 4-7 members, containing up to one other heteroatom selected from O, S, or NR³.

In some embodiments, p=0, 1, 2, 3, or 4.

In some embodiments, x=0, 1, or 2.

In some embodiments, L is a linker selected from a group consisting of —(CH₂)ₘ—, —NH(CH₂)ₘ—, —NH(CH₂CH₂O)ₙ—, —(CH₂CH₂O)ₙ—, —(CH₂)ₘCO—, —(CH₂CH₂O)ₙCO—,

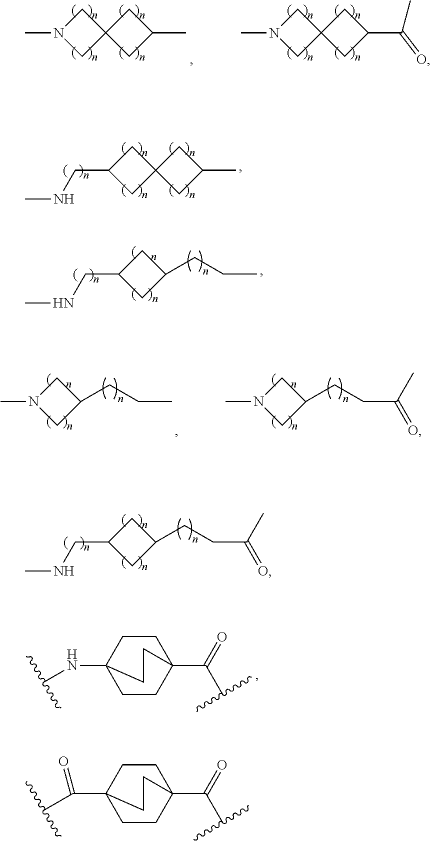

—NH(CH₂)ₘC≡C, and —NH(CH₂CH₂O)ₙC≡C; wherein m=0, 1, 2, 3, 4, 5, 6, 7, 8, 9, or 10; and wherein n=0, 1, 2, 3, 4, 5, or 6.

In some embodiments, Z is a radical of an E3 ligase ligand selected from the group consisting of.

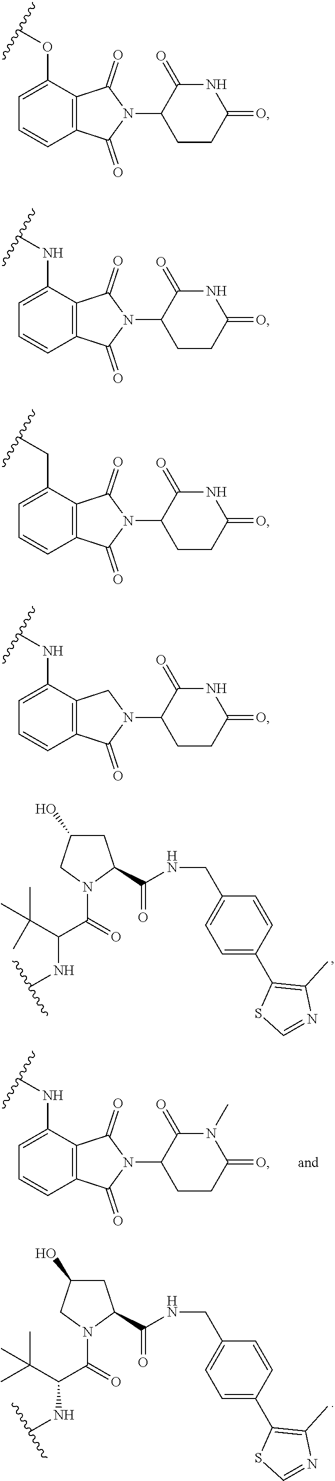

In a particular embodiment, compounds encompassed within Formula IV are provided:

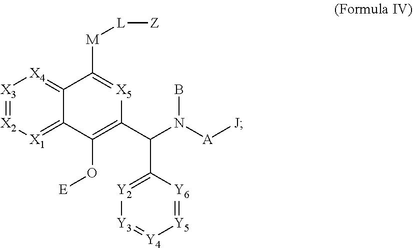

including pharmaceutically acceptable salts, solvates, and/or prodrugs thereof.

Formula IV is not limited to a particular chemical moiety for X₁, X₂, X₃, X₄, X₅, Y₂, Y₃, Y₄, Y₅, Y₆, A, B, E, M, J, L and Z. In some embodiments, the particular chemical moiety for X₁, X₂, X₃, X₄, X₅, Y₂, Y₃, Y₄, Y₅, Y₆, A, B, E, M, J, L and Z permits the resulting compound capable of one or more of
inhibiting GRP78 activity;
serving as an effective therapeutic agent for treating, ameliorating, and preventing various forms of cancer, viral infections, and inflammatory diseases;
inducing ER stress-mediated apoptosis in the tumor cells implanted in mice without major toxicity to normal tissues; and
inducing ER stress and triggers UPR by inhibiting GRP78.

In some embodiments, X₁ is either CH or N.

In some embodiments, X₂, X₃, X₄, X₅ are independently selected from CR¹ and N, with the proviso that at least three of them must be CR¹.

In some embodiments, A is selected from the group consisting of CO, SO, and SO₂.

In some embodiments, Y₂, Y₃, Y₄, Y₅ are independently selected from the group consisting of CH, CR² or N.

In some embodiments, M is selected from the group consisting of NH and CO.

In some embodiments, B, E and J are each independently selected from the group consisting of H and R³.

In some embodiments, R¹ is independently selected from the group consisting of H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano, CF₃, OCF₃, SOR⁷, SO₂R⁷, NO₂, COR⁴, C₁₋₆ alkyl-COR⁴, N(R⁷)C₂₋₆ alkyl-NR⁷R⁷, —N(R⁷)C₂₋₆ alkyl-R⁴, N(C₂₋₆ alkyl)₂-NR⁷, —O(CH₂)ₚR⁴, —S(CH₂)ₚR⁴, and —N(R⁷)C(═O)(CH₂)ₚR⁴, with a proviso that not more than three R¹ can be other than H.

In some embodiments, R² is independently selected from the group consisting of H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano,

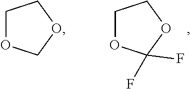 [SMILES: C1COCO1 ; FC1(F)OCCO1]

CF₃, OCF₃, SOR⁷, SO₂R⁷, NO₂, COR⁴, C₁₋₆ alkyl-COR⁴, N(R⁷)C₂₋₆ alkyl-NR⁷R⁷, N(C₂₋₆ alkyl)₂-NR⁷, CF₃, CO₂Et, CO₂H, —N(R⁷)C₂₋₆ alkyl-R⁴, —O(CH₂)ₚR⁴, —S(CH₂)ₚR⁴, and —N(R⁷)C(═O)(CH₂)ₚR⁴, with a proviso that not more than two R² can be other than H.

In some embodiments, R³ is independently selected from the group consisting of C₁₋₆ alkyl, C₃₋₆ alkenyl, C₃₋₆ alkynyl, C₃₋₇ cycloalkyl, C₄₋₇ heterocycloalkyl, phenyl, naphthyl, 5-10 membered mono- or bicyclic heteroaryl, C₁₋₆ alkyl-C₃₋₇ cycloalkyl, C₁₋₆ alkyl-C₄₋₇ heterocycloalkyl, and C₁₋₆ R⁴.

In some embodiments, R⁴ is independently selected from the group consisting of OH, NR⁵R⁶, O(CH₂)_{q}NR⁵R⁶, C₁₋₆ alkoxy, C₁₋₆ alkoxy-C₁₋₆ alkoxy, C₂₋₆ hydroxyalkoxy, cyclopropyl, oxetanyl, oxetanyloxy, oxetanylamino, oxolanyl, oxolanyloxy, oxolanylamino, oxanyl oxanyloxy, oxanylamino, oxepanyl, oxepanyloxy, oxepanylamino, azetidinyl, azetidinyloxy, azetidylamino, pyrrolidinyl, pyrolidinyloxy, pyrrolidinylamino, piperidinyl, piperidinyloxy, piperidinylamino, azepanyl, azepanyloxy, azepanylamino, dioxolanyl, dioxanyl, morpholino, thiomorpholino, thiomorpholino-S,S-dioxide, piperazino, dioxepanyl, dioxepanyloxy, dioxepanylamino, oxazepanyl, oxazepanyloxy, oxazepanylamino, diazepanyl, diazepanyloxy, and diazepanylamino, all of which may be optionally substituted with OH, OR⁷, oxo, halogen, R⁶, CH₂OR⁶, CH₂NR⁵R⁶ or CH₂CH₂CONR⁵R⁶.

In some embodiments, R⁵ and R⁶ are each independently selected from the group consisting of H, —CD₃, C₁₋₆ alkyl, C₃₋₆ alkenyl, C₃₋₆ alkynyl, C₃₋₈ cycloalkyl, —(C₁₋₃ alkyl)-(C₃₋₈ cycloalkyl), C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, and 5-11 membered heteroaryl; wherein R⁵ and R⁶ may be further independently substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo; or alternatively, R⁵ and R⁶, taken together with the N atom to which they are both attached, form a heterocycloalkyl ring of 4-7 members, containing up to one other heteroatom selected from O, S, or NR³, or a heterobicycloalkyl ring of 6-12 members which may be fused, bridged or spiro, and contain up to two other heteroatoms chosen from O, S(O)ₓ, or NR³.

In some embodiments, each R⁷ is independently selected from the group consisting of H, —CD₃, C₁₋₆ alkyl, C₃₋₆ cycloalkyl, phenyl, naphthyl, 5-10 membered mono- or bicyclo-heteroaryl, C₂₋₆ hydroxyalkyl, —SO₂-alkyl, NH—C₂₋₆ alkyl-NR⁵R⁶, C₁₋₆ alkoxy-C₁₋₆ alkyl, and C₂₋₆ alkyl-NR⁵R⁶; alternatively, two R⁷ taken together with the same N atom to which they are both attached, form a heterocyclic ring of 4-7 members, containing up to one other heteroatom selected from O, S, or NR³.

In some embodiments, p=0, 1, 2, 3, or 4.

In some embodiments, x=0, 1, or 2.

In some embodiments, L is a linker selected from a group consisting of —CO(CH₂)ₘ—, —NH(CH₂)ₘ—, —NH(CH₂CH₂O)ₙ—, —CO(CH₂CH₂O)ₙ—, —NH(CH₂)ₘCO—,

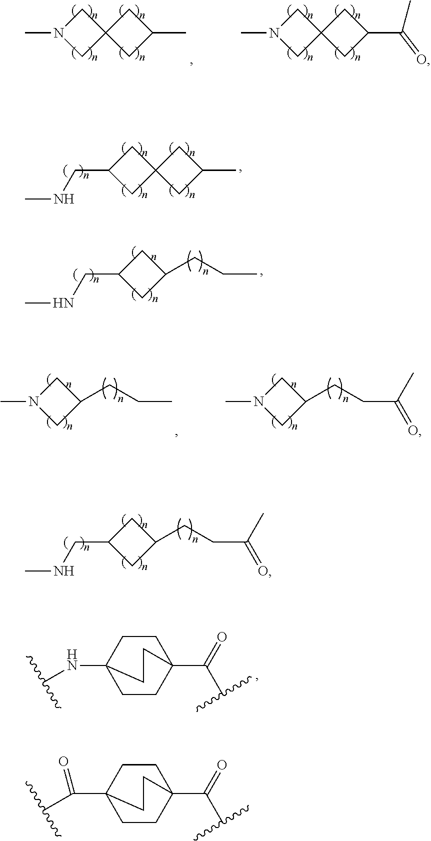

—NH(CH₂)ₘC≡C, —NH(CH₂CH₂O)ₙC≡C —CO(CH₂)ₘC≡C, and —CO(CH₂CH₂O)ₙC≡C; wherein m=0, 1, 2, 3, 4, 5, 6, 7, 8, 9, or 10, and wherein n=0, 1, 2, 3, 4, 5, or 6.

In some embodiments, Z is a radical of an E3 ligase ligand selected from the group consisting of:

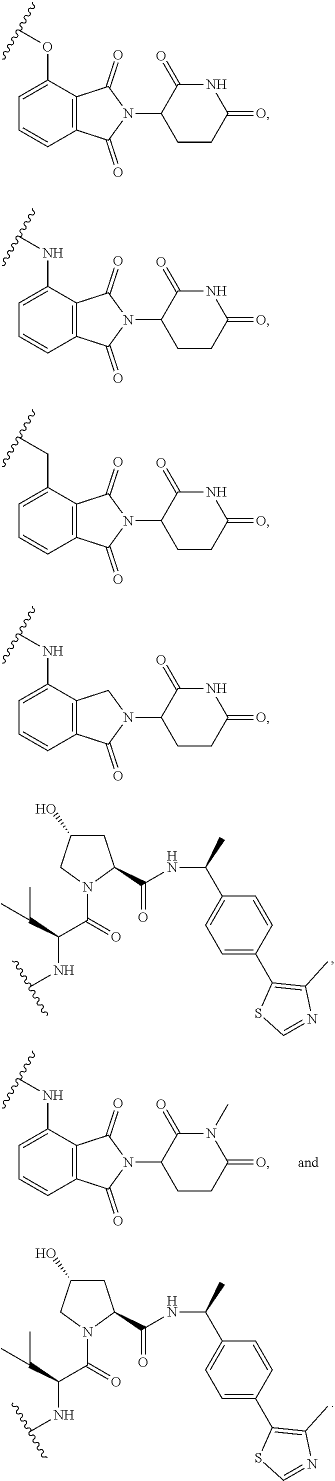

In some embodiments, compounds shown in Table II are contemplated for Formulas II, III and IV.

In some embodiments, compounds shown in Table II are contemplated for Formulas II, III and IV.

In some embodiments, the compositions and methods of the present invention are used to treat diseased cells, tissues, organs, or pathological conditions and/or disease states in an animal (e.g., a mammalian patient including, but not limited to, humans and veterinary animals). In this regard, various diseases and pathologies are amenable to treatment or prophylaxis using the present methods and compositions. A non-limiting exemplary list of these diseases and conditions includes, but is not limited to, cancer associated with GRP78 activity (e.g., pancreatic cancer, leukemia, colon cancer, CNS cancers (e.g. glioblastoma), non-small lung cancer, melanoma, ovarian cancer, renal cancer, breast cancer, prostate cancer, esophageal cancer, cervical cancer and colorectal cancer), viral infections associated with GRP78 activity (e.g., SARS-CoV-2), inflammatory diseases associated with GRP78 activity, and any type of condition related to GRP78 activity.

A non-limiting exemplary list of cancers include, but are not limited to, pancreatic cancer, breast cancer, prostate cancer, lymphoma, skin cancer, colon cancer, melanoma, malignant melanoma, ovarian cancer, brain cancer, primary brain carcinoma, head and neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma, and the like. In some embodiments, the cancer cells being treated are metastatic. In other embodiments, the cancer cells being treated are resistant to anticancer agents.

Some embodiments of the present invention provide methods for administering an effective amount of a compound of the invention and at least one additional therapeutic agent (including, but not limited to, chemotherapeutic antineoplastics, apoptosis-modulating agents, antimicrobials, antivirals, antifungals, and anti-inflammatory agents) and/or therapeutic technique (e.g., surgical intervention, and/or radiotherapies).

A number of suitable anticancer agents are contemplated for use in the methods of the present invention. Indeed, the present invention contemplates, but is not limited to, administration of numerous anticancer agents such as: agents that induce apoptosis; polynucleotides (e.g., anti-sense, ribozymes, siRNA); polypeptides (e.g., enzymes and antibodies); biological mimetics; alkaloids; alkylating agents; antitumor antibiotics; antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-α) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteosome inhibitors: NF-KB modulators; anti-CDK compounds; HDAC inhibitors; and the like. Numerous other examples of chemotherapeutic compounds and anticancer therapies suitable for co-administration with the disclosed compounds are known to those skilled in the art.

In certain embodiments, anticancer agents comprise agents that induce or stimulate apoptosis. Agents that induce apoptosis include, but are not limited to, radiation (e.g., X-rays, gamma rays, UV); tumor necrosis factor (TNF)-related factors (e.g., TNF family receptor proteins, TNF family ligands, TRAIL, antibodies to TRAIL-R1 or TRAIL-R2); kinase inhibitors (e.g., epidermal growth factor receptor (EGFR) kinase inhibitor, vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; antibodies (e.g., HERCEPTIN, RITUXAN, ZEVALIN, and AVASTIN); anti-estrogens (e.g., raloxifene and tamoxifen); antiandrogens (e.g., flutamide, bicalutamide, finasteride, aminoglutethamide, ketoconazole, and corticosteroids); cyclooxygenase 2 (COX-2) inhibitors (e.g., celecoxib, meloxicam, NS-398, and non-steroidal anti-inflammatory drugs (NSAIDs)); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine (DTIC), dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine, and the like.

In still other embodiments, the compositions and methods of the present invention provide a compound of the invention and at least one anti-hyperproliferative or antineoplastic agent selected from alkylating agents, antimetabolites, and natural products (e.g., herbs and other plant and/or animal derived compounds).

Alkylating agents suitable for use in the present compositions and methods include, but are not limited to: 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3) alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (DTIC; dimethyltriazenoimid-azolecarboxamide).

In some embodiments, antimetabolites suitable for use in the present compositions and methods include, but are not limited to: 1) folic acid analogs (e.g., methotrexate (amethopterin)); 2) pyrimidine analogs (e.g., fluorouracil (5-fluorouracil; 5-FU), floxuridine (fluorode-oxyuridine; FudR), and cytarabine (cytosine arabinoside)); and 3) purine analogs (e.g., mercaptopurine (6-mercaptopurine; 6-MP), thioguanine (6-thioguanine; TG), and pentostatin (2′-deoxycoformycin)).

In still further embodiments, chemotherapeutic agents suitable for use in the compositions and methods of the present invention include, but are not limited to: 1) vinca alkaloids (e.g., vinblastine (VLB), vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin (cis-DDP) and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide).

Any oncolytic agent that is routinely used in a cancer therapy context finds use in the compositions and methods of the present invention. For example, the U.S. Food and Drug Administration maintains a formulary of oncolytic agents approved for use in the United States. International counterpart agencies to the U.S.F.D.A. maintain similar formularies. Table 3 provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

TABLE 3

| | | |
|---|---|---|
| Aldesleukin | Proleukin | Chiron Corp., Emeryville, CA |
| (des-alanyl-1, serine-125 human interleukin-2) | | |
| Alemtuzumab | Campath | Millennium and ILEX |
| (IgG1κ anti CD52 antibody) | | Partners, LP, Cambridge, MA |
| Alitretinoin | Panretin | Ligand Pharmaceuticals, Inc., |
| (9-cis-retinoic acid) | | San Diego CA |
| Allopurinol | Zyloprim | GlaxoSmithKline, Research |
| (1,5-dihydro-4 H-pyrazolo[3,4-d]pyrimidin-4-one | | Triangle Park, NC |
| monosodium salt) | | |
| Altretamine | Hexalen | US Bioscience, West |
| (N,N,N′,N′,N″,N″,-hexamethyl-1,3,5-triazine-2,4,6- | | Conshohocken, PA |
| triamine) | | |
| Amifostine | Ethyol | US Bioscience |
| (ethanethiol, 2-[(3-aminopropyl)amino]-, dihydrogen | | |
| phosphate (ester)) | | |
| Anastrozole | Arimidex | AstraZeneca Pharmaceuticals, |
| (1,3-Benzenediacetonitrile, a,a,a′,a′-tetramethyl-5-(1H- | | LP, Wilmington, DE |
| 1,2,4-triazol-1-ylmethyl)) | | |
| Arsenic trioxide | Trisenox | Cell Therapeutic, Inc., Seattle, |
| | | WA |
| Asparaginase | Elspar | Merck & Co., Inc., |
| (L-asparagine amidohydrolase, type EC-2) | | Whitehouse Station, NJ |
| BCG Live | TICE BCG | Organon Teknika, Corp., |
| (lyophilized preparation of an attenuated strain of | | Durham, NC |
| Mycobacterium bovis (Bacillus Calmette-Gukin [BCG], | | |
| substrain Montreal) | | |
| bexarotene capsules | Targretin | Ligand Pharmaceuticals |
| (4-[1-(5,6,7,8-tetrahydro-3,5,5,8,8-pentamethyl-2- | | |
| napthalenyl) ethenyl] benzoic acid) | | |
| bexarotene gel | Targretin | Ligand Pharmaceuticals |
| Bleomycin | Blenoxane | Bristol-Myers Squibb Co., |
| (cytotoxic glycopeptide antibiotics produced by | | NY, NY |
| Streptomyces verticillus; bleomycin A₂ and | | |
| bleomycin B₂) | | |
| Capecitabine | Xeloda | Roche |
| (5′-deoxy-5-fluoro-N-[(pentyloxy)carbonyl]-cytidine) | | |
| Carboplatin | Paraplatin | Bristol-Myers Squibb |
| (platinum, diammine [1,1-cyclobutanedicarboxylato(2-)- | | |
| 0,0′]-, (SP-4-2)) | | |
| Carmustine | BCNU, BiCNU | Bristol-Myers Squibb |
| (1,3-bis(2-chloroethyl)-1-nitrosourea) | | |
| Carmustine with Polifeprosan 20 Implant | Gliadel Wafer | Guilford Pharmaceuticals, |
| | | Inc., Baltimore, MD |
| Celecoxib | Celebrex | Searle Pharmaceuticals, |
| (as 4-[5-(4-methylphenyl)-3-(trifluoromethyl)-1H- | | England |
| pyrazol-1-yl] benzenesulfonamide) | | |
| Chlorambucil | Leukeran | GlaxoSmithKline |
| (4-[bis(2chlorethyl)amino]benzenebutanoic acid) | | |
| Cisplatin | Platinol | Bristol-Myers Squibb |
| (PtCl₂H₆N₂) | | |
| Cladribine | Leustatin, 2-CdA | R. W. Johnson Pharmaceutical |
| (2-chloro-2′-deoxy-b-D-adenosine) | | Research Institute, Raritan, NJ |
| Cyclophosphamide | Cytoxan, Neosar | Bristol-Myers Squibb |
| (2-[bis(2-chloroethyl)amino] tetrahydro-2H-13,2- | | |
| oxazaphosphorine 2-oxide monohydrate) | | |
| Cytarabine | Cytosar-U | Pharmacia & Upjohn |
| (1-b-D-Arabinofuranosylcytosine, C₉H₁₃N₃O₅) | | Company |
| cytarabine liposomal | DepoCyt | Skye Pharmaceuticals, Inc., |
| | | San Diego, CA |
| Dacarbazine | DTIC-Dome | Bayer AG, Leverkusen, |
| (5-(3,3-dimethyl-1-triazeno)-imidazole-4-carboxamide | | Germany |
| (DTIC)) | | |
| Dactinomycin, actinomycin D | Cosmegen | Merck |
| (actinomycin produced by Streptomyces parvullus, | | |
| C₆₂H₈₆N₁₂O₁₆) | | |
| Darbepoetin alfa | Aranesp | Amgen, Inc., Thousand Oaks, |
| (recombinant peptide) | | CA |
| daunorubicin liposomal | DanuoXome | Nexstar Pharmaceuticals, Inc., |
| ((8S-cis)-8-acetyl-10-[(3-amino-2,3,6-trideoxy-á-L-lyxo- | | Boulder, CO |
| hexopyranosyl)oxy]-7,8,9,10-tetrahydro-6,8,11- | | |
| trihydroxy-1-methoxy-5,12-naphthacenedione | | |
| hydrochloride) | | |
| Daunorubicin HCl, daunomycin | Cerubidine | Wyeth Ayerst, Madison, NJ |
| ((1S,3S)-3-Acetyl-1,2,3,4,6,11-hexahydro-3,5,12- | | |
| trihydroxy-10-methoxy-6,11-dioxo-1-naphthacenyl 3- | | |
| amino-2,3,6-trideoxy-(alpha)-L-lyxo-hexopyranoside | | |
| hydrochloride) | | |
| Denileukin diftitox | Ontak | Seragen, Inc., Hopkinton, MA |
| (recombinant peptide) | | |
| Dexrazoxane | Zinecard | Pharmacia & Upjohn |
| ((S)-4,4′-(1-methyl-1,2-ethanediyl)bis-2,6- | | Company |
| piperazinedione) | | |
| Docetaxel | Taxotere | Aventis Pharmaceuticals, Inc., |
| ((2R,3S)-N-carboxy-3-phenylisoserine, N-tert-butyl ester, | | Bridgewater, NJ |
| 13-ester with 5b-20-epoxy-12a,4,7b,10b,13a- | | |
| hexahydroxytax-11-en-9-one 4-acetate 2-benzoate, | | |
| trihydrate) | | |
| Doxorubicin HCl | Adriamycin, Rubex | Pharmacia & Upjohn |
| (8S,10S)-10-[(3-amino-2,3,6-trideoxy-a-L-lyxo- | | Company |
| hexopyranosyl)oxy]-8-glycolyl-7,8,9,10-tetrahydro- | | |
| 6,8,11-trihydroxy-1-methoxy-5,12-naphthacenedione | | |
| hydrochloride) | | |
| doxorubicin | Adriamycin PFS | Pharmacia & Upjohn |
| | Intravenous injection | Company |
| doxorubicin liposomal | Doxil | Sequus Pharmaceuticals, Inc., |
| | | Menlo park, CA |
| dromostanolone propionate | Dromostanolone | Eli Lilly & Company, |
| (17b-Hydroxy-2a-methyl-5a-androstan-3-one propionate) | | Indianapolis, IN |
| dromostanolone propionate | Masterone injection | Syntex, Corp., Palo Alto, CA |
| Elliott's B Solution | Elliott's B Solution | Orphan Medical, Inc |
| Epirubicin | Ellence | Pharmacia & Upjohn |
| ((8S-cis)-10-[(3-amino-2,3,6-trideoxy-a-L-arabino- | | Company |
| hexopyranosyl)oxy]-7,8,9,10-tetrahydro-6,8,11- | | |
| trihydroxy-8-(hydroxyacetyl)-1-methoxy-5,12- | | |
| naphthacenedione hydrochloride) | | |
| Epoetin alfa | Epogen | Amgen, Inc |
| (recombinant peptide) | | |
| Estramustine | Emcyt | Pharmacia & Upjohn |
| (estra-1,3,5(10)-triene-3,17-diol(17(beta))-, 3-[bis(2- | | Company |
| chloroethyl)carbamate] 17-(dihydrogen phosphate), | | |
| disodium salt, monohydrate, or estradiol 3-[bis(2- | | |
| chloroethyl)carbamate] 17-(dihydrogen phosphate), | | |
| disodium salt, monohydrate) | | |
| Etoposide phosphate | Etopophos | Bristol-Myers Squibb |
| (4′-Demethylepipodophyllotoxin 9-[4,6-O-(R)- | | |
| ethylidene-(beta)-D-glucopyranoside], 4′-(dihydrogen | | |
| phosphate)) | | |
| etoposide, VP-16 | Vepesid | Bristol-Myers Squibb |
| (4′-demethylepipodophyllotoxin 9-[4,6-0-(R)-ethylidene- | | |
| (beta)-D-glucopyranoside]) | | |
| Exemestane | Aromasin | Pharmacia & Upjohn |
| (6-methylenandrosta-1,4-diene-3,17-dione) | | Company |
| Filgrastim | Neupogen | Amgen, Inc |
| (r-metHuG-CSF) | | |
| floxuridine (intraarterial) | FUDR | Roche |
| (2′-deoxy-5-fluorouridine) | | |
| Fludarabine | Fludara | Berlex Laboratories, Inc., |
| (fluorinated nucleotide analog of the antiviral agent | | Cedar Knolls, NJ |
| vidarabine, 9-b-D-arabinofuranosyladenine (ara-A)) | | |
| Fluorouracil, 5-FU | Adrucil | ICN Pharmaceuticals, Inc., |
| (5-fluoro-2,4(1H,3H)-pyrimidinedione) | | Humacao, Puerto Rico |
| Fulvestrant | Faslodex | IPR Pharmaceuticals, |
| (7-alpha-[9-(4,4,5,5,5-penta fluoropentylsulphinyl) | | Guayama, Puerto Rico |
| nonyl]estra-1,3,5-(10)-triene-3,17-beta-diol) | | |
| Gemcitabine | Gemzar | Eli Lilly |
| (2′-deoxy-2′,2′-difluorocytidine monohydrochloride (b- | | |
| isomer)) | | |
| Gemtuzumab Ozogamicin | Mylotarg | Wyeth Ayerst |
| (anti-CD33 hP67.6) | | |
| Goserelin acetate | Zoladex Implant | AstraZeneca Pharmaceuticals |
| Hydroxyurea | Hydrea | Bristol-Myers Squibb |
| Ibritumomab Tiuxetan | Zevalin | Biogen IDEC, Inc., |
| (immunoconjugate resulting from a thiourea covalent | | Cambridge MA |
| bond between the monoclonal antibody Ibritumomab and | | |
| the linker-chelator tiuxetan [N-[2- | | |
| bis(carboxymethyl)amino]-3-(p-isothiocyanatophenyl)- | | |
| propyl]-[N-[2-bis(carboxymethyl)amino]-2-(methyl)- | | |
| ethyl]glycine) | | |
| Idarubicin | Idamycin | Pharmacia & Upjohn |
| (5,12-Naphthacenedione, 9-acetyl-7-[(3-amino-2,3,6- | | Company |
| trideoxy-(alpha)-L-lyxo-hexopyranosyl)oxy]-7,8,9,10- | | |
| tetrahydro-6,9,11-trihydroxyhydrochloride, (7S-cis)) | | |
| Ifosfamide | IFEX | Bristol-Myers Squibb |
| (3-(2-chloroethyl)-2-[(2-chloroethyl)amino]tetrahydro- | | |
| 2H-1,3,2-oxazaphosphorine 2-oxide) | | |
| Imatinib Mesilate | Gleevec | Novartis AG, Basel, |
| (4-[(4-Methyl-1-piperazinyl)methyl]-N-[4-methyl-3-[[4- | | Switzerland |
| (3-pyridinyl)-2-pyrimidinyl]amino]-phenyl]benzamide | | |
| methanesulfonate) | | |
| Interferon alfa-2a | Roferon-A | Hoffmann-La Roche, Inc., |
| (recombinant peptide) | | Nutley, NJ |
| Interferon alfa-2b | Intron A (Lyophilized | Schering AG, Berlin, |
| (recombinant peptide) | Betaseron) | Germany |
| Irinotecan HCl | Camptosar | Pharmacia & Upjohn |
| ((4S)-4,11-diethyl-4-hydroxy-9-[(4- | | Company |
| piperidinopiperidino)carbonyloxy]-1H-pyrano[3′,4′:6,7] | | |
| indolizino[1,2-b] quinoline-3,14(4H,12H) | | |
| dione hydrochloride trihydrate) | | |
| Letrozole | Femara | Novartis |
| (4,4′-(1H-1,2,4-Triazol-1-ylmethylene) dibenzonitrile) | | |
| Leucovorin | Wellcovorin, Leucovorin | Immunex, Corp., Seattle, WA |
| (L-Glutamic acid, N[4[[(2amino-5-formyl-1,4,5,6,7,8 | | |
| hexahydro4oxo6-pteridinyl)methyl]amino]benzoyl], | | |
| calcium salt (1:1)) | | |
| Levamisole HCl | Ergamisol | Janssen Research Foundation, |
| ((−)-(S)-2,3,5,6-tetrahydro-6-phenylimidazo [2,1-b] | | Titusville, NJ |
| thiazole monohydrochloride C₁₁H₁₂N₂S•HCl) | | |
| Lomustine | CeeNU | Bristol-Myers Squibb |
| (1-(2-chloro-ethyl)-3-cyclohexyl-1-nitrosourea) | | |
| Meclorethamine, nitrogen mustard | Mustargen | Merck |
| (2-chloro-N-(2-chloroethyl)-N-methylethanamine | | |
| hydrochloride) | | |
| Megestrol acetate | Megace | Bristol-Myers Squibb |
| 17α(acetyloxy)-6-methylpregna-4,6-diene-3,20-dione | | |
| Melphalan, L-PAM | Alkeran | GlaxoSmithKline |
| (4-[bis(2-chloroethyl) amino]-L-phenylalanine) | | |
| Mercaptopurine, 6-MP | Purinethol | GlaxoSmithKline |
| (1,7-dihydro-6 H-purine-6-thione monohydrate) | | |
| Mesna | Mesnex | Asta Medica |
| (sodium 2-mercaptoethane sulfonate) | | |
| Methotrexate | Methotrexate | Lederle Laboratories |
| (N-[4-[[(2,4-diamino-6- | | |
| pteridinyl)methyl]methylamino]benzoyl]-L-glutamic | | |
| acid) | | |
| Methoxsalen | Uvadex | Therakos, Inc., Way Exton, Pa |
| (9-methoxy-7H-furo[3,2-g][1]-benzopyran-7-one) | | |
| Mitomycin C | Mutamycin | Bristol-Myers Squibb |
| mitomycin C | Mitozytrex | SuperGen, Inc., Dublin, CA |
| Mitotane | Lysodren | Bristol-Myers Squibb |
| (1,1-dichloro-2-(o-chlorophenyl)-2-(p-chlorophenyl) | | |
| ethane) | | |
| Mitoxantrone | Novantrone | Immunex Corporation |
| (1,4-dihydroxy-5,8-bis[[2-[(2- | | |
| hydroxyethyl)amino]ethyl]amino]-9,10-anthracenedione | | |
| dihydrochloride) | | |
| Nandrolone phenpropionate | Durabolin-50 | Organon, Inc., West Orange, |
| | | NJ |
| Nofetumomab | Verluma | Boehringer Ingelheim Pharma |
| | | KG, Germany |
| Oprelvekin | Neumega | Genetics Institute, Inc., |
| (IL-11) | | Alexandria, VA |
| Oxaliplatin | Eloxatin | Sanofi Synthelabo, Inc., NY, NY |
| (cis-[(1R,2R)-1,2-cyclohexanediamine-N,N′] | | |
| [oxalato(2-)-O,O′] platinum) | | |
| Paclitaxel | TAXOL | Bristol-Myers Squibb |
| (5β,20-Epoxy-1,2a,4,7β,10β,13a-hexahydroxytax-11- | | |
| en-9-one 4,10-diacetate 2-benzoate 13-ester with (2R,3S)- | | |
| N-benzoyl-3-phenylisoserine) | | |
| Pamidronate | Aredia | Novartis |
| (phosphonic acid (3-amino-1-hydroxypropylidene) bis-, | | |
| disodium salt, pentahydrate, (APD)) | | |
| Pegademase | Adagen (Pegademase | Enzon Pharmaceuticals, Inc., |
| ((monomethoxypolyethylene glycol succinimidyl) 11-17- | Bovine) | Bridgewater, NJ |
| adenosine deaminase) | | |
| Pegaspargase | Oncaspar | Enzon |
| (monomethoxypolyethylene glycol succinimidyl | | |
| L-asparaginase) | | |
| Pegfilgrastim | Neulasta | Amgen, Inc |
| (covalent conjugate of recombinant methionyl human G- | | |
| CSF (Filgrastim) and monomethoxypolyethylene glycol) | | |
| Pentostatin | Nipent | Parke-Davis Pharmaceutical |
| | | Co., Rockville, MD |
| Pipobroman | Vercyte | Abbott Laboratories, Abbott |
| | | Park, IL |
| Plicamycin, Mithramycin | Mithracin | Pfizer, Inc., NY, NY |
| (antibiotic produced by Streptomyces plicatus) | | |
| Porfimer sodium | Photofrin | QLT Phototherapeutics, Inc., |
| | | Vancouver, Canada |
| Procarbazine | Matulane | Sigma Tau Pharmaceuticals, |
| (N-isopropyl-μ-(2-methylhydrazino)-p-toluamide | | Inc., Gaithersburg, MD |
| monohydrochloride) | | |
| Quinacrine | Atabrine | Abbott Labs |
| (6-chloro-9-(1-methyl-4-diethyl-amine) butylamino-2- | | |
| methoxyacridine) | | |
| Rasburicase | Elitek | Sanofi-Synthelabo, Inc., |
| (recombinant peptide) | | |
| Rituximab | Rituxan | Genentech, Inc., South San |
| (recombinant anti-CD20 antibody) | | Francisco, CA |
| Sargramostim | Prokine | Immunex Corp |
| (recombinant peptide) | | |
| Streptozocin | Zanosar | Pharmacia & Upjohn |
| (streptozocin 2-deoxy-2- | | Company |
| [[(methylnitrosoamino)carbonyl]amino]-a(and b)-D- | | |
| glucopyranose and 220 mg citric acid anhydrous) | | |
| Talc | Sclerosol | Bryan, Corp., Woburn, MA |
| (Mg₃Si₄O₁₀ (OH)₂) | | |
| Tamoxifen | Nolvadex | AstraZeneca Pharmaceuticals |
| ((Z)2-[4-(1,2-diphenyl-1-butenyl) phenoxy]-N,N- | | |
| dimethylethanamine 2-hydroxy-1,2,3- | | |
| propanetricarboxylate (1:1)) | | |
| Temozolomide | Temodar | Schering |
| (3,4-dihydro-3-methyl-4-oxoimidazo[5,1-d]-as-tetrazine- | | |
| 8-carboxamide) | | |
| teniposide, VM-26 | Vumon | Bristol-Myers Squibb |
| (4′-demethylepipodophyllotoxin 9-[4,6-0-(R)-2- | | |
| thenylidene-(beta)-D-glucopyranoside]) | | |
| Testolactone | Teslac | Bristol-Myers Squibb |
| (13-hydroxy-3-oxo-13,17-secoandrosta-1,4-dien-17-oic | | |
| acid [dgr]-lactone) | | |
| Thioguanine, 6-TG | Thioguanine | GlaxoSmithKline |
| (2-amino-1,7-dihydro-6 H-purine-6-thione) | | |
| Thiotepa | Thioplex | Immunex Corporation |
| (Aziridine,1,1′,1″-phosphinothioylidynetris-, or Tris (1- | | |
| aziridinyl) phosphine sulfide) | | |
| Topotecan HCl | Hycamtin | GlaxoSmithKline |
| ((S)-10-[(dimethylamino) methyl]-4-ethyl-4,9-dihydroxy- | | |
| 1H-pyrano[3′,4′:6,7] indolizino [1,2-b] quinoline-3,14- | | |
| 4H,12H)-dione monohydrochloride) | | |
| Toremifene | Fareston | Roberts Pharmaceutical Corp., |
| (2-(p-[(Z)-4-chloro-1,2-diphenyl-1-butenyl]-phenoxy)- | | Eatontown, NJ |
| N,N-dimethylethylamine citrate (1:1)) | | |
| Tositumomab, I 131 Tositumomab | Bexxar | Corixa Corp., Seattle, WA |
| (recombinant murine immunotherapeutic monoclonal | | |
| IgG₂ₐ lambda anti-CD20 antibody (I 131 is a | | |
| radioimmunotherapeutic antibody)) | | |
| Trastuzumab | Herceptin | Genentech, Inc |
| (recombinant monoclonal IgG₁ kappa anti-HER2 | | |
| antibody) | | |
| Tretinoin, ATRA | Vesanoid | Roche |
| (all-trans retinoic acid) | | |
| Uracil Mustard | Uracil Mustard Capsules | Roberts Labs |
| Valrubicin, N-trifluoroacetyladriamycin-14-valerate | Valstar | Anthra --> Medeva |
| ((2S-cis)-2-[1,2,3,4,6,11-hexahydro-2,5,12-trihydroxy-7 | | |
| methoxy-6,11-dioxo-[[4 2,3,6-trideoxy-3- | | |
| [(trifluoroacetyl)-amino-α-L-lyxo-hexopyranosyl]oxyl]-2- | | |
| naphthacenyl]-2-oxoethyl pentanoate) | | |
| Vinblastine, Leurocristine | Velban | Eli Lilly |
| (C₄₆H₅₆N₄O₁₀•H₂SO₄) | | |
| Vincristine | Oncovin | Eli Lilly |
| (C₄₆H₅₆N₄O₁₀•H₂SO₄) | | |
| Vinorelbine | Navelbine | GlaxoSmithKline |
| (3′,4′-didehydro-4′-deoxy-C′-norvincaleukoblastine [R- | | |
| (R*,R*)-2,3-dihydroxybutanedioate (1:2)(salt)]) | | |
| Zoledronate, Zoledronic acid | Zometa | Novartis |
| ((1-Hydroxy-2-imidazol-1-yl-phosphonoethyl) | | |
| phosphonic acid monohydrate) | | |

Anticancer agents further include compounds which have been identified to have anticancer activity. Examples include, but are not limited to, 3-AP, 12-O-tetradecanoylphorbol-13-acetate, 17AAG, 852A, ABI-007, ABR-217620, ABT-751, ADI-PEG 20, AE-941, AG-013736, AGRO100, alanosine, AMG 706, antibody G250, antineoplastons, AP23573, apaziquone, APC8015, atiprimod, ATN-161, atrasenten, azacitidine, BB-10901, BCX-1777, bevacizumab, BG00001, bicalutamide, BMS 247550, bortezomib, bryostatin-1, buserelin, calcitriol, CCI-779, CDB-2914, cefixime, cetuximab, CG0070, cilengitide, clofarabine, combretastatin A4 phosphate, CP-675,206, CP-724,714, CpG 7909, curcumin, decitabine, DENSPM, doxercalciferol, E7070, E7389, ecteinascidin 743, efaproxiral, eflornithine, EKB-569, enzastaurin, erlotinib, exisulind, fenretinide, flavopiridol, fludarabine, flutamide, fotemustine, FR901228, G17DT, galiximab, gefitinib, genistein, glufosfamide, GTI-2040, histrelin, HKI-272, homoharringtonine, HSPPC-96, hu14.18-interleukin-2 fusion protein, HuMax-CD4, iloprost, imiquimod, infliximab, interleukin-12, IPI-504, irofulven, ixabepilone, lapatinib, lenalidomide, lestaurtinib, leuprolide, LMB-9 immunotoxin, lonafarnib, luniliximab, mafosfamide, MB07133, MDX-010, MLN2704, monoclonal antibody 3F8, monoclonal antibody J591, motexafin, MS-275, MVA-MUC1-IL2, nilutamide, nitrocamptothecin, nolatrexed dihydrochloride, nolvadex, NS-9, O6-benzylguanine, oblimersen sodium, ONYX-015, oregovomab, OSI-774, panitumumab, paraplatin, PD-0325901, pemetrexed, PHY906, pioglitazone, pirfenidone, pixantrone, PS-341, PSC 833, PXD101, pyrazoloacridine, R115777, RAD001, ranpirnase, rebeccamycin analogue, rhuAngiostatin protein, rhuMab 2C₄, rosiglitazone, rubitecan, S-1, 5-8184, satraplatin, SB-, 15992, SGN-0010, SGN-40, sorafenib, SR31747A, ST1571, SU011248, suberoylanilide hydroxamic acid, suramin, talabostat, talampanel, tariquidar, temsirolimus, TGFa-PE38 immunotoxin, thalidomide, thymalfasin, tipifarnib, tirapazamine, TLK286, trabectedin, trimetrexate glucuronate, TroVax, UCN-1, valproic acid, vinflunine, VNP40101M, volociximab, vorinostat, VX-680, ZD1839, ZD6474, zileuton, and zosuquidar trihydrochloride.

The present invention provides methods for administering a compound of the invention with radiation therapy. The invention is not limited by the types, amounts, or delivery and administration systems used to deliver the therapeutic dose of radiation to an animal. For example, the animal may receive photon radiotherapy, particle beam radiation therapy, other types of radiotherapies, and combinations thereof. In some embodiments, the radiation is delivered to the animal using a linear accelerator. In still other embodiments, the radiation is delivered using a gamma knife.

The source of radiation can be external or internal to the animal. External radiation therapy is most common and involves directing a beam of high-energy radiation to a tumor site through the skin using, for instance, a linear accelerator. While the beam of radiation is localized to the tumor site, it is nearly impossible to avoid exposure of normal, healthy tissue. However, external radiation is usually well tolerated by animals. Internal radiation therapy involves implanting a radiation-emitting source, such as beads, wires, pellets, capsules, particles, and the like, inside the body at or near the tumor site including the use of delivery systems that specifically target cancer cells (e.g., using particles attached to cancer cell binding ligands). Such implants can be removed following treatment, or left in the body inactive. Types of internal radiation therapy include, but are not limited to, brachytherapy, interstitial irradiation, intracavity irradiation, radioimmunotherapy, and the like.

The animal may optionally receive radiosensitizers (e.g., metronidazole, misonidazole, intra-arterial Budr, intravenous iododeoxyuridine (IudR), nitroimidazole, 5-substituted-4-nitroimidazoles, 2H-isoindolediones, [[(2-bromoethyl)-amino]methyl]-nitro-1H-imidazole-1-ethanol, nitroaniline derivatives, DNA-affinic hypoxia selective cytotoxins, halogenated DNA ligand, 1,2,4 benzotriazine oxides, 2-nitroimidazole derivatives, fluorine-containing nitroazole derivatives, benzamide, nicotinamide, acridine-intercalator, 5-thiotretrazole derivative, 3-nitro-1,2,4-triazole, 4,5-dinitroimidazole derivative, hydroxylated texaphrins, cisplatin, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide, paclitaxel, heat (hyperthermia), and the like), radioprotectors (e.g., cysteamine, aminoalkyl dihydrogen phosphorothioates, amifostine (WR 2721), IL-1, IL-6, and the like). Radiosensitizers enhance the killing of tumor cells. Radioprotectors protect healthy tissue from the harmful effects of radiation.

Any type of radiation can be administered to an animal, so long as the dose of radiation is tolerated by the animal without unacceptable negative side-effects. Suitable types of radiotherapy include, for example, ionizing (electromagnetic) radiotherapy (e.g., X-rays or gamma rays) or particle beam radiation therapy (e.g., high linear energy radiation). Ionizing radiation is defined as radiation comprising particles or photons that have sufficient energy to produce ionization, i.e., gain or loss of electrons (as described in, for example, U.S. Pat. No. 5,770,581 incorporated herein by reference in its entirety). The effects of radiation can be at least partially controlled by the clinician. In one embodiment, the dose of radiation is fractionated for maximal target cell exposure and reduced toxicity.

In one embodiment, the total dose of radiation administered to an animal is about 0.01 Gray (Gy) to about 100 Gy. In another embodiment, about 10 Gy to about 65 Gy (e.g., about 15 Gy, 20 Gy, 25 Gy, 30 Gy, 35 Gy, 40 Gy, 45 Gy, 50 Gy, 55 Gy, or 60 Gy) are administered over the course of treatment. While in some embodiments a complete dose of radiation can be administered over the course of one day, the total dose is ideally fractionated and administered over several days. Desirably, radiotherapy is administered over the course of at least about 3 days, e.g., at least 5, 7, 10, 14, 17, 21, 25, 28, 32, 35, 38, 42, 46, 52, or 56 days (about 1-8 weeks). Accordingly, a daily dose of radiation will comprise approximately 1-5 Gy (e.g., about 1 Gy, 1.5 Gy, 1.8 Gy, 2 Gy, 2.5 Gy, 2.8 Gy, 3 Gy, 3.2 Gy, 3.5 Gy, 3.8 Gy, 4 Gy, 4.2 Gy, or 4.5 Gy), or 1-2 Gy (e.g., 1.5-2 Gy). The daily dose of radiation should be sufficient to induce destruction of the targeted cells. If stretched over a period, in one embodiment, radiation is not administered every day, thereby allowing the animal to rest and the effects of the therapy to be realized. For example, radiation desirably is administered on 5 consecutive days, and not administered on 2 days, for each week of treatment, thereby allowing 2 days of rest per week. However, radiation can be administered 1 day/week, 2 days/week, 3 days/week, 4 days/week, 5 days/week, 6 days/week, or all 7 days/week, depending on the animal's responsiveness and any potential side effects. Radiation therapy can be initiated at any time in the therapeutic period. In one embodiment, radiation is initiated in week 1 or week 2, and is administered for the remaining duration of the therapeutic period. For example, radiation is administered in weeks 1-6 or in weeks 2-6 of a therapeutic period comprising 6 weeks for treating, for instance, a solid tumor. Alternatively, radiation is administered in weeks 1-5 or weeks 2-5 of a therapeutic period comprising 5 weeks. These exemplary radiotherapy administration schedules are not intended, however, to limit the present invention.

Antimicrobial therapeutic agents may also be used as therapeutic agents in the present invention. Any agent that can kill, inhibit, or otherwise attenuate the function of microbial organisms may be used, as well as any agent contemplated to have such activities. Antimicrobial agents include, but are not limited to, natural and synthetic antibiotics, antibodies, inhibitory proteins (e.g., defensins), antisense nucleic acids, membrane disruptive agents and the like, used alone or in combination. Indeed, any type of antibiotic may be used including, but not limited to, antibacterial agents, antiviral agents, antifungal agents, and the like.

In some embodiments of the present invention, a compound of the invention and one or more therapeutic agents or anticancer agents are administered to an animal under one or more of the following conditions: at different periodicities, at different durations, at different concentrations, by different administration routes, etc. In some embodiments, the compound is administered prior to the therapeutic or anticancer agent, e.g., 0.5, 1, 2, 3, 4, 5, 10, 12, or 18 hours, 1, 2, 3, 4, 5, or 6 days, or 1, 2, 3, or 4 weeks prior to the administration of the therapeutic or anticancer agent. In some embodiments, the compound is administered after the therapeutic or anticancer agent, e.g., 0.5, 1, 2, 3, 4, 5, 10, 12, or 18 hours, 1, 2, 3, 4, 5, or 6 days, or 1, 2, 3, or 4 weeks after the administration of the anticancer agent. In some embodiments, the compound and the therapeutic or anticancer agent are administered concurrently but on different schedules, e.g., the compound is administered daily while the therapeutic or anticancer agent is administered once a week, once every two weeks, once every three weeks, or once every four weeks. In other embodiments, the compound is administered once a week while the therapeutic or anticancer agent is administered daily, once a week, once every two weeks, once every three weeks, or once every four weeks.

Compositions within the scope of this invention include all compositions wherein the compounds of the present invention are contained in an amount which is effective to achieve its intended purpose. While individual needs vary, determination of optimal ranges of effective amounts of each component is within the skill of the art. Typically, the compounds may be administered to mammals, e.g. humans, orally at a dose of 0.0025 to 50 mg/kg, or an equivalent amount of the pharmaceutically acceptable salt thereof, per day of the body weight of the mammal being treated for disorders responsive to induction of apoptosis. In one embodiment, about 0.01 to about 25 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the compound. The unit dose may be administered one or more times daily as one or more tablets or capsules each containing from about 0.1 to about 10 mg, conveniently about 0.25 to 50 mg of the compound or its solvates.

In a topical formulation, the compound may be present at a concentration of about 0.01 to 100 mg per gram of carrier. In a one embodiment, the compound is present at a concentration of about 0.07-1.0 mg/ml, for example, about 0.1-0.5 mg/ml, and in one embodiment, about 0.4 mg/ml.

In addition to administering the compound as a raw chemical, the compounds of the invention may be administered as part of a pharmaceutical preparation containing suitable pharmaceutically acceptable carriers comprising excipients and auxiliaries which facilitate processing of the compounds into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active compound(s), together with the excipient.

The pharmaceutical compositions of the invention may be administered to any patient which may experience the beneficial effects of the compounds of the invention. Foremost among such patients are mammals, e.g., humans, although the invention is not intended to be so limited. Other patients include veterinary animals (cows, sheep, pigs, horses, dogs, cats and the like).

The compounds and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762; each herein incorporated by reference in its entirety.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXPERIMENTAL

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention. As used herein, the use of personal pronouns such as “we”, “I”, and/or “our” refers to the inventors.

### Example I

This example demonstrates that YUM70 is cytotoxic to pancreatic cancer cells.

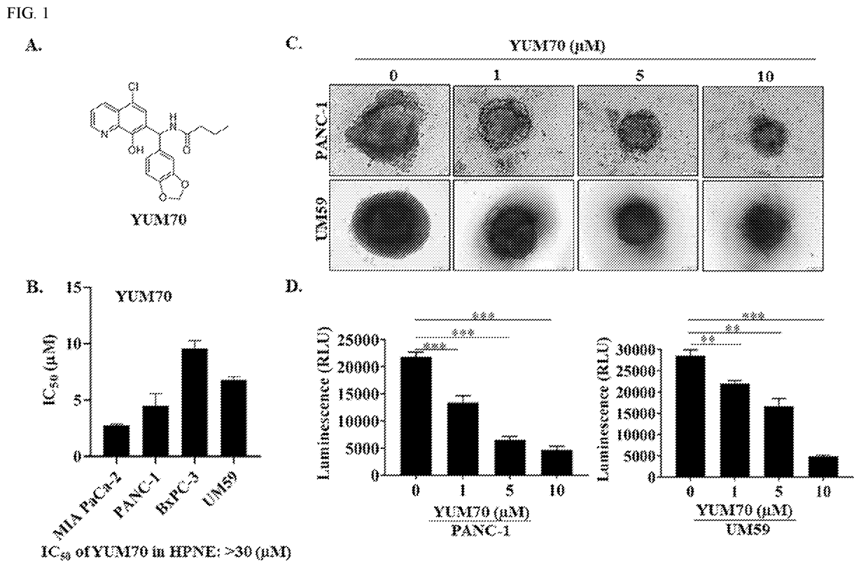

YUM70, a derivative of 8-hydroxyquinoline (FIG. 1A), was identified in a phenotypic screen for cytotoxicity from 40,000 in-house drug-like compounds (22). YUM70 showed selective cytotoxicity for pancreatic cancer cell lines and pancreatic cancer 3D spheroids generated from PANC-1 and UM59 cell lines over normal pancreatic tissue-derived HPNE cells (FIGS. 1B, 1C, and 1D). UM59 is a patient-derived cell line with a KRAS mutation (23). Both PANC-1 and UM59 cells formed compact spheroids. We measured ATP by luminescence using CellTiter-Glo® 3D cell viability assay, we observed that viability was reduced 4.7 and 5.8 times (p-value<0.0001, n=6) for PANC-1 and UM59, respectively. These results demonstrate that YUM70 is not only cytotoxic to pancreatic cancer cells grown as a monolayer but also cytotoxic to tumorspheres. Interestingly, we observed IC₅₀ of YUM70 varied among pancreatic cancer cell lines. IC₅₀ of YUM70 in BxPC-3 is approximately three times higher than MIA PaCa-2 cells. PANC-1 and UM59 cells are also more sensitive to YUM70 than BxPC-3 cells. MIA PaCa-2, PANC-1, and UM59 cells have mutated KRAS while BxPC-3 cells have wild-type KRAS (23). Since more than 90% of pancreatic cancer patients have mutated KRAS, we primarily focused on pancreatic cancer.

### Example II

This example demonstrates that YUM70 targets GRP78.

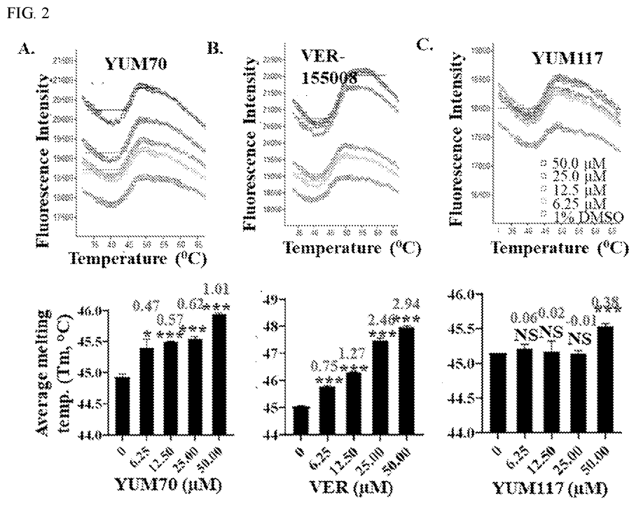

Since GRP78 dissociation from ER sensors is a crucial event to initiate the UPR and YUM70 treatment produces a gene signature similar to GRP78 knock-down, we hypothesized that GRP78 may be the target of YUM70. To determine whether YUM70 binds to GRP78, we performed a thermal shift assay using purified full-length GRP78. YUM70 binds to full-length GRP78 and causes a positive shift in the melting temperature (Tm) of GRP78 in a dose-dependent manner (FIG. 2A). The positive control VER155008 (VER), an HSP70/GRP78 inhibitor, stabilized GRP78 as expected (FIG. 2B). In contrast, YUM117, an inactive analog of YUM70, did not produce any Tm shift (FIG. 2C).

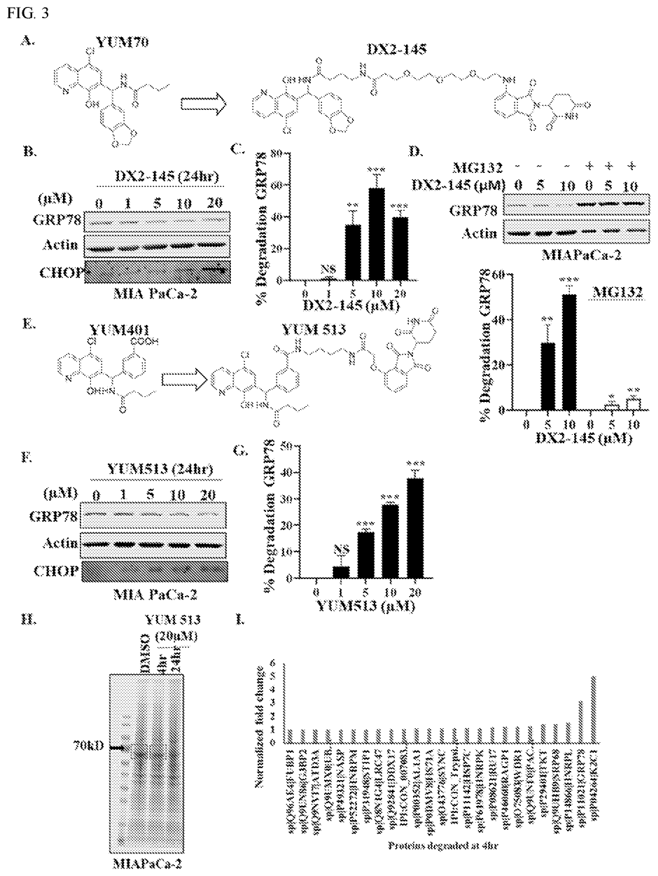

Proteolysis targeting chimera (PROTAC) is a powerful technology for targeted protein degradation (24). To selectively degrade GRP78, we synthesized a PROTAC by incorporating YUM70, a linker, and an E3-ligase recruiting ligand. To evaluate the extent of GRP78 degradation, we treated MIA PaCa-2 cells with YUM70-PROTAC (DX2-145, FIG. 3A) for 24 hr at various doses and the level of GRP78 protein was assessed by Western blot. DX2-145 elicited a concentration-dependent degradation of GRP78 (FIGS. 3B and 3C) and increased CHOP expression (FIG. 3B). We observed maximum degradation at 10 μM and a decrease in degradation at 20 μM due to the hook effect, a typical property of PROTACs. In a separate experiment, we show that MG132, a proteasome inhibitor, blocked DX2-145-mediated degradation of GRP78 (FIG. 3D). This data demonstrates that DX2-145 degrades GRP78 in a proteasome-dependent manner. We observed a similar pattern of degradation with the PROTAC YUM513 (YUM401-PROTAC) (FIGS. 3E-G). In a separate experiment, we treated MIA PaCa-2 cells with YUM513 for 4 hr or 24 hr and observed a decrease in intensity of a band of approximately 70 kD by SDS-PAGE followed by Coomassie blue staining (FIG. 3H). Analysis of this band using LC-MS/MS confirmed the degradation of GRP78 (FIG. 3I). DX2-145 is more effective in degrading GRP78 than YUM513 and was selected for further studies.

### Example III

This example demonstrates YUM70 reduces tumor growth in a MIA PaCa-2 xenograft model.

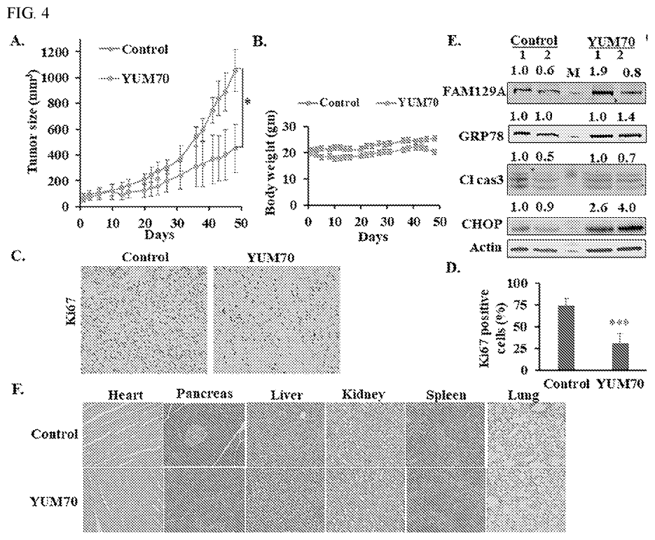

To evaluate the in vivo efficacy of YUM70, we performed xenograft studies in NCr nude female mice. Subcutaneous human pancreatic cancer xenografts were established using MIA PaCa-2 cells on the dorsal flank of immune-deficient mice. The mice were injected intraperitoneally with either YUM70 (30 mg/kg) or vehicle (10% DMSO, 70% PG, 20% saline) 5 days a week for 7 weeks. A significant tumor growth delay (p<0.05) was observed (FIG. 4A) with no significant change in body weight during the course of treatment (FIG. 4B). Consistent with our in vitro data, YUM70 treatment suppressed tumor cell proliferation as confirmed by the significant decrease in Ki67 staining (FIGS. 4C and D) and the increased expression levels of CHOP and FAM129A in the treatment group (FIG. 4E). No gross toxicity was observed in the heart, pancreas, liver, kidney, lung, and spleen (FIG. 4F). These results demonstrate that YUM70 treatment induced ER stress-mediated apoptosis in the tumor cells implanted in mice without major toxicity to normal tissues.

### Example IV

This example demonstrates that YUM70 induces synergistic cell death with topotecan and vorinostat.

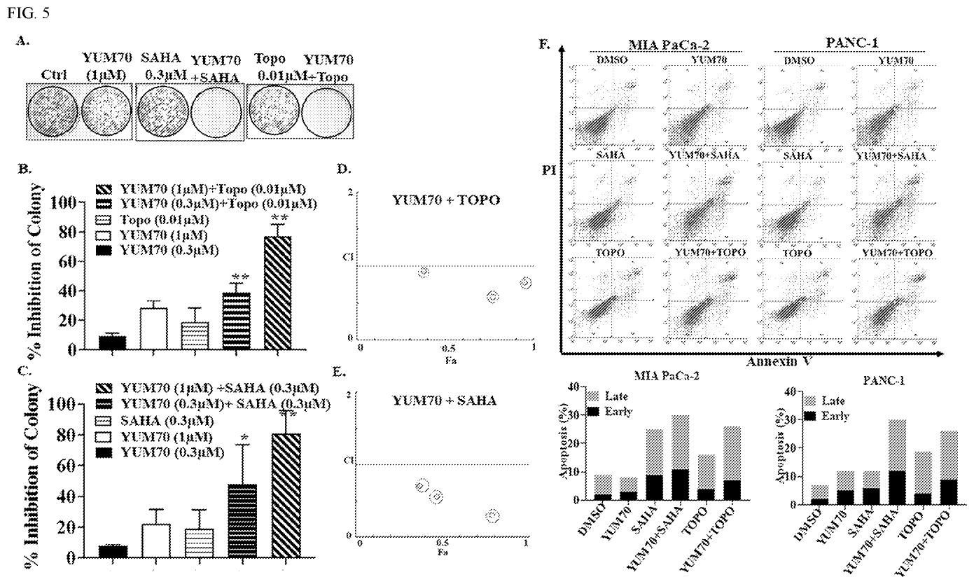

YUM70 showed moderate potency in vitro and in vivo as a single agent. To find novel and effective therapies for pancreatic cancer, we performed clonogenic survival assays using combination of YUM70 with several clinically approved drugs, drugs in clinical trials, and preclinical agents. YUM70 showed a synergistic effect when combined with topotecan or vorinostat (FIGS. 5A-E), and an additive effect with 5-FU (fluorouracil), tosedostat, and DFO (deferoxamine mesylate) (data not shown). We did not observe a significant additive effect or synergy by combining YUM70 with gemcitabine.

YUM70-topotecan combination showed strong synergy with a combination index (CI) of 0.59 at 1 μM YUM70 and 0.01 μM topotecan (FIG. 5D). YUM70-SAHA combination was also able to significantly enhance cytotoxicity with CI 0.29 at a dose of 1 μM YUM70 and 0.3 μM vorinostat (FIG. 5E). The synergistic effect of YUM70 and topotecan, vorinostat, can be attributed to apoptosis enhancement. To test this hypothesis, we performed annexin V apoptosis assays (FIG. 5F) and found an increase in early and late-stage apoptosis for each drug combination. Collectively, the reported study and our data suggest that the synergistic effect of these combinations was due to apoptosis enhancement. Such combinations can be further optimized for future clinical applications.

Despite recent advancements in the molecular, pathological, and biological understanding of pancreatic cancer, it remains a devastating disease with limited options for effective treatments. Thus, there is an urgent need to develop new therapies for this disease. In this study, we identified YUM70 as a lead compound showing in vitro cytotoxicity in both 2D and 3D pancreatic cancer culture systems and significant synergy with topotecan and vorinostat. Importantly, YUM70 showed significant anticancer activity in an in vivo pancreatic cancer xenograft model with no observed toxicity to normal tissues.

Mechanistically, we demonstrate that YUM70 suppresses proliferation and induces ER stress and apoptosis in pancreatic cancer cells. Acute ER stress leads to transient activation of the UPR signaling network to restore ER homeostasis. However, prolonged UPR activation promotes cell death by activating apoptosis. CHOP, a transcription factor known to be involved in ER stress-induced apoptosis, is distinctly overexpressed in response to ER stress through IRE1-, PERK- and ATF6-dependent transcriptional induction (25). These factors are activated by GRP78 when it dissociates from these ER sensors/receptors that in turn increase expression of GRP78.

Previously, several small molecules were identified that are non-selective inhibitors of GRP78 (26,27). YUM70 is selective for GRP78 over other ER proteins including GSTO1, PDI and HSP70. We demonstrated in multiple complementary assays that YUM70 binds to recombinant GRP78. Importantly, using YUM70 as a warhead, we synthesized the first PROTAC to degrade GRP78 through a cereblon-mediated E3 ligase mechanism. Using LC-MS/MS-based proteomics, we confirmed the degradation of GRP78 (FIGS. 3H, and I) by our PROTAC. DX2-145 degrades GRP78 in a dose-dependent manner, and MG132 completely blocked its degradation. We did not observe an increased cytotoxicity of DX2-145 over YUM70, suggesting that our first-generation PROTAC needs further optimization. The potency of PROTAC can be improved by optimizing the ligand of E3 ubiquitin ligase, or the linker, or using a more potent warhead. Since YUM70 treatment increases GRP78 levels, a degradation strategy via a PROTAC will be a more efficient approach to target GRP78 in tumors.

Current evidence suggests that GRP78 haploinsufficiency has no major deleterious effect on organ homeostasis in young as well as aged mice (28) consistent with the notion that normal organ function requires only a low basal level of GRP78 for maintenance, while cancer cells require high levels of active GRP78 for growth, survival, invasion and therapeutic resistance. This is in agreement with our observation that YUM70 preferentially blocks the growth of cancer but not normal pancreatic cells. To our knowledge, YUM70 is among the first small molecule inhibitors that directly bind to GRP78, suppresses its ATPase activity, and causes its dissociation from the ER stress sensors, leading to activation of the UPR and apoptosis. Another compound, HA15 that targets GRP78 among other proteins, triggers ER stress and autophagy and overcomes BRAF inhibitor resistance in melanoma and other cancer cells (29). A ruthenium compound, KP1339 (IT-139) that inhibits GRP78 and disrupt ER homeostasis, is currently under Phase I clinical investigation (30). Thus, ER stress inducers and GRP78 inhibitors hold promise for the treatment of pancreatic cancer (31).

In this study, we observed a synergistic cell killing effect of YUM70 with topotecan, a topoisomerase I inhibitor, and vorinostat, an HDAC inhibitor. Topotecan induces apoptosis by p53 activation (32). It is currently used to treat small cell lung cancer and ovarian cancer (33) and is the first topoisomerase I inhibitor approved for oral use (Hycamtin Capsules, GlaxoSmithKline). Irinotecan, another topoisomerase I inhibitor, is part of the FOLFIRINOX regimen approved for the treatment of advanced pancreatic cancer. Therefore, combination of GRP78 inhibitors and topotecan could be a new treatment option for pancreatic cancer. Treatment strategies combining HDAC inhibitors with gemcitabine, radiation therapy, 5-FU or bortezomib have failed to improve survival outcomes. YUM70 in combination with vorinostat showed a promising synergistic cell killing effect in vitro. Although the underlying mechanism of this synergy was not assessed in this study, a strong similarity in molecular pathways was observed in CMap analysis (Table S15). HDAC inhibitors (e.g. vorinostat) were reported to cause GRP78 acetylation that disrupts GRP78 function and induces ER stress (17). Moreover, previous studies showed that vorinostat also promotes cell cycle arrest, inhibits growth, and induces apoptosis in cancer (34) by ATF4 and CHOP upregulation (35). Thus, these novel combinations including YUM70 may improve pancreatic cancer treatment response.

In conclusion, we demonstrated that YUM70 treatment induces ER stress and triggers UPR by inhibiting GRP78. As a result, eIF2α is phosphorylated leading to the induction of CHOP and apoptosis in both cell culture and xenograft models. Importantly, YUM70 slowed tumor growth in a pancreatic cancer xenograft model. Although YUM70 is moderately effective as a single agent in pancreatic cancer, it can be safely combined with topotecan and vorinostat. YUM70 is an excellent tool compound to further interrogate the role of GRP78 inhibition in pancreatic cancer. Altogether, these results indicate GRP78 as a promising target to treat KRAS mutant pancreatic cancer and YUM70 as a novel anticancer agent that could be used in combination with select drugs to improve treatment efficacy and overcome drug resistance.

### Example V

### General Chemical Methods:

All commercial chemicals and solvents were reagent grade and were used without further purification unless otherwise specified. Analytical thin layer chromatography was performed on Merck precoated plates (silica gel 60 F254) to follow the course of the reactions. ¹H and ¹³C NMR spectra were recorded on a Bruker Ultrashield 300 MHz, a Bruker Ascend 400 MHz, or 500 MHz Varian NMR spectrometer. Chemical shifts (6) of NMR are reported in parts per million (ppm) units relative to the residual undeuterated solvent. The following abbreviations were used to describe peak splitting patterns when appropriate: s (singlet), d (doublet), t (triplet), q (quartet), m (multiplet), bs (broad singlet), dd (doublet of doublets), td (triplet of doublets). Coupling constants (J) are expressed in hertz unit (Hz). Mass spectra were obtained on a Shimadzu LCMS-2020 liquid chromatography mass spectrometer or on a Thermo-Scientific LCQ Fleet mass spectrometer. The purity of compounds was determined by UPLC or HPLC analysis. The Waters Acquity H class analytical UPLC was used for UV detection at 230 and 254 nm wavelengths. The reverse phase column was used in the Acquity UPLC BEH (C18-1.7 μm, 2.1 mm×50 mm). The analytical HPLC Shimadzu HPLC Test Kit C18 column (3 μm, 4.6×50 mm) was used under the following gradient elution condition: mobile phase A of acetonitrile/water (10-95%) or mobile phase B of methanol/water (10-95%). The purity was established by integration of the areas of major peaks detected at 254 nm. All tested compounds had >95% purity unless mentioned. For some compounds, flash chromatography was performed using a Biotage Isolera One flash purification system.

### Representative Scheme 1 for Synthesis of Formula I Compounds.

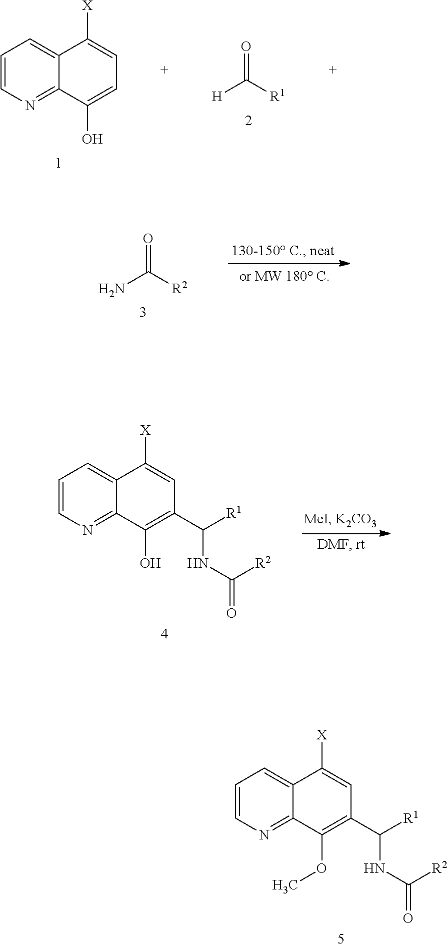

The synthesis of Formula I compounds was accomplished by employing the Betti reaction as a modified type of Mannich reaction (43).

### General Protocol a for Betti Reaction

A mixture of substituted hydroxyquinoline (1 equiv), appropriate amine (2.5-3.0 equiv), and amide (1-2 equiv) were stirred neat at 130-150° C. for 6-12 hrs. For some examples ethanol was used as a solvent. Upon heating, the reaction mixture melted and solid was formed after completion of the reaction. The solid was isolated by multiple trituration with ethyl acetate and diethyl ether, and the crude product was purified by recrystallization from ethanol. For some examples HPLC was used to purify the crude product obtained after trituration.

### General Protocol for Suzuki Coupling

A solution of bromo quinoline (1.0 eq), CsCO₃ (3.0 eq) in dioxane (5:1) was degassed and flushed with argon for three times. Then Pd(PPh₃)₄ (0.02 eq) and corresponding boronic acid (1.2 eq) were added subsequently. Then it was degassed and flushed with argon for three times again before it was heated at reflux overnight. The mixture was concentrated and purified with flash chromatography (20% EtOAc in hexane) to give the corresponding analogs.

### General Protocol for Buchwald-Hartwig Coupling

A solution of bromo quinoline (1.0 eq), NaO^{t}Bu (1.4 eq) in toluene was degassed and flushed with argon for three times. Then Pd₂(dba)₃ (0.003 eq), SPhos (0.008 eq), and secondary amine compound (1.5 eq) were added subsequently. Then it was degassed and flushed with argon for three times again before it was heated at reflux overnight. The mixture was concentrated and purified with flash chromatography (20% EtOAc in hexane) to give the corresponding product.

General Protocol for Replacement of Chlorine with Amino Chains

To a solution of appropriate chloro compound (1.0 e.q.) and triethylamine (2.0 e. q.) was added the corresponding amine (1.0 e.q.) portionwise. The mixture was heated at 80° C. for 12 h. The mixture was concentrated and purified with flash chromatography (10% EtOAc in hexane) to give the corresponding analogs.

### N-((5-chloro-8-hydroxyquinolin-7-yl)(pyridin-3-yl)methyl)acetamide (G1)

Synthesized following the general protocol A, using 5-chloro-8-hydroxyquinoline (98 mg, 0.55 mmol), nicotinaldehyde (160 mg, 1.5 mmol), and acetamide (32 mg, 0.55 mmol) to yield G1 as a white solid (41 mg, 22%). ¹H NMR (300 MHz, Chloroform-d) δ 8.86 (dd, J=4.2, 1.5 Hz, 1H), 8.62 (dd, J=2.1, 1.1 Hz, 1H), 8.55 (dd, J=8.6, 1.5 Hz, 1H), 8.52 (dd, J=5.0, 1.6 Hz, 1H), 7.68 (dddd, J=8.0, 2.4, 1.7, 0.8 Hz, 1H), 7.61 (dd, J=8.6, 4.2 Hz, 1H), 7.58 (s, 1H), 7.28-7.22 (m, 1H), 7.15 (d, J=8.8 Hz, 1H), 6.56 (d, J=8.7 Hz, 1H), 2.13 (s, 3H). MS (ESI) m/z=328 (M+H)⁺.

### N-((5-chloro-8-hydroxyquinolin-7-yl)(pyridin-3-yl)methyl)propionamide (G2)

Synthesized following the general protocol A, using 5-chloro-8-hydroxyquinoline (48 mg, 0.27 mmol), nicotinaldehyde (75 mg, 0.7 mmol), and propionamide (20 mg, 0.27 mmol) to yield G2 as a pale-yellow solid (34 mg, 37%). ¹H NMR (300 MHz, Chloroform-d) δ 8.86 (s, 1H), 8.59 (d, J=17.1 Hz, 1H), 8.52 (s, 1H), 7.68 (d, J=7.5 Hz, 1H), 7.64-7.52 (m, 2H), 7.26 (s, 1H), 7.17 (s, 1H), 6.57 (d, J=8.8 Hz, 1H), 2.37 (q, J=7.7 Hz, 2H), 1.23 (t, J=7.5 Hz, 3H). MS (ESI) m/z=342 (M+H)⁺.

### N-((5-chloro-8-hydroxyquinolin-7-yl)(pyridin-3-yl)methyl)pentanamide (G3)

Synthesized following the general protocol A, using 5-chloro-8-hydroxyquinoline (32 mg, 0.18 mmol), nicotinaldehyde (53 mg, 0.5 mmol), and pentanamide (18 mg, 0.18 mmol) to yield G3 as a white solid (25 mg, 37%). ¹H NMR (400 MHz, DMSO-d₆) δ 8.97 (d, J=4.0 Hz, 1H), 8.54-8.48 (m, 1H), 8.45 (d, J=4.6 Hz, 1H), 7.74 (d, J=9.4 Hz, 2H), 7.63 (d, J=7.7 Hz, 1H), 7.35 (dd, J=8.0, 4.7 Hz, 1H), 6.70 (d, J=8.0 Hz, 1H), 6.54 (s, 1H), 2.25 (t, J=7.4 Hz, 2H), 1.57-1.46 (m, 2H), 1.34-1.22 (m, 2H), 0.88 (t, J=7.3 Hz, 3H). MS (ESI) m z=370 (M+H)⁺.

### N-((5-chloro-8-hydroxyquinolin-7-yl)(pyridin-3-yl)methyl)acrylamide (G4)

Synthesized following the general protocol A, using 5-chloro-8-hydroxyquinoline (98 mg, 0.55 mmol), nicotinaldehyde (160 mg, 1.5 mmol), and acrylamide (39 mg, 0.55 mmol) as reactants to yield G4 as a white solid (61 mg, 33%). ¹H NMR (300 MHz, Chloroform-d) δ 8.86 (d, J=4.3 Hz, 1H), 8.63 (s, 1H), 8.54 (t, J=7.9 Hz, 2H), 7.71 (d, J=8.3 Hz, 1H), 7.67-7.57 (m, 2H), 6.64 (d, J=8.7 Hz, 1H), 6.39 (d, J=16.9 Hz, 1H), 6.23 (dd, J=17.0, 10.1 Hz, 1H), 5.75 (d, J=10.0 Hz, 1H). MS (ESI) m/z=340 (M+H)⁺.

1-((5-chloro-8-hydroxyquinolin-7-yl)(pyridin-3-yl)methyl)-3-methylurea (G5) Synthesized following the general protocol A, using 5-chloro-8-hydroxyquinoline (98 mg, 0.55 mmol), nicotinaldehyde (160 mg, 1.5 mmol), and 1-methylurea (40 mg, 0.55 mmol) as reactants to yield G5 as a brown solid (37 mg, 19%). ¹H NMR (300 MHz, Chloroform-d) δ 8.85 (s, 1H), 8.68 (d, J=20.4 Hz, 1H), 8.53 (d, J=11.0 Hz, 2H), 7.66 (d, J=29.4 Hz, 4H), 6.43 (d, J=10.4 Hz, 1H), 5.89 (s, 1H), 2.84 (d, J=5.6 Hz, 3H). MS (ESI) m/z=343 (M+H)⁺.

### N-((5-chloro-8-hydroxyquinolin-7-yl)(pyridin-3-yl)methyl)cyclopropanecarboxamide (G6)

Synthesized following the general protocol A, using 5-chloro-8-hydroxyquinoline (32 mg, 0.18 mmol), nicotinaldehyde (53 mg, 0.5 mmol), and cyclopropane carboxamide (16 mg, 0.18 mmol) as reactants to yield G6 as a yellow solid (26 mg, 40%). MS (ESI) m/z=354 (M+H)⁺.

### N-((5-bromo-8-hydroxyquinolin-7-yl)(pyridin-3-yl)methyl)butyramide (G7)

Synthesized following the general protocol A, using 5-bromo-8-hydroxyquinoline (123 mg, 0.55 mmol), nicotinaldehyde (160 mg, 1.5 mmol), and butyramide (48 mg, 0.55 mmol) as reactants to yield G7 as a white solid (91 mg, 41%). ¹H NMR (300 MHz, Chloroform-d) δ 8.84 (d, J=4.2 Hz, 1H), 8.62 (s, 1H), 8.50 (d, J=8.2 Hz, 2H), 7.77 (s, 1H), 7.68 (d, J=7.8 Hz, 1H), 7.61 (dd, J=8.6, 4.6 Hz, 1H), 7.12 (d, J=8.6 Hz, 1H), 6.58 (d, J=8.8 Hz, 1H), 2.31 (t, J=7.4 Hz, 2H), 1.74 (q, J=7.6 Hz, 2H), 1.03-0.85 (m, 3H). MS (ESI) m/z=400 (M+H)⁺.

### 5-(tert-butyl)quinolin-8-ol

To glycerol (570 mmol, 6.2 mmol) preheated to 160° C. for 1 hour, and cooled down to 110° C., substituted 4-(tert-butyl)phenol (345 mg, 2.3 mmol) and sodium iodide (8 mg, 0.048 mmol) were added. The mixture was vigorously stirred, heated to 150° C. and sulfuric acid 95-98% (5.3 mmol) was added drop wise. After 45 minutes at 150° C., the mixture was allowed to reach room temperature, and then distributed in CH₂Cl₂ and water. The organic layer was separated, washed with water, brine solution, dried over Na₂SO₄ and evaporated. The crude product was purified through column chromatography (100 mg, yield 21%).

### N-((5-(tert-butyl)-8-hydroxyquinolin-7-yl)(pyridin-3-yl)methyl)butyramide (G8)

Synthesized following the general protocol A, using 5-tert-butyl-8-hydroxyquinoline (55 mg, 0.27 mmol), nicotinaldehyde (80 mg, 0.75 mmol), and butyramide (24 mg, 0.27 mmol) to yield G8 as a brown solid (40 mg, 39%). ¹H NMR (300 MHz, Chloroform-d) δ 8.88-8.74 (m, 2H), 8.61 (d, J=2.5 Hz, 1H), 8.52-8.38 (m, 1H), 7.73 (d, J=7.7 Hz, 1H), 7.59-7.45 (m, 2H), 7.44 (s, 1H), 7.24 (dd, J=7.9, 4.7 Hz, 1H), 6.53 (d, J=8.8 Hz, 1H), 2.31 (t, J=7.5 Hz, 2H), 1.74 (q, J=7.5 Hz, 2H), 1.59 (s, 9H), 0.98 (dd, J=8.3, 6.4 Hz, 3H). MS (ESI) m z=378 (M+H)⁺.

### 5-(trifluoromethyl)quinolin-8-ol

To glycerol (570 mmol, 6.2 mmol) preheated to 160° C. for 1 hour, and cooled down to 110° C., substituted 4-(trifluoromethyl)phenol (372 mg, 23.0 mmol) and sodium iodide (8 mg, 0.048 mmol) were added. The mixture was vigorously stirred, heated to 150° C. and sulfuric acid 95-98% (5.3 mmol) was added drop wise. After 45 minutes at 150° C., the mixture was allowed to reach room temperature, and then distributed in CH₂Cl₂ and water. The organic layer was separated, washed with water, brine solution, dried over Na₂SO₄ and evaporated. The crude product was purified through column chromatography (44 mg, yield 9%).

### N-((8-hydroxy-5-(trifluoromethyl)quinolin-7-yl)(pyridin-3-yl)methyl)butyramide (G9)

Synthesized following the general protocol A, using 5-(trifluoromethyl)quinolin-8-ol (29 mg, 0.14 mmol), nicotinaldehyde (40 mg, 0.37 mmol), and butyramide (12 mg, 0.14 mmol) as reactants to yield G9 as a brown solid (24 mg, 44%). ¹H NMR (300 MHz, DMSO-d₆) δ 9.03-8.99 (m, 1H), 8.96 (d, J=8.4 Hz, 1H), 8.50 (d, J=2.3 Hz, 1H), 8.48-8.42 (m, 2H), 8.06 (s, 1H), 7.78 (dd, J=8.8, 4.1 Hz, 1H), 7.63 (d, J=7.9 Hz, 1H), 7.36 (dd, J=7.9, 4.7 Hz, 1H), 6.70 (d, J=8.2 Hz, 1H), 2.22 (t, J=7.3 Hz, 2H), 1.56 (q, J=7.2 Hz, 2H), 0.86 (t, J=7.4 Hz, 3H). MS (ESI) m/z=390 (M+H)⁺.

### N-((5-acetyl-8-hydroxyquinolin-7-yl)(pyridin-3-yl)methyl)butyramide (G10)

Synthesized following the general protocol A, using 1-(8-hydroxyquinolin-5-yl)ethan-1-one (103 mg, 0.55 mmol), nicotinaldehyde (160 mg, 1.5 mmol), and butyramide (48 mg, 0.55 mmol) as reactants to yield G10 as a yellow solid (51 mg, 25%). ¹H NMR (300 MHz, DMSO-d₆) δ 9.43-9.33 (m, 1H), 9.02-8.83 (m, 2H), 8.54 (d, J=2.2 Hz, 1H), 8.50-8.40 (m, 2H), 7.77-7.58 (m, 2H), 7.36 (dd, J=8.1, 4.8 Hz, 1H), 6.72 (d, J=8.5 Hz, 1H), 2.67 (s, 3H), 2.25 (t, J=7.3 Hz, 2H), 1.58 (q, J=7.4 Hz, 2H), 0.87 (t, J=7.4 Hz, 3H). MS (ESI) m z=364 (M+H)⁺.

### N-((5-cyclopropyl-8-hydroxyquinolin-7-yl)(pyridin-3-yl)methyl)butyramide (G11)

5-cyclopropyl-8-methoxyquinoline was synthesized following the general protocol for Suzuki coupling as mentioned above using cyclopropyl boronic acid (51 mg, 0.6 mmol) and 5-bromo-8-methoxyquinoline (120 mg, 0.5 mmol). The product obtained (70 mg, 0.35 mmol) was treated with boron tribromide in DCM (2 equiv) at 0° C., stirring continued at room temperature until completion of the reaction. Water (2 mL) was added to quench the reaction, followed by concentration and purification by column chromatography to obtain 5-cyclopropyl-8-hydroxyquinoline as white solid (51 mg, Yield 78%).

Starting from 5-cyclopropyl-8-hydroxyquinoline (51 mg, 0.27 mmol), nicotinaldehyde (75 mg, 0.7 mmol), and butyramide (24 mg, 0.27 mmol) as reactants following general protocol A, G1l was synthesized as a brown solid (18 mg, 19%). ¹H NMR (300 MHz, Methanol-d₄) δ 8.87 (t, J=7.0 Hz, 2H), 8.69-8.52 (m, 2H), 8.20 (d, J=7.8 Hz, 1H), 7.75 (dd, J=8.0, 5.3 Hz, 1H), 7.65 (dd, J=8.6, 4.4 Hz, 2H), 7.32 (s, 1H), 6.79 (s, 1H), 2.37 (t, J=7.3 Hz, 3H), 1.71 (q, J=7.3 Hz, 2H), 1.08 (dd, J=7.9, 4.5 Hz, 2H), 0.98 (t, J=7.4 Hz, 3H), 0.71 (t, J=5.1 Hz, 2H). MS (ESI) m/z=362 (M+H)⁺.

### N-((8-hydroxy-5-isopropylquinolin-7-yl)(pyridin-3-yl)methyl)butyramide (G12)

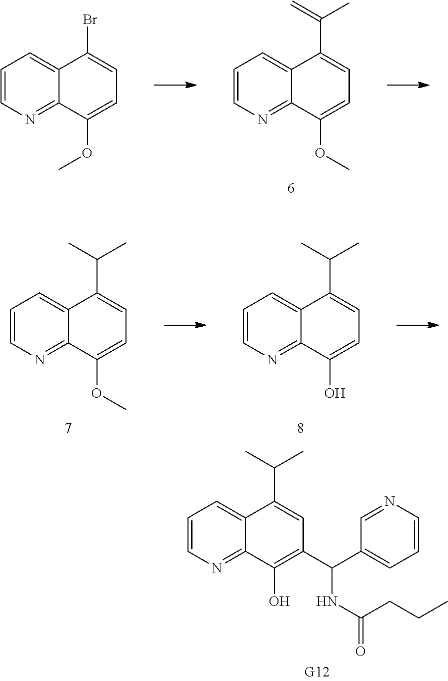 [SMILES: COc1ccc(Br)c2cccnc12 ; C=C(C)c1ccc(OC)c2ncccc12 ; COc1ccc(C(C)C)c2cccnc12 ; CC(C)c1ccc(O)c2ncccc12 ; CCCC(=O)NC(c1cccnc1)c1cc(C(C)C)c2cccnc2c1O]

5-isopropyl-8-hydroxyquinoline was synthesized following the general protocol for Suzuki coupling as mentioned above using 4,4,5,5-tetramethyl-2-(prop-1-en-2-yl)-1,3,2-dioxaborolane (302 mg, 1.8 mmol), 5-bromo-8-methoxyquinoline (338 mg, 1.5 mmol), Pd(OAc)₂ (2 mg), XPhos (7 mg, 0.015 mmol) and CsCO₃ (1.4 g, 4.5 mmol) to afford compound 6 (154 mg, 0.77 mmol). Compound 6 was subjected to reduction of the alkene with H₂ Pd/C (100 mg). The product obtained (68 mg, 0.34 mmol) was treated with boron tribromide in DCM (2 equiv) at 0° C., stirring continued at room temperature until completion of the reaction. Water (2 mL) was added to quench the reaction, followed by concentration and purification by column chromatography to obtain 5-isopropyl-8-hydroxyquinoline as white solid (40 mg, Yield 21%).

Starting from 5-isopropyl-8-hydroxyquinoline (26 mg, 0.14 mmol), nicotinaldehyde (40 mg, 0.38 mmol), and butyramide (12 mg, 0.14 mmol) as reactants following general protocol A, G12 was obtained as a brown solid (17 mg, 35%). ¹H NMR (300 MHz, Methanol-d₄) δ 8.90 (dd, J=4.4, 1.5 Hz, 1H), 8.81-8.66 (m, 3H), 8.42 (d, J=8.2 Hz, 1H), 7.94 (dd, J=8.2, 5.6 Hz, 1H), 7.71 (dd, J=8.7, 4.4 Hz, 1H), 7.52 (s, 1H), 6.83 (s, 1H), 3.72 (p, J=6.8 Hz, 1H), 2.39 (t, J=7.3 Hz, 2H), 1.72 (q, J=7.4 Hz, 2H), 1.39 (t, J=6.5 Hz, 6H), 0.98 (t, J=7.4 Hz, 3H). MS (ESI) m/z=364 (M+H)⁺.

### N-((5-(cyclopent-1-en-1-yl)-8-hydroxyquinolin-7-yl)(pyridin-3-yl)methyl)butyramide (G13)

5-(cyclopent-1-en-1-yl)-8-methoxyquinoline was synthesized following the general protocol for Suzuki coupling as mentioned above using cyclopropyl boronic acid (51 mg, 0.6 mmol) and 5-bromo-8-methoxyquinoline (120 mg, 0.5 mmol). The product obtained (85 mg, 0.37 mmol) was treated with boron tribromide in DCM (2 equiv) at 0° C., stirring continued at room temperature until completion of the reaction. Water (2 mL) was added to quench the reaction, followed by concentration and purification by column chromatography to obtain 5-(cyclopenten-1-yl)quinolin-8-ol as white solid (60 mg, Yield 76%).

Starting from 5-(cyclopent-1-enyl)quinolin-8-ol (58 mg, 0.27 mmol), nicotinaldehyde (80 mg, 0.7 mmol), and butyramide (24 mg, 0.27 mmol) following general protocol A, G13 was obtained as a pale yellow solid (18 mg, 17%). ¹H NMR (300 MHz, DMSO-d₆) δ 9.01-8.80 (m, 2H), 8.54 (d, J=9.0 Hz, 1H), 8.48 (s, 1H), 7.72 (s, 1H), 7.62-7.55 (m, 1H), 7.53 (s, 1H), 7.42 (s, 1H), 6.73 (d, J=8.8 Hz, 1H), 5.92 (s, 1H), 2.74 (s, 2H), 2.30-2.12 (m, 2H), 2.12-1.89 (m, 2H), 1.56 (q, J=7.2 Hz, 2H), 0.86 (t, J=7.4 Hz, 3H) (CH₂ overlapped with DMSO-d₆). MS (ESI) m/z=388 (M+H)⁺.

### N-((8-hydroxy-4-methylquinolin-7-yl)(pyridin-3-yl)methyl)butyramide (G15)

Synthesized following the general protocol A, using 4-methylquinolin-8-ol (43 mg, 0.27 mmol), nicotinaldehyde (75 mg, 0.7 mmol), and butyramide (24 mg, 0.27 mmol) as reactants to yield G15 as a white solid (39 mg, 43%) ¹H NMR (300 MHz, DMSO-d₆) δ 8.82 (d, J=8.6 Hz, 1H), 8.72 (d, J=4.3 Hz, 1H), 8.48 (d, J=2.2 Hz, 1H), 8.43 (dd, J=4.8, 1.7 Hz, 1H), 7.59 (dd, J=8.7, 5.4 Hz, 3H), 7.42 (d, J=4.4 Hz, 1H), 7.33 (dd, J=8.0, 4.7 Hz, 1H), 6.70 (d, J=8.6 Hz, 1H), 2.66 (s, 3H), 2.22 (t, J=7.2 Hz, 2H), 1.55 (q, J=7.3 Hz, 2H), 0.86 (t, J=7.4 Hz, 3H). MS (ESI) m/z=336 (M+H)⁺.

### N-((5-fluoro-8-hydroxyquinolin-7-yl)(pyridin-3-yl)methyl)butyramide (G16)

Synthesized following the general protocol A, using 5-fluoroquinolin-8-ol (44 mg, 0.27 mmol), nicotinaldehyde (75 mg, 0.7 mmol), and butyramide (24 mg, 0.27 mmol) as reactants to yield G16 as a pale-yellow solid (23 mg, 25%). ¹H NMR (300 MHz, Methanol-d₄) δ 8.94 (dd, J=4.2, 1.6 Hz, 1H), 8.75 (d, J=2.2 Hz, 1H), 8.69 (dd, J=5.6, 1.5 Hz, 1H), 8.47 (dd, J=8.5, 1.6 Hz, 1H), 8.38 (dt, J=8.1, 2.2 Hz, 1H), 7.96-7.82 (m, 1H), 7.65 (dd, J=8.5, 4.3 Hz, 1H), 7.30 (d, J=10.7 Hz, 1H), 6.84 (s, 1H), 2.38 (t, J=7.4 Hz, 2H), 1.71 (q, J=7.4 Hz, 2H), 0.98 (t, J=7.4 Hz, 3H). MS (ESI) m/z=340 (M+H)⁺.

### N-((5-chloro-8-hydroxyquinolin-7-yl)(pyridin-3-yl)methyl)butyramide (G17)

Synthesized following the general protocol A, using 5-chloroquinolin-8-ol (48 mg, 0.27 mmol), nicotinaldehyde (75 mg, 0.7 mmol), and butyramide (24 mg, 0.27 mmol) as reactants to yield G17 as a white solid (44 mg, 46%). ¹H NMR (300 MHz, DMSO-d₆) δ 10.51 (s, 1H), 8.97 (s, 1H), 8.86 (d, J=8.7 Hz, 1H), 8.47 (d, J=13.8 Hz, 3H), 7.75 (s, 2H), 7.63 (s, 1H), 7.36 (s, 1H), 6.71 (d, J=8.8 Hz, 1H), 2.23 (s, 2H), 1.56 (d, J=8.5 Hz, 2H), 0.88 (d, J=8.4 Hz, 3H). MS (ESI) m/z=356 (M+H)⁺.

### N-((5-chloro-8-hydroxyquinolin-7-yl)(3-(trifluoromethyl)phenyl)methyl)butyramide (G18)

Synthesized following the general protocol A, using 5-chloroquinolin-8-ol (48 mg, 0.27 mmol), 3-(trifluoromethyl)benzaldehyde (121 mg, 0.7 mmol), and butyramide (24 mg, 0.27 mmol) as reactants to yield G18 as a white solid (44 mg, 38%). MS (ESI) m/z=423 (M+H)⁺.

1-ethyl-3-((8-hydroxy-5-methylquinolin-7-yl)(pyridin-3-yl)methyl)urea (G20) Synthesized following the general protocol A, using 5-methylquinolin-8-ol (43 mg, 0.27 mmol), nicotinaldehyde (75 mg, 0.7 mmol), and 2-(methylamino)acetamide (24 mg, 0.27 mmol) as reactants to yield G20 as a brown solid (21 mg, 23%). ¹H NMR (300 MHz, Methanol-d₄) δ 8.89 (dd, J=4.4, 1.5 Hz, 1H), 8.81-8.72 (m, 1H), 8.68 (d, J=5.5 Hz, 1H), 8.54 (dd, J=8.6, 1.5 Hz, 1H), 8.46 (d, J=8.1 Hz, 1H), 7.91 (dd, J=8.2, 5.5 Hz, 1H), 7.65 (dd, J=8.5, 4.3 Hz, 1H), 7.41 (s, 1H), 6.56 (s, 1H), 3.21 (q, J=7.2 Hz, 2H), 2.64 (d, J=0.9 Hz, 3H), 1.22-1.02 (m, 3H). MS (ESI) m/z=337 (M+H)⁺.

### 3-((5-chloro-8-hydroxyquinolin-7-yl)(pyridin-3-yl)methyl)-1,1-dimethylurea (G21)

Synthesized following the general protocol A, using 5-chloroquinolin-8-ol (25 mg, 0.14 mmol), nicotinaldehyde (40 mg, 0.4 mmol), and 1,1-dimethylurea (12 mg, 0.14 mmol) to yield G21 as a white solid (14 mg, 28%). ¹H NMR (300 MHz, DMSO-d₆) δ 10.39 (s, 1H), 8.97 (dd, J=4.2, 1.6 Hz, 1H), 8.50 (dd, J=8.6, 1.7 Hz, 2H), 8.43 (dd, J=4.7, 1.7 Hz, 1H), 7.89 (s, 1H), 7.81-7.66 (m, 1H), 7.64 (d, J=8.2 Hz, 1H), 7.33 (dd, J=8.0, 4.8 Hz, 1H), 7.14 (d, J=8.7 Hz, 1H), 6.63 (d, J=8.6 Hz, 1H), 2.87 (s, 6H). MS (ESI) m/z=357 (M+H)⁺.

### N-((5-chloro-8-hydroxyquinolin-7-yl)(pyrimidin-5-yl)methyl)butyramide (G22)

Synthesized following the general protocol A, using 5-chloroquinolin-8-ol (25 mg, 0.14 mmol), pyrimidine-5-carbaldehyde (39 mg, 0.37 mmol), and butyramide (12 mg, 0.14 mmol) as reactants to yield G22 as a white solid (7 mg, 15%). ¹H NMR (300 MHz, DMSO-d₆) δ 9.09 (s, 1H), 8.97 (dd, J=4.3, 1.5 Hz, 1H), 8.92 (d, J=8.3 Hz, 1H), 8.70 (s, 2H), 8.51 (dd, J=8.6, 1.6 Hz, 1H), 7.77 (s, 1H), 7.76-7.69 (m, 1H), 6.66 (d, J=8.3 Hz, 1H), 2.25 (t, J=7.2 Hz, 2H), 1.57 (q, J=7.3 Hz, 2H), 0.87 (t, J=7.3 Hz, 3H). MS (ESI) m/z=357 (M+H)⁺.

### N-((8-hydroxy-5-methylquinolin-7-yl)(pyridin-3-yl)methyl)butyramide (G23)

Synthesized following the general protocol A, using 5-methylquinolin-8-ol (88 mg, 0.55 mmol), nicotinaldehyde (160 mg, 1.5 mmol), and butyramide (48 mg, 0.55 mmol) as reactants to yield G23 as a brown solid (114 mg, 62%). ¹H NMR (300 MHz, Methanol-d₄) δ 8.91-8.78 (m, 1H), 8.52 (d, J=2.2 Hz, 1H), 8.46-8.34 (m, 2H), 7.85-7.74 (m, 1H), 7.56 (ddd, J=8.5, 4.2, 1.7 Hz, 1H), 7.41 (td, J=6.5, 6.0, 2.9 Hz, 1H), 7.30 (s, 1H), 6.79 (s, 1H), 2.61 (d, J=1.7 Hz, 3H), 2.43-2.24 (m, 2H), 1.71 (qd, J=7.4, 1.7 Hz, 2H), 0.98 (td, J=7.4, 1.7 Hz, 3H). MS (ESI) m/z=336 (M+H)⁺.

### 2-chloro-N-((5-chloro-8-hydroxyquinolin-7-yl)(pyridin-3-yl)methyl)acetamide (G24)

Synthesized following the general protocol A, using 5-chloroquinolin-8-ol (98 mg, 0.55 mmol), nicotinaldehyde (160 mg, 1.5 mmol), and 2-chloroacetamide (51 mg, 0.55 mmol) as reactants to yield G24 as a pale-yellow solid (65 mg, 32%). MS (ESI) m/z=362 (M+H)⁺.

### N-((5-chloro-8-hydroxyquinolin-7-yl)(pyridin-3-yl)methyl)-2-(diethylamino)acetamide (G25)

Synthesized following the general protocol A, using 5-chloroquinolin-8-ol (98 mg, 0.55 mmol), nicotinaldehyde (160 mg, 1.5 mmol), and 2-(diethylamino)acetamide (72 mg, 0.55 mmol) as reactants to yield G25 as a white solid (18 mg, 8%). MS (ESI) m/z=399 (M+H)⁺.

### (2S)-2-amino-N-((5-chloro-8-hydroxyquinolin-7-yl)(pyridin-3-yl)methyl)-3,3-dimethylbutanamide (G26)

Synthesized following the general protocol A, using 5-chloroquinolin-8-ol (98 mg, 0.55 mmol), nicotinaldehyde (160 mg, 1.5 mmol), and tert-butyl carbamate (64 mg, 0.55 mmol) as reactants to yield tert-butyl ((5-chloro-8-hydroxyquinolin-7-yl)(pyridin-3-yl)methyl)carbamate as a brown solid (69 mg). To a solution of tert-butyl ((5-chloro-8-hydroxyquinolin-7-yl)(pyridin-3-yl)methyl)carbamate in DCM, 1 mL of TFA was added and stirred for 2 hrs. On completion of the reaction, product was purified by HPLC to afford 7-(amino(pyridin-3-yl)methyl)-5-chloroquinolin-8-ol as white solid (48 mg).

To a solution of 7-(amino(pyridin-3-yl)methyl)-5-chloroquinolin-8-ol (48 mg, 0.17 mmol) and (S)-2-((tert-butoxycarbonyl)amino)-3,3-dimethylbutanoic acid (44 mg, 0.19 mmol), HATU (91 mg, 0.24 mmol) and DIEA (59 μL, 0.34 mmol) were added at rt. On completion of the reaction as monitored by tlc analysis, HPLC purification was done. tert-butyl ((25)-1-(((5-chloro-8-hydroxyquinolin-7-yl)(pyridin-3-yl)methyl)amino)-3,3-dimethyl-1-oxobutan-2-yl)carbamate obtained was treated with TFA (0.5 mL) in DCM to generate the final product. G26 was obtained as white solid in 42 mg (19%) yield (over two steps).

¹H NMR (300 MHz, Methanol-d₄) δ 8.93 (dd, J=4.2, 1.5 Hz, 1H), 8.61 (d, J=2.3 Hz, 1H), 8.57 (dd, J=8.6, 1.5 Hz, 1H), 8.48 (dd, J=4.9, 1.6 Hz, 1H), 7.85 (dt, J=8.1, 2.0 Hz, 1H), 7.75 (s, 1H), 7.68 (dd, J=8.6, 4.2 Hz, 1H), 7.45 (dd, J=8.1, 4.9 Hz, 1H), 6.85 (s, 1H), 3.75 (s, 1H), 1.13 (s, 9H). MS (ESI) m/z=399 (M+H)⁺.

### N-((5-chloro-8-hydroxyquinolin-7-yl)(pyridin-3-yl)methyl)azetidine-3-carboxamide (G27)

To a stirred solution of 7-(amino(pyridin-3-yl)methyl)-5-chloroquinolin-8-ol (20 mg, 0.07 mmol), 1-(tert-butoxycarbonyl)azetidine-3-carboxylic acid (20 mg, 0.1 mmol) in DCM (4 mL), HATU (53 mg, 0.14 mmol) and DIEA (36 μL, 0.21 mmol) were added and stirred at room temperature for 4 h. On completion of the reaction the product was purified by HPLC, to obtain tert-butyl 3-(((5-chloro-8-hydroxyquinolin-7-yl)(pyridin-3-yl)methyl)carbamoyl)azetidine-1-carboxylate (10 mg, 0.02 mmol). To a solution of this in DCM (1 mL), TFA (0.5 mL) was added and stirred for 1 h to generate the final product. G27 was obtained as a white solid (7 mg, 27% over two steps).

¹H NMR (300 MHz, Methanol-d₄) δ 8.94 (dd, J=4.2, 1.5 Hz, 1H), 8.78 (d, J=2.1 Hz, 1H), 8.67 (dd, J=5.4, 1.5 Hz, 1H), 8.57 (dd, J=8.6, 1.5 Hz, 1H), 8.36-8.25 (m, 1H), 7.82 (dd, J=8.2, 5.4 Hz, 1H), 7.70 (dd, J=8.6, 4.2 Hz, 1H), 7.64 (s, 1H), 6.85 (s, 1H), 4.27 (ddd, J=18.1, 8.2, 5.4 Hz, 4H), 3.89 (td, J=8.4, 6.7 Hz, 1H). MS (ESI) m/z=369 (M+H)⁺.

### N-((5-chloro-8-hydroxyquinolin-7-yl)(pyridin-3-yl)methyl)piperidine-4-carboxamide (G28)

To a stirred solution of 7-(amino(pyridin-3-yl)methyl)-5-chloroquinolin-8-ol (20 mg, 0.07 mmol), 1-(tert-butoxycarbonyl)piperidine-4-carboxylic acid (23 mg, 0.1 mmol) in DCM (4 mL), HATU (53 mg, 0.14 mmol) and DIEA (36 μL, 0.21 mmol) were added and stirred at room temperature for 4 h. On completion of the reaction the product was purified by HPLC, to obtain tert-butyl 4-(((5-chloro-8-hydroxyquinolin-7-yl)(pyridin-3-yl)methyl)carbamoyl)piperidine-1-carboxylate (12 mg, 0.02 mmol). To a solution of this in DCM (1 mL), TFA (0.5 mL) was added and stirred for 1 h. On purification by HPLC G28 was obtained as a white solid (9 mg, 32% over two steps).

¹H NMR (300 MHz, Methanol-d₄) δ 8.94 (dd, J=4.2, 1.5 Hz, 1H), 8.78 (d, J=2.1 Hz, 1H), 8.70 (dd, J=5.5, 1.5 Hz, 1H), 8.57 (dd, J=8.6, 1.5 Hz, 1H), 8.36 (d, J=8.1 Hz, 1H), 7.88 (dd, J=8.1, 5.4 Hz, 1H), 7.70 (dd, J=8.6, 4.2 Hz, 1H), 7.65 (s, 1H), 6.81 (s, 1H), 3.84-3.60 (m, 1H), 3.46 (ddt, J=12.9, 7.7, 4.0 Hz, 2H), 3.15-3.00 (m, 2H), 2.21-1.83 (m, 4H). MS (ESI) m/z=397 (M+H)⁺.

### 2-((3R,4S)-3-amino-4-fluoropyrrolidin-1-yl)-N-((5-chloro-8-hydroxyquinolin-7-yl)(pyridin-3-yl)methyl)acetamide (G29)

To a solution of 2-chloro-N-((5-chloro-8-hydroxyquinolin-7-yl)(pyridin-3-yl)methyl)acetamide (G24) (18 mg, 0.05 mmol) and triethylamine (28 μL, 0.2 mmol) was added tert-butyl ((3R,4S)-4-fluoropyrrolidin-3-yl)carbamate (16 mg, 0.08 mmol) portion wise. The mixture was heated at 80° C. for 12 h. The mixture was concentrated and purified with flash chromatography (10% EtOAc in hexane) to afford tert-butyl ((3R, 4S)-1-(2-(((5-chloro-8-hydroxyquinolin-7-yl)(pyridin-3-yl)methyl)amino)-2-oxoethyl)-4-fluoropyrrolidin-3-yl)carbamate (15 mg, 0.03 mmol). To a solution of tert-butyl ((3R,4S)-1-(2-(((5-chloro-8-hydroxyquinolin-7-yl)(pyridin-3-yl)methyl)amino)-2-oxoethyl)-4-fluoropyrrolidin-3-yl)carbamate in DCM (1 mL), TFA (0.5 mL) was added and stirred for 1 h. On purification by HPLC G29 was obtained as a yellow solid (13 mg, 63% over two steps).

¹H NMR (300 MHz, Methanol-d₄) δ 8.95 (dd, J=4.2, 1.5 Hz, 1H), 8.77 (d, J=2.2 Hz, 1H), 8.68 (d, J=5.4 Hz, 1H), 8.59 (dd, J=8.6, 1.6 Hz, 1H), 8.31 (d, J=8.1 Hz, 1H), 7.82 (dd, J=8.2, 5.5 Hz, 1H), 7.71 (dd, J=8.6, 4.2 Hz, 1H), 7.65 (s, 1H), 6.83 (d, J=4.2 Hz, 1H), 5.45 (t, J=5.1 Hz, 1H), 5.28 (t, J=5.0 Hz, 1H), 4.03 (dd, J=16.2, 6.4 Hz, 1H), 3.66 (t, J=3.1 Hz, 2H), 3.17 (q, J=5.4, 4.2 Hz, 3H). MS (ESI) m/z=430 (M+H)⁺.

### 2-(6-amino-3-azabicyclo[3.1.0]hexan-3-yl)-N-((5-chloro-8-hydroxyquinolin-7-yl)(pyridin-3-yl)methyl)acetamide (G30)

To a solution of 2-chloro-N-((5-chloro-8-hydroxyquinolin-7-yl)(pyridin-3-yl)methyl)acetamide (G24) (20 mg, 0.05 mmol) and triethylamine (28 μL, 0.2 mmol) was added tert-butyl (3-azabicyclo[3.1.0]hexan-6-yl)carbamate (16 mg, 0.08 mmol) portion wise.

The mixture was heated at 80° C. for 12 h. The mixture was concentrated and purified with flash chromatography (10% EtOAc in hexane) to afford tert-butyl (3-(2-(((5-chloro-8-hydroxyquinolin-7-yl)(pyridin-3-yl)methyl)amino)-2-oxoethyl)-3-azabicyclo[3.1.0]hexan-6-yl)carbamate (18 mg, 0.03 mmol). To a solution of this in DCM (1 mL), TFA (0.5 mL) was added and stirred for 1 h. On purification by HPLC G30 was obtained as a yellow solid (15 mg, 70% over two steps).

¹H NMR (300 MHz, Methanol-d₄) δ 8.95 (dd, J=4.2, 1.5 Hz, 1H), 8.80 (d, J=2.2 Hz, 1H), 8.70 (dd, J=5.5, 1.4 Hz, 1H), 8.58 (dd, J=8.6, 1.6 Hz, 1H), 8.36 (d, J=8.1 Hz, 1H), 7.86 (dd, J=8.2, 5.4 Hz, 1H), 7.71 (dd, J=8.6, 4.2 Hz, 1H), 7.65 (s, 1H), 6.82 (s, 1H), 4.29-4.06 (m, 2H), 3.69 (s, 4H), 3.00 (t, J=2.6 Hz, 1H), 2.32 (s, 2H). MS (ESI) m/z=424 (M+H)⁺.

### 2-(2,5-diazabicyclo[2.2.1]heptan-2-yl)-N-((5-chloro-8-hydroxyquinolin-7-yl)(pyridin-3-yl)methyl)acetamide (G31)

To a solution of 2-chloro-N-((5-chloro-8-hydroxyquinolin-7-yl)(pyridin-3-yl)methyl)acetamide (G24) (20 mg, 0.05 mmol) and triethylamine (28 μL, 0.2 mmol) was added tert-butyl 2,5-diazabicyclo[2.2.1]heptane-2-carboxylate (16 mg, 0.08 mmol) portion wise. The mixture was heated at 80° C. for 12 h. The mixture was concentrated and purified with flash chromatography (10% EtOAc in hexane) to afford tert-butyl 5-(2-(((5-chloro-8-hydroxyquinolin-7-yl)(pyridin-3-yl)methyl)amino)-2-oxoethyl)-2,5-diazabicyclo[2.2.1]heptane-2-carboxylate (11 mg, 0.02 mmol). To a solution of this in DCM (1 mL), TFA (0.5 mL) was added and stirred for 1 h. On purification by HPLC G31 was obtained as a yellow solid (8 mg, 37% over two steps).

¹H NMR (300 MHz, Methanol-d₄) δ 8.95 (d, J=4.2 Hz, 1H), 8.71 (d, J=2.5 Hz, 1H), 8.60 (t, J=7.0 Hz, 2H), 8.18 (d, J=8.2 Hz, 1H), 7.74-7.64 (m, 3H), 6.74 (d, J=5.8 Hz, 1H), 4.31 (s, 1H), 3.88 (s, 1H), 3.70-3.41 (m, 4H), 3.12 (dd, J=24.9, 10.7 Hz, 2H), 2.27 (d, J=11.6 Hz, 1H), 1.92 (d, J=11.3 Hz, 1H). MS (ESI) m/z=424 (M+H)⁺.

### N-((5-chloro-8-hydroxyquinolin-7-yl)(pyridin-3-yl)methyl)-1-methylpiperidine-2-carboxamide (G32)

To a stirred solution of 7-(amino(pyridin-3-yl)methyl)-5-chloroquinolin-8-ol (20 mg, 0.07 mmol), 1-methylpiperidine-2-carboxylic acid (14 mg, 0.1 mmol) in DCM (4 mL), HATU (53 mg, 0.14 mmol) and DIEA (36 μL, 0.21 mmol) were added and stirred at room temperature for 4 h. On completion of the reaction the product was purified by HPLC, to obtain G32 as a white solid (9 mg, 31%).

¹H NMR (300 MHz, Methanol-d₄) δ 8.94 (dt, J=4.2, 1.3 Hz, 1H), 8.56 (dt, J=8.6, 2.1 Hz, 2H), 8.50 (dt, J=4.9, 1.4 Hz, 1H), 7.88-7.77 (m, 1H), 7.68 (dd, J=8.6, 4.3 Hz, 1H), 7.54 (d, J=2.9 Hz, 1H), 7.51-7.39 (m, 1H), 6.78 (d, J=5.4 Hz, 1H), 3.49 (s, 1H), 3.36 (s, 3H), 2.89-2.70 (m, 1H), 2.24-2.03 (m, 1H), 1.99-1.65 (m, 4H), 1.62-1.45 (m, 1H), 1.39 (d, J=6.6 Hz, 1H). MS (ESI) m/z=411 (M+H)⁺.

### 6-amino-N-((5-chloro-8-hydroxyquinolin-7-yl)(pyridin-3-yl)methyl)spiro[3.3]heptane-2-carboxamide (G33)

To a stirred solution of 7-(amino(pyridin-3-yl)methyl)-5-chloroquinolin-8-ol (40 mg, 0.13 mmol), 6-((tert-butoxycarbonyl)amino)spiro[3.3]heptane-2-carboxylic acid (33 mg, 0.13 mmol) in DCM (4 mL), HATU (98 mg, 0.26 mmol) and DIEA (67 μL, 0.39 mmol) were added and stirred at room temperature for 4 h. On completion of the reaction the product was purified by HPLC, to obtain tert-butyl (6-(((5-chloro-8-hydroxyquinolin-7-yl)(pyridin-3-yl)methyl)carbamoyl)spiro[3.3]heptan-2-yl)carbamate (31 mg, 0.06 mmol). To a solution of this in DCM (1 mL), TFA (0.5 mL) was added and stirred for 1 h. On purification by HPLC G33 was obtained as a white solid (25 mg, 45% over two steps). MS (ESI) m/z=423 (M+H)⁺.

### N-(benzo[d][1,3]dioxol-5-yl(5-chloro-8-hydroxyquinolin-7-yl)methyl)-2-chloroacetamide (G34)

Synthesized following the general protocol A, using 5-chloroquinolin-8-ol (98 mg, 0.55 mmol), benzo[d][1,3]dioxole-5-carbaldehyde (225 mg, 1.5 mmol), and 2-chloroacetamide (51 mg, 0.55 mmol) to yield G34 as a pale yellow solid (125 mg, 56%). ¹H NMR (300 MHz, DMSO-d₆) δ 10.47 (s, 1H), 9.12 (d, J=8.6 Hz, 1H), 9.01-8.91 (m, 1H), 8.50 (dd, J=8.5, 1.6 Hz, 1H), 7.74 (dd, J=8.5, 4.2 Hz, 1H), 7.68 (s, 1H), 6.86 (d, J=7.7 Hz, 2H), 6.78-6.68 (m, 1H), 6.56 (d, J=8.4 Hz, 1H), 5.98 (s, 2H), 4.20 (d, J=1.2 Hz, 2H). MS (ESI) m/z=405 (M+H)⁺.

### N-(benzo[d][1,3]dioxol-5-yl(5-chloro-8-hydroxyquinolin-7-yl)methyl)-2-((2-(dimethylamino)ethyl)amino)acetamide (G35)

To a solution of N-(benzo[d][1,3]dioxol-5-yl(5-chloro-8-hydroxyquinolin-7-yl)methyl)-2-chloroacetamide (G34) (25 mg, 0.06 mmol) and triethylamine (42 μL, 0.3 mmol) was added N,N-dimethylethane-1,2-diamine (9 mg, 0.10 mmol) portion wise. The mixture was heated at 80° C. for 12 h. On completion the mixture was concentrated and purified with flash chromatography (DCM:MeOH 5:1) to afford G35 as yellow liquid (14 mg, 51%). MS (ESI) m/z=457 (M+H)⁺.

### N-(benzo[d][1,3]dioxol-5-yl(5-chloro-8-hydroxyquinolin-7-yl)methyl)-2-((5-(diethylamino)pentan-2-yl)amino)acetamide (G36)

To a solution of N-(benzo[d][1,3]dioxol-5-yl(5-chloro-8-hydroxyquinolin-7-yl)methyl)-2-chloroacetamide (G34) (25 mg, 0.06 mmol) and triethylamine (42 μL, 0.3 mmol) was added N,N-diethylpentane-1,4-diamine (16 mg, 0.10 mmol) portion wise. The mixture was heated at 80° C. for 12 h. On completion the mixture was concentrated and purified with flash chromatography (DCM:MeOH 5:1) to afford G36 as brown liquid (15 mg, 47%). ¹H NMR (300 MHz, Methanol-d₄) δ 8.98-8.84 (m, 1H), 8.55 (dd, J=8.5, 1.4 Hz, 1H), 7.66 (dd, J=8.6, 4.2 Hz, 1H), 7.55 (d, J=2.0 Hz, 1H), 6.91-6.72 (m, 2H), 6.63 (d, J=2.5 Hz, 1H), 5.94 (s, 2H), 3.60-3.34 (m, 1H), 3.24 (dt, J=13.6, 6.8 Hz, 1H), 3.12-2.99 (m, 2H), 2.91 (qd, J=7.9, 7.2, 2.0 Hz, 2H), 2.87-2.61 (m, 2H), 1.89-1.59 (m, 4H), 1.50 (dq, J=14.2, 7.3 Hz, 1H), 1.30 (p, J=6.6, 6.1 Hz, 3H), 1.24-1.14 (m, 6H), 1.12 (s, 2H). MS (ESI) m/z=527 (M+H)⁺.

### 2-((3S,4R)-3-amino-4-fluoropyrrolidin-1-yl)-N-(benzo[d][1,3]dioxol-5-yl(5-chloro-8-hydroxyquinolin-7-yl)methyl)acetamide (G37)

To a solution of N-(benzo[d][1,3]dioxol-5-yl(5-chloro-8-hydroxyquinolin-7-yl)methyl)-2-chloroacetamide (G34) (25 mg, 0.06 mmol) and triethylamine (42 μL, 0.3 mmol) was added tert-butyl ((3S,4R)-4-fluoropyrrolidin-3-yl)carbamate (20 mg, 0.10 mmol) portion wise. The mixture was heated at 80° C. for 12 h. On completion the mixture was concentrated and purified with flash chromatography (DCM:MeOH 10:1) to afford tert-butyl ((3S,4R)-1-(2-((benzo[d][1,3]dioxol-5-yl(5-chloro-8-hydroxyquinolin-7-yl)methyl)amino)-2-oxoethyl)-4-fluoropyrrolidin-3-yl)carbamate (19 mg, 0.03 mmol). To a solution of this in DCM (1 mL), TFA (0.5 mL) was added and stirred for 1 h. On purification by HPLC G37 was obtained as a brown liquid (17 mg, 60% over two steps).

¹H NMR (300 MHz, Methanol-d₄) δ 8.92 (d, J=4.3 Hz, 1H), 8.54 (d, J=8.6 Hz, 1H), 7.66 (dd, J=8.6, 4.2 Hz, 1H), 7.50 (s, 1H), 6.83 (s, 1H), 6.78 (s, 2H), 6.65 (s, 1H), 5.94 (s, 2H), 4.26 (s, 1H), 4.18 (s, 1H), 4.12 (d, J=3.1 Hz, 1H), 3.85-3.65 (m, 1H), 2.75 (dd, J=13.0, 9.8 Hz, 2H), 2.54-2.36 (m, 2H). MS (ESI) m/z=473 (M+H)⁺.

### 2-(3-aminopiperidin-1-yl)-N-(benzo[d][1,3]dioxol-5-yl(5-chloro-8-hydroxyquinolin-7-yl)methyl)acetamide (G38)

To a solution of N-(benzo[d][1,3]dioxol-5-yl(5-chloro-8-hydroxyquinolin-7-yl)methyl)-2-chloroacetamide (G34) (25 mg, 0.06 mmol) and triethylamine (42 μL, 0.3 mmol) was added tert-butyl piperidin-3-ylcarbamate (20 mg, 0.10 mmol) portion wise. The mixture was heated at 80° C. for 12 h. On completion the mixture was concentrated and purified with flash chromatography (DCM:MeOH 10:1) to afford tert-butyl (1-(2-((benzo[d][1,3]dioxol-5-yl(5-chloro-8-hydroxyquinolin-7-yl)methyl)amino)-2-oxoethyl)piperidin-3-yl)carbamate (17 mg, 0.03 mmol). To a solution of this in DCM (1 mL), TFA (0.5 mL) was added and stirred for 1 h. On purification by HPLC G38 was obtained as a brown liquid (14 mg, 49% over two steps).

¹H NMR (300 MHz, Methanol-d₄) δ 8.92 (d, J=4.1 Hz, 1H), 8.54 (d, J=8.6 Hz, 1H), 7.66 (dd, J=8.6, 4.1 Hz, 1H), 7.54 (d, J=3.6 Hz, 1H), 6.85 (s, 1H), 6.79 (d, J=2.1 Hz, 2H), 6.62 (d, J=1.5 Hz, 1H), 5.93 (s, 2H), 3.44 (s, 1H), 2.93 (d, J=11.6 Hz, 1H), 2.62 (d, J=9.4 Hz, 3H), 2.00-1.49 (m, 4H), 1.45-1.23 (m, 1H). MS (ESI) m/z=469 (M+H)⁺.

### N-((8-hydroxyquinolin-7-yl)(pyridin-3-yl)methyl)butyramide (G40)

Synthesized following the general protocol A, using quinolin-8-ol (39 mg, 0.27 mmol), nicotinaldehyde (72 mg, 0.67 mmol), and acetamide (32 mg, 0.55 mmol) to yield G40 as a white solid (41 mg, 47%).

¹H NMR (300 MHz, Methanol-d₄) δ 8.89 (dd, J=4.4, 1.6 Hz, 1H), 8.79 (s, 1H), 8.73 (d, J=5.9 Hz, 1H), 8.48 (d, J=8.2 Hz, 1H), 8.41 (dd, J=8.3, 1.6 Hz, 1H), 7.97 (dd, J=8.2, 5.6 Hz, 1H), 7.68-7.58 (m, 1H), 7.55 (d, J=1.7 Hz, 1H), 6.85 (s, 1H), 2.39 (dd, J=8.0, 6.7 Hz, 2H), 1.71 (q, J=7.4 Hz, 2H), 0.98 (t, J=7.4 Hz, 3H). MS (ESI) m/z=322 (M+H)⁺.

### N-((8-hydroxy-5-methylquinolin-7-yl)(pyridin-3-yl)methyl)-2-methoxyacetamide (G41)

Synthesized following the general protocol A, using 5-methylquinolin-8-ol (22 mg, 0.14 mmol), nicotinaldehyde (37 mg, 0.35 mmol), and 2-methoxyacetamide (25 mg, 0.28 mmol) as reactants to yield G41 as a white solid (4 mg, 9%).

¹H NMR (300 MHz, Methanol-d₄) δ 8.89 (d, J=4.3 Hz, 1H), 8.74 (s, 1H), 8.66 (d, J=5.5 Hz, 1H), 8.56-8.45 (m, 1H), 8.37 (d, J=8.1 Hz, 1H), 7.84 (dd, J=8.3, 5.6 Hz, 1H), 7.64 (dd, J=8.5, 4.3 Hz, 1H), 7.44 (s, 1H), 6.77 (s, 1H), 4.18-3.93 (m, 2H), 3.48 (s, 3H), 2.65 (d, J=1.0 Hz, 3H). MS (ESI) m/z=338 (M+H)⁺.

### N-((8-hydroxy-5-nitroquinolin-7-yl)(pyridin-3-yl)methyl)butyramide (G42)

Synthesized following the general protocol A, using 5-nitroquinolin-8-ol (51 mg, 0.27 mmol), nicotinaldehyde (72 mg, 0.67 mmol), and acetamide (32 mg, 0.55 mmol) to yield G42 as a white solid (69 mg, 70%). 1H NMR (300 MHz, DMSO-d₆) δ 9.22 (dd, J=9.0, 1.6 Hz, 1H), 9.05-8.96 (m, 2H), 8.71 (s, 1H), 8.61-8.43 (m, 3H), 7.91 (dd, J=8.9, 4.2 Hz, 1H), 7.67 (dd, J=8.2, 2.0 Hz, 1H), 7.37 (dd, J=7.9, 4.8 Hz, 1H), 6.67 (d, J=8.3 Hz, 1H), 2.23 (d, J=14.5 Hz, 2H), 1.63-1.47 (m, 2H), 0.86 (t, J=7.4 Hz, 3H). MS (ESI) m/z=367 (M+H)⁺.

N-((8-methoxy-5-methylquinolin-7-yl)(pyridin-3-yl)methyl)-N-methylbutyramide (G43)

To a stirring solution of G23 (20 mg, 0.06 mmol) in DMF (˜2 mL) at room temperature was added potassium carbonate (11 mg, 0.08 mmol) followed by methyl iodide (11 mg, 0.07 mmol). The reaction mixture was stirred for 4 hrs, and then concentrated. The resulting residue after extraction was purified by column chromatography (n-hexane:EtOAc=3:1, EtOAc) to yield G43 as a yellow oil (15 mg, 67%). MS (ESI) m/z=364 (M+H)⁺.

### N-((5-chloro-8-hydroxyquinolin-7-yl)(phenyl)methyl)butyramide (G45)

Synthesized following the general protocol A, using 5-chloro-8-hydroxyquinoline (103 mg, 0.55 mmol), benzaldehyde (162 mg, 1.5 mmol), and butyramide (44 mg, 0.50 mmol) as reactants to yield G45 as a shiny brown solid (106 mg, 60%). 1H NMR (500 MHz, DMSO-d₆): δ 10.32 (bs, 1H), 8.95 (dd, J=4.2, 1.5 Hz, 1H), 8.74 (d, J=8.8 Hz, 1H), 8.47 (dd, J=8.5, 1.5 Hz, 1H), 7.72-7.69 (m, 1H), 7.69 (s, 1H), 7.32-7.29 (m, 2H), 7.25-7.20 (m, 3H), 6.70 (d, J=8.7 Hz, 1H), 2.19 (t, J=7.2 Hz, 2H), 1.53 (sextet, J=7.4 Hz, 2H), 0.85 (t, J=7.4 Hz, 3H). ¹³C NMR (101 MHz, DMSO-d₆) δ: 171.93, 149.69, 149.66, 142.43, 139.14, 132.98, 128.85, 127.42, 127.38, 126.75, 125.98, 125.29, 123.41, 119.03, 49.93, 37.71, 19.26, 14.09. MS (ESI) m/z=355 (M+H)⁺.

### 2-Chloro-N-((5-chloro-8-hydroxyquinolin-7-yl)(4-methoxyphenyl)methyl)acetamide (G46)

Synthesized following the general protocol A, using 5-chloro-8-hydroxyquinoline (196 mg, 1.04 mmol), 3-methoxybenzaldehyde (393 mg, 2.82 mmol), and 2-chloroacetaamide (90 mg, 0.94 mmol) as reactants to yield G46 as an ivory solid (98 mg, 27%). ¹H NMR (400 MHz, DMSO-d₆) δ: 10.38 (bs, 1H), 9.12 (d, J=8.4 Hz, 1H), 8.97 (dd, J=4.0, 1.2 Hz, 1H), 8.50 (dd, J=8.4, 1.6 Hz, 1H), 7.73 (dd, J=8.4, 4.0 Hz, 1H), 7.69 (s, 1H), 7.19 (d, J=8.4 Hz, 2H), 6.90 (d, J=8.8 Hz, 2H), 6.60 (d, J=8.4 Hz, 1H), 4.19 (d, J=1.2 Hz, 2H), 3.72 (s, 3H). ¹³C NMR (101 MHz, DMSO-d₆) δ: 165.76, 158.85, 149.75, 149.71, 139.16, 133.52, 133.00, 128.62, 126.52, 125.44, 125.37, 123.48, 119.10, 114.35, 55.58, 50.32, 43.21. MS (ESI) m z=391 (M+H)⁺.

### N-((5-chloro-8-hydroxyquinolin-7-yl)(4-chlorophenyl)methyl)butyramide (G47)

Synthesized following the general protocol A, using 5-chloro-8-hydroxyquinoline (116 mg, 0.61 mmol), 4-chlorobenzaldehyde (261 mg, 1.83 mmol), and butyramide (59 mg, 0.67 mmol) as reactants to yield G47 as an off-white solid (102 mg, 43%). ¹H NMR (400 MHz, DMSO-d₆) δ: 10.4 (bs, 1H), 8.97 (dd, J=4.4, 1.6 Hz, 1H), 8.77 (d, J=8.4 Hz, 1H), 8.49 (dd, J=8.8, 1.6 Hz, 1H), 7.74 (dd, J=8.8, 4.4 Hz, 1H), 7.71 (s, 1H), 7.39 (d, J=8.8 Hz, 2H), 7.27 (d, J=8.4 Hz, 2H), 6.69 (d, J=8.8 Hz, 1H), 2.21 (t, J=7.6 Hz, 2H), 1.56 (sextet, J=7.6 Hz, 2H), 0.87 (t, J=7.6 Hz, 3H). ¹³C NMR (101 MHz, DMSO-d₆) δ: 170.02, 149.76, 149.73, 141.44, 139.14, 133.00, 132.01, 129.28, 128.84, 126.56, 125.41, 123.50, 119.17, 49.49, 37.69, 19.23, 14.08. MS (ESI) m/z=389 (M+H)⁺.

### N-((5-chloro-8-hydroxyquinolin-7-yl)(3-chlorophenyl)methyl)butyramide (G48)

Synthesized following the general protocol A, using 5-chloro-8-hydroxyquinoline (173 mg, 0.92 mmol), 3-chlorobenzaldehyde (388 mg, 2.76 mmol), and butyramide (90 mg, 1.01 mmol) as reactants to yield G48 as a white cotton (180 mg, 50%). ¹H NMR (400 MHz, DMSO-d₆) δ: 10.4 (bs, 1H), 8.97 (dd, J=4.4, 1.6 Hz, 1H), 8.80 (d, J=8.8 Hz, 1H), 8.49 (dd, J=8.4, 1.6 Hz, 1H), 7.74 (dd, J=8.4, 4.0 Hz, 1H), 7.72 (s, 1H), 7.38-7.30 (m, 3H), 7.24-7.22 (m, 1H), 6.71 (d, J=8.8 Hz, 1H), 2.22 (t, J=7.2 Hz, 2H), 1.56 (sextet, J=7.2 Hz, 2H), 0.87 (t, J=7.2 Hz, 3H). ¹³C NMR (101 MHz, DMSO-d₆) δ:172.08, 149.80, 149.76, 145.00, 139.15, 133.54, 133.01, 130.85, 127.43, 127.01, 126.48, 126.20, 125.46, 125.19, 123.54, 119.22, 49.68, 37.68, 19.22, 14.04. MS (ESI) m/z=389 (M+H)⁺.

### N-((5-chloro-8-hydroxyquinolin-7-yl)(2-chlorophenyl)methyl)butyramide (G49)

Synthesized following the general protocol A, using 5-chloro-8-hydroxyquinoline (116 mg, 0.61 mmol), 2-chlorobenzaldehyde (265 mg, 1.83 mmol), and butyramide (60 mg, 0.67 mmol) as reactants to yield G49 as an off-white solid (45 mg, 19%). ¹H NMR (400 MHz, CDCl₃) δ: 8.83 (dd, J=4.4, 1.6 Hz, 1H), 8.51 (dd, J=8.4, 1.2 Hz, 1H), 7.59 (s, 1H), 7.57 (dd, J=8.4, 4.0 Hz, 1H), 7.50-7.48 (m, 1H), 7.41-7.39 (m, 1H), 7.26-7.23 (m, 2H), 6.87 (d, J=8.0 Hz, 1H), 6.68 (d, J=8.0 Hz, 1H), 2.29 (t, J=7.2 Hz, 2H), 1.74 (sextet, J=7.6 Hz, 2H), 0.99 (t, J=7.2 Hz, 3H). ¹³C NMR (101 MHz, DMSO-d₆) δ: 171.72, 150.47, 149.69, 139.50, 139.08, 133.44, 133.00, 130.08, 129.45, 129.20, 127.63, 126.82, 125.50, 124.06, 123.53, 118.60, 48.50, 37.54, 19.28, 14.06. MS (ESI) m/z=389 (M+H)⁺.

### N-((5-chloro-8-hydroxyquinolin-7-yl)(3-methoxyphenyl)methyl)butyramide (G50)

Synthesized following the general protocol A, using 5-chloro-8-hydroxyquinoline (116 mg, 0.61 mmol), 3-methoxybenzaldehyde (254 mg, 1.83 mmol), and butyramide (60 mg, 0.67 mmol) as reactants to yield G50 as a white fluffy solid (153 mg, 65%). ¹H NMR (400 MHz, DMSO-d₆) δ: 10.32 (s, 1H), 8.96 (dd, J=5.6, 1.6 Hz, 1H), 8.73 (d, J=8.8 Hz, 1H), 8.48 (dd, J=8.4, 1.6 Hz, 1H), 7.72 (dd, J=8.8, 4.4 Hz, 1H), 7.70 (s, 1H), 7.23 (t, J=8.0 Hz, 1H), 6.84-6.80 (m, 3H), 6.70 (d, J=8.8 Hz, 1H), 3.71 (s, 3H), 2.21 (t, J=7.2 Hz, 2H), 1.59 (sextet, J=7.6 Hz, 2H), 0.87 (t, J=7.6 Hz, 3H). ¹³C NMR (101 MHz, DMSO-d₆) δ:171.94, 159.76, 149.69, 149.66, 144.12, 139.16, 132.97, 129.96, 126.73, 125.91, 125.29, 123.42, 119.64, 118.99, 113.42, 112.32, 55.47, 49.79, 37.72, 19.28, 14.08. MS (ESI) m/z=385 (M+H)⁺.

### N-((5-chloro-8-hydroxyquinolin-7-yl)(3-hydroxyphenyl)methyl)butyramide (G51)

Synthesized following the general protocol A, using 5-chloro-8-hydroxyquinoline (250 mg, 1.40 mmol), 3-hydroxybenzaldehyde (512 mg, 4.19 mmol), and butyramide (134 mg, 1.54 mmol) as reactants. The product obtained from recrystalization was further purified by column chromatography (n-hexane:EtOAc=1:1) to yield G51 as a beige solid (185 mg, 36%).

¹H NMR (400 MHz, CDCl₃) δ: 8.81 (d, J=4.0 Hz, 1H), 8.49 (d, J=8.4 Hz, 1H), 7.55 (dd, J=8.4, 4.0 Hz, 1H), 7.51 (s, 1H), 7.14 (t, J=7.6 Hz, 1H), 7.09 (d, J=8.8 Hz, 1H), 6.88-6.84 (m, 2H), 6.72 (d, J=7.6 Hz, 1H), 6.50 (d, J=8.4 Hz, 1H), 2.28 (t, J=7.6 Hz, 2H), 1.70 (sextet, J=7.6 Hz, 2H), 0.94 (t, J=7.6 Hz, 3H). ¹³C NMR (101 MHz, DMSO-d₆) δ:171.99, 157.77, 149.64, 149.61, 143.83, 139.10, 132.96, 129.81, 126.83, 125.99, 125.26, 123.38, 119.00, 118.10, 114.44, 114.32, 49.76, 37.70, 19.28, 14.06. MS (ESI) m/z=371 (M+H)⁺.

### 3-(Butyramido(5-chloro-8-hydroxyquinolin-7-yl)methyl)benzoic acid (G52)

Synthesized following the general protocol A, using 5-chloro-8-hydroxyquinoline (350 mg, 1.95 mmol), 3-carboxybenzaldehyde (586 mg, 3.9 mmol), and butyramide (186 mg, 2.14 mmol) to obtain G52 as an off-white solid (461 mg, 59%) after washing the crude product with diethy ether and methanol. ¹H NMR (300 MHz, DMSO-d₆) δ: 12.98 (s, 1H), 10.43 (s, 1H), 8.97 (d, J=3.3 Hz, 1H), 8.85 (d, J=8.8 Hz, 1H), 8.50 (d, J=8.6 Hz, 1H), 7.88-7.79 (m, 2H), 7.77-7.70 (m, 2H), 7.55-7.42 (m, 2H), 6.77 (d, J=8.6 Hz, 1H), 2.22 (t, J=7.2 Hz, 2H), 1.56 (sextet, J=7.2 Hz, 2H), 0.87 (t, J=7.3 Hz, 3H). ¹³C NMR (75 MHz, DMSO-d₆) δ: 172.05, 167.60, 149.77, 142.93, 139.10, 133.02, 132.01, 131.34, 129.24, 128.42, 128.08, 126.54, 125.40, 123.53, 119.13, 49.77, 37.67, 19.25, 14.05. MS (ESI) m/z=399 (M+H)⁺, 397 (M−H)⁻.

### N-((5-Chloro-8-hydroxyquinolin-7-yl)(3-cyanophenyl)methyl)butyramide (G53)

Synthesized following the general protocol A, using 5-chloro-8-hydroxyquinoline (200 mg, 1.11 mmol), 3-cyanobenzaldehyde (292 mg, 2.227 mmol), and butyramide (106 mg, 1.22 mmol) to obtain YUM402 as an ivory solid (8 mg, 2%) after purification by column chromatography (n-Haxane:EtOAc=3:1) followed by recrystallization from ethanol. ¹H NMR (300 MHz, CDCl₃+2 drops of MeOD) δ: 8.85 (d, J=4.0 Hz, 1H), 8.54 (d, J=8.4 Hz, 1H), 7.64-7.57 (m, 3H), 7.56-7.51 (m, 2H), 7.41 (t, J=8.0 Hz, 1H), 6.53 (s, 1H), 2.30 (t, J=7.5 Hz, 2H), 1.72 (d, J=7.5 Hz, 2H), 0.97 (t, J=7.4 Hz, 3H). ¹³C NMR (75 MHz, DMSO-d₆) δ: 172.20, 149.90, 149.80, 143.95, 139.14, 133.04, 132.49, 131.37, 130.65, 130.27, 126.37, 125.54, 124.77, 123.62, 119.28, 119.18, 111.83, 49.78, 37.63, 19.18, 14.04. MS (ESI) m/z=380 (M+H)⁺.

### N-(1-(5-chloro-8-hydroxyquinolin-7-yl)butyl)butyramide (G54)

Synthesized following the general protocol A, using 5-chloro-8-hydroxyquinoline (100 mg, 0.53 mmol), butyraldehyde (177 mg, 2.4 mmol), and butyramide (43 mg, 0.48 mmol) as reactants. The product obtained from recrystalization was further purified by column chromatography (n-hexane:EtOAc=5:1, EtOAc) to yield G54 as a beige solid (25 mg, 16%). 1H NMR (500 MHz, DMSO-d6) δ: 10.09 (bs, 1H), 8.93 (dd, J=4.2, 1.5 Hz, 1H), 8.44 (dd, J=8.7, 1.5 Hz, 1H), 8.24 (d, J=8.5 Hz, 1H), 7.68 (dd, J=8.5, 4.1 Hz, 1H), 7.63 (s, 1H), 5.40-5.36 (m, 1H), 2.11 (td, J=7.2, 3.2 Hz, 2H), 1.63 (q, J=7.6 Hz, 2H), 1.50 (sextet, J=7.4 Hz, 2H), 1.39-1.33 (m, 1H), 1.28-1.23 (m, 1H), 0.87 (t, J=7.4 Hz, 3H), 0.83 (t, J=7.4 Hz, 3H). ¹³C NMR (101 MHz, DMSO-d6) δ: 172.02, 149.46, 149.12, 139.02, 132.88, 127.90, 126.01, 124.91, 123.06, 118.88, 46.63, 37.85, 37.69, 19.68, 19.29, 14.07, 14.00. MS (ESI) m/z=343 (M+Na)⁺.

### Methyl 2-(3-(butyramido(5-chloro-8-hydroxyquinolin-7-yl)methyl)phenoxy)acetate (G56)

G51 (130 mg, 0.35 mmol), methyl chloroacetate (0.034 mL, 0.385 mmol), and potassium carbonate (58 mg, 0.42 mmol) were added in anhydous DMF (2 mL), and the reaction mixture was heated at 80° C. for overnight. After cooling to room temperature, the residue was partitioned between water and ethyl acetate, and the aqueous layer was extracted with EtOAc. The combined organic phase was dried over Na₂SO₄ and concentrated in vacuo. The resulting residue was purified using column chromatography (DCM:EtOAc=10:1, 5:1, n-Hexane:EtOAc=1:1, EtOAc) to obtain G56 as a beige crystal (72 mg, 46%). ¹H NMR (300 MHz, CDCl₃) δ: 8.86 (d, J=3.0 Hz, 1H), 8.52 (d, J=8.4 Hz, 1H), 8.04 (d, J=9.0 Hz, 1H), 7.62 (s, 1H), 7.49 (dd, J=8.4, 3.9 Hz, 1H), 7.05 (t, J=7.8 Hz, 1H), 6.77 (s, 1H), 6.69-6.66 (m, 2H), 6.48 (d, J=9.0 Hz, 1H), 5.27 (d, J=15.9 Hz, 1H), 4.53 (d, J=15.9 Hz, 1H), 3.74 (s, 3H), 2.30 (td, J=7.5, 3.0 Hz, 2H), 1.70 (sextet, J=7.5 Hz, 2H), 0.92 (t, J=7.5 Hz, 3H). ¹³C NMR (75 MHz, CDCl₃) δ: 173.46, 170.53, 156.77, 150.63, 149.67, 142.64, 142.30, 133.71, 133.55, 129.49, 127.99, 126.89, 126.30, 122.04, 117.80, 114.70, 113.85, 70.53, 54.36, 52.17, 38.56, 19.18, 13.82. MS (ESI) m/z=443 (M+H)⁺, 441 (M−H)⁻.

### 2-(3-(Butyramido(5-chloro-8-hydroxyquinolin-7-yl)methyl)phenoxy)acetic acid (G55)

To a solution of G56 (43 mg, 0.097 mmol) in MeOH (2 mL) was added 50% NaOH (40 drops), and the mixture was stirred at room temperature for 1 hr. After neutralization with 3 eq of 1N HCl, the precipitate was filtered, washed with ice-water, and dried to obtain G55 as a beige solid (14 mg, 34%). ¹H NMR (400.1 MHz, CDCl₃) δ: 8.97 (d, J=2.4 Hz, 1H), 8.56 (dd, J=8.8, 1.6 Hz, 1H), 7.63 (s, 1H), 7.53 (dd, J=8.8, 4.4 Hz, 1H), 7.24 (t, J=8.0 Hz, 1H), 6.86-6.74 (m, 4H), 6.59 (d, J=8.4 Hz, 1H), 3.93 (s, 3H), 3.78 (s, 3H), 2.29 (td, J=7.6, 2.4 Hz, 2H), 1.74 (sextet, J=7.6 Hz, 2H), 0.99 (t, J=7.2 Hz, 3H). ¹³C NMR (100.6 MHz, CDCl₃) δ:172.29, 159.81, 152.82, 150.03, 143.48, 142.87, 134.23, 133.36, 129.61, 127.07, 126.84, 126.14, 121.96, 118.97, 112.98, 112.19, 62.54, 55.26, 53.40, 38.77, 19.15, 13.83. MS (ESI) m/z=429 (M+H)⁺, 427 (M−H)⁻.

### N-((5-Chloro-8-(((2E,6E)-3,7,11-trimethyldodeca-2,6,10-trien-1-yl)oxy)quinolin-7-yl)(3-chlorophenyl)methyl)butyramide (G57)

To a dried flask were added YUM194 (97 mg, 0.25 mmol), triphenylphosphine (72 mg, 0.275 mmol), and trans-famesol (61 mg, 0.275 mmol) in THF with stirring, and the flask was covered by rubber septum. While stirring, DEAD (40% in toluene, 120 mg, 0.275 mmol) was added dropwise, and the reaction mixture was heated at 60° C. for overnight. After cooling, the resulting residue was concentrated under reduced pressure, and then purified by column chromatography (n-Haxane:EtOAc=5:1) to afford G57 as an ivory solid (24 mg, 28%). ¹H NMR (400 MHz, CDCl₃) δ: 8.96 (dd, J=4.0, 1.6 Hz, 1H), 8.55 (dd, J=8.4, 1.6 Hz, 1H), 7.61 (s, 1H), 7.53 (dd, J=8.4, 4.0 Hz, 1H), 7.27 (s, 1H), 7.23-7.21 (m, 2H), 7.15-7.12 (m, 1H), 6.99 (d, J=8.8 Hz, 1H), 6.56 (d, J=8.4 Hz, 1H), 5.39 (td, J=6.8, 0.8 Hz, 1H), 5.16-5.09 (m, 3H), 4.39 (dd, J=10.8, 6.8 Hz, 1H), 2.28 (td, J=8.0, 4.4 Hz, 2H), 2.11-1.97 (m, 8H), 1.78-1.71 (m, 2H), 1.70 (s, 3H), 1.62 (s, 9H), 0.98 (t, J=7.2 Hz, 3H). MS (ESI) m z=593 (M+H)⁺, 615 (M+Na)⁺.

### 2-Chloro-N-((5-chloro-8-hydroxyquinolin-7-yl)(phenyl)methyl)acetamide (G58)

The same procedure for the synthesis of G44 was followed using 5-chloro-8-hydroxyquinoline (100 mg, 0.53 mmol), benzaldehyde (156 mg, 1.4 mmol), and 2-chloroacetamide (46 mg, 0.48 mmol) as reactants to yield G58 as a brown solid (59 mg, 34%). ¹H NMR (500 MHz, DMSO-d6) δ: 10.43 (bs, 1H), 9.17 (d, J=8.5 Hz, 1H), 8.95 (dd, J=4.4, 1.5 Hz, 1H), 8.49-8.46 (m, 1H), 7.72 (dd, J=8.5, 4.2 Hz, 1H), 7.67 (s, 1H), 7.34-7.31 (m, 2H), 7.27-7.22 (m, 3H), 6.65 (d, J=8.4 Hz, 1H), 4.19 (d, J=2.6 Hz, 2H). ¹³C NMR (101 MHz, DMSO-d6) δ:165.89, 149.89, 149.76, 141.58, 139.16, 133.02, 128.97, 127.65, 127.40, 126.63, 125.45, 125.05, 123.56, 119.16, 50.79, 43.19. MS (ESI) m/z=383 (M+Na)⁺.

### 2-Chloro-N-((5-chloro-8-hydroxyquinolin-7-yl)(3-methoxyphenyl)methyl)acetamide (G59)

Synthesized following the general protocol A, using 5-chloro-8-hydroxyquinoline (196 mg, 1.04 mmol), 3-methoxybenzaldehyde (392 mg, 2.82 mmol), and 2-chloroacetamide (90 mg, 0.94 mmol) as reactants to yield G59 as an ivory solid (52 mg, 14%). ¹H NMR (400 MHz, DMSO-d6) δ: 10.43 (bs, 1H), 9.16 (d, J=8.4 Hz, 1H), 8.97 (dd, J=4.4, 1.6 Hz, 1H), 8.49 (dd, J=8.4, 1.6 Hz, 1H), 7.74 (dd, J=8.4, 4.4 Hz, 1H), 7.66 (s, 1H), 7.25 (t, J=8.0 Hz, 1H), 6.85-6.82 (m, 3H), 6.64 (d, J=8.8 Hz, 1H), 4.20 (d, J=3.6 Hz, 2H), 3.72 (s, 3H). ¹³C NMR (101 MHz, DMSO-d6) δ: 165.88, 159.82, 149.89, 149.76, 143.24, 139.18, 133.02, 130.10, 126.62, 125.45, 125.00, 123.57, 119.59, 119.13, 113.43, 112.58, 55.52, 50.64, 43.20. MS (ESI) m/z=391 (M+H)⁺.

### N-(Benzo[d][1,3]dioxol-5-yl(8-hydroxyquinolin-7-yl)methyl)butyramide (G60)

8-Hydroxyquinoline (183 mg, 1.26 mmol), piperonal (518 mg, 3.45 mmol), butyramide (100 mg, 1.15 mmol) and ethyl acetate were placed in a microwave tube, and the sealed vessel was heated by microwave to 180° C. for 30 min. After cooling to room temperature, the mixture was triturated with ethyl acetate and haxane. The crude solid was purified by column chromatography (n-hexane:EtOAc=3:1) to yield G60 as an ivory solid (60 mg, 14%). ¹H NMR (400 MHz, DMSO-d₆) δ: 9.89 (bs, 1H), 8.85 (dd, J=4.4, 1.6 Hz, 1H), 8.61 (d, J=8.8 Hz, 1H), 8.30 (dd, J=8.4, 1.6 Hz, 1H), 7.55-7.52 (m, 2H), 7.41 (d, J=8.4 Hz, 1H), 6.81 (d, J=8.0 Hz, 1H), 6.79 (d, J=1.6 Hz, 1H), 6.70 (dd, J=8.0, 1.2 Hz, 1H), 6.61 (d, J=8.4 Hz, 1H), 5.95 (d, J=0.8 Hz, 2H), 2.18 (td, J=7.2, 2.8 Hz, 2H), 1.53 (sextet, J=7.2 Hz, 2H), 0.85 (t, J=7.2 Hz, 3H). ¹³C NMR (75 MHz, DMSO-d6) δ:171.76, 149.76, 148.79, 147.63, 146.35, 138.47, 137.02, 136.48, 127.90, 126.64, 125.39, 122.17, 120.57, 117.77, 108.40, 107.89, 101.33, 49.91, 37.68, 19.28, 14.12. MS (ESI) m/z=365 (M+H)⁺, 363 (M−H)⁻.

### N-(Benzo[d][1,3]dioxol-5-yl(5-bromo-8-hydroxyquinolin-7-yl)methyl)butyramide (G61)

Synthesized following the general protocol A, using 5-bormoquinoline-8-ol (300 mg, 1.339 mmol), piperonal (402 mg, 2.678 mmol), and butyramide (128 mg, 1.473 mmol) to obtain G61 as a tan solid (14 mg, 2%). ¹H NMR (300 MHz, DMSO-d₆) δ: 10.39 (bs, 1H), 8.93 (s, 1H), 8.68 (d, J=8.3 Hz, 1H), 8.41 (d, J=7.5 Hz, 1H), 7.87 (s, 1H), 7.71 (d, J=4.0 Hz, 1H), 6.91-6.76 (m, 2H), 6.70 (d, J=7.5 Hz, 1H), 6.60 (d, J=7.5 Hz, 1H), 5.96 (s, 2H), 2.19 (t, J=6.6 Hz, 2H), 1.54 (sextet, J=7.2 Hz, 2H), 0.85 (t, J=6.6 Hz, 3H). ¹³C NMR (75 MHz, DMSO-d₆) δ: 171.83, 150.11, 149.66, 147.74, 146.56, 139.35, 136.36, 135.42, 129.96, 126.83, 126.54, 123.73, 120.54, 108.92, 108.51, 107.81, 101.42, 49.64, 37.68, 19.25, 14.07. MS (ESI) m/z=444 (M+H)⁺, 442 (M−H)⁻.

### N-(Benzo[d][1,3]dioxol-5-yl(5-chloro-8-methoxyquinolin-7-yl)methyl)butyramide (G62)

To a stirring solution of N-(benzo[d][1,3]dioxol-5-yl(5-chloro-8-hydroxyquinolin-7-yl)methyl)butyramide (33 mg, 0.0827 mmol) in DMF (˜4 mL) at room temperature was added potassium carbonate (14 mg, 0.0992 mmol) followed by methyl iodide (13 mg, 0.0910 mmol). The reaction mixture was stirred for 4 hrs, and then concentrated. The resulting residue after extraction was purified by column chromatography (n-hexane:EtOAc=3:1, 1:1) to yield G62 as a white solid (24 mg, 70%). ¹H NMR (400 MHz, CDCl₃) δ: 8.95 (dd, J=4.4, 1.6 Hz, 1H), 8.54 (dd, J=8.4, 1.2 Hz, 1H), 7.58 (s, 1H), 7.51 (dd, J=8.4, 4.0 Hz, 1H), 6.75-6.69 (m, 3H), 6.64 (d, J=8.0 Hz, 1H), 6.50 (d, J=8.0 Hz, 1H), 5.92 (d, J=2.0 Hz, 2H), 3.97 (s, 3H), 2.26 (td, J=7.6, 2.8 Hz, 2H), 1.71 (sextet, J=7.2 Hz, 2H), 0.96 (t, J=7.2 Hz, 3H). ¹³C NMR (101 MHz, CDCl₃) δ: 172.26, 152.73, 150.08, 147.94, 146.84, 143.50, 135.14, 134.21, 133.34, 127.04, 126.60, 126.19, 121.98, 119.90, 108.20, 107.42, 101.17, 62.62, 53.25, 38.75, 19.12, 13.83. MS (ESI) m/z=413 (M+H)⁺.

### N-((5-chloro-8-methoxyquinolin-7-yl)(3-methoxyphenyl)methyl)butyramide (G63)

To a stirring solution of G50 (50 mg, 0.13 mmol) in DMF (˜4 mL) at room temperature was added potassium carbonate (22 mg, 0.16 mmol) followed by methyl iodide (21 mg, 0.14 mmol). The reaction mixture was stirred for 4 hrs, and then concentrated. The resulting residue after extraction was purified by column chromatography (n-hexane:EtOAc=3:1, EtOAc) to yield G63 as a yellow oil (33 mg, 64%). ¹H NMR (400 MHz, CDCl₃) δ: 8.97 (d, J=2.4 Hz, 1H), 8.56 (dd, J=8.8, 1.6 Hz, 1H), 7.63 (s, 1H), 7.53 (dd, J=8.8, 4.4 Hz, 1H), 7.24 (t, J=8.0 Hz, 1H), 6.86-6.74 (m, 4H), 6.59 (d, J=8.4 Hz, 1H), 3.93 (s, 3H), 3.78 (s, 3H), 2.29 (td, J=7.6, 2.4 Hz, 2H), 1.74 (sextet, J=7.6 Hz, 2H), 0.99 (t, J=7.2 Hz, 3H). ¹³C NMR (101 MHz, CDCl₃) δ: 172.29, 159.81, 152.82, 150.03, 143.48, 142.87, 134.23, 133.36, 129.61, 127.07, 126.84, 126.14, 121.96, 118.97, 112.98, 112.19, 62.54, 55.26, 53.40, 38.77, 19.15, 13.83. MS (ESI) m/z=399 (M+H)⁺.

### 2-Chloro-N-((5-chloro-8-methoxyquinolin-7-yl)(3-methoxyphenyl)methyl)acetamide (G64)

To a stirring solution of G59 (39 mg, 0.10 mmol) in DMF (˜4 mL) at room temperature was added potassium carbonate (17 mg, 0.12 mmol) followed by methyl iodide (16 mg, 0.11 mmol) dropwise. The reaction mixture was stirred for 4 hrs, and then concentrated. The resulting residue was diluted with EtOAc, and the organic layer was washed with water (20 mL), sat. NaHCO₃ (aq.) (20 mL), and brine (20 mL), and then concentrated in vacuo. The residue was purified by column chromatography (n-hexane:EtOAc=3:1) to yield G64 as a white crystal (28 mg, 69%). ¹H NMR (400 MHz, DMSO-d6) δ: 9.22 (d, J=8.4 Hz, 1H), 9.03 (dd, J=4.0, 1.6 Hz, 1H), 8.53 (dd, J=8.8, 1.6 Hz, 1H), 7.72 (dd, J=8.4, 4.0 Hz, 1H), 7.71 (s, 1H), 7.27 (td, J=7.6, 1.6 Hz, 1H), 6.86-6.82 (m, 3H), 6.66 (d, J=8.4 Hz, 1H), 4.24-4.16 (m, 2H), 4.03 (s, 3H), 3.72 (s, 3H). ¹³C NMR (101 MHz, CDCl₃) δ: 165.38, 159.90, 152.99, 150.08, 143.43, 141.98, 133.37, 133.07, 129.80, 127.24, 126.67, 126.22, 122.12, 118.79, 112.93, 112.42, 62.74, 55.29, 53.93, 42.71. MS (ESI) m/z=405 (M+H)⁺.

### N-((5-chloro-8-hydroxyquinolin-7-yl)(3-methoxyphenyl)methyl)butyramide (G66)

Synthesized following the general protocol A, using 5-chloro-8-hydroxyquinoline (48 mg, 0.27 mmol), 3-methoxybenzaldehyde (91 mg, 0.67 mmol), and butyramide (48 mg, 0.55 mmol) as reactants to yield G66 as a white solid (41 mg, 39%). ¹H NMR (300 MHz, Chloroform-d) δ 8.83 (dd, J=4.3, 1.5 Hz, 1H), 8.52 (dd, J=8.5, 1.6 Hz, 1H), 7.58 (q, J=4.5 Hz, 2H), 7.23 (d, J=7.9 Hz, 1H), 7.04-6.88 (m, 3H), 6.81 (d, J=8.7 Hz, 1H), 6.54 (d, J=8.7 Hz, 1H), 2.29 (t, J=7.6 Hz, 2H), 1.72 (p, J=7.5 Hz, 4H), 1.04-0.86 (m, 3H). MS (ESI) m/z=385 (M+H)⁺.

### N-((5-(tert-butyl)-8-hydroxyquinolin-7-yl)(pyridin-3-yl)methyl)-3-methylbutanamide (G67)

Synthesized following the general protocol A, using 5-(tert-butyl)quinolin-8-ol (27 mg, 0.14 mmol), nicotinaldehyde (36 mg, 0.33 mmol), and 3-methylbutanamide (27 mg, 0.27 mmol) as reactants to yield G67 as a pale yellow solid (21 mg, 38%). ¹H NMR (300 MHz, DMSO-d₆) δ 8.97 (d, J=8.3 Hz, 1H), 8.09 (d, J=8.2 Hz, 1H), 7.83-7.68 (m, 1H), 7.62 (dd, J=8.8, 4.2 Hz, 1H), 7.54 (s, 1H), 6.74 (d, J=8.1 Hz, 1H), 2.14 (d, J=7.0 Hz, 2H), 2.09-1.90 (m, 1H), 1.52 (s, 9H), 0.88 (t, J=6.0 Hz, 6H). MS (ESI) m/z=392 (M+H)⁺.

### 3-((5-(tert-butyl)-8-hydroxyquinolin-7-yl)(butyramido)methyl)benzoic acid (G69)

Synthesized following the general protocol A, using 5-(tert-butyl)quinolin-8-ol (56 mg, 0.27 mmol), 3-formylbenzoic acid (98 mg, 0.66 mmol), and butyramide (48 mg, 0.55 mmol) as reactants to yield G69 as a pale yellow solid (81 mg, 71%). MS (ESI) m/z=421 (M+H)⁺.

### N-((5-chloro-8-hydroxyquinolin-7-yl)(pyridin-2-yl)methyl)butyramide (G70)

Synthesized following the general protocol A, using 5-chloroquinolin-8-ol (48 mg, 0.27 mmol), picolinaldehyde (75 mg, 0.7 mmol), and butyramide (24 mg, 0.27 mmol) as reactants to yield G70 as a white solid (44 mg, 46%). ¹H NMR (300 MHz, DMSO-d₆) δ 8.96 (d, J=3.8 Hz, 2H), 8.87 (d, J=8.2 Hz, 1H), 8.57 (d, J=4.7 Hz, 1H), 8.49 (d, J=8.5 Hz, 1H), 7.91 (t, J=7.7 Hz, 2H), 7.83-7.66 (m, 2H), 7.50 (d, J=8.1 Hz, 1H), 7.47-7.33 (m, 1H), 6.74 (d, J=8.1 Hz, 1H), 2.23 (t, J=7.3 Hz, 2H), 1.70-1.38 (m, 2H), 0.85 (dd, J=8.6, 6.1 Hz, 3H). MS (ESI) m/z=356 (M+H)⁺.

### N-((3-(dimethylamino)phenyl)(8-hydroxy-5-methylquinolin-7-yl)methyl)butyramide (G71)

Synthesized following the general protocol A, using 5-methylquinolin-8-ol (44 mg, 0.28 mmol), 3-(dimethylamino)benzaldehyde (104 mg, 0.7 mmol), and butyramide (48 mg, 0.56 mmol) as reactants to yield G71 as brown solid (48 mg, 46%). ¹H NMR (400 MHz, Methanol-d₄) δ 8.96 (dd, J=4.8, 1.5 Hz, 1H), 8.84 (dd, J=8.5, 1.5 Hz, 1H), 7.84 (dd, J=8.5, 4.8 Hz, 1H), 7.52-7.37 (m, 2H), 7.24 (dd, J=4.9, 2.6 Hz, 2H), 7.18 (d, J=7.7 Hz, 1H), 6.78 (s, 1H), 3.13 (s, 6H), 2.66 (d, J=1.0 Hz, 3H), 2.35 (td, J=7.3, 4.0 Hz, 2H), 1.70 (q, J=7.4 Hz, 2H), 0.97 (t, J=7.4 Hz, 3H). MS (ESI) m/z=378 (M+H)⁺.

### N-((2,2-difluorobenzo[d][1,3]dioxol-5-yl)(8-hydroxy-5-methylquinolin-7-yl)methyl)butyramide (G73)

Synthesized following the general protocol A, using 5-methylquinolin-8-ol (44 mg, 0.28 mmol), 2,2-difluorobenzo[d][1,3]dioxole-5-carbaldehyde (130 mg, 0.7 mmol), and butyramide (48 mg, 0.56 mmol) as reactants to yield G73 as a white solid (57 mg, 50%). ¹H NMR (400 MHz, DMSO-d₆) δ 9.79 (s, 1H), 8.88 (dd, J=4.2, 1.6 Hz, 1H), 8.67 (d, J=8.5 Hz, 1H), 8.40 (dd, J=8.5, 1.6 Hz, 1H), 7.60 (dd, J=8.5, 4.2 Hz, 1H), 7.40-7.24 (m, 3H), 7.09 (dd, J=8.4, 1.8 Hz, 1H), 6.74-6.66 (m, 1H), 2.54 (s, 3H), 2.22 (td, J=7.2, 3.3 Hz, 2H), 1.65-1.52 (m, 2H), 0.95-0.78 (m, 3H). MS (ESI) m/z=415 (M+H)⁺.

### N-((8-hydroxy-5-methylquinolin-7-yl)(2-oxo-2,3-dihydro-1H-benzo[d]imidazol-5-yl)methyl)butyramide (G75)

Synthesized following the general protocol A, using 5-methylquinolin-8-ol (22 mg, 0.14 mmol), 2-oxo-2,3-dihydro-1H-benzo[d]imidazole-5-carbaldehyde (57 mg, 0.35 mmol), and butyramide (24 mg, 0.28 mmol) as reactants to yield G75 as a white solid (48 mg, 46%). ¹H NMR (300 MHz, DMSO-d₆) δ 10.53 (d, J=17.7 Hz, 2H), 8.88 (d, J=4.2 Hz, 1H), 8.67 (d, J=8.7 Hz, 1H), 8.46 (d, J=8.6 Hz, 1H), 7.63 (dd, J=8.9, 4.8 Hz, 1H), 7.41 (s, 1H), 6.84 (s, 2H), 6.78 (s, 1H), 6.66 (d, J=8.6 Hz, 1H), 2.54 (s, 3H), 2.19 (t, J=7.3 Hz, 2H), 1.52 (dt, J=15.2, 7.6 Hz, 2H), 0.85 (t, J=7.4 Hz, 3H). MS (ESI) m/z=391 (M+H)⁺.

### N-((1H-benzo[d]imidazol-6-yl)(8-hydroxy-5-methylquinolin-7-yl)methyl)butyramide (G76)

Synthesized following the general protocol A, using 5-methylquinolin-8-ol (22 mg, 0.14 mmol), 1H-benzo[d]imidazole-6-carbaldehyde (51 mg, 0.35 mmol), and butyramide (24 mg, 0.28 mmol) as reactants to yield G76 as a white solid (13 mg, 25%). ¹H NMR (400 MHz, DMSO-d₆) δ 9.40 (s, 1H), 8.89 (dd, J=4.2, 1.6 Hz, 1H), 8.80 (d, J=8.6 Hz, 1H), 8.60 (s, 1H), 8.42 (dd, J=8.6, 1.6 Hz, 1H), 7.85-7.71 (m, 2H), 7.68-7.58 (m, 2H), 7.48 (t, J=7.6 Hz, 1H), 7.40 (s, 1H), 6.88 (d, J=8.6 Hz, 1H), 6.68 (t, J=7.8 Hz, 1H), 2.54 (d, J=0.9 Hz, 3H), 2.25 (td, J=7.2, 2.3 Hz, 2H), 1.60-1.50 (m, 2H), 0.88 (q, J=7.6 Hz, 3H). MS (ESI) m/z=375 (M+H)⁺.

### N-((4-(diethylamino)phenyl)(8-hydroxy-5-methylquinolin-7-yl)methyl)butyramide (G80)

Synthesized following the general protocol A, using 5-methylquinolin-8-ol (22 mg, 0.14 mmol), 4-(diethylamino)benzaldehyde (58 mg, 0.33 mmol), and butyramide (24 mg, 0.27 mmol) as reactants to yield G80 as a pale yellow solid (8 mg, 14%). ¹H NMR (300 MHz, DMSO-d₆) δ 8.89 (dd, J=4.2, 1.5 Hz, 1H), 8.69 (s, 1H), 8.52-8.35 (m, 1H), 7.63 (dd, J=8.5, 4.2 Hz, 2H), 7.40 (s, 1H), 7.28 (s, 2H), 6.67 (d, J=8.2 Hz, 1H), 2.53 (d, J=8.5 Hz, 4H), 2.21 (t, J=7.2 Hz, 2H), 1.55 (q, J=7.3 Hz, 2H), 0.99 (t, J=7.0 Hz, 6H), 0.86 (t, J=7.4 Hz, 3H). MS (ESI) m/z=406 (M+H)⁺.

### N-(benzo[d][1,3]dioxol-5-yl(5-chloro-8-hydroxyquinolin-7-yl)methyl)butyramide (G96)

A mixture of 5-chloro-8-hydroxyquinoline (486 mg, 2.57 mmol), piperonal (1.06 g, 7.02 mmol), and butyramide (200 mg, 2.34 mmol) were stirred neat at 130-150° C. for 6-12 hrs. Upon heating, the reaction mixture melted and solid was formed after completion of the reaction. The solid was isolated by multiple trituration with ethyl acetate and diethyl ether, and the crude product was purified by recrystallization from ethanol to yield G96 as a tan solid (302 mg, 32%). 1H NMR (500 MHz, DMSO-d6) δ: 10.30 (bs, 1H), 8.94 (dd, J=4.1, 1.5 Hz, 1H), 8.66 (d, J=8.8 Hz, 1H), 8.46 (dd, J=8.5, 1.5 Hz, 1H), 7.72-7.69 (m, 1H), 7.70 (s, 1H), 6.84-6.80 (m, 2H), 6.70 (d, J=8.1 Hz, 1H), 6.60 (d, J=8.7 Hz, 1H), 5.95 (s, 2H), 2.18 (t, J=7.2 Hz, 2H), 1.51 (sextet, J=7.2 Hz, 2H), 0.84 (t, J=7.3 Hz, 3H). ¹³C NMR (101 MHz, DMSO-d6) δ: 171.85, 149.64, 149.52, 147.78, 146.58, 139.14, 136.40, 132.96, 126.53, 126.18, 125.26, 123.38, 120.57, 119.05, 108.51, 107.84, 101.43, 49.72, 37.71, 19.25, 14.08. MS (ESI) m/z=399 (M+H)⁺.

### N-((5-(cyclohex-1-en-1-yl)-8-hydroxyquinolin-7-yl)(pyridin-3-yl)methyl)butyramide (G97)

5-(cyclohex-1-en-1-yl)-8-methoxyquinoline was synthesized following the general protocol for Suzuki coupling as mentioned above using cyclohexyl boronic acid (77 mg, 0.6 mmol) and 5-bromo-8-methoxyquinoline (120 mg, 0.5 mmol). The product obtained (82 mg, 0.34 mmol) was treated with boron tribromide in DCM (2 equiv) at 0° C., stirring continued at room temperature until completion of the reaction. Water (2 mL) was added to quench the reaction, followed by concentration and purification by column chromatography to obtain 5-(cyclohex-1-en-1-yl)quinolin-8-ol as white solid (68 mg).

Starting from 5-(cyclohex-1-en-1-yl)quinolin-8-ol (61 mg, 0.27 mmol), nicotinaldehyde (80 mg, 0.7 mmol), and butyramide (24 mg, 0.27 mmol) following general protocol A, G97 was obtained as a pale yellow solid (15 mg, 14%). ¹H NMR (300 MHz, DMSO-d₆) δ 8.92-8.76 (m, 2H), 8.50 (d, J=2.4 Hz, 1H), 8.43 (dd, J=4.8, 1.6 Hz, 1H), 8.34 (dd, J=8.7, 1.6 Hz, 1H), 7.63 (d, J=8.2 Hz, 1H), 7.55 (dd, J=8.5, 4.2 Hz, 1H), 7.43 (s, 1H), 7.34 (dd, J=7.9, 4.8 Hz, 1H), 6.74 (d, J=8.7 Hz, 1H), 5.68 (s, 1H), 2.24 (q, J=10.7, 7.3 Hz, 6H), 1.75 (d, J=21.9 Hz, 4H), 1.55 (q, J=7.3 Hz, 2H), 0.85 (t, J=7.4 Hz, 3H). MS (ESI) m/z=402 (M+H)⁺.

### N-((5-Chloro-8-hydroxyquinolin-7-yl)(3-(((2E,6E)-3,7,11-trimethyldodeca-2,6,10-trien-1-yl)oxy)phenyl)methyl)butyramide (G98)

To a dried flask were added G51 (100 mg, 0.269 mmol), triphenylphosphine (78 mg, 0.296 mmol), and trans-farnesol (66 mg, 0.296 mmol) in THF with stirring, and the flask was covered by rubber septum. While stirring DEAD (40% in toluene, 129 mg, 0.296 mmol) was added dropwise, and the reaction mixture was heated at 60° C. for overnight. After cooling, the resulting residue was concentrated under reduced pressure, and then purified by column chromatography (n-Haxane:EtOAc=5:1) to obtain G98 as a yellow solid (55 mg, 36%). ¹H NMR (400 MHz, CDCl₃) δ: 8.92 (dd, J=4.0, 1.6 Hz, 1H), 8.52 (dd, J=8.4, 1.6 Hz, 1H), 7.56 (s, 1H), 7.49 (dd, J=8.8, 4.4 Hz, 1H), 7.27 (s, 1H), 7.13 (t, J=8.0 Hz, 1H), 6.96 (d, J=8.4 Hz, 1H), 6.77 (d, J=7.6 Hz, 1H), 6.70-6.68 (m, 2H), 6.50 (d, J=8.8 Hz, 1H), 5.71 (s, 1H), 5.42 (t, J=6.8 Hz, 1H), 5.11-5.02 (m, 3H), 4.42 (dd, J=10.8, 6.8 Hz, 1H), 2.24 (td, J=7.6, 4.4 Hz, 2H), 2.06-1.95 (m, 8H), 1.75-1.68 (m, 5H), 1.59 (s, 9H), 0.95 (t, J=7.2 Hz, 3H). MS (ESI) m/z=575.19 (M+H)⁺.

General Scheme for PROTACs

### Series A

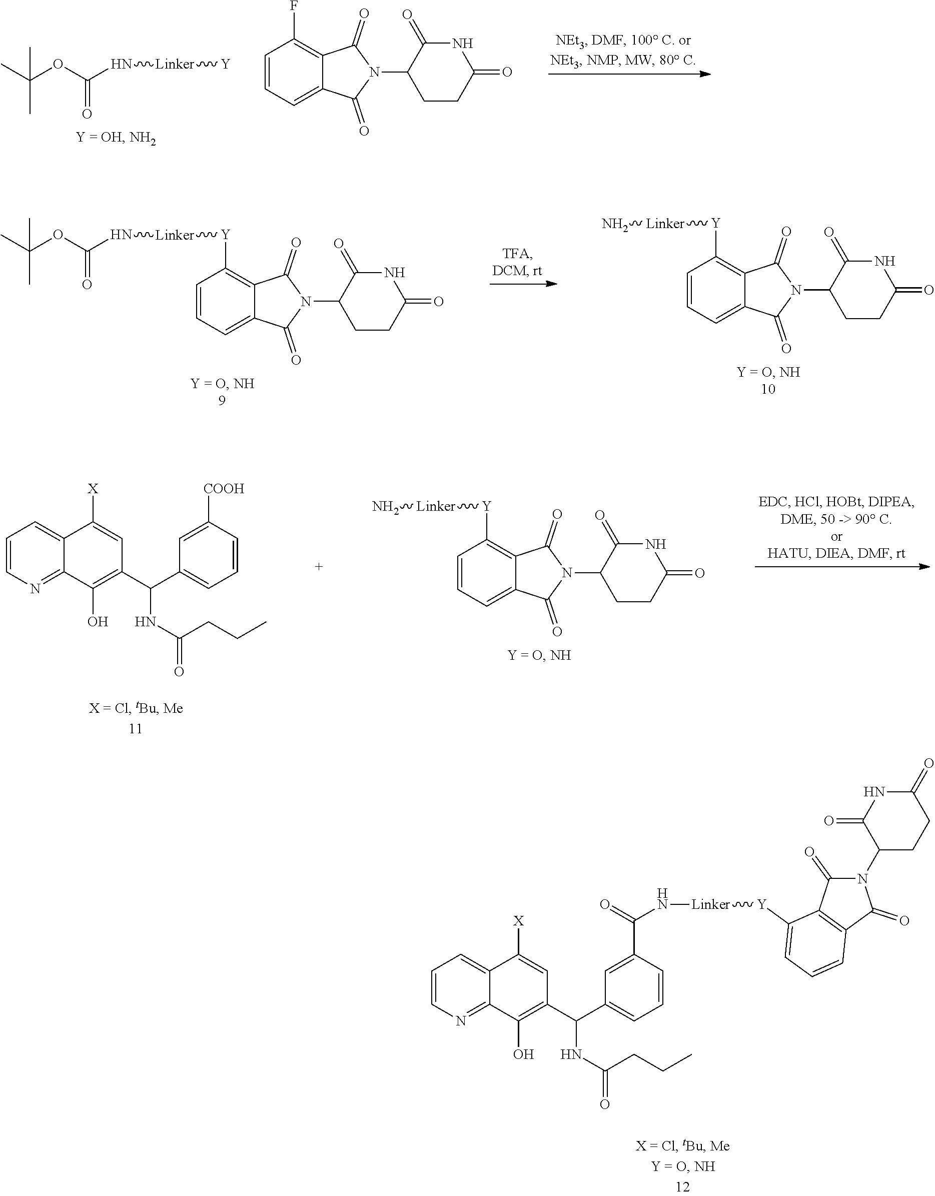

### Series B

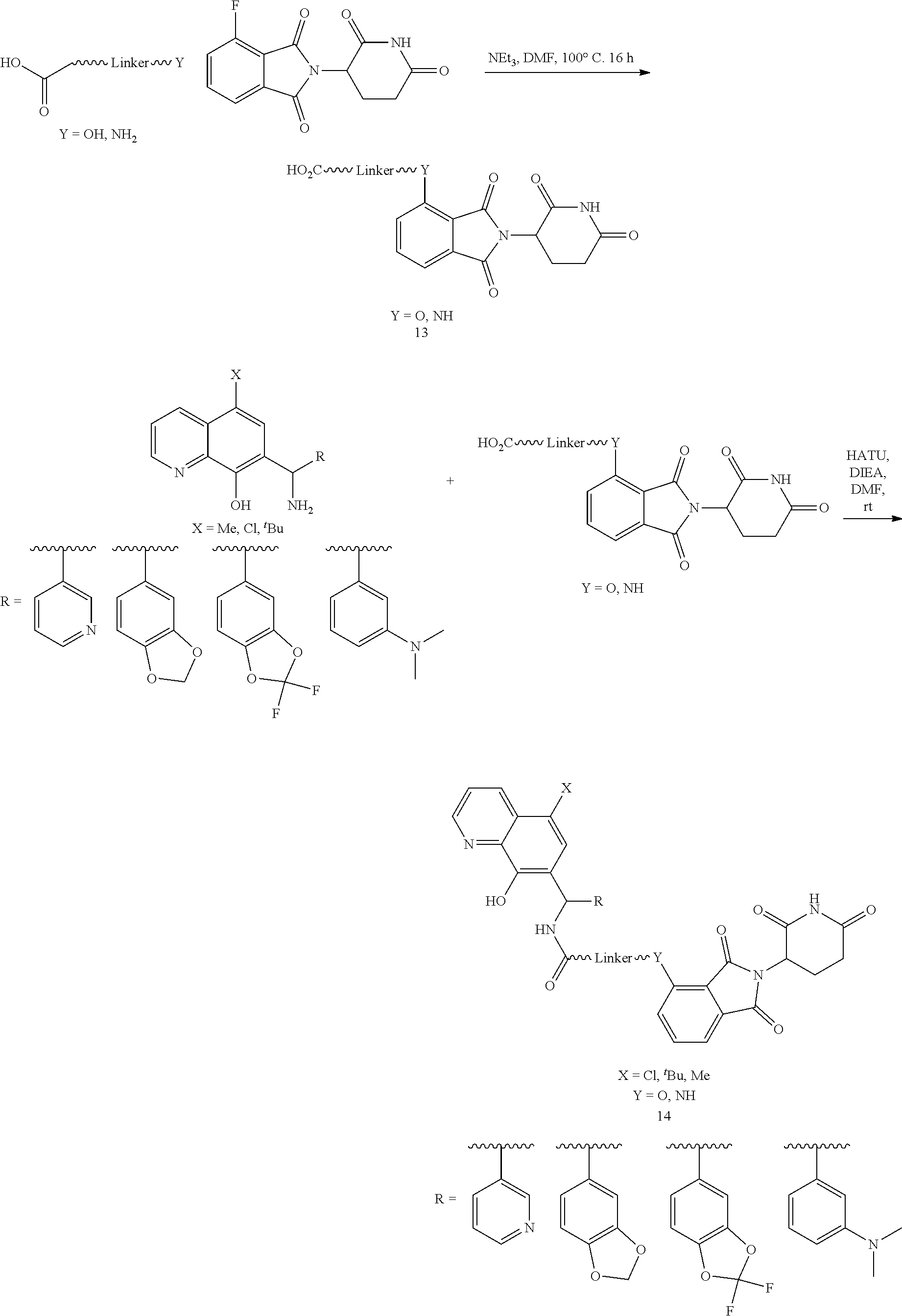

### Series C

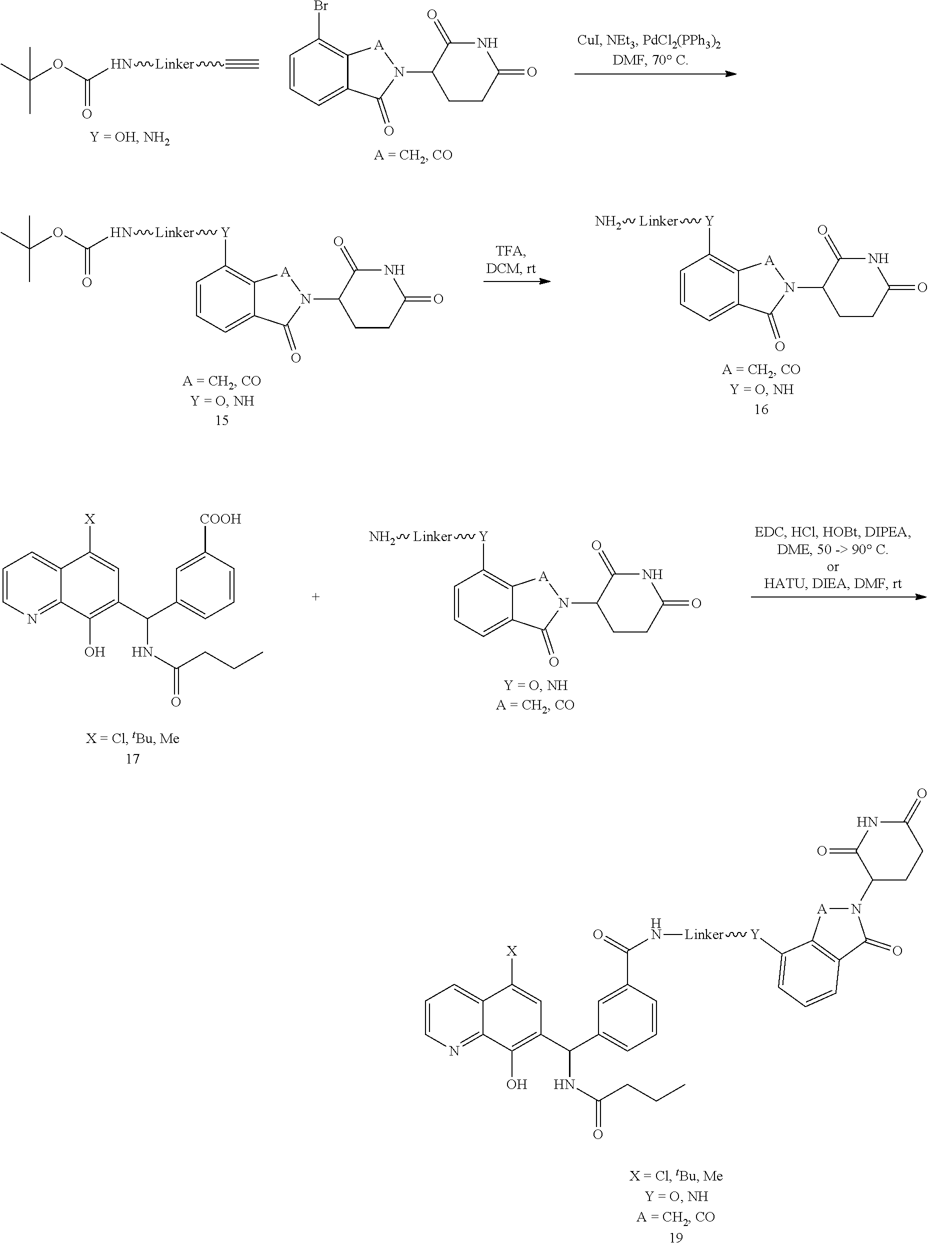

### Series D

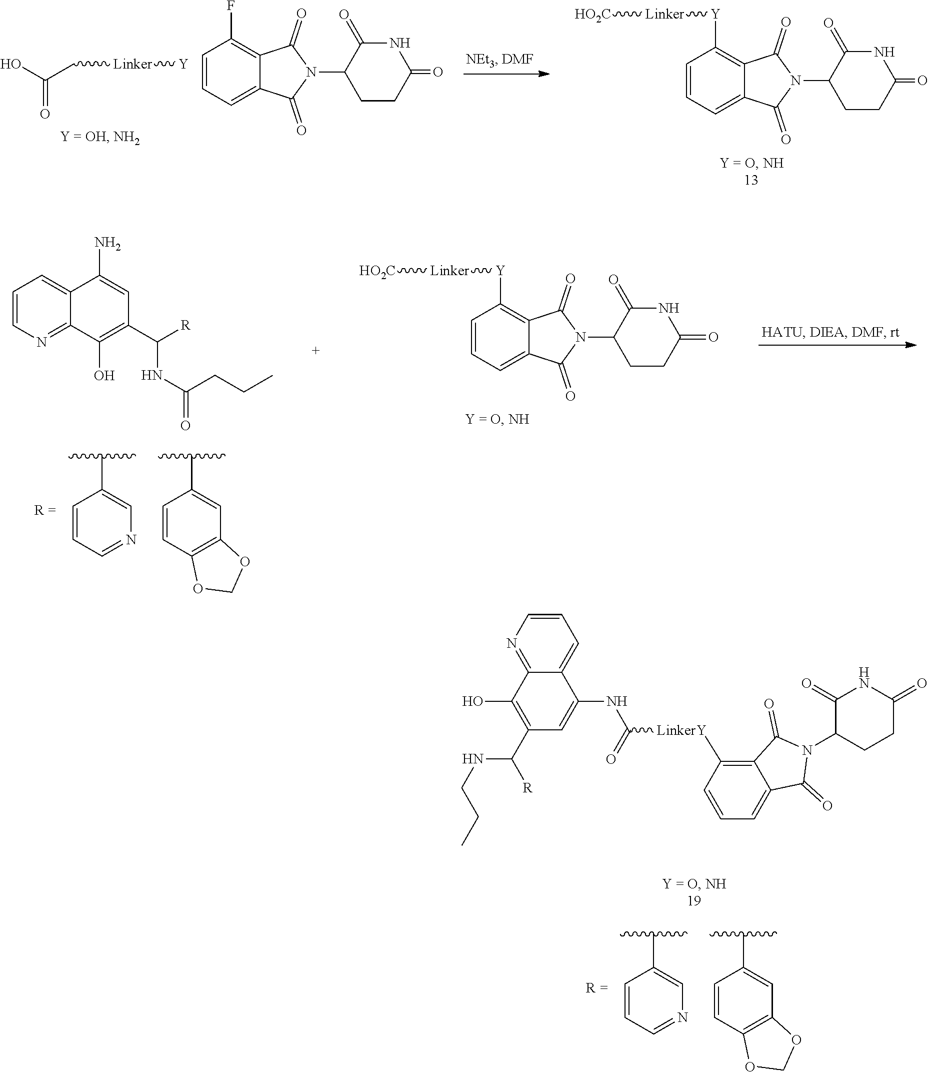

### Series E

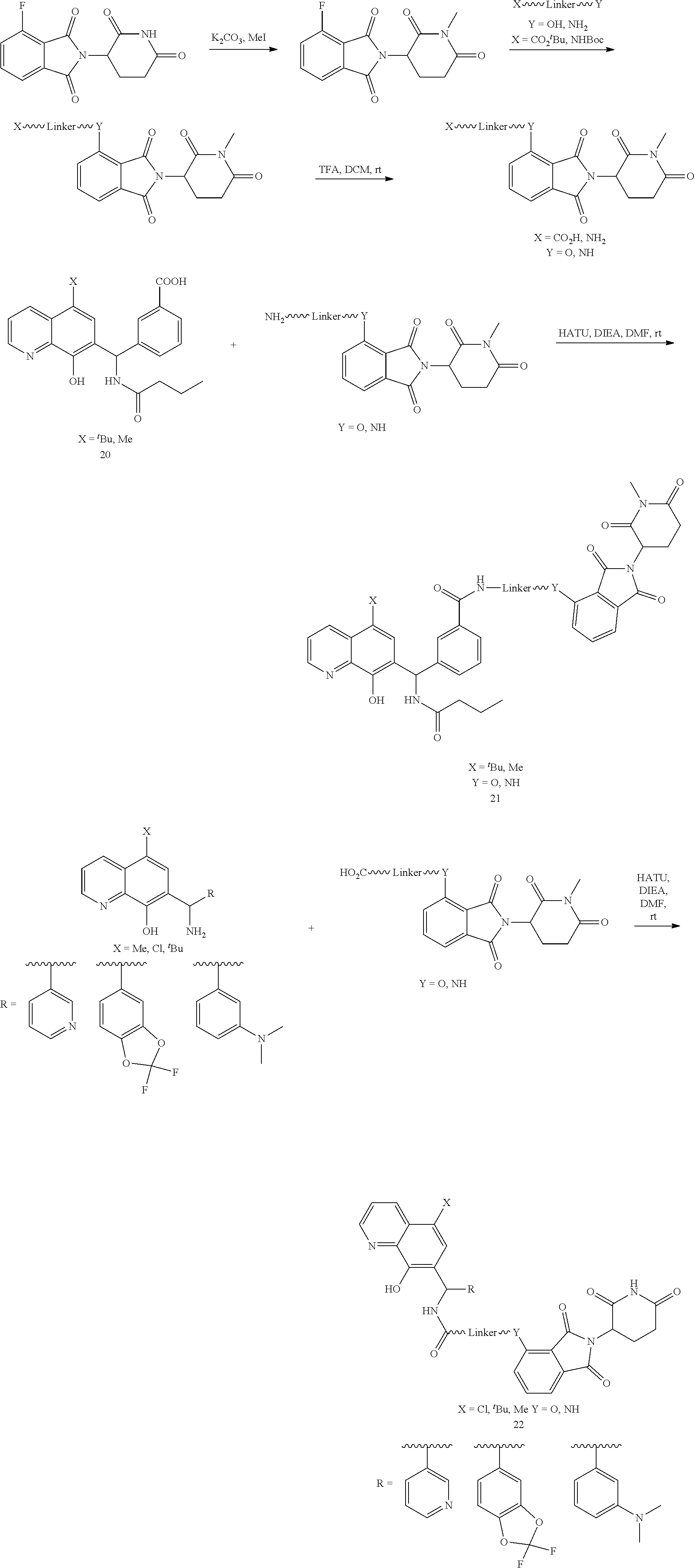

### Series F

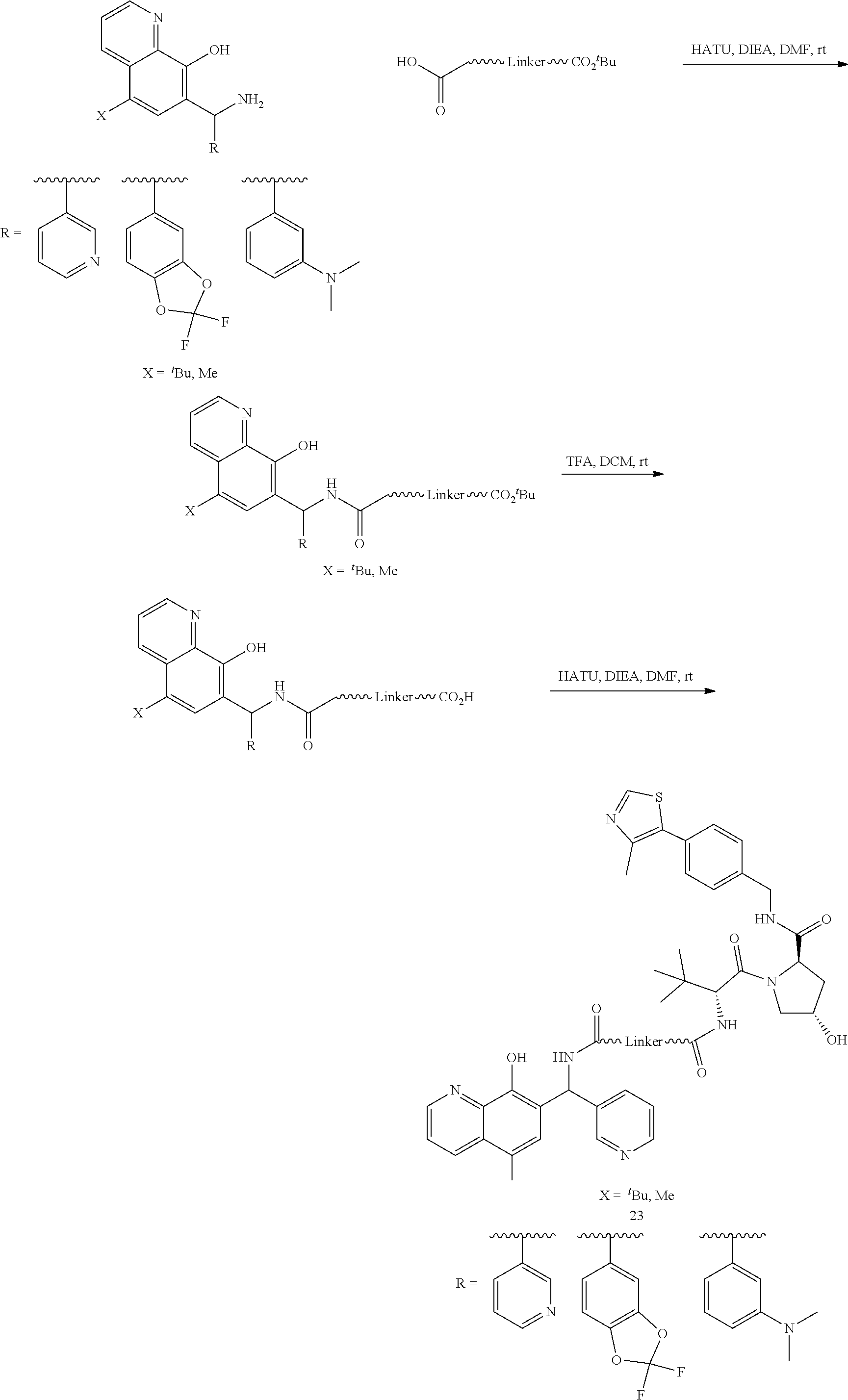

### Series G

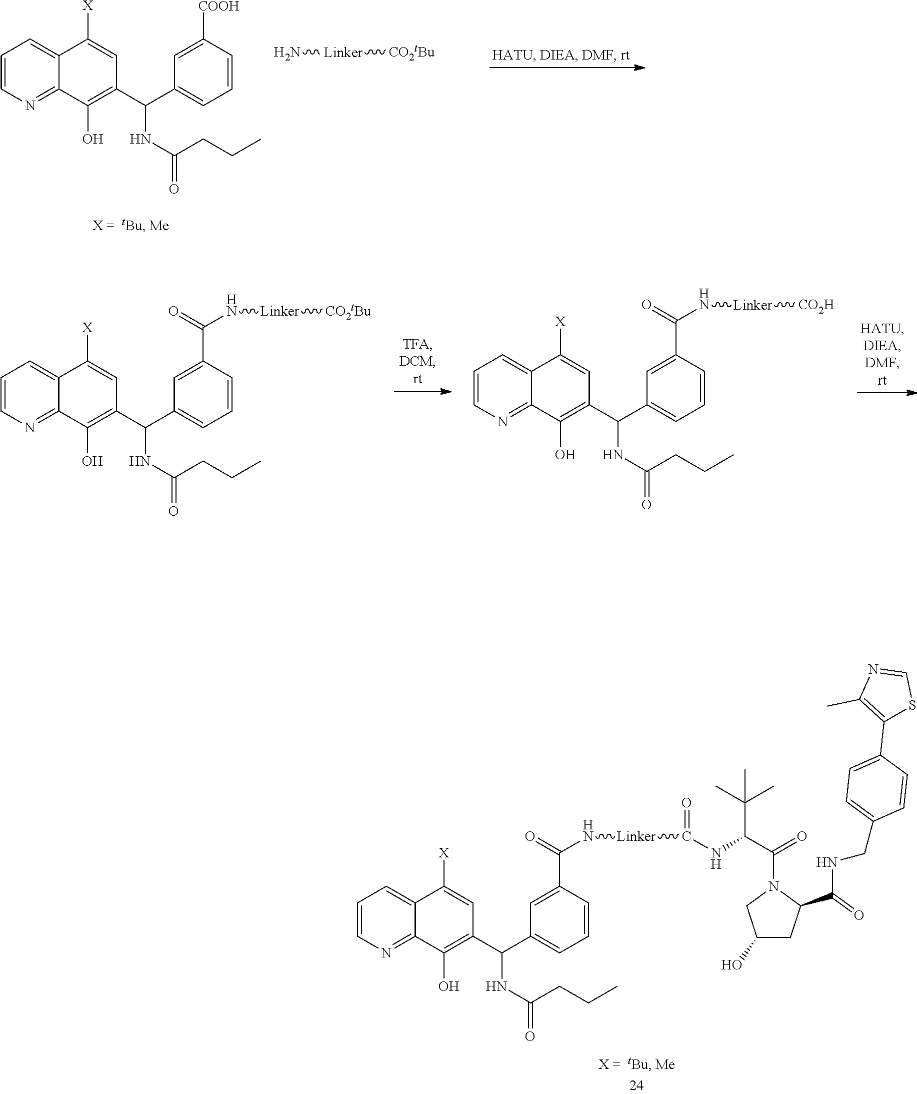

### General Procedure of Reaction of 2-(2,6-dioxopiperidin-3-yl)-4-fluoroisoindoline-1,3-dione with appropriate linkers

To a solution of 2-(2,6-dioxopiperidin-3-yl)-4-fluoroisoindoline-1,3-dione (1 equiv) and appropriate linker amines/aliphatic alcohol (1.5 equiv) in DMF (4 mL) was added DIEA (2 equiv) and the mixture was heated at 90° C. overnight. The mixture was then diluted with MeOH and purified by HPLC (10-95% CH₃CN in H₂, 0.05% formic acid as additive) to obtain the desired linker-E3ligase as a yellow solid.

General Procedure for Sonogashira Coupling.

To a solution of alkyne (1 equiv.) and bromide (2 equiv.) in dry DMF (2 mL) was added copper iodide (0.2 equiv.) and Pd(PPh₃)Cl₂ (0.1 equiv.). The solution was purged and refilled with Argon 3 times. Triethylamine (2 mL) was then added, and the solution was purged again with Argon. The reaction mixture was stirred at 70° C. overnight. The mixture was diluted with EtOAc, washed with water and brine, dried over Na₂SO₄, and concentrated. The crude product was purified by column chromatography

### 3-(Butyramido(5-chloro-8-hydroxyquinolin-7-yl)methyl)-N-(4-(2-((2-(2,6-dioxopiperidin-3-yl)-1,3-dioxoisoindolin-4-yl)oxy)acetamido)butyl)benzamide (P1) (YUM513)

To a solution of 3-(butyramido(5-chloro-8-hydroxyquinolin-7-yl)methyl)benzoic acid (G52) (15 mg, 0.037 mmol), EDC·HCl (7.2 mg, 0.037 mmol), HOBt (6.1 mg, 0.045 mmol) and DIPEA (19 μL, 0.112 mmol) in DMF was added N-(4-aminobutyl)-2-((2-(2,6-dioxopiperidin-3-yl)-1,3-dioxoisoindolin-4-yl)oxy)acetamide (19 mg, 0.037 mmol). The reaction mixture was heated at 50° C. for 16 hrs, and then cooled down to room temperature. The mixture was diluted with EtOAc, washed with water and brine, dried over Na₂SO₄, and concentrated. The crude product was purified by column chromatography (DCM, DCM:0.5M NH₄ in MeOH=10:1) to obtain P1. MS (ESI) m/z=783 (M+H)⁺, 781 (M−H)⁻.

### 3-((5-(tert-butyl)-8-hydroxyquinolin-7-yl)(butyramido)methyl)-N-(2-((2-(2,6-dioxopiperidin-3-yl)-1,3-dioxoisoindolin-4-yl)amino)ethyl)benzamide (P2)

To a solution of 3-((5-(tert-butyl)-8-hydroxyquinolin-7-yl)(butyramido)methyl)benzoic acid (G69) (16 mg, 0.04 mmol), HATU (22 mg, 0.06 mmol), and DIPEA (20 μL, 0.12 mmol) in DMF was added 4-((2-aminoethyl)amino)-2-(2,6-dioxopiperidin-3-yl)isoindoline-1,3-dione (13 mg, 0.04 mmol). The reaction mixture was stirred at rt for 2 hrs. On completion of the reaction. the mixture was diluted with DCM, washed with water and brine, dried over Na₂SO₄, and concentrated. The crude product was purified by HPLC (10-95% CH₃CN in H₂O, 0.05% formic acid as additive) to obtain P2 as yellow solid (9 mg, 31%).

¹H NMR (300 MHz, Methanol-d₄) δ 9.53 (d, J=8.9 Hz, 1H), 8.99 (d, J=5.0 Hz, 1H), 7.97 (dd, J=9.1, 5.1 Hz, 1H), 7.75 (d, J=13.3 Hz, 2H), 7.63 (s, 1H), 7.55-7.41 (m, 3H), 7.16 (d, J=8.6 Hz, 1H), 6.99 (d, J=7.1 Hz, 1H), 6.83 (s, 1H), 5.06 (dd, J=12.1, 5.4 Hz, 1H), 3.61 (d, J=6.7 Hz, 4H), 2.78 (dq, J=23.9, 11.9, 10.0 Hz, 4H), 2.42-2.24 (m, 2H), 1.69 (q, J=7.4 Hz, 2H), 1.56 (s, 9H), 0.95 (t, J=7.4 Hz, 3H). MS (ESI) m/z=719 (M+H)³⁰

### 3-((5-(tert-butyl)-8-hydroxyquinolin-7-yl)(butyramido)methyl)-N-(7-((2-(2,6-dioxopiperidin-3-yl)-1,3-dioxoisoindolin-4-yl)amino)heptyl)benzamide (P3)

To a solution of 3-((5-(tert-butyl)-8-hydroxyquinolin-7-yl)(butyramido)methyl)benzoic acid (G69) (25 mg, 0.06 mmol), HATU (38 mg, 0.1 mmol), and DIPEA (31 μL, 0.112 mmol) in DMF was added 4-((7-aminoheptyl)amino)-2-(2,6-dioxopiperidin-3-yl)isoindoline-1,3-dione (27 mg, 0.07 mmol). The reaction mixture was stirred at rt for 2 hrs. On completion of the reaction. the mixture was diluted with DCM, washed with water and brine, dried over Na₂SO₄, and concentrated. The crude product was purified by HPLC (10-95% CH₃CN in H₂O, 0.05% formic acid as additive) to obtain P3 as yellow solid (12 mg, 25%).

¹H NMR (300 MHz, Methanol-d₄) δ 9.46 (d, J=8.9 Hz, 1H), 9.01-8.90 (m, 1H), 7.92 (dd, J=8.9, 4.9 Hz, 1H), 7.83-7.71 (m, 2H), 7.62 (s, 1H), 7.57-7.44 (m, 4H), 7.08-6.95 (m, 3H), 6.83 (s, 1H), 5.06 (dd, J=12.2, 5.4 Hz, 1H), 2.78 (td, J=19.2, 18.8, 10.9 Hz, 3H), 2.42-2.28 (m, 2H), 2.11 (d, J=4.7 Hz, 1H), 1.67 (td, J=14.3, 13.9, 7.1 Hz, 7H), 1.56 (s, 9H), 1.42 (s, 9H), 0.95 (t, J=7.4 Hz, 3H). MS (ESI) m/z=789 (M+H)⁺

### 3-((5-(tert-butyl)-8-hydroxyquinolin-7-yl)(butyramido)methyl)-N-(10-((2-(2,6-dioxopiperidin-3-yl)-1,3-dioxoisoindolin-4-yl)amino)decyl)benzamide (P4)

To a solution of 3-((5-(tert-butyl)-8-hydroxyquinolin-7-yl)(butyramido)methyl)benzoic acid (G69) (16 mg, 0.04 mmol), HATU (22 mg, 0.06 mmol), and DIPEA (19 μL, 0.112 mmol) in DMF was added 4-((10-aminodecyl)amino)-2-(2,6-dioxopiperidin-3-yl)isoindoline-1,3-dione (17 mg, 0.04 mmol). The reaction mixture was stirred at rt for 2 hrs. On completion of the reaction. the mixture was diluted with DCM, washed with water and brine, dried over Na₂SO₄, and concentrated. The crude product was purified by HPLC (10-95% CH₃CN in H₂O, 0.05% formic acid as additive) to obtain P4 as yellow solid (9 mg, 27%). ¹H NMR (499 MHz, Methanol-d₄) δ 8.94 (s, 1H), 8.79 (dd, J=3.7, 2.1 Hz, 1H), 7.80-7.76 (m, 2H), 7.66 (d, J=7.6 Hz, 1H), 7.57 (d, J=7.1 Hz, 1H), 7.51 (dd, J=8.6, 7.1 Hz, 1H), 7.48-7.43 (m, 2H), 7.39 (t, J=7.7 Hz, 1H), 7.01 (d, J=7.0 Hz, 1H), 6.97 (d, J=8.5 Hz, 1H), 6.73 (s, 1H), 5.01 (dd, J=12.4, 5.4 Hz, 1H), 3.35-3.32 (m, 2H), 3.27 (t, J=7.0 Hz, 2H), 2.85-2.66 (m, 4H), 2.31 (td, J=7.2, 1.1 Hz, 2H), 2.10 (ddd, J=10.4, 6.1, 2.5 Hz, 1H), 1.71-1.54 (m, 9H), 1.52 (s, 9H), 1.33 (s, 6H), 0.94 (t, J=7.4 Hz, 5H). MS (ESI) m/z=831 (M+H)⁺

### N-((5-chloro-8-hydroxyquinolin-7-yl)(3-((6-(2-((2-(2,6-dioxopiperidin-3-yl)-1,3-dioxoisoindolin-4-yl)oxy)acetamido)hexyl)oxy)phenyl)methyl)butyramide (P5)

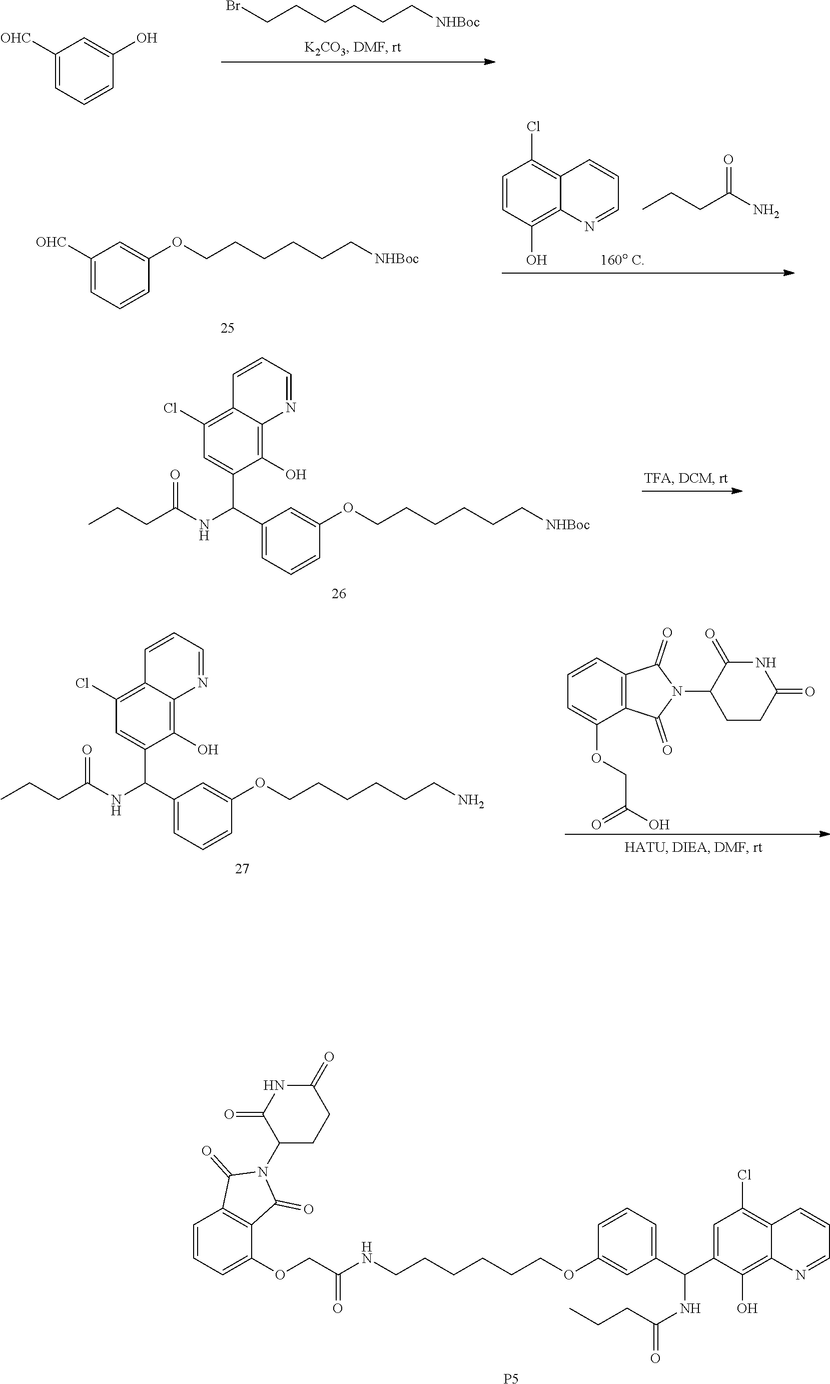

### Tert-Butyl (6-(3-formylphenoxy)hexyl)carbamate 25

To a stirred solution of 3-hydroxybenzaldehyde (195 mg, 1.6 mmol) in DMF, K₂CO₃ (441 mg, 3.2 mmol) and tert-butyl (6-(3-formylphenoxy)hexyl)carbamate (540 mg, 1.92 mmol) were added at room temperature and stirred over 12 h. On completion of the reaction the mixture was diluted with EtOAc, washed with water and brine, dried over Na₂SO₄, and concentrated. The crude product was purified by column chromatography (DCM:MeOH=20:1) to obtain tert-butyl (6-(3-formylphenoxy)hexyl)carbamate (280 mg, 0.87 mmol).

### N-((3-((6-aminohexyl)oxy)phenyl)(5-chloro-8-hydroxyquinolin-7-yl)methyl)butyramide 26

Synthesized following the general protocol A, using 5-chloroquinolin-8-ol (49 mg, 0.27 mmol), tert-butyl (6-(3-formylphenoxy)hexyl)carbamate (154 mg, 0.7 mmol), and butyramide (24 mg, 0.27 mmol) as reactants to yield tert-butyl (6-(3-(butyramido(5-chloro-8-hydroxyquinolin-7-yl)methyl)phenoxy)hexyl)carbamate which was purified by trituration with diethyl ether. The product obtained was dissolved in DCM, to it 1 mL TFA was added, followed by purification by HPLC to afford N-((3-((6-aminohexyl)oxy)phenyl)(5-chloro-8-hydroxyquinolin-7-yl)methyl)butyramide as a white solid (47 mg, 0.1 mmol).

To a solution of 2-((2-(2,6-dioxopiperidin-3-yl)-1,3-dioxoisoindolin-4-yl)oxy)acetic acid (14 mg, 0.04 mmol), HATU (22 mg, 0.06 mmol), and DIPEA (19 μL, 0.112 mmol) in DMF was added N-((3-((6-aminohexyl)oxy)phenyl)(5-chloro-8-hydroxyquinolin-7-yl)methyl)butyramide (19 mg, 0.04 mmol). The reaction mixture was stirred at rt for 2 hrs. On completion of the reaction. the mixture was diluted with DCM, washed with water and brine, dried over Na₂SO₄, and concentrated. The crude product was purified by HPLC (10-95% CH₃CN in H₂O, 0.05% formic acid as additive) to obtain P5 as yellow solid (9 mg, 29%).

¹H NMR (300 MHz, DMSO-d₆) δ 11.13 (s, 1H), 8.96 (dd, J=4.2, 1.5 Hz, 1H), 8.73 (d, J=8.8 Hz, 1H), 8.48 (dd, J=8.6, 1.5 Hz, 1H), 7.95 (t, J=5.9 Hz, 1H), 7.80 (d, J=7.9 Hz, 1H), 7.77-7.67 (m, 2H), 7.48 (d, J=7.3 Hz, 1H), 7.38 (d, J=8.5 Hz, 1H), 7.21 (t, J=7.9 Hz, 1H), 6.91-6.74 (m, 3H), 6.68 (d, J=8.7 Hz, 1H), 5.12 (dd, J=12.6, 5.3 Hz, 1H), 3.89 (t, J=6.4 Hz, 2H), 3.21-3.08 (m, 2H), 2.87 (d, J=11.9 Hz, 1H), 2.58 (d, J=16.7 Hz, 2H), 2.20 (t, J=7.2 Hz, 2H), 2.09-1.94 (m, 1H), 1.64 (d, J=7.3 Hz, 2H), 1.53 (p, J=7.2 Hz, 2H), 1.48-1.23 (m, 6H), 0.86 (t, J=7.3 Hz, 3H). MS (ESI) m/z=784 (M+H)⁺

### N-(Benzo[d][1,3]dioxol-5-yl(5-chloro-8-hydroxyquinolin-7-yl)methyl)-4-(3-(2-(2-(2-((2-(2,6-dioxopiperidin-3-yl)-1,3-dioxoisoindolin-4-yl)amino)ethoxy)ethoxy)ethoxy)propanamido)butanamide (P6)

To a solution of 3-(2-(2-(2-((2-(2,6-Dioxopiperidin-3-yl)-1,3-dioxoisoindolin-4-yl)amino)ethoxy)ethoxy)ethoxy)propanoic acid (57 mg, 0.12 mmol) and HATU (68 mg, 0.18 mmol) in DMF (2 mL) was added 4-Amino-N-(benzo[d][1,3]dioxol-5-yl(5-chloro-8-hydroxyquinolin-7-yl)methyl)butanamide (49 mg, 0.12 mmol) followed by DIEA (46 mg, 0.16 mmol). The resulting mixture was stirred at room temperature overnight. The mixture was then diluted with EtOAc, washed with water, brine, dried over anhydrous Na₂SO₄, and purified with preparative HPLC (10-95% CH₃CN in H₂O, 0.05% formic acid as additive) to give P6 (57 mg, 37%) as a white solid. ¹H NMR (400 MHz, MeOD) (8.88 (dt, J=4.1, 1.9 Hz, 1H), 8.49 (ddd, J=8.6, 2.7, 1.5 Hz, 1H), 7.61 (dddd, J=8.6, 4.3, 2.3, 0.9 Hz, 1H), 7.55 (s, 1H), 7.51 (dddd, J=8.4, 7.1, 3.6, 1.2 Hz, 1H), 7.02 (td, J=6.9, 2.8 Hz, 2H), 6.84-6.74 (m, 3H), 6.68-6.65 (m, 1H), 5.91 (d, J=1.2 Hz, 2H), 5.04 (ddt, J=12.4, 5.4, 1.1 Hz, 1H), 3.72-3.64 (m, 4H), 3.64-3.54 (m, 8H), 3.44 (td, J=5.3, 2.4 Hz, 2H), 3.24 (t, J=6.8 Hz, 2H), 2.91-2.63 (m, 3H), 2.44-2.33 (m, 4H), 2.14-2.06 (m, 1H), 1.83 (p, J=7.2 Hz, 2H). MS (ESI) m/z=873.3 [M+H]⁺.

### 6-((2-(2,6-dioxopiperidin-3-yl)-1,3-dioxoisoindolin-4-yl)amino)-N-((8-hydroxy-5-methylquinolin-7-yl)(pyridin-3-yl)methyl)hexanamide (P7)

To a solution of 6-((2-(2,6-dioxopiperidin-3-yl)-1,3-dioxoisoindolin-4-yl)amino)hexanoic acid (42 mg, 0.11 mmol) and HATU (45 mg, 0.12 mmol) in DMF (1 mL) was added 7-(amino(pyridin-3-yl)methyl)-5-methylquinolin-8-ol (25 mg, 0.09 mmol) followed by DIEA (31 μL, 0.18 mmol). The resulting mixture was stirred at room temperature overnight. The mixture was then diluted with DCM, washed with water, brine, dried over anhydrous Na₂SO₄, and purified with preparative HPLC (10-95% CH₃CN in H₂O, 0.05% formic acid as additive) to give P7 (28 mg, 40%) as a yellow solid.

¹H NMR (300 MHz, Methanol-d₄) δ 8.89 (d, J=4.5 Hz, 1H), 8.76 (s, 1H), 8.72 (d, J=5.5 Hz, 1H), 8.59 (s, 1H), 8.48 (d, J=8.0 Hz, 1H), 7.97 (d, J=7.3 Hz, 1H), 7.69 (s, 1H), 7.61-7.46 (m, 1H), 7.39 (s, 1H), 7.01 (dd, J=9.8, 7.8 Hz, 2H), 6.81 (s, 1H), 5.06 (dd, J=12.4, 5.4 Hz, 1H), 3.27 (d, J=6.8 Hz, 2H), 2.93-2.70 (m, 3H), 2.63 (s, 3H), 2.45 (t, J=7.2 Hz, 2H), 2.12 (s, 1H), 1.71 (dq, J=22.6, 7.3 Hz, 3H), 1.50 (d, J=7.5 Hz, 2H) (peaks overlapped with DMSO). MS (ESI) m/z=635.2 [M+H]⁺.

### 1-((2-(2,6-dioxopiperidin-3-yl)-1,3-dioxoisoindolin-4-yl)amino)-N-((8-hydroxy-5-methylquinolin-7-yl)(pyridin-3-yl)methyl)-3,6,9,12-tetraoxapentadecan-15-amide (P8)

To a solution of 1-((2-(2,6-dioxopiperidin-3-yl)-1,3-dioxoisoindolin-4-yl)amino)-3,6,9,12-tetraoxapentadecan-15-oic acid (26 mg, 0.05 mmol) and HATU (23 mg, 0.06 mmol) in DMF (1 mL) was added 7-(amino(pyridin-3-yl)methyl)-5-methylquinolin-8-ol (13 mg, 0.05 mmol) followed by DIEA (24 mg, 0.18 mmol). The resulting mixture was stirred at room temperature overnight. The mixture was then diluted with DCM, washed with water, brine, dried over anhydrous Na₂SO₄, and purified with preparative HPLC (10-95% CH₃CN in H₂O, 0.05% trifluoroacetic acid as additive) to give P8 (11 mg, 28%) as a yellow solid. ¹H NMR (300 MHz, Methanol-d₄) δ 9.04-8.88 (m, 2H), 8.83-8.69 (m, 2H), 8.46 (d, J=8.2 Hz, 1H), 7.98 (dd, J=8.2, 5.7 Hz, 1H), 7.81 (ddd, J=8.6, 4.6, 1.4 Hz, 1H), 7.48 (ddd, J=8.7, 7.1, 1.7 Hz, 1H), 7.43 (d, J=3.6 Hz, 1H), 7.08-6.92 (m, 2H), 6.83 (d, J=4.9 Hz, 1H), 5.07-5.00 (m, 1H), 4.00-3.65 (m, 11H), 3.60-3.52 (m, 2H), 3.42-3.36 (m, 2H), 2.94-2.79 (m, 2H), 2.79-2.67 (m, 3H), 2.64 (s, 3H), 2.62-2.45 (m, 2H), 2.16-2.02 (m, 1H). MS (ESI) m z=769 [M+H]⁺.

### 3-((5-(tert-butyl)-8-hydroxyquinolin-7-yl)(butyramido)methyl)-N-(5-(2-(2,6-dioxopiperidin-3-yl)-1-oxoisoindolin-4-yl)pent-4-yn-1-yl)benzamide (P9)

To a solution of 3-((5-(tert-butyl)-8-hydroxyquinolin-7-yl)(butyramido)methyl)benzoic acid (G69) (16 mg, 0.04 mmol), HATU (22 mg, 0.06 mmol), and DIPEA (19 μL, 0.112 mmol) in DMF was added 3-(4-(5-aminopent-1-yn-1-yl)-1-oxoisoindolin-2-yl)piperidine-2,6-dione (13 mg, 0.04 mmol). The reaction mixture was stirred at rt for 2 hrs. On completion of the reaction, the mixture was diluted with DCM, washed with water and brine, dried over Na₂SO₄, and concentrated. The crude product was purified by HPLC (10-95% CH₃CN in H₂O, 0.05% formic acid as additive) to obtain P9 as yellow solid (10 mg, 34%). ¹H NMR (300 MHz, Methanol-d₄) δ 9.50 (d, J=8.9 Hz, 1H), 8.98 (d, J=4.9 Hz, 1H), 7.95 (dd, J=8.9, 4.9 Hz, 1H), 7.83-7.67 (m, 3H), 7.63 (s, 1H), 7.55 (dd, J=7.4, 3.9 Hz, 1H), 7.47 (d, J=6.7 Hz, 3H), 6.82 (s, 1H), 5.17 (dd, J=13.3, 5.2 Hz, 1H), 4.49 (d, J=7.5 Hz, 2H), 3.56 (t, J=6.9 Hz, 2H), 2.77 (s, 3H), 2.67-2.45 (m, 4H), 2.35 (td, J=7.2, 2.4 Hz, 2H), 2.17 (s, 1H), 2.02-1.87 (m, 2H), 1.69 (p, J=7.4 Hz, 2H), 1.56 (s, 9H), 0.95 (t, J=7.4 Hz, 3H). MS (ESI) m/z=728 (M+H)⁺

### N-((8-((10-((2-(2,6-dioxopiperidin-3-yl)-1,3-dioxoisoindolin-4-yl)amino)decyl)oxy)-5-methylquinolin-7-yl)(pyridin-3-yl)methyl)butyramide (P10)

To a solution of G23 (30 mg, 0.09 mmol) in THF, diethyl azodicarboxylate (17 mg, 0.1 mmol), triphenylphosphine (26 mg, 0.1 mmol) and 2-(2,6-dioxopiperidin-3-yl)-4-((10-hydroxydecyl)amino)isoindoline-1,3-dione (42 mg, 0.1 mmol) were added and refluxed overnight. On completion of the reaction, the contents were concentrated, and crude product was purified by HPLC (10-95% CH₃CN in H₂O, 0.05% formic acid as additive) to obtain P10 as brown solid (7 mg, 10%).

¹H NMR (300 MHz, Methanol-d₄) δ 9.05 (d, J=4.6 Hz, 1H), 8.88 (d, J=8.6 Hz, 1H), 8.62 (d, J=10.5 Hz, 2H), 8.14 (d, J=8.2 Hz, 1H), 7.85 (dd, J=8.5, 4.7 Hz, 1H), 7.78 (d, J=6.6 Hz, 1H), 7.61-7.43 (m, 2H), 7.10-6.99 (m, 2H), 6.97 (s, 1H), 5.07 (dd, J=12.0, 5.4 Hz, 4H), 4.26 (ddt, J=27.1, 13.4, 6.9 Hz, 5H), 2.95-2.63 (m, 5H), 2.48-2.26 (m, 2H), 2.12 (s, 1H), 2.03-1.86 (m, 2H), 1.76-1.59 (m, 3H), 1.33 (dd, J=13.8, 6.6 Hz, 15H), 0.98 (t, J=7.4 Hz, 2H). MS (ESI) m/z=747.3 [M+H]⁺.

### 3-((5-(tert-butyl)-8-hydroxyquinolin-7-yl)(butyramido)methyl)-N-(5-((2-(2,6-dioxopiperidin-3-yl)-1,3-dioxoisoindolin-4-yl)amino)pentyl)benzamide (P11)

To a solution of 3-((5-(tert-butyl)-8-hydroxyquinolin-7-yl)(butyramido)methyl)benzoic acid (G69) (17 mg, 0.04 mmol), HATU (15 mg, 0.04 mmol), and DIPEA (19 μL, 0.112 mmol) in DMF was added 4-((5-aminopentyl)amino)-2-(2,6-dioxopiperidin-3-yl)isoindoline-1,3-dione (14 mg, 0.04 mmol). The reaction mixture was stirred at rt for 1 hr. On completion of the reaction. the mixture was diluted with DCM, washed with water and brine, dried over Na₂SO₄, and concentrated. The crude product was purified by HPLC (10-95% CH₃CN in H₂O, 0.05% formic acid as additive) to obtain P11 as yellow solid (9 mg, 29%).

¹H NMR (300 MHz, Methanol-d₄) δ 8.91 (d, J=8.9 Hz, 1H), 8.81 (d, J=4.3 Hz, 1H), 7.81 (s, 1H), 7.68 (d, J=7.5 Hz, 1H), 7.62-7.36 (m, 4H), 7.10-6.95 (m, 2H), 6.78 (s, 1H), 5.04 (dd, J=12.2, 5.4 Hz, 1H), 3.39 (t, J=7.1 Hz, 5H), 2.96-2.56 (m, 3H), 2.34 (t, J=7.3 Hz, 2H), 2.25 (t, J=7.3 Hz, 1H), 2.09 (s, 1H), 1.70 (q, J=7.1 Hz, 6H), 1.54 (s, 9H), 0.97 (t, J=7.4 Hz, 3H). MS (ESI) m/z=761 (M+H)⁺.

### 3-((5-(tert-butyl)-8-hydroxyquinolin-7-yl)(butyramido)methyl)-N-(4-((2-(2,6-dioxopiperidin-3-yl)-1,3-dioxoisoindolin-4-yl)amino)butyl)benzamide (P12)

To a solution of 3-((5-(tert-butyl)-8-hydroxyquinolin-7-yl)(butyramido)methyl)benzoic acid (G69) (17 mg, 0.04 mmol), HATU (15 mg, 0.04 mmol), and DIPEA (19 μL, 0.112 mmol) in DMF was added 4-((4-aminobutyl)amino)-2-(2,6-dioxopiperidin-3-yl)isoindoline-1,3-dione (14 mg, 0.04 mmol). The reaction mixture was stirred at rt for 1 hr. On completion of the reaction. the mixture was diluted with DCM, washed with water and brine, dried over Na₂SO₄, and concentrated. The crude product was purified by HPLC (10-95% CH₃CN in H₂O, 0.05% formic acid as additive) to obtain P12 as yellow solid (7 mg, 23%). MS (ESI) m/z=747 (M+H)⁺.

### 4-((2-(2,6-dioxopiperidin-3-yl)-1,3-dioxoisoindolin-4-yl)amino)-N-((8-hydroxy-5-methylquinolin-7-yl)(pyridin-3-yl)methyl)butanamide (P15)

To a solution of 4-((2-(2,6-dioxopiperidin-3-yl)-1,3-dioxoisoindolin-4-yl)amino)butanoic acid (21 mg, 0.06 mmol) and HATU (23 mg, 0.06 mmol) in DMF (1 mL) was added 7-(amino(pyridin-3-yl)methyl)-5-methylquinolin-8-ol (16 mg, 0.06 mmol) followed by DIEA (24 mg, 0.18 mmol). The resulting mixture was stirred at room temperature for 1 h. The mixture was then diluted with DCM, washed with water, brine, dried over anhydrous Na₂SO₄, and purified with preparative HPLC (10-95% CH₃CN in H₂O, 0.05% trifluoracetic acid as additive) to give P15 (6 mg, 16%) as a yellow solid. 1H NMR (300 MHz, Methanol-d₄) δ 8.84 (s, 1H), 8.51 (s, 1H), 8.44-8.35 (m, 2H), 7.79 (d, J=8.3 Hz, 1H), 7.56 (dd, J=8.4, 4.1 Hz, 1H), 7.38 (dd, J=7.9, 4.5 Hz, 2H), 7.27 (s, 1H), 6.95 (t, J=7.2 Hz, 2H), 6.79 (s, 1H), 3.79-3.70 (m, 1H), 3.29-3.20 (m, 1H), 2.88-2.68 (m, 4H), 2.56 (s, 3H), 2.51 (t, J=7.3 Hz, 2H), 2.13-1.96 (m, 3H). MS (ESI) m/z=607 (M+H)⁺

### 5-((2-(2,6-dioxopiperidin-3-yl)-1,3-dioxoisoindolin-4-yl)amino)-N-((8-hydroxy-5-methylquinolin-7-yl)(pyridin-3-yl)methyl)pentanamide (P16)

To a solution of 5-((2-(2,6-dioxopiperidin-3-yl)-1,3-dioxoisoindolin-4-yl)amino)pentanoic acid (23 mg, 0.06 mmol) and HATU (23 mg, 0.06 mmol) in DMF (1 mL) was added 7-(amino(pyridin-3-yl)methyl)-5-methylquinolin-8-ol (16 mg, 0.06 mmol) followed by DIEA (23 mg, 0.18 mmol). The resulting mixture was stirred at room temperature for 1 h. The mixture was then diluted with DCM, washed with water, brine, dried over anhydrous Na₂SO₄, and purified with preparative HPLC (10-95% CH₃CN in H₂O, 0.05% trifluoracetic acid as additive) to give P16 (7 mg, 18%) as a yellow solid. ¹H NMR (400 MHz, Methanol-d₄) δ 8.87-8.79 (m, 1H), 8.57 (d, J=2.2 Hz, 1H), 8.50 (d, J=4.5 Hz, 1H), 8.42 (d, J=8.5 Hz, 1H), 7.98 (d, J=8.1 Hz, 1H), 7.61-7.50 (m, 2H), 7.45 (ddd, J=8.9, 7.1, 2.1 Hz, 1H), 7.31 (s, 1H), 7.01-6.91 (m, 2H), 6.79 (s, 1H), 5.06 (dd, J=13.1, 5.8 Hz, 1H), 3.75 (p, J=6.6 Hz, 2H), 3.25 (q, J=7.5 Hz, 2H), 2.58 (d, J=0.9 Hz, 3H), 2.46 (t, J=7.1 Hz, 2H), 2.29-2.16 (m, 1H), 2.15-2.00 (m, 2H), 1.80 (q, J=7.4 Hz, 2H), 1.74-1.63 (m, 2H). MS (ESI) m/z=621 (M+H)⁺.

### 3-((5-(tert-butyl)-8-hydroxyquinolin-7-yl)(butyramido)methyl)-N-(4-(2-((2-(2,6-dioxopiperidin-3-yl)-1,3-dioxoisoindolin-4-yl)oxy)acetamido)butyl)benzamide (P36)

To a solution of 2-((2-(2,6-dioxopiperidin-3-yl)-1,3-dioxoisoindolin-4-yl)oxy)acetic acid (50 mg, 0.15 mmol), HATU (57 mg, 0.15 mmol), and DIPEA (0.2 mL) in DMF was added tert-butyl (4-aminobutyl)carbamate (28 mg, 0.15 mmol). The reaction mixture was stirred at rt for 2 hrs. On completion of the reaction, the mixture was diluted with DCM, washed with water and brine, dried over Na₂SO₄, and concentrated. The crude product was dissolved in DCM, to it 1 mL of TFA was added. The free amine N-(4-aminobutyl)-2-((2-(2,6-dioxopiperidin-3-yl)-1,3-dioxoisoindolin-4-yl)oxy)acetamide was purified by HPLC and subjected to the next reaction.

To a solution of 3-((5-(tert-butyl)-8-hydroxyquinolin-7-yl)(butyramido)methyl)benzoic acid (G69) (16 mg, 0.04 mmol), HATU (14 mg, 0.04 mmol), and DIPEA (19 μL, 0.112 mmol) in DMF was added N-(4-aminobutyl)-2-((2-(2,6-dioxopiperidin-3-yl)-1,3-dioxoisoindolin-4-yl)oxy)acetamide (16 mg, 0.04 mmol). The reaction mixture was stirred at rt for 2 hrs. On completion of the reaction, the mixture was diluted with DCM, washed with water and brine, dried over Na₂SO₄, and concentrated. The crude product was purified by HPLC to obtain P36 as brown solid (8 mg, 20%).

¹H NMR (300 MHz, Methanol-d₄) δ 9.38 (d, J=8.9 Hz, 1H), 8.94 (d, J=4.8 Hz, 1H), 8.15 (s, 1H), 7.87 (dd, J=8.9, 4.8 Hz, 1H), 7.82-7.70 (m, 3H), 7.60 (s, 1H), 7.54-7.38 (m, 3H), 6.82 (s, 1H), 5.16-5.04 (m, 1H), 4.76 (s, 2H), 3.37 (d, J=8.0 Hz, 2H), 2.94-2.50 (m, 4H), 2.42-2.26 (m, 2H), 2.16-1.97 (m, 1H), 1.69 (dd, J=15.4, 8.0 Hz, 6H), 1.60-1.45 (m, 9H), 1.39 (dd, J=6.5, 3.0 Hz, 1H), 0.95 (t, J=7.4 Hz, 3H). MS (ESI) m/z=805 (M+H)³⁰

### N-((5-(tert-butyl)-8-hydroxyquinolin-7-yl)(3-(4-((2-(2,6-dioxopiperidin-3-yl)-1-oxoisoindolin-4-yl)ethynyl)piperidine-1-carbonyl)phenyl)methyl)butyramide (P37)

To a solution of 3-((5-(tert-butyl)-8-hydroxyquinolin-7-yl)(butyramido)methyl)benzoic acid (G69) (17 mg, 0.04 mmol), HATU (15 mg, 0.04 mmol), and DIPEA (19 μL, 0.112 mmol) in DMF was added 3-(1-oxo-4-(piperidin-4-ylethynyl)isoindolin-2-yl)piperidine-2,6-dione (14 mg, 0.04 mmol). The reaction mixture was stirred at rt for 2 hrs. On completion of the reaction, the mixture was diluted with DCM, washed with water and brine, dried over Na₂SO₄, and concentrated. The crude product was purified by HPLC (10-95% CH₃CN in H₂O, 0.05% formic acid as additive) to obtain P37 as brown solid (5 mg, 17%).

¹H NMR (300 MHz, Methanol-d₄) δ 9.27 (d, J=10.3 Hz, 1H), 8.91 (s, 1H), 7.79 (t, J=6.9 Hz, 2H), 7.67-7.43 (m, 5H), 7.34 (d, J=35.8 Hz, 2H), 6.83 (s, 1H), 5.27-5.06 (m, 1H), 4.54 (dd, J=22.6, 6.0 Hz, 2H), 4.13 (s, 1H), 3.75-3.41 (m, 3H), 3.15-2.71 (m, 3H), 2.55 (d, J=10.9 Hz, 1H), 2.44-2.25 (m, 3H), 2.22 (s, 1H), 2.03 (s, 1H), 1.83-1.62 (m, 3H), 1.61-1.29 (m, 9H), 0.96 (t, J=7.1 Hz, 3H). MS (ESI) m/z=755 (M+H)⁺.

### 3-(butyramido(8-hydroxy-5-methylquinolin-7-yl)methyl)-N-(6-(2-(2,6-dioxopiperidin-3-yl)-1,3-dioxoisoindolin-4-yl)hex-5-yn-1-yl)benzamide (P38)

To a solution of 3-(butyramido(8-hydroxy-5-methylquinolin-7-yl)methyl)benzoic acid (15 mg, 0.04 mmol), HATU (15 mg, 0.04 mmol), and DIPEA (19 μL, 0.112 mmol) in DMF was added 4-(6-aminohex-1-yn-1-yl)-2-(2,6-dioxopiperidin-3-yl)isoindoline-1,3-dione (14 mg, 0.04 mmol). The reaction mixture was stirred at rt for 1 hr. On completion of the reaction, the mixture was diluted with DCM, washed with water and brine, dried over Na₂SO₄, and concentrated. The crude product was purified by HPLC (10-95% CH₃CN in H₂O, 0.05% trifluoroacetic acid as additive) to obtain P38 as brown solid (6 mg, 22%). MS (ESI) m z=714 (M+H)⁺

### 3-((5-(tert-butyl)-8-hydroxyquinolin-7-yl)(butyramido)methyl)-N-(6-((2-(2,6-dioxopiperidin-3-yl)-1-oxoisoindolin-4-yl)amino)hexyl)benzamide (P39)

To a stirred solution of tert-butyl (6-hydroxyhexyl)carbamate (300 mg, 1.38 mmol) in DCM (2 mL), DMP (850 mg, 2 mmol) was added and stirred at room temperature for 4 h. On completion of the reaction, contents were filtered, and filtrate was diluted with DCM, washed with water, brine and dried over Na₂SO₄. The product was concentrated, tert-butyl (6-oxohexyl)carbamate (250 mg, 1.17 mmol) was added to a solution of 3-(4-amino-1-oxoisoindolin-2-yl)piperidine-2,6-dione (332 mg, 1.28 mmol) in DCM and stirred for 30 mins. To this mixture then Na(OAc)BH₃ (506 mg, 2.4 mmol) was added and stirred at room temperature overnight. tert-butyl (6-((2-(2,6-dioxopiperidin-3-yl)-1-oxoisoindolin-4-yl)amino)hexyl)carbamate (185 mg, 0.4 mmol) obtained was treated with trifluoracetic acid (1 mL) in DCM (2 mL). The product obtained was purified by HPLC. To a solution of 3-(4-((6-aminohexyl)amino)-1-oxoisoindolin-2-yl)piperidine-2,6-dione (20 mg, 0.06 mmol), HATU (19 mg, 0.05 mmol), and DIPEA (19 μL, 0.112 mmol) in DMF was added 3-((5-(tert-butyl)-8-hydroxyquinolin-7-yl)(butyramido)methyl)benzoic acid (21 mg, 0.06 mmol). The reaction mixture was stirred at rt for 1 hr. On completion of the reaction, the mixture was diluted with DCM, washed with water and brine, dried over Na₂SO₄, and concentrated. The crude product was purified by HPLC (10-95% CH₃CN in H₂O, 0.05% trifluoroacetic acid as additive) to obtain P39 as brown solid (4 mg, 18%). MS (ESI) m/z=762 (M+H)⁺

### 4-(2-((2-(2,6-dioxopiperidin-3-yl)-1,3-dioxoisoindolin-4-yl)oxy)acetamido)-N-((8-hydroxy-5-methylquinolin-7-yl)(pyridin-3-yl)methyl)butanamide (P40)

To a solution of 7-(amino(pyridin-3-yl)methyl)-5-methylquinolin-8-ol (25 mg, 0.08 mmol), HATU (27 mg, 0.07 mmol), and DIPEA (0.2 mL) in DMF was 4-((tert-butoxycarbonyl)amino)butanoic acid (15 mg, 0.08 mmol). The reaction mixture was stirred at rt for 2 hrs. On completion of the reaction, the mixture was diluted with DCM, washed with water and brine, dried over Na₂SO₄, and concentrated. The crude product was dissolved in DCM, to it 1 mL of TFA was added. The free amine 4-amino-N-((8-hydroxy-5-methylquinolin-7-yl)(pyridin-3-yl)methyl)butanamide was purified by HPLC and subjected to the next reaction.

To a solution of 2-((2-(2,6-dioxopiperidin-3-yl)-1,3-dioxoisoindolin-4-yl)oxy)acetic acid (10 mg, 0.03 mmol), HATU (9 mg, 0.024 mmol), and DIPEA (0.1 mL) in DMF 4-amino-N-((8-hydroxy-5-methylquinolin-7-yl)(pyridin-3-yl)methyl)butanamide (10 mg, 0.03 mmol) was added. The reaction mixture was stirred at rt for 2 hrs. On completion of the reaction, the mixture was diluted with DCM, washed with water and brine, dried over Na₂SO₄, and concentrated. The crude product was purified by HPLC to obtain P40 as brown solid (4 mg, 20%).

¹H NMR (300 MHz, Methanol-d₄) δ 8.89-8.76 (m, 1H), 8.63 (s, 1H), 8.52 (d, J=5.1 Hz, 1H), 8.40 (d, J=8.3 Hz, 1H), 8.06 (d, J=8.2 Hz, 1H), 7.86-7.67 (m, 2H), 7.67-7.54 (m, 2H), 7.50 (t, J=5.5 Hz, 1H), 7.41-7.23 (m, 2H), 6.74 (s, 1H), 5.12 (dt, J=11.8, 6.0 Hz, 1H), 3.74 (p, J=6.7 Hz, 2H), 3.40 (s, 1H), 3.28-3.17 (m, 2H), 2.93-2.61 (m, 3H), 2.61-2.50 (m, 3H), 2.47 (d, J=7.2 Hz, 2H), 2.13 (s, 1H), 2.03-1.87 (m, 1H). MS (ESI) m/z=665 (M+H)⁺

### 6-(2-((2-(2,6-dioxopiperidin-3-yl)-1,3-dioxoisoindolin-4-yl)oxy)acetamido)-N-((8-hydroxy-5-methylquinolin-7-yl)(pyridin-3-yl)methyl)spiro[3.3]heptane-2-carboxamide (P41)

To a solution of 6-((tert-butoxycarbonyl)amino)spiro[3.3]heptane-2-carboxylic acid (20 mg, 0.08 mmol) and HATU (30 mg, 0.08 mmol) in DMF (1 mL) was added 7-(amino(pyridin-3-yl)methyl)-5-methylquinolin-8-ol (25 mg, 0.09 mmol) followed by DIEA (51 mg, 0.18 mmol). The resulting mixture was stirred at room temperature overnight. The mixture was then diluted with DCM, washed with water, brine, dried over anhydrous Na₂SO₄, and purified with preparative HPLC to give tert-butyl (6-(((8-hydroxy-5-methylquinolin-7-yl)(pyridin-3-yl)methyl)carbamoyl)spiro[3.3]heptan-2-yl)carbamate. To a solution of the latter in DCM, TFA (1 mL) was added and stirred for 2 h, the free amine 6-amino-N-((8-hydroxy-5-methylquinolin-7-yl)(pyridin-3-yl)methyl)spiro[3.3]heptane-2-carboxamide formed was purified by HPLC. To a solution of 2-((2-(2,6-dioxopiperidin-3-yl)-1,3-dioxoisoindolin-4-yl)oxy)acetic acid (10 mg, 0.03 mmol) and HATU (9 mg, 0.03 mmol) in DMF (0.5 mL) was added 6-amino-N-((8-hydroxy-5-methylquinolin-7-yl)(pyridin-3-yl)methyl)spiro[3.3]heptane-2-carboxamide (12 mg, 0.03 mmol) followed by DIEA (0.1 mL). The resulting mixture was stirred at room temperature for 2 h. The mixture was then diluted with DCM, washed with water, brine, dried over anhydrous Na₂SO₄, and purified with preparative HPLC (10-95% CH₃CN in H₂O, 0.05% trifluoroacetic acid as additive) to give P41 (5 mg, 24%) as a white solid. MS (ESI) m/z=717 [M+H]⁺.

### 6-((2-(2,6-dioxopiperidin-3-yl)-1-oxoisoindolin-4-yl)amino)-N-((8-hydroxy-5-methylquinolin-7-yl)(pyridin-3-yl)methyl)hexanamide (P42)

To a solution of 6-((2-(2,6-dioxopiperidin-3-yl)-1-oxoisoindolin-4-yl)amino)hexanoic acid (18 mg, 0.05 mmol) and HATU (19 mg, 0.05 mmol) in DMF (1 mL) was added 7-(amino(pyridin-3-yl)methyl)-5-methylquinolin-8-ol (14 mg, 0.05 mmol) followed by DIEA (15 mg, 0.18 mmol). The resulting mixture was stirred at room temperature for 2 h. The mixture was then diluted with DCM, washed with water, brine, dried over anhydrous Na₂SO₄, and purified with preparative HPLC to give P42 (12 mg, 39%) as a brown solid. ¹H NMR (300 MHz, Methanol-d₄) δ 8.88 (d, J=4.2 Hz, 1H), 8.73 (s, 1H), 8.68 (s, 1H), 8.53 (t, J=8.9 Hz, 1H), 8.40 (d, J=8.4 Hz, 1H), 7.88 (s, 1H), 7.65 (dd, J=8.3, 4.1 Hz, 1H), 7.38 (s, 1H), 7.31 (t, J=7.8 Hz, 1H), 7.07 (dd, J=7.5, 3.6 Hz, 1H), 6.78 (dd, J=8.4, 3.3 Hz, 2H), 5.20-5.09 (m, 1H), 4.25 (d, J=5.2 Hz, 1H), 3.74 (t, J=6.6 Hz, 1H), 3.57 (d, J=5.2 Hz, 1H), 3.22 (dt, J=21.0, 7.2 Hz, 3H), 3.01 (s, 1H), 2.88 (d, J=0.7 Hz, 1H), 2.82 (s, 1H), 2.61 (d, J=8.1 Hz, 3H), 2.44 (t, J=7.2 Hz, 2H), 2.17 (s, 1H), 1.71 (dq, J=20.9, 7.3 Hz, 3H), 1.49 (d, J=7.6 Hz, 2H). MS (ESI) m z=621 [M+H]⁺.

### 4-((2-(2,6-dioxopiperidin-3-yl)-1-oxoisoindolin-4-yl)amino)-N-((8-hydroxy-5-methylquinolin-7-yl)(pyridin-3-yl)methyl)butanamide (P43)

To a solution of 4-((2-(2,6-dioxopiperidin-3-yl)-1-oxoisoindolin-4-yl)amino)butanoic acid (14 mg, 0.04 mmol) and HATU (15 mg, 0.04 mmol) in DMF (1 mL) was added 7-(amino(pyridin-3-yl)methyl)-5-methylquinolin-8-ol (11 mg, 0.04 mmol) followed by DIEA (15 mg, 0.18 mmol). The resulting mixture was stirred at room temperature overnight. The mixture was then diluted with DCM, washed with water, brine, dried over anhydrous Na₂SO₄, and purified with preparative HPLC to give P43 (4 mg, 17%) as a brown solid. ¹H NMR (400 MHz, Methanol-d₄) δ 8.88 (d, J=4.4 Hz, 1H), 8.70 (d, J=7.6 Hz, 1H), 8.64 (t, J=5.0 Hz, 1H), 8.50 (t, J=9.2 Hz, 1H), 8.34 (t, J=8.8 Hz, 1H), 7.89-7.74 (m, 1H), 7.64 (dt, J=8.3, 3.9 Hz, 1H), 7.33 (d, J=2.5 Hz, 1H), 7.22 (td, J=7.7, 4.1 Hz, 1H), 7.00 (t, J=6.9 Hz, 1H), 6.86-6.76 (m, 1H), 6.75 (s, 1H), 5.12 (ddd, J=18.0, 13.2, 5.1 Hz, 1H), 4.20 (d, J=37.2 Hz, 2H), 3.32-3.25 (m, 2H), 3.01-2.68 (m, 3H), 2.65-2.50 (m, 4H), 2.42 (ddd, J=22.2, 13.3, 4.7 Hz, 1H), 2.06 (p, J=6.8 Hz, 2H), 1.33 (d, J=17.3 Hz, 2H). MS (ESI) m/z=593 (M+H)⁺.

### 6-((2-(2,6-dioxopiperidin-3-yl)-1,3-dioxoisoindolin-5-yl)amino)-N-((8-hydroxy-5-methylquinolin-7-yl)(pyridin-3-yl)methyl)hexanamide (P44)

To a solution of 6-((2-(2,6-dioxopiperidin-3-yl)-1,3-dioxoisoindolin-5-yl)amino)hexanoic acid (19 mg, 0.05 mmol), HATU (15 mg, 0.04 mmol), and DIPEA (0.1 mL) in DMF was added 7-(amino(pyridin-3-yl)methyl)-5-methylquinolin-8-ol (13 mg, 0.05 mmol). The reaction mixture was stirred at rt for 1 h. On completion of the reaction, the mixture was diluted with DCM, washed with water and brine, dried over Na₂SO₄, and concentrated. The crude product was purified by HPLC to obtain P44 as white solid (16 mg, 53%). ¹H NMR (400 MHz, Methanol-d₄) δ 8.87 (d, J=3.9 Hz, 1H), 8.70 (s, 1H), 8.65 (d, J=5.4 Hz, 1H), 8.50 (d, J=8.6 Hz, 1H), 8.31 (d, J=8.4 Hz, 1H), 7.82 (dd, J=8.3, 5.4 Hz, 1H), 7.63 (dd, J=8.6, 4.2 Hz, 1H), 7.53 (d, J=8.4 Hz, 1H), 7.37 (s, 1H), 6.90 (d, J=2.1 Hz, 1H), 6.83-6.73 (m, 1H), 5.06 (dd, J=12.3, 5.5 Hz, 1H), 3.20-3.12 (m, 1H), 2.92-2.68 (m, 3H), 2.61 (d, J=0.9 Hz, 3H), 2.44 (t, J=7.2 Hz, 2H), 2.16-2.01 (m, 2H), 1.79-1.72 (m, 1H), 1.71-1.63 (m, 1H), 1.51-1.44 (m, 1H), 1.33 (d, J=17.4 Hz, 3H). MS (ESI) m/z=635 (M+H)⁺.

### N-((2,2-difluorobenzo[d][1,3]dioxol-5-yl)(8-hydroxy-5-methylquinolin-7-yl)methyl)-6-((2-(2,6-dioxopiperidin-3-yl)-1,3-dioxoisoindolin-4-yl)amino)hexanamide (P45)

To a solution of 6-((2-(2,6-dioxopiperidin-3-yl)-1,3-dioxoisoindolin-4-yl)amino)hexanoic acid (18 mg, 0.05 mmol) and HATU (19 mg, 0.05 mmol) in DMF (1 mL) was added 7-(amino(2,2-difluorobenzo[d][1,3]dioxol-5-yl)methyl)-5-methylquinolin-8-ol (14 mg, 0.04 mmol) followed by DIEA (0.1 mL). The resulting mixture was stirred at room temperature for 1 h. The mixture was then diluted with DCM, washed with water, brine, dried over anhydrous Na₂SO₄, and purified with preparative HPLC to give P45 (19 mg, 54%) as a yellow solid. ¹H NMR (300 MHz, Methanol-d₄) δ 8.82 (d, J=4.2 Hz, 1H), 8.38 (d, J=9.1 Hz, 1H), 7.63-7.44 (m, 2H), 7.25 (s, 1H), 7.19-7.07 (m, 2H), 7.00 (dd, J=14.6, 7.9 Hz, 2H), 6.73 (s, 1H), 4.63 (s, 1H), 3.54 (s, 1H), 3.25 (d, J=7.0 Hz, 1H), 2.97-2.65 (m, 3H), 2.58 (d, J=0.9 Hz, 3H), 2.39 (t, J=7.2 Hz, 2H), 1.84-1.58 (m, 4H), 1.48 (d, J=7.4 Hz, 2H), 1.31 (s, 1H). MS (ESI) m/z=714 [M+H]³⁰.

### 3-(butyramido(8-hydroxy-5-methylquinolin-7-yl)methyl)-N-(4-(((2-(2,6-dioxopiperidin-3-yl)-1-oxoisoindolin-4-yl)amino)methyl)cyclohexyl)benzamide (P46)

To a solution of 3-(butyramido(8-hydroxy-5-methylquinolin-7-yl)methyl)benzoic acid (20 mg, 0.05 mmol), HATU (15 mg, 0.04 mmol), and DIPEA (19 μL, 0.112 mmol) in DMF was added 3-(4-(((4-aminocyclohexyl)methyl)amino)-1-oxoisoindolin-2-yl)piperidine-2,6-dione (18 mg, 0.05 mmol). The reaction mixture was stirred at rt for 1 hr. On completion of the reaction, the mixture was diluted with DCM, washed with water and brine, dried over Na₂SO₄, and concentrated. The crude product was purified by HPLC (10-95% CH₃CN in H₂O, 0.05% trifluoroacetic acid as additive) to obtain P46 as white solid (5 mg, 14%). MS (ESI) m z=731 (M+H)⁺

### N-((3-(4-(((2-(2,6-dioxopiperidin-3-yl)-1-oxoisoindolin-4-yl)amino)methyl)piperidine-1-carbonyl)phenyl)(8-hydroxy-5-methylquinolin-7-yl)methyl)butyramide (P47)

To a solution of 3-(butyramido(8-hydroxy-5-methylquinolin-7-yl)methyl)benzoic acid (20 mg, 0.05 mmol), HATU (15 mg, 0.04 mmol), and DIPEA (19 μL, 0.112 mmol) in DMF was added 3-(1-oxo-4-((piperidin-4-ylmethyl)amino)isoindolin-2-yl)piperidine-2,6-dione (18 mg, 0.05 mmol). The reaction mixture was stirred at rt for 1 hr. On completion of the reaction, the mixture was diluted with DCM, washed with water and brine, dried over Na₂SO₄, and concentrated. The crude product was purified by HPLC (10-95% CH₃CN in H₂O, 0.05% trifluoroacetic acid as additive) to obtain P47 as brown solid (4 mg, 11%). MS (ESI) m z=717 (M+H)⁺

### N-((3-(dimethylamino)phenyl)(8-hydroxy-5-methylquinolin-7-yl)methyl)-6-((2-(2,6-dioxopiperidin-3-yl)-1,3-dioxoisoindolin-4-yl)amino)hexanamide (P48)

To a solution of 6-((2-(2,6-dioxopiperidin-3-yl)-1,3-dioxoisoindolin-4-yl)amino)hexanoic acid (30 mg, 0.08 mmol) and HATU (26 mg, 0.07 mmol) in DMF (1 mL) was added 7-(amino(3-(dimethylamino)phenyl)methyl)-5-methylquinolin-8-ol (24 mg, 0.08 mmol) followed by DIEA (0.1 mL). The resulting mixture was stirred at room temperature for 1 h. The mixture was then diluted with DCM, washed with water, brine, dried over anhydrous Na₂SO₄, and purified with preparative HPLC to give P48 (24 mg, 44%) as a yellow solid. ¹H NMR (400 MHz, Methanol-d₄) δ 8.92 (d, J=4.6 Hz, 1H), 8.79 (d, J=8.6 Hz, 1H), 7.79 (dd, J=8.4, 4.6 Hz, 1H), 7.51 (dd, J=8.5, 7.1 Hz, 1H), 7.45-7.38 (m, 2H), 7.22 (d, J=6.8 Hz, 2H), 7.18-7.12 (m, 1H), 7.01 (d, J=7.0 Hz, 1H), 6.96 (d, J=8.5 Hz, 1H), 6.78 (s, 1H), 5.05 (dd, J=12.5, 5.5 Hz, 1H), 3.25 (tt, J=7.4, 4.0 Hz, 3H), 3.17-3.04 (m, 6H), 2.81-2.67 (m, 2H), 2.66-2.58 (m, 3H), 2.41 (td, J=7.1, 2.1 Hz, 2H), 2.14-2.06 (m, 1H), 1.71 (dp, J=34.8, 7.2 Hz, 4H), 1.48 (dt, J=8.8, 5.4 Hz, 2H). MS (ESI) m/z=677 [M+H]⁺.

### N-((5-(tert-butyl)-8-hydroxyquinolin-7-yl)(2,2-difluorobenzo[d][1,3]dioxol-5-yl)methyl)-6-((2-(2,6-dioxopiperidin-3-yl)-1,3-dioxoisoindolin-4-yl)amino)hexanamide (P49)

To a solution of 6-((2-(2,6-dioxopiperidin-3-yl)-1,3-dioxoisoindolin-4-yl)amino)hexanoic acid (50 mg, 0.13 mmol) and HATU (38 mg, 0.1 mmol) in DMF (2 mL) was added 7-(amino(2,2-difluorobenzo[d][1,3]dioxol-5-yl)methyl)-5-(tert-butyl)quinolin-8-ol (46 mg, 0.12 mmol) followed by DIEA (0.1 mL). The resulting mixture was stirred at room temperature for 1 h. The mixture was then diluted with DCM, washed with water, brine, dried over anhydrous Na₂SO₄, and purified with preparative HPLC to give P49 (38 mg, 41%) as a yellow solid. ¹H NMR (400 MHz, Methanol-d₄) δ 9.34 (d, J=8.9 Hz, 1H), 8.92 (dd, J=4.9, 1.3 Hz, 1H), 7.83 (ddd, J=8.9, 4.8, 1.6 Hz, 1H), 7.58 (d, J=0.9 Hz, 1H), 7.51 (ddd, J=8.3, 7.1, 1.0 Hz, 1H), 7.18 (d, J=8.4 Hz, 1H), 7.15 (d, J=1.8 Hz, 1H), 7.08 (ddd, J=8.3, 1.8, 0.8 Hz, 1H), 6.98 (ddd, J=13.0, 7.8, 2.2 Hz, 2H), 6.77 (s, 1H), 5.05 (dd, J=12.4, 5.5 Hz, 1H), 3.30-3.23 (m, 1H), 2.94-2.61 (m, 3H), 2.41 (td, J=7.2, 3.5 Hz, 2H), 2.15-2.05 (m, 1H), 1.85-1.61 (m, 3H), 1.57 (s, 9H), 1.53-1.40 (m, 2H), 1.41-1.22 (m, 2H). MS (ESI) mz=756[M+H]⁺.

### N-((2,2-difluorobenzo[d][1,3]dioxol-5-yl)(8-hydroxy-5-methylquinolin-7-yl)methyl)-3-(2-((2-(2,6-dioxopiperidin-3-yl)-1,3-dioxoisoindolin-4-yl)amino)ethoxy)propanamide (P50)

To a solution of 3-(2-((2-(2,6-dioxopiperidin-3-yl)-1,3-dioxoisoindolin-4-yl)amino)ethoxy)propanoic acid (27 mg, 0.07 mmol) and HATU (22 mg, 0.06 mmol) in DMF (1 mL) was added 7-(amino(2,2-difluorobenzo[d][1,3]dioxol-5-yl)methyl)-5-methylquinolin-8-ol (27 mg, 0.07 mmol) followed by DIEA (0.1 mL). The resulting mixture was stirred at room temperature for 1 h. The mixture was then diluted with DCM, washed with water, brine, dried over anhydrous Na₂SO₄, and purified with preparative HPLC to give P50 (25 mg, 49%) as a yellow solid. MS (ESI) m/z=716 (M+H)⁺

### N-((2,2-difluorobenzo[d][1,3]dioxol-5-yl)(8-hydroxy-5-methylquinolin-7-yl)methyl)-6-((2-(2,6-dioxopiperidin-3-yl)-1,3-dioxoisoindolin-4-yl)amino)spiro[3.3]heptane-2-carboxamide (P51)

To a solution of 6-((2-(2,6-dioxopiperidin-3-yl)-1,3-dioxoisoindolin-4-yl)amino)spiro[3.3]heptane-2-carboxylic acid (24 mg, 0.06 mmol) and HATU (22 mg, 0.06 mmol) in DMF (1 mL) was added 7-(amino(2,2-difluorobenzo[d][1,3]dioxol-5-yl)methyl)-5-methylquinolin-8-ol (20 mg, 0.06 mmol) followed by DIEA (0.1 mL). The resulting mixture was stirred at room temperature for 1 h. The mixture was then diluted with DCM, washed with water, brine, dried over anhydrous Na₂SO₄, and purified with preparative HPLC to give P51 (16 mg, 37%) as a yellow solid. ¹H NMR (300 MHz, Methanol-d₄) δ 8.95 (s, 1H), 8.79 (s, 1H), 7.81 (s, 1H), 7.61-7.49 (m, 1H), 7.38 (s, 1H), 7.21-7.13 (m, 2H), 7.09 (t, J=7.2 Hz, 2H), 6.93 (dd, J=8.5, 3.7 Hz, 1H), 6.74 (s, 1H), 5.10-5.03 (m, 1H), 4.02 (q, J=7.3 Hz, 1H), 3.53-3.43 (m, 1H), 3.38 (p, J=1.7 Hz, 1H), 3.28-3.09 (m, 2H), 2.74 (t, J=13.4 Hz, 2H), 2.66 (d, J=4.0 Hz, 3H), 2.47-2.33 (m, 2H), 2.34-2.16 (m, 2H), 2.16-1.99 (m, 3H), 1.99-1.85 (m, 1H). MS (ESI) m/z=738 (M+H)⁺

### N-((2,2-difluorobenzo[d][1,3]dioxol-5-yl)(8-hydroxy-5-methylquinolin-7-yl)methyl)-4-((2-(2,6-dioxopiperidin-3-yl)-1,3-dioxoisoindolin-4-yl)amino)bicyclo[2.2.2]octane-1-carboxamide (P52)

To a solution of 4-((2-(2,6-dioxopiperidin-3-yl)-1,3-dioxoisoindolin-4-yl)amino)bicyclo[2.2.2]octane-1-carboxylic acid (25 mg, 0.06 mmol) and HATU (22 mg, 0.06 mmol) in DMF (1 mL) was added 7-(amino(2,2-difluorobenzo[d][1,3]dioxol-5-yl)methyl)-5-methylquinolin-8-ol (20 mg, 0.06 mmol) followed by DIEA (0.1 mL). The resulting mixture was stirred at room temperature for 1 h. The mixture was then diluted with DCM, washed with water, brine, dried over anhydrous Na₂SO₄, and purified with preparative HPLC to give P52 (19 mg, 42%) as a yellow solid. ¹H NMR (300 MHz, Methanol-d₄) δ 8.87 (d, J=4.3 Hz, 1H), 8.46 (d, J=8.5 Hz, 1H), 8.28 (d, J=8.5 Hz, 1H), 7.60 (dd, J=8.6, 4.2 Hz, 1H), 7.52 (t, J=7.8 Hz, 1H), 7.32 (d, J=5.2 Hz, 2H), 7.16 (s, 1H), 7.12 (s, 2H), 7.05 (d, J=7.1 Hz, 1H), 6.60 (d, J=6.6 Hz, 1H), 2.93-2.66 (m, 4H), 2.61 (s, 3H), 2.05 (s, 12H). MS (ESI) m/z=752 (M+H)⁺

### N—((R)-1-((2R,4S)-4-hydroxy-2-((4-(4-methylthiazol-5-yl)benzyl)carbamoyl)pyrrolidin-1-yl)-3,3-dimethyl-1-oxobutan-2-yl)-N⁷-((8-hydroxy-5-methylquinolin-7-yl)(pyridin-3-yl)methyl)heptanediamide (P53)

To a solution of 7-(amino(pyridin-3-yl)methyl)-5-methylquinolin-8-ol (20 mg, 0.07 mmol), HATU (22 mg, 0.06 mmol), and DIPEA (0.1 mL) in DMF was added 7-(tert-butoxy)-7-oxoheptanoic acid (16 mg, 0.07 mmol). The reaction mixture was stirred at rt for 2 hrs. On completion of the reaction, the mixture was diluted with DCM, washed with water and brine, dried over Na₂SO₄, and concentrated. The crude product was purified by HPLC and subsequently treated with TFA to obtain the free acid 7-(((8-hydroxy-5-methylquinolin-7-yl)(pyridin-3-yl)methyl)amino)-7-oxoheptanoic acid (15 mg).

To a solution of 7-(((8-hydroxy-5-methylquinolin-7-yl)(pyridin-3-yl)methyl)amino)-7-oxoheptanoic acid (15 mg, 0.04 mmol), HATU (12 mg, 0.03 mmol), and DIPEA (0.05 mL) in DMF was added (2R,4S)-1-((R)-2-amino-3,3-dimethylbutanoyl)-4-hydroxy-N-(4-(4-methylthiazol-5-yl)benzyl)pyrrolidine-2-carboxamide (16 mg, 0.04 mmol). The reaction mixture was stirred at rt for 2 hrs. On completion of the reaction. the mixture was diluted with DCM, washed with water and brine, dried over Na₂SO₄, and concentrated. The crude product was purified by HPLC to obtain P53 as white solid (13 mg, 40%). MS (ESI) m z=820 [M+H]⁺.

### N¹-((2,2-difluorobenzo[d][1,3]dioxol-5-yl)(8-hydroxy-5-methylquinolin-7-yl)methyl)-NM-((R)-1-((2R,4S)-4-hydroxy-2-((4-(4-methylthiazol-5-yl)benzyl)carbamoyl)pyrrolidin-1-yl)-3,3-dimethyl-1-oxobutan-2-yl)octanediamide (P54)

To a solution of 8-(((2,2-difluorobenzo[d][1,3]dioxol-5-yl)(8-hydroxy-5-methylquinolin-7-yl)methyl)amino)-8-oxooctanoic acid (50 mg, 0.1 mmol), HATU (35 mg, 0.08 mmol), and DIPEA (0.1 mL) in DMF was added (2R,4S)-1-((R)-2-amino-3,3-dimethylbutanoyl)-4-hydroxy-N-(4-(4-methylthiazol-5-yl)benzyl)pyrrolidine-2-carboxamide (43 mg, 0.1 mmol). The reaction mixture was stirred at rt for 2 hrs. On completion of the reaction. the mixture was diluted with DCM, washed with water and brine, dried over Na₂SO₄, and concentrated. The crude product was purified by HPLC to obtain P54 as white solid (29 mg, 33%). ¹H NMR (400 MHz, Methanol-d₄) δ 9.08 (dt, J=8.6, 1.7 Hz, 1H), 9.02 (d, J=4.2 Hz, 2H), 7.99 (dd, J=8.6, 5.1 Hz, 1H), 7.50 (d, J=3.0 Hz, 2H), 7.47 (d, J=2.9 Hz, 1H), 7.46-7.39 (m, 2H), 7.21-7.14 (m, 2H), 7.10 (dd, J=8.3, 1.8 Hz, 1H), 6.80 (s, 1H), 4.70-4.55 (m, 2H), 4.55-4.47 (m, 1H), 4.37 (dd, J=15.5, 3.1 Hz, 1H), 3.92 (d, J=11.0 Hz, 1H), 3.82 (dd, J=11.0, 3.9 Hz, 1H), 2.71 (d, J=1.1 Hz, 3H), 2.49 (s, 3H), 2.36 (td, J=7.3, 4.2 Hz, 2H), 2.29-2.17 (m, 3H), 2.10 (ddd, J=13.3, 9.1, 4.4 Hz, 1H), 1.70-1.51 (m, 4H), 1.39 (dd, J=6.7, 3.7 Hz, 3H), 1.34 (dq, J=7.6, 4.8 Hz, 4H), 1.04 (s, 9H). MS (ESI) m/z=913 [M+H]⁺.

### N¹—((R)-1-((2R,4S)-4-hydroxy-2-((4-(4-methylthiazol-5-yl)benzyl)carbamoyl)pyrrolidin-1-yl)-3,3-dimethyl-1-oxobutan-2-yl)-N-((8-hydroxy-5-methylquinolin-7-yl)(pyridin-3-yl)methyl)octanediamide (P55)

To a solution of 7-(amino(pyridin-3-yl)methyl)-5-methylquinolin-8-ol (20 mg, 0.07 mmol), HATU (22 mg, 0.06 mmol), and DIPEA (0.1 mL) in DMF was added 8-(tert-butoxy)-8-oxooctanoic acid (16 mg, 0.07 mmol). The reaction mixture was stirred at rt for 2 hrs. On completion of the reaction, the mixture was diluted with DCM, washed with water and brine, dried over Na₂SO₄, and concentrated. The crude product was purified by HPLC and subsequently treated with TFA to obtain the free acid 8-(((8-hydroxy-5-methylquinolin-7-yl)(pyridin-3-yl)methyl)amino)-8-oxooctanoic acid (25 mg).

To a solution of 8-(((8-hydroxy-5-methylquinolin-7-yl)(pyridin-3-yl)methyl)amino)-8-oxooctanoic acid (25 mg, 0.06 mmol), HATU (19 mg, 0.05 mmol), and DIPEA (0.1 mL) in DMF was added (2R,4S)-1-((R)-2-amino-3,3-dimethylbutanoyl)-4-hydroxy-N-(4-(4-methylthiazol-5-yl)benzyl)pyrrolidine-2-carboxamide (25 mg, 0.06 mmol). The reaction mixture was stirred at rt for 2 hrs. On completion of the reaction, the mixture was diluted with DCM, washed with water and brine, dried over Na₂SO₄, and concentrated. The crude product was purified by HPLC to obtain P55 as white solid (18 mg, 36%). ¹H NMR (400 MHz, Methanol-d₄) δ 9.11-8.84 (m, 2H), 8.85-8.63 (m, 3H), 8.50 (d, J=8.0 Hz, 1H), 8.00 (d, J=7.0 Hz, 1H), 7.76 (s, 1H), 7.53-7.35 (m, 5H), 6.84 (d, J=9.7 Hz, 1H), 4.68-4.46 (m, 4H), 4.37 (dd, J=15.5, 3.4 Hz, 1H), 3.92 (d, J=11.0 Hz, 1H), 3.81 (dd, J=10.9, 3.8 Hz, 1H), 2.66 (s, 3H), 2.49 (s, 3H), 2.40 (q, J=7.5 Hz, 2H), 2.25 (dh, J=13.0, 6.7, 6.1 Hz, 3H), 2.09 (ddt, J=14.2, 9.7, 4.9 Hz, 1H), 1.63 (dt, J=38.1, 7.0 Hz, 4H), 1.43-1.20 (m, 4H), 1.04 (s, 9H). MS (ESI) m/z=834 [M+H]⁺.

### N¹-((2,2-difluorobenzo[d][1,3]dioxol-5-yl)(8-hydroxy-5-methylquinolin-7-yl)methyl)-N⁷—(R)-1-((2R,4S)-4-hydroxy-2-((4-(4-methylthiazol-5-yl)benzyl)carbamoyl)pyrrolidine-1-yl)-3,3-dimethyl-1-oxobutan-2-yl)heptanediamide (P56)

To a solution of 7-(amino(2,2-difluorobenzo[d][1,3]dioxol-5-yl)methyl)-5-methylquinolin-8-ol (50 mg, 0.15 mmol), HATU (45 mg, 0.12 mmol), and DIPEA (0.1 mL) in DMF was added 7-(tert-butoxy)-7-oxoheptanoic acid (30 mg, 0.15 mmol). The reaction mixture was stirred at rt for 2 hrs. On completion of the reaction, the mixture was diluted with DCM, washed with water and brine, dried over Na₂SO₄, and concentrated. The crude product was purified by HPLC and subsequently treated with TFA to obtain 7-(((2,2-difluorobenzo[d][1,3]dioxol-5-yl)(8-hydroxy-5-methylquinolin-7-yl)methyl)amino)-7-oxoheptanoic acid (15 mg).

To a solution of 7-(((2,2-difluorobenzo[d][1,3]dioxol-5-yl)(8-hydroxy-5-methylquinolin-7-yl)methyl)amino)-7-oxoheptanoic acid (15 mg, 0.03 mmol), HATU (12 mg, 0.03 mmol), and DIPEA (0.05 mL) in DMF was added (2R,4S)-1-((R)-2-amino-3,3-dimethylbutanoyl)-4-hydroxy-N-(4-(4-methylthiazol-5-yl)benzyl)pyrrolidine-2-carboxamide (13 mg, 0.03 mmol). The reaction mixture was stirred at rt for 2 hrs. On completion of the reaction. the mixture was diluted with DCM, washed with water and brine, dried over Na₂SO₄, and concentrated. The crude product was purified by HPLC to obtain P56 as white solid (11 mg, 40%). ¹H NMR (400 MHz, Methanol-d₄) δ 9.06 (dt, J=8.6, 1.6 Hz, 1H), 9.01 (dd, J=5.1, 1.4 Hz, 1H), 8.99 (s, 1H), 7.98 (ddd, J=8.7, 5.1, 1.4 Hz, 1H), 7.53-7.45 (m, 3H), 7.45-7.37 (m, 2H), 7.22-7.15 (m, 2H), 7.10 (ddd, J=8.3, 1.9, 0.8 Hz, 1H), 6.78 (d, J=2.2 Hz, 1H), 4.65-4.47 (m, 4H), 4.37 (d, J=15.6 Hz, 1H), 3.90 (d, J=11.0 Hz, 1H), 3.81 (dd, J=11.0, 3.9 Hz, 1H), 2.75-2.66 (m, 3H), 2.49 (s, 3H), 2.41-2.18 (m, 5H), 2.12 (dd, J=8.9, 4.4 Hz, 1H), 1.77-1.60 (m, 4H), 1.02 (d, J=5.3 Hz, 9H) (1H merged with solvent). MS (ESI) m/z=899 [M+H]⁺.

### N²-((2,2-difluorobenzo[d][1,3]dioxol-5-yl)(8-hydroxy-5-methylquinolin-7-yl)methyl)-M-((R)-1-((2R,4S)-4-hydroxy-2-((4-(4-methylthiazol-5-yl)benzyl)carbamoyl)pyrrolidin-1-yl)-3,3-dimethyl-1-oxobutan-2-yl)spiro[3.3]heptane-2,6-dicarboxamide (P57)

To a solution of 7-(amino(2,2-difluorobenzo[d][1,3]dioxol-5-yl)methyl)-5-methylquinolin-8-ol (40 mg, 0.12 mmol), HATU (36 mg, 0.09 mmol), and DIPEA (0.1 mL) in DMF was added 6-(tert-butoxycarbonyl)spiro[3.3]heptane-2-carboxylic acid (22 mg, 0.12 mmol). The reaction mixture was stirred at rt for 2 hrs. On completion of the reaction, the mixture was diluted with DCM, washed with water and brine, dried over Na₂SO₄, and concentrated. The crude product was purified by HPLC and subsequently treated with TFA to obtain 6-(((2,2-difluorobenzo[d][1,3]dioxol-5-yl)(8-hydroxy-5-methylquinolin-7-yl)methyl)carbamoyl)spiro[3.3]heptane-2-carboxylic acid (30 mg).

To a solution of 6-(((2,2-difluorobenzo[d][1,3]dioxol-5-yl)(8-hydroxy-5-methylquinolin-7-yl)methyl)carbamoyl)spiro[3.3]heptane-2-carboxylic acid (30 mg, 0.06 mmol), HATU (18 mg, 0.05 mmol), and DIPEA (0.1 mL) in DMF was added (2R,4S)-1-((R)-2-amino-3,3-dimethylbutanoyl)-4-hydroxy-N-(4-(4-methylthiazol-5-yl)benzyl)pyrrolidine-2-carboxamide (26 mg, 0.06 mmol). The reaction mixture was stirred at rt for 2 hrs. On completion of the reaction. the mixture was diluted with DCM, washed with water and brine, dried over Na₂SO₄, and concentrated. The crude product was purified by HPLC to obtain P57 as white solid (15 mg, 27%). ¹H NMR (400 MHz, Chloroform-d) δ 8.90 (s, 1H), 8.87 (dd, J=4.5, 1.6 Hz, 1H), 8.57-8.43 (m, 1H), 7.68-7.56 (m, 1H), 7.38 (d, J=3.7 Hz, 4H), 7.33 (s, 1H), 7.13-7.02 (m, 2H), 6.96 (dd, J=8.3, 2.1 Hz, 1H), 6.43 (d, J=8.7 Hz, 1H), 6.08 (t, J=7.7 Hz, 1H), 4.73 (t, J=8.0 Hz, 1H), 4.59 (dt, J=17.4, 6.6 Hz, 2H), 4.52-4.44 (m, 1H), 4.36 (dd, J=15.0, 5.2 Hz, 1H), 4.15 (d, J=11.6 Hz, 1H), 3.71-3.60 (m, 1H), 3.05-2.93 (m, 1H), 2.93-2.83 (m, 1H), 2.64 (s, 3H), 2.55 (s, 3H), 2.40-2.07 (m, 9H), 1.01-0.81 (m, 9H). MS (ESI) m/z=923 [M+H]⁺.

### N¹-((2,2-difluorobenzo[d][1,3]dioxol-5-yl)(8-hydroxy-5-methylquinolin-7-yl)methyl)-N⁴—((R)-1-((2R,4S)-4-hydroxy-2-((4-(4-methylthiazol-5-yl)benzyl)carbamoyl)pyrrolidin-1-yl)-3,3-dimethyl-1-oxobutan-2-yl)bicyclo[2.2.2]octane-1,4-dicarboxamide (P58)

To a solution of 7-(amino(2,2-difluorobenzo[d][1,3]dioxol-5-yl)methyl)-5-methylquinolin-8-ol (40 mg, 0.12 mmol), HATU (36 mg, 0.09 mmol), and DIPEA (0.1 mL) in DMF was added 4-(tert-butoxycarbonyl)bicyclo[2.2.2]octane-1-carboxylic acid (24 mg, 0.12 mmol). The reaction mixture was stirred at rt for 2 hrs. On completion of the reaction, the mixture was diluted with DCM, washed with water and brine, dried over Na₂SO₄, and concentrated. The crude product was purified by HPLC and subsequently treated with TFA to obtain tert-butyl 4-(((2,2-difluorobenzo[d][1,3]dioxol-5-yl)(8-hydroxy-5-methylquinolin-7-yl)methyl)carbamoyl)bicyclo[2.2.2]octane-1-carboxylate (35 mg).

To a solution of tert-butyl 4-(((2,2-difluorobenzo[d][1,3]dioxol-5-yl)(8-hydroxy-5-methylquinolin-7-yl)methyl)carbamoyl)bicyclo[2.2.2]octane-1-carboxylate (30 mg, 0.06 mmol), HATU (18 mg, 0.05 mmol), and DIPEA (0.1 mL) in DMF was added (2R,4S)-1-((R)-2-amino-3,3-dimethylbutanoyl)-4-hydroxy-N-(4-(4-methylthiazol-5-yl)benzyl)pyrrolidine-2-carboxamide (26 mg, 0.06 mmol). The reaction mixture was stirred at rt for 2 hrs. On completion of the reaction. the mixture was diluted with DCM, washed with water and brine, dried over Na₂SO₄, and concentrated. The crude product was purified by HPLC to obtain P58 as white solid (12 mg, 21%). ¹H NMR (400 MHz, Chloroform-d) δ 8.93 (s, 1H), 8.87 (dd, J=4.5, 1.5 Hz, 1H), 8.50 (dd, J=8.5, 1.5 Hz, 1H), 7.80 (dd, J=8.7, 2.2 Hz, 1H), 7.64 (dd, J=8.5, 4.5 Hz, 1H), 7.38 (s, 4H), 7.32 (d, J=9.2 Hz, 2H), 7.11-7.04 (m, 2H), 6.96 (d, J=8.2 Hz, 1H), 6.39 (d, J=8.6 Hz, 1H), 6.22 (d, J=8.5 Hz, 1H), 4.74 (t, J=8.0 Hz, 1H), 4.63-4.53 (m, 2H), 4.47 (d, J=8.5 Hz, 1H), 4.37 (dd, J=15.0, 5.3 Hz, 1H), 4.13 (d, J=11.4 Hz, 1H), 3.61 (dd, J=11.4, 3.4 Hz, 1H), 2.64 (d, J=0.9 Hz, 3H), 2.56 (s, 4H), 2.16 (dd, J=13.9, 8.0 Hz, 1H), 1.90-1.77 (m, 10H), 0.94 (s, 9H). MS (ESI) m/z=938 [M+H]⁺.

### 3-((5-(tert-butyl)-8-hydroxyquinolin-7-yl)(butyramido)methyl)-N-(2-((2-(1-methyl-2,6-dioxopiperidin-3-yl)-1,3-dioxoisoindolin-4-yl)amino)ethyl)benzamide (P59)

To a solution of 3-((5-(tert-butyl)-8-hydroxyquinolin-7-yl)(butyramido)methyl)benzoic acid (10 mg, 0.02 mmol), HATU (8 mg, 0.02 mmol), and DIPEA (0.05 mL) in DMF was added 4-((2-aminoethyl)amino)-2-(1-methyl-2,6-dioxopiperidin-3-yl)isoindoline-1,3-dione (8 mg, 0.02 mmol). The reaction mixture was stirred at rt for 2 hrs. On completion of the reaction, the mixture was diluted with DCM, washed with water and brine, dried over Na₂SO₄, and concentrated. The crude product was purified by HPLC to obtain P59 as yellow solid (5 mg, 34%). ¹H NMR (400 MHz, Methanol-d₄) δ 9.02 (d, J=9.3 Hz, 1H), 8.85 (d, J=4.4 Hz, 1H), 7.81 (s, 1H), 7.68 (d, J=7.7 Hz, 1H), 7.66-7.58 (m, 1H), 7.54-7.40 (m, 4H), 7.18 (d, J=8.7 Hz, 1H), 7.00 (d, J=7.1 Hz, 1H), 6.77 (s, 1H), 5.14-5.01 (m, 1H), 3.60 (s, 4H), 3.14 (s, 3H), 2.86 (t, J=7.4 Hz, 2H), 2.69 (dd, J=12.5, 6.5 Hz, 1H), 2.33 (t, J=7.3 Hz, 2H), 2.14-1.95 (m, 1H), 1.70 (h, J=7.4 Hz, 2H), 1.62-1.46 (m, 9H), 0.97 (q, J=7.2 Hz, 3H). MS (ESI) m/z=733 [M+H]⁺.

### N-((2,2-difluorobenzo[d][1,3]dioxol-5-yl)(8-hydroxy-5-methylquinolin-7-yl)methyl)-6-((2-(1-methyl-2,6-dioxopiperidin-3-yl)-1,3-dioxoisoindolin-4-yl)amino)hexanamide (P60)

To a solution of 6-((2-(1-methyl-2,6-dioxopiperidin-3-yl)-1,3-dioxoisoindolin-4-yl)amino)hexanoic acid (24 mg, 0.06 mmol), HATU (19 mg, 0.05 mmol), and DIPEA (0.1 mL) in DMF was added 7-(amino(2,2-difluorobenzo[d][1,3]dioxol-5-yl)methyl)-5-methylquinolin-8-ol (20 mg, 0.06 mmol). The reaction mixture was stirred at rt for 2 hrs. On completion of the reaction, the mixture was diluted with DCM, washed with water and brine, dried over Na₂SO₄, and concentrated. The crude product was purified by HPLC to obtain P60 as yellow solid (11 mg, 25%). ¹H NMR (400 MHz, Methanol-d₄) δ 8.94 (dd, J=4.9, 1.4 Hz, 1H), 8.86 (dt, J=8.7, 2.1 Hz, 1H), 7.84 (ddd, J=8.6, 4.9, 2.0 Hz, 1H), 7.51 (dd, J=8.6, 7.1 Hz, 1H), 7.43 (d, J=1.1 Hz, 1H), 7.19-7.14 (m, 2H), 7.09 (ddd, J=8.4, 1.7, 0.7 Hz, 1H), 6.98 (dd, J=17.2, 7.8 Hz, 2H), 6.78 (s, 1H), 5.08 (dd, J=12.9, 5.4 Hz, 1H), 3.26 (t, J=7.0 Hz, 2H), 3.15 (d, J=1.3 Hz, 3H), 2.95-2.80 (m, 2H), 2.76-2.67 (m, 1H), 2.65 (d, J=0.9 Hz, 3H), 2.40 (td, J=7.2, 3.0 Hz, 2H), 2.14-2.02 (m, 1H), 1.71 (dt, J=31.3, 7.4 Hz, 3H), 1.55-1.42 (m, 2H), 1.39 (dd, J=6.7, 3.6 Hz, 1H). MS (ESI) m/z=728 [M+H]⁺.

### 3-(butyramido(8-hydroxy-5-methylquinolin-7-yl)methyl)-N-(2-((2-(2,6-dioxopiperidin-3-yl)-1,3-dioxoisoindolin-4-yl)amino)ethyl)benzamide (P61)

To a solution of 3-(butyramido(8-hydroxy-5-methylquinolin-7-yl)methyl)benzoic acid (20 mg, 0.05 mmol), HATU (15 mg, 0.04 mmol), and DIPEA (0.1 mL) in DMF was added 4-((2-aminoethyl)amino)-2-(2,6-dioxopiperidin-3-yl)isoindoline-1,3-dione (16 mg, 0.05 mmol). The reaction mixture was stirred at rt for 2 hrs. On completion of the reaction, the mixture was diluted with DCM, washed with water and brine, dried over Na₂SO₄, and concentrated. The crude product was purified by HPLC to obtain P61 as yellow solid (9 mg, 26%). ¹H NMR (400 MHz, Methanol-d₄) δ 8.91 (dd, J=4.6, 1.5 Hz, 1H), 8.69 (d, J=8.6 Hz, 1H), 7.78 (s, 1H), 7.74 (dd, J=8.6, 4.6 Hz, 1H), 7.68 (d, J=7.6 Hz, 1H), 7.56-7.39 (m, 3H), 7.35 (s, 1H), 7.16 (d, J=8.5 Hz, 1H), 7.03-6.97 (m, 1H), 6.80 (s, 1H), 5.05 (ddd, J=12.3, 5.4, 2.5 Hz, 1H), 3.59 (s, 4H), 2.90-2.77 (m, 3H), 2.76 (s, 3H), 2.34 (td, J=7.3, 2.9 Hz, 2H), 2.14-2.03 (m, 1H), 1.69 (q, J=7.4 Hz, 2H), 0.96 (t, J=7.4 Hz, 3H). MS (ESI) m/z=677 [M+H]⁺.

### N¹-((2,2-difluorobenzo[d][1,3]dioxol-5-yl)(8-hydroxy-5-methylquinolin-7-yl)methyl)-NS—((R)-1-((2R,4R)-4-hydroxy-2-((4-(4-methylthiazol-5-yl)benzyl)carbamoyl)pyrrolidin-1-yl)-3,3-dimethyl-1-oxobutan-2-yl)octanediamide (P62)

To a solution of 7-(amino(2,2-difluorobenzo[d][1,3]dioxol-5-yl)methyl)-5-methylquinolin-8-ol (30 mg, 0.08 mmol), HATU (26 mg, 0.07 mmol), and DIPEA (0.1 mL) in DMF was added 8-(tert-butoxy)-8-oxooctanoic acid (20 mg, 0.08 mmol). The reaction mixture was stirred at rt for 2 hrs. On completion of the reaction, the mixture was diluted with DCM, washed with water and brine, dried over Na₂SO₄, and concentrated. The crude product was purified by HPLC, followed by treatment of TFA to afford 8-(((2,2-difluorobenzo[d][1,3]dioxol-5-yl)(8-hydroxy-5-methylquinolin-7-yl)methyl)amino)-8-oxooctanoic acid as white solid (25 mg). To a solution of 8-(((2,2-difluorobenzo[d][1,3]dioxol-5-yl)(8-hydroxy-5-methylquinolin-7-yl)methyl)amino)-8-oxooctanoic acid (25 mg, 0.05 mmol), HATU (15 mg, 0.04 mmol), and DIPEA (0.1 mL) in DMF was added (2R,4R)-1-((R)-2-amino-3,3-dimethylbutanoyl)-4-hydroxy-N-(4-(4-methylthiazol-5-yl)benzyl)pyrrolidine-2-carboxamide (21 mg, 0.05 mmol). The reaction mixture was stirred at rt for 2 hrs. On completion of the reaction, the mixture was diluted with DCM, washed with water and brine, dried over Na₂SO₄, and concentrated. The crude product was purified by HPLC to obtain P62 as white solid (18 mg, 39%). ¹H NMR (400 MHz, Methanol-d₄) δ 9.02 (dd, J=17.1, 12.3 Hz, 2H), 7.98 (s, 1H), 7.52-7.38 (m, 4H), 7.27-7.11 (m, 2H), 7.12-7.03 (m, 1H), 6.79 (s, 1H), 4.56-4.48 (m, 2H), 4.47-4.33 (m, 2H), 4.05 (dd, J=10.5, 5.2 Hz, 1H), 3.72 (dd, J=10.7, 3.8 Hz, 1H), 3.38 (p, J=1.7 Hz, 1H), 2.93 (d, J=6.0 Hz, 1H), 2.71 (s, 2H), 2.49 (d, J=1.0 Hz, 3H), 2.39 (dp, J=27.3, 7.3, 6.8 Hz, 3H), 2.30-2.17 (m, 2H), 2.00 (d, J=13.1 Hz, 1H), 1.70-1.50 (m, 4H), 1.35 (s, 4H), 1.15-0.91 (m, 9H). MS (ESI) m/z=913 [M+H]⁺.

### INCORPORATION BY REFERENCE

The entire disclosure of each of the patent documents and scientific articles referred to herein is incorporated by reference for all purposes. The following references are herein incorporated by reference in their entireties:
1. Rahib L, Smith B D, Aizenberg R, Rosenzweig A B, Fleshman J M, Matrisian L M. Projecting cancer incidence and deaths to 2030: the unexpected burden of thyroid, liver, and pancreas cancers in the United States. Cancer Res 2014; 74:2913-21
2. Raufi A G, Manji G A, Chabot J A, Bates S E. Neoadjuvant treatment for pancreatic cancer. Semin Oncol 2019; 46:19-27
3. Kamisawa T, Wood L D, Itoi T, Takaori K. Pancreatic cancer. Lancet 2016; 388:73-85
4. Binet F, Sapieha P. ER stress and angiogenesis. Cell Metab 2015; 22:560-75
5. Walter P, Ron D. The unfolded protein response: from stress pathway to homeostatic regulation. Science 2011; 334:1081-6
6. Chevet E, Hetz C, Samali A. Endoplasmic reticulum stress-activated cell reprogramming in oncogenesis. Cancer Discov 2015; 5:586-97
7. Luo B, Lee A S. The critical roles of endoplasmic reticulum chaperones and unfolded protein response in tumorigenesis and anticancer therapies. Oncogene 2013; 32:805-18
8. Wang M, Kaufman R J. Protein misfolding in the endoplasmic reticulum as a conduit to human disease. Nature 2016; 529:326-35
9. Xu C, Bailly-Maitre B, Reed J C. Endoplasmic reticulum stress: cell life and death decisions. J Clin Invest 2005; 115:2656-64
10. Calfon M, Zeng H, Urano F, Till J H, Hubbard S R, Harding H P, et al. IRE1 couples endoplasmic reticulum load to secretory capacity by processing the XBP-1 mRNA. Nature 2002; 415:92-6
11. Yoshida H, Matsui T, Yamamoto A, Okada T, Mon K. XBP1 mRNA is induced by ATF6 and spliced by IRE1 in response to ER stress to produce a highly active transcription factor. Cell 2001; 107:881-91
12. Ye J, Rawson R B, Komuro R, Chen X, Dave U P, Prywes R, et al. ER stress induces cleavage of membrane-bound ATF6 by the same proteases that process SREBPs. Mol Cell 2000; 6:1355-64
13. Lee A S. Glucose-regulated proteins in cancer: molecular mechanisms and therapeutic potential. Nat Rev Cancer 2014; 14:263-76
14. Reddy R K, Mao C, Baumeister P, Austin R C, Kaufman R J, Lee A S. Endoplasmic reticulum chaperone protein GRP78 protects cells from apoptosis induced by topoisomerase inhibitors: role of ATP binding site in suppression of caspase-7 activation. J Biol Chem 2003; 278:20915-24
15. Pyrko P, Schonthal A H, Hofman F M, Chen T C, Lee A S. The unfolded protein response regulator GRP78/BiP as a novel target for increasing chemosensitivity in malignant gliomas. Cancer Res 2007; 67:9809-16
16. Jiang C C, Chen L H, Gillespie S, Wang Y F, Kiejda K A, Zhang X D, et al. Inhibition of MEK sensitizes human melanoma cells to endoplasmic reticulum stress-induced apoptosis. Cancer Res 2007; 67:9750-61
17. Garg A D, Maes H, van Vliet A R, Agostinis P. Targeting the hallmarks of cancer with therapy-induced endoplasmic reticulum (ER) stress. Mol Cell Oncol 2015; 2:e975089
18. Shen J, Ha D P, Zhu G, Rangel D F, Kobielak A, Gill P S, et al. GRP78 haploinsufficiency suppresses acinar-to-ductal metaplasia, signaling, and mutant Kras-driven pancreatic tumorigenesis in mice. Proc Natl Acad Sci USA 2017; 114:E4020-E9
19. Clarke W R, Amundadottir L, James M A. CLPTM1L/CRR9 ectodomain interaction with GRP78 at the cell surface signals for survival and chemoresistance upon ER stress in pancreatic adenocarcinoma cells. Int J Cancer 2019; 144:1367-78
20. Niu Z, Wang M, Zhou L, Yao L, Liao Q, Zhao Y. Elevated GRP78 expression is associated with poor prognosis in patients with pancreatic cancer. Sci Rep 2015; 5:16067
21. Lee E, Nichols P, Spicer D, Groshen S, Yu M C, Lee A S. GRP78 as a novel predictor of responsiveness to chemotherapy in breast cancer. Cancer Res 2006; 66:7849-53
22. Serrao E, Xu Z L, Debnath B, Christ F, Debyser Z, Long Y Q, et al. Discovery of a novel 5-carbonyl-1H-imidazole-4-carboxamide class of inhibitors of the HIV-1 integrase-LEDGF/p75 interaction. Bioorg Med Chem 2013; 21:5963-72
23. Maust J D, Frankowski-McGregor C L, Bankhead A, 3rd, Simeone D M, Sebolt-Leopold J S. Cyclooxygenase-2 Influences Response to Cotargeting of MEK and CDK4/6 in a Subpopulation of Pancreatic Cancers. Mol Cancer Ther 2018; 17:2495-506
24. Burslem G M, Smith B E, Lai A C, Jaime-Figueroa S, McQuaid D C, Bondeson D P, et al. The advantages of targeted protein degradation over inhibition: an RTK case study. Cell Chem Biol 2018; 25:67-77 e3
25. Oyadomari S, Mori M. Roles of CHOP/GADD153 in endoplasmic reticulum stress. Cell Death Differ 2004; 11:381-9
26. Martin S, Lamb H K, Brady C, Lefkove B, Bonner M Y, Thompson P, et al. Inducing apoptosis of cancer cells using small-molecule plant compounds that bind to GRP78. Br J Cancer 2013; 109:433-43
27. Chen T C, Wang W, Golden E B, Thomas S, Sivakumar W, Hofman F M, et al. Green tea epigallocatechin gallate enhances therapeutic efficacy of temozolomide in orthotopic mouse glioblastoma models. Cancer Lett 2011; 302:100-8
28. Lee A S, Brandhorst S, Rangel D F, Navarrete G, Cohen P, Longo V D, et al. Effects of Prolonged GRP78 Haploinsufficiency on Organ Homeostasis, Behavior, Cancer and Chemotoxic Resistance in Aged Mice. Sci Rep 2017; 7:40919
29. Cerezo M, Lehraiki A, Millet A, Rouaud F, Plaisant M, Jaune E, et al. Compounds Triggering ER Stress Exert Anti-Melanoma Effects and Overcome BRAF Inhibitor Resistance. Cancer Cell 2016; 29:805-19
30. Bakewell S J, Rangel D F, Ha D P, Sethuraman J, Crouse R, Hadley E, et al. Suppression of stress induction of the 78-kilodalton glucose regulated protein (GRP78) in cancer by IT-139, an anti-tumor ruthenium small molecule inhibitor. Oncotarget 2018; 9:29698-714
31. Garcia-Carbonero N, Li W, Cabeza-Morales M, Martinez-Useros J, Garcia-Foncillas J. New hope for pancreatic ductal adenocarcinoma treatment targeting endoplasmic reticulum stress response: a systematic review. Int J Mol Sci 2018; 19:2468
32. Parmakhtiar B, Burger R A, Kim J H, Fruehauf J P. HIF inactivation of p53 in ovarian cancer can be reversed by topotecan, restoring cisplatin and paclitaxel sensitivity. Mol Cancer Res 2019; 17:1675-86
33. Thomas A, Pommier Y. Targeting topoisomerase I in the era of precision medicine. Clin Cancer Res 2019; 25:6581-9
34. Zagni C, Floresta G, Monciino G, Rescifina A. The Search for Potent, Small-Molecule HDACIs in Cancer Treatment: A Decade After Vorinostat. Med Res Rev 2017; 37:1373-428
35. Kikuchi S, Suzuki R, Ohguchi H, Yoshida Y, Lu D, Cottini F, et al. Class IIa HDAC inhibition enhances ER stress-mediated cell death in multiple myeloma. Leukemia 2015; 29:1918-27.
36. Burkard C, Verheije M H, Wicht O, van Kasteren S I, van Kuppeveld F J, Haagmans B L, Pelkmans L, Rottier P J M, Bosch B J, de Haan C A M. Coronavirus Cell Entry Occurs through the Endo-/Lysosomal Pathway in a Proteolysis-Dependent Manner. PLoS Pathog. 2014; 10:1004502.
37. Shang J, Wan Y, Luo C, Ye G, Geng Q, Auerbach A, Li F. Cell Entry Mechanisms of SARS-CoV-2. Proc. Natl. Acad. Sci. U.S.A 2020; 117:11727-34.
38. Ibrahim I M, Abdelmaleka D H, Elshahat M E, Elfiky A A. COVID-19 spike-host cell receptor GRP78 binding site prediction. J Infect. 2020; 80:554-562.
39. Rangel H R, Ortega J T, Maksoud S, Pujol F H, Serrano M L. SARS-CoV-2 host tropism: An in silico analysis of the main cellular factors. Virus Res. 2020; 289:198154-64.
40. Köseler A, Sabirli R, Goren T, Turkguer I, Kurt O. Endoplasmic Reticulum stress markers in SARS-COV-2 infection and Pneumonia: Case-control study. In Vivo. 2020; 34:1645-50.
41. Cuervo N Z, Grandvaux N. ACE2: Evidence of role as entry receptor for SARS-CoV-2 and implications in comorbidities. eLife. 2020; 1-25.
42. Balmeh N, Mahmoudi S, Mohammadi N, Karabedianhajiabadi A. Predicted therapeutic targets for COVID-19 disease by inhibiting SARS-CoV-2 and its related receptors. Inform. Med. Unlocked. 2020; 20:100407-415.
43. Cardellicchioa C, Capozzi M. A. M, Naso F. The Betti base: the awakening of a sleeping beauty. Tetrahedron-Asymmetry 2010; 21:507-17.

### EQUIVALENTS

The invention may be embodied in other specific forms without departing from the spirit or essential characteristics thereof The foregoing embodiments are therefore to be considered in all respects illustrative rather than limiting the invention described herein.

Scope of the invention is thus indicated by the appended claims rather than by the foregoing description, and all changes that come within the meaning and range of equivalency of the claims are intended to be embraced therein.

What is claimed is:

1. A compound described by Formula I: 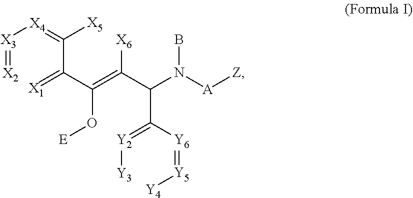
including pharmaceutically acceptable salts (e.g., 2,2,2-trifluoroacetate (TFA) salts and other salts) (e.g., physiologically tolerated acid addition salts), solvates, and/or prodrugs thereof; wherein A, B, E, X₁, X₂, X₃, X₄, X₅, X₆, Y₂, Y₃, Y₄, Y₅, Y₆ and Z independently include any chemical moiety that permits the resulting compound capable of inhibiting GRP78.

2. The compound of claim 1, wherein A, B, E, X₁, X₂, X₃, X₄, X₅, X₆, Y₂, Y₃, Y₄, Y₅, Y₆ and Z independently include any chemical moiety that permits the resulting compound capable of one or more of:
serving as an effective therapeutic agent for treating, ameliorating, and preventing various forms of cancer, viral infections, and inflammatory diseases;
inducing ER stress-mediated apoptosis in the tumor cells implanted in mice without major toxicity to normal tissues; and
inducing ER stress and triggers UPR by inhibiting GRP78.

3. The compound of claim 1, wherein:
X₁ is either CH or N;
X₂, X₃, X₄, X₅ and X₆ are each independently selected from CR¹ or N, with the proviso that at least three of them must be CR¹;
A is selected from CO, SO, and SO₂;
Y², Y³, Y⁴, Y⁵, Y⁶ are each independently selected from CH, CR² and N;
Y⁵ is a bond, in which case one of Y³, Y⁴, or Y⁶ is NR², O, or S, while the other two may be CR² or N;
B, E are each independently selected from hydrogen and R³;
Z is R³;
R¹ is independently selected from the group consisting of H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano, CH₂F, CHF₂, CF₃, OCF₃, SOR⁷, SO₂R⁷, NO₂, COR⁴, C₁₋₆ alkyl-COR⁴, N(R⁷)C₂₋₆ alkyl-NR⁷R⁷, —N(R⁷)C₂₋₆ alkyl-R⁴, N(C₂₋₆ alkyl)₂-NR⁷, —O(CH₂)ₚR⁴, —S(CH₂)ₚR⁴, and —N(R⁷)C(═O)(CH₂)ₚR⁴, with a proviso that not more than three R¹ can be other than H;
R² is independently selected from the group consisting of H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano, 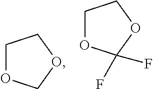
CF₃, OCF₃, SOR⁷, SO₂R⁷, NO₂, COR⁴, C₁₋₆ alkyl-COR⁴, N(R⁷)C₂₋₆ alkyl-NR⁷R⁷, N(C₂₋₆ alkyl)₂-NR⁷, CF₃, CO₂Et, CO₂H, R⁴, —N(R⁷)C₂₋₆ alkyl-R⁴, —O(CH₂)ₚR⁴, —S(CH₂)ₚR⁴, and —N(R⁷)C(═O)(CH₂)ₚR4, with a proviso that not more than two R² can be other than H;
R³ is independently selected from the group consisting of C₁₋₆ alkyl, C₃₋₆ alkenyl, C₃₋₆ alkynyl, C₃₋₇ cycloalkyl, C₄₋₇ heterocycloalkyl, phenyl, naphthyl, 5-10 membered mono- or bicyclic heteroaryl, C₁₋₆ alkyl-C₃₋₇ cycloalkyl, C₁₋₆ alkyl-C₄₋₇ heterocycloalkyl, C₀₋₆ R⁴, and —N(R⁷)C₂₋₆ alkyl-R⁴;
R⁴ is independently selected from the group consisting of OH, NR⁵R⁶, O(CH₂)_{q}NR⁵R⁶—, C₁₋₆ alkoxy, C₁₋₆ alkoxy-C₁₋₆ alkoxy, C₂₋₆ hydroxyalkoxy, cyclopropyl, oxetanyl, oxetanyloxy, oxetanylamino, oxolanyl, oxolanyloxy, oxolanylamino, oxanyl oxanyloxy, oxanylamino, oxepanyl, oxepanyloxy, oxepanylamino, azetidinyl, azetidinyloxy, azetidylamino, pyrrolidinyl, pyrolidinyloxy, pyrrolidinylamino, piperidinyl, piperidinyloxy, piperidinylamino, azepanyl, azepanyloxy, azepanylamino, dioxolanyl, dioxanyl, morpholino, thiomorpholino, thiomorpholino-S,S-dioxide, piperazino, dioxepanyl, dioxepanyloxy, dioxepanylamino, oxazepanyl, oxazepanyloxy, oxazepanylamino, diazepanyl, diazepanyloxy, and diazepanylamino, all of which may be optionally substituted with OH, OR⁷, oxo, halogen, R⁶, CH₂OR⁶, CH₂NR⁵R⁶ or CH₂CH₂CONR⁵R⁶;
R⁵ and R⁶ are each independently selected from the group consisting of H, —CD₃, C₁₋₆ alkyl, C₃₋₆ alkenyl, C₃₋₆ alkynyl, C₃-s cycloalkyl, —(C₁₋₃ alkyl)-(C₃₋₈ cycloalkyl), C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, and 5-11 membered heteroaryl; wherein R⁵ and R⁶ may be further independently substituted with up to three substituents selected from the group consisting of hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano, and halo; or alternatively, R⁵ and R⁶, taken together with the N atom to which they are both attached, may form a heterocycloalkyl ring of 4-7 members, containing up to one other heteroatom selected from O, S, or NR³, or a heterobicycloalkyl ring of 6-12 members which may be fused, bridged or spiro, and contain up to two other heteroatoms chosen from O, S(O)ₓ, or NR³;
each R⁷ is independently selected from H, —CD₃, C₁₋₆ alkyl, C₃₋₆ cycloalkyl, phenyl, naphthyl, 5-10 membered mono- or bicyclo-heteroaryl, C₂₋₆ hydroxyalkyl, —SO₂-alkyl, NH-C₂₋₆ alkyl-NR⁵R⁶, C₁₋₆ alkoxy-C₁₋₆ alkyl, and C₂₋₆ alkyl-NR⁵R⁶; alternatively, two R⁷ taken together with the same N atom to which they are both attached, form a heterocyclic ring of 4-7 members, containing up to one other heteroatom selected from O, S, or NR³;
p=0, 1, 2, 3, or 4;
x=0, 1, or 2.

4. A compound described by Formula I: 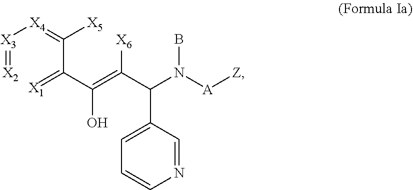
including pharmaceutically acceptable salts (e.g., 2,2,2-trifluoroacetate (TFA) salts and other salts) (e.g., physiologically tolerated acid addition salts), solvates, and/or prodrugs thereof; wherein A, B, E, X₁, X₂, X₃, X₄, X₅, X₆, Y₂, Y₃, Y₄, Y₅, Y₆ and Z independently include any chemical moiety that permits the resulting compound capable of inhibiting GRP78.

5. The compound of claim 4, wherein A, B, X₁, X₂, X₃, X₄, X₅, X₆, and Z independently include any chemical moiety that permits the resulting compound capable of one or more of:
serving as an effective therapeutic agent for treating, ameliorating, and preventing various forms of cancer, viral infections, and inflammatory diseases;
inducing ER stress-mediated apoptosis in the tumor cells implanted in mice without major toxicity to normal tissues; and
inducing ER stress and triggers UPR by inhibiting GRP78.

6. The compound of claim 4, wherein:
X₁ is either CH or N;
X₂, X₃, X₄, X₅ and X₆ are independently selected from CR¹ or N, with the proviso that at least three of them must be CR¹;
A is selected from CO, SO, and SO₂;
B and Z are each independently selected from hydrogen and R³;
R¹ is independently selected from the group consisting of H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano, CF₃, OCF₃, SOR⁷, SO₂R⁷, NO₂, COR⁴, C₁₋₆ alkyl-COR⁴, N(R⁷)C₂₋₆ alkyl-NR⁷R⁷, —N(R⁷)C₂₋₆ alkyl-R⁴, N(C₂₋₆ alkyl)₂-NR⁷, —O(CH₂)ₚR⁴, —S(CH₂)ₚR⁴, and —N(R⁷)C(═O)(CH₂)ₚR⁴, with a proviso that not more than three R¹ can be other than H;
R⁴ is independently selected from the group consisting of OH, NR⁵R⁶, O(CH₂)_{q}NR⁵R⁶, C₁₋₆ alkoxy, C₁₋₆ alkoxy-C₁₋₆ alkoxy, C₂₋₆ hydroxyalkoxy, cyclopropyl, oxetanyl, oxetanyloxy, oxetanylamino, oxolanyl, oxolanyloxy, oxolanylamino, oxanyl oxanyloxy, oxanylamino, oxepanyl, oxepanyloxy, oxepanylamino, azetidinyl, azetidinyloxy, azetidylamino, pyrrolidinyl, pyrolidinyloxy, pyrrolidinylamino, piperidinyl, piperidinyloxy, piperidinylamino, azepanyl, azepanyloxy, azepanylamino, dioxolanyl, dioxanyl, morpholino, thiomorpholino, thiomorpholino-S,S-dioxide, piperazino, dioxepanyl, dioxepanyloxy, dioxepanylamino, oxazepanyl, oxazepanyloxy, oxazepanylamino, diazepanyl, diazepanyloxy, and diazepanylamino, all of which may be optionally substituted with OH, OR⁷, oxo, halogen, R⁶, CH₂OR⁶, CH₂NR⁵R⁶ or CH₂CH₂CONR⁵R⁶;
R⁵ and R⁶ are each independently selected from the group consisting of H, —CD₃, C₁₋₆ alkyl, C₃₋₆ alkenyl, C₃₋₆ alkynyl, C₃-s cycloalkyl, —(C₁₋₃ alkyl)-(C₃-s cycloalkyl), C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, and 5-11 membered heteroaryl; wherein R⁵ and R⁶ may be further independently substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo; or alternatively, R⁵ and R⁶, taken together with the N atom to which they are both attached, form a heterocycloalkyl ring of 4-7 members, containing up to one other heteroatom selected from O, S, or NR³, or a heterobicycloalkyl ring of 6-12 members which may be fused, bridged or spiro, and contain up to two other heteroatoms chosen from O, S(O)ₓ, or NR³;
each R⁷ is independently selected from the group consisting of H, —CD₃, C₁₋₆ alkyl, C₃₋₆ cycloalkyl, phenyl, naphthyl, 5-10 membered mono- or bicyclo-heteroaryl, C₂₋₆ hydroxyalkyl, —SO₂-alkyl, NH—C₂₋₆ alkyl-NR⁵R⁶, C₁₋₆ alkoxy-C₁₋₆ alkyl, and C₂₋₆ alkyl-NR⁵R⁶;
alternatively, two R⁷ taken together with the same N atom to which they are both attached, form a heterocyclic ring of 4-7 members, containing up to one other heteroatom selected from O, S, or NR³;
p=0, 1, 2, 3, or 4;
x=0, 1, or 2.

7. A compound described by Formula Ib: 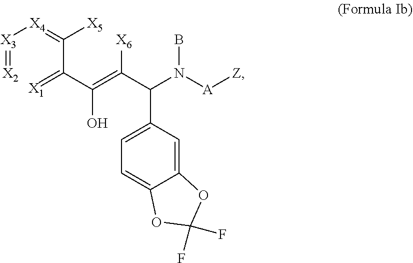
including pharmaceutically acceptable salts (e.g., 2,2,2-trifluoroacetate (TFA) salts and other salts) (e.g., physiologically tolerated acid addition salts), solvates, and/or prodrugs thereof; wherein A, B, X₁, X₂, X₃, X₄, X₅, X₆ and Z independently include any chemical moiety that permits the resulting compound capable of inhibiting GRP78.

8. The compound of claim 7, wherein A, B, X₁, X₂, X₃, X₄, X₅, X₆, and Z independently include any chemical moiety that permits the resulting compound capable of one or more of:
serving as an effective therapeutic agent for treating, ameliorating, and preventing various forms of cancer, viral infections, and inflammatory diseases;
inducing ER stress-mediated apoptosis in the tumor cells implanted in mice without major toxicity to normal tissues; and
inducing ER stress and triggers UPR by inhibiting GRP78.

9. The compound of claim 7, wherein:
X₁ is either CH or N;
X₂, X₃, X₄, X₅ and X₆ are independently selected from CR¹ or N, with the proviso that at least three of them must be CR¹;
A is selected from the group consisting of CO, SO, and SO₂;
B, Z are each independently hydrogen or R³;
R¹ is independently selected from the group consisting of H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano, CF₃, OCF₃, SOR⁷, SO₂R⁷, NO₂, COR⁴, C₁₋₆ alkyl-COR⁴, N(R⁷)C₂₋₆ alkyl-NR⁷R⁷, —N(R⁷)C₂₋₆ alkyl-R⁴, N(C₂₋₆ alkyl)₂-NR⁷, —O(CH₂)ₚR⁴, —S(CH₂)ₚR⁴, and —N(R⁷)C(═O)(CH₂)ₚR⁴, with a proviso that not more than three R¹ can be other than H;
R⁴ is independently selected from the group consisting of OH, NR⁵R⁶, O(CH₂)_{q}NR⁵R⁶, C₁₋₆ alkoxy, C₁₋₆ alkoxy-C₁₋₆ alkoxy, C₂₋₆ hydroxyalkoxy, cyclopropyl, oxetanyl, oxetanyloxy, oxetanylamino, oxolanyl, oxolanyloxy, oxolanylamino, oxanyl oxanyloxy, oxanylamino, oxepanyl, oxepanyloxy, oxepanylamino, azetidinyl, azetidinyloxy, azetidylamino, pyrrolidinyl, pyrolidinyloxy, pyrrolidinylamino, piperidinyl, piperidinyloxy, piperidinylamino, azepanyl, azepanyloxy, azepanylamino, dioxolanyl, dioxanyl, morpholino, thiomorpholino, thiomorpholino-S,S-dioxide, piperazino, dioxepanyl, dioxepanyloxy, dioxepanylamino, oxazepanyl, oxazepanyloxy, oxazepanylamino, diazepanyl, diazepanyloxy, and diazepanylamino, all of which may be optionally substituted with OH, OR⁷, oxo, halogen, R⁶, CH₂OR⁶, CH₂NR⁵R⁶ or CH₂CH₂CONR⁵R⁶;
wherein R⁵ and R⁶ are each independently selected from the group consisting of H, —CD₃, C₁₋₆ alkyl, C₃₋₆ alkenyl, C₃₋₆ alkynyl, C₃-s cycloalkyl, —(C₁₋₃ alkyl)-(C₃₋₈ cycloalkyl), C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, and 5-11 membered heteroaryl; wherein R⁵ and R⁶ may be further independently substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo; or alternatively, R⁵ and R⁶, taken together with the N atom to which they are both attached, form a heterocycloalkyl ring of 4-7 members, containing up to one other heteroatom selected from O, S, or NR³, or a heterobicycloalkyl ring of 6-12 members which may be fused, bridged or spiro, and contain up to two other heteroatoms chosen from O, S(O)ₓ, or NR³;
R⁷ is independently selected from the group consisting of H, —CD₃, C₁₋₆ alkyl, C₃₋₆ cycloalkyl, phenyl, naphthyl, 5-10 membered mono- or bicyclo-heteroaryl, C₂₋₆ hydroxyalkyl, —SO₂-alkyl, NH—C₂₋₆ alkyl-NR⁵R⁶, C₁₋₆ alkoxy-C₁₋₆ alkyl, and C₂₋₆ alkyl-NR⁵R⁶; alternatively, two R⁷ taken together with the same N atom to which they are both attached, form a heterocyclic ring of 4-7 members, containing up to one other heteroatom selected from O, S, or NR³;
p=0, 1, 2, 3, or 4;
x=0, 1, or 2.

10. A compound described by Formula II: 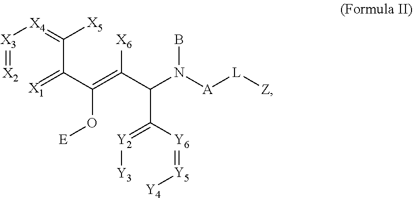
including pharmaceutically acceptable salts (e.g., 2,2,2-trifluoroacetate (TFA) salts and other salts) (e.g., physiologically tolerated acid addition salts), solvates, and/or prodrugs thereof; wherein X₁, X₂, X₃, X₄, X₅, X₆, Y₂, Y₃, Y₄, Y₅, Y₆, A, B, E, L and Z independently include any chemical moiety that permits the resulting compound capable of inhibiting and/or degrading GRP78.

11. The compound of claim 10, wherein X₁, X₂, X₃, X₄, X₅, X₆, Y₂, Y₃, Y₄, Y₅, Y₆, A, B, E, L and Z independently include any chemical moiety that permits the resulting compound capable of one or more of:
serving as an effective therapeutic agent for treating, ameliorating, and preventing various forms of cancer, viral infections, and inflammatory diseases;
inducing ER stress-mediated apoptosis in the tumor cells implanted in mice without major toxicity to normal tissues; and
inducing ER stress and triggers UPR by inhibiting GRP78.

12. The compound of claim 10, wherein:
X₁ is either CH or N;
X₂, X₃, X₄, X₅ and X₆ are each independently selected from CR¹ and N, with the proviso that at least three of them must be CR¹;
A is selected from the group consisting of CO, SO, and SO₂;
Y², Y³, Y⁴, Y⁵, Y⁶ are each independently selected from the group consisting of CH, CR² and N;
Y⁵ is a bond, in which case one of Y³, Y⁴, or Y⁶ is NR², O, or S, while the other two may be CR² or N;
B, E are each independently selected from H and R³;
R¹ is independently selected from the group consisting of H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano, CF₃, OCF₃, SOR⁷, SO₂R⁷, NO₂, COR⁴, C₁₋₆ alkyl-COR⁴, N(R⁷)C₂₋₆ alkyl-NR⁷R⁷, —N(R⁷)C₂₋₆ alkyl-R⁴, N(C₂₋₆ alkyl)₂-NR⁷, —O(CH₂)ₚR⁴, —S(CH₂)ₚR⁴, and —N(R⁷)C(═O)(CH₂)ₚR⁴, with a proviso that not more than three R¹ can be other than H;
R² is independently selected from the group consisting of H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano, 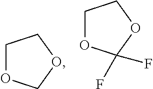
CF₃, OCF₃, SOR⁷, SO₂R⁷, NO₂, COR⁴, C₁₋₆ alkyl-COR⁴, N(R⁷)C₂₋₆ alkyl-NR⁷R⁷, N(C₂₋₆ alkyl)₂-NR⁷, CF₃, CO₂Et, CO₂H, —N(R⁷)C₂₋₆ alkyl-R⁴, —O(CH₂)ₚR⁴, —S(CH₂)ₚR⁴, and —N(R⁷)C(═O)(CH₂)ₚR⁴, with a proviso that not more than two R² can be other than H;
R³ is independently selected from the group consisting of C₁₋₆ alkyl, C₃₋₆ alkenyl, C₃₋₆ alkynyl, C₃₋₇ cycloalkyl, C₄₋₇ heterocycloalkyl, phenyl, naphthyl, 5-10 membered mono- or bicyclic heteroaryl, C₁₋₆ alkyl-C₃₋₇ cycloalkyl, C₁₋₆ alkyl-C₄₋₇ heterocycloalkyl, and C₁₋₆ R⁴;
R⁴ is independently selected from the group consisting of OH, NR⁵R6, O(CH₂)_{q}NR⁵R⁶, C₁₋₆ alkoxy, C₁₋₆ alkoxy-C₁₋₆ alkoxy, C₂₋₆ hydroxyalkoxy, cyclopropyl, oxetanyl, oxetanyloxy, oxetanylamino, oxolanyl, oxolanyloxy, oxolanylamino, oxanyl oxanyloxy, oxanylamino, oxepanyl, oxepanyloxy, oxepanylamino, azetidinyl, azetidinyloxy, azetidylamino, pyrrolidinyl, pyrolidinyloxy, pyrrolidinylamino, piperidinyl, piperidinyloxy, piperidinylamino, azepanyl, azepanyloxy, azepanylamino, dioxolanyl, dioxanyl, morpholino, thiomorpholino, thiomorpholino-S,S-dioxide, piperazino, dioxepanyl, dioxepanyloxy, dioxepanylamino, oxazepanyl, oxazepanyloxy, oxazepanylamino, diazepanyl, diazepanyloxy, and diazepanylamino, all of which may be optionally substituted with OH, OR⁷, oxo, halogen, R⁶, CH₂OR⁶, CH₂NR⁵R⁶ or CH₂CH₂CONR⁵R⁶;
R⁵ and R⁶ are each independently selected from the group consisting of H, —CD₃, C₁₋₆ alkyl, C₃₋₆ alkenyl, C₃₋₆ alkynyl, C₃-s cycloalkyl, —(C₁₋₃ alkyl)-(C₃-s cycloalkyl), C₃-s cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, and 5-11 membered heteroaryl; wherein R⁵ and R⁶ may be further independently substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo; or alternatively, R5 and R⁶, taken together with the N atom to which they are both attached, form a heterocycloalkyl ring of 4-7 members, containing up to one other heteroatom selected from 0, S, or NR³, or a heterobicycloalkyl ring of 6-12 members which may be fused, bridged or spiro, and contain up to two other heteroatoms chosen from O, S(O)ₓ, or NR³;
R⁷ is independently selected from the group consisting of H, —CD₃, C₁₋₆ alkyl, C₃₋₆ cycloalkyl, phenyl, naphthyl, 5-10 membered mono- or bicyclo-heteroaryl, C₂₋₆ hydroxyalkyl, —SO₂-alkyl, NH—C₂₋₆ alkyl-NR⁵R⁶, C₁₋₆ alkoxy-C₁₋₆ alkyl, and C₂₋₆ alkyl-NR⁵R⁶; alternatively, two R⁷ taken together with the same N atom to which they are both attached, form a heterocyclic ring of 4-7 members, containing up to one other heteroatom selected from O, S, or NR³;
p=0, 1, 2, 3, or 4;
x=0, 1, or 2;
L is a linker selected from a group consisting of —(CH₂)m-, —(CH₂CH₂O)n-, 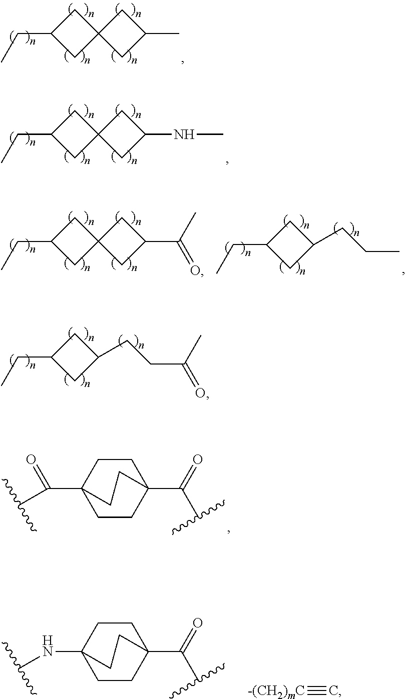
—(CH₂CH₂O)ₙC≡C;
m=0, 1, 2, 3, 4, 5, 6, 7, 8, 9, or 10;
n=0, 1, 2, 3, 4, 5, or 6;
Z is a radical of an E3 ligase ligand selected from the group consisting of:

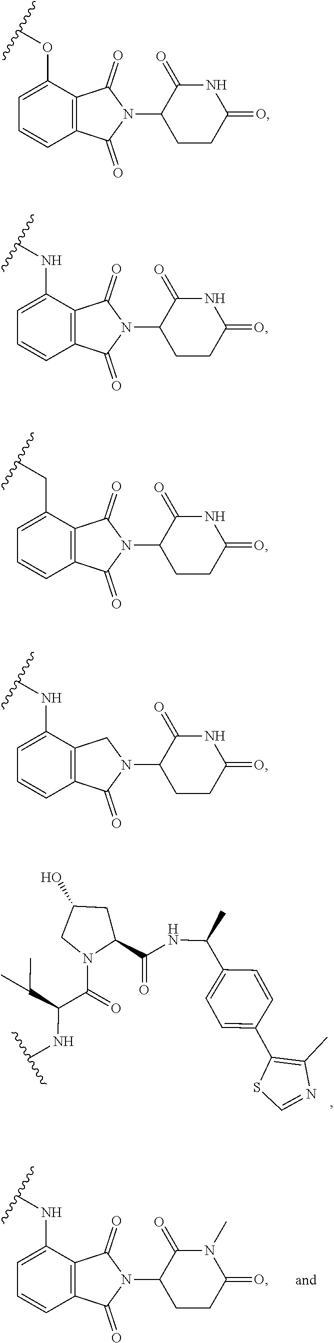

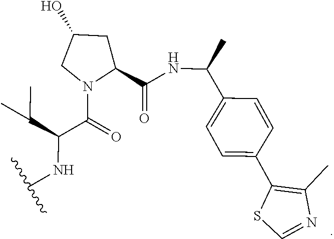

13. A compound described by Formula III: 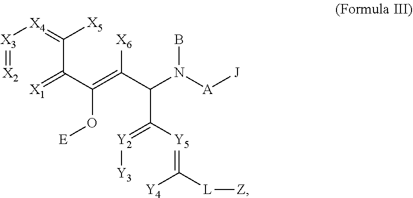
including pharmaceutically acceptable salts (e.g., 2,2,2-trifluoroacetate (TFA) salts and other salts) (e.g., physiologically tolerated acid addition salts), solvates, and/or prodrugs thereof; wherein X₁, X₂, X₃, X₄, X₅, X₆, Y₂, Y₃, Y₄, Y₅, A, B, E, J, L and Z independently include any chemical moiety that permits the resulting compound capable of inhibiting GRP78.

14. The compound of claim 13, wherein X₁, X₂, X₃, X₄, X₅, X₆, Y₂, Y₃, Y₄, Y₅, A, B, E, J, L and Z independently include any chemical moiety that permits the resulting compound capable of one or more of:
serving as an effective therapeutic agent for treating, ameliorating, and preventing various forms of cancer, viral infections, and inflammatory diseases;
inducing ER stress-mediated apoptosis in the tumor cells implanted in mice without major toxicity to normal tissues; and
inducing ER stress and triggers UPR by inhibiting GRP78.

15. The compound of claim 13, wherein:
X₁ is either CH or N;
X₂, X₃, X₄, X₅ and X₆ are each independently selected from CR¹ and N, with the proviso that at least three of them must be CR¹;
A is selected from CO, SO, and SO₂;
Y₂, Y₃, Y₄, Y₅ are independently selected from CH, CR² and N;
B, E and J are each independently selected from H and R³;
R¹ is independently selected from the group consisting of H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano, CF₃, OCF₃, SOR⁷, SO₂R⁷, NO₂, COR⁴, C₁₋₆ alkyl-COR⁴, N(R⁷)C₂₋₆ alkyl-NR⁷R⁷, —N(R⁷)C₂₋₆ alkyl-R⁴, N(C₂₋₆ alkyl)₂-NR⁷, —O(CH₂)ₚR⁴, —S(CH₂)ₚR⁴, and —N(R⁷)C(═O)(CH₂)ₚR⁴, with a proviso that not more than three R¹ can be other than H;
R² is independently selected from the group consisting of H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C1-₆ acyl, C₁₋₆ acylamino, cyano, CF₃, OCF₃, SOR⁷, SO₂R⁷, NO₂, COR⁴, C₁₋₆ alkyl-COR⁴, N(R⁷)C₂₋₆ alkyl-NR⁷R⁷, N(C₂₋₆ alkyl)₂-NR⁷, CF₃, CO₂Et, CO₂H, —N(R⁷)C₂₋₆ alkyl-R⁴, —O(CH₂)ₚR⁴, —S(CH₂)ₚR⁴, and —N(R⁷)C(═O)(CH₂)ₚR⁴, with a proviso that not more than two R² can be other than H;
R³ is independently selected from the group consisting of C₁₋₆ alkyl, C₃₋₆ alkenyl, C₃₋₆ alkynyl, C₃₋₇ cycloalkyl, C₄₋₇ heterocycloalkyl, phenyl, naphthyl, 5-10 membered mono- or bicyclic heteroaryl, C₁₋₆ alkyl-C₃₋₇ cycloalkyl, or C₁₋₆ alkyl-C₄₋₇ heterocycloalkyl, and C₁₋₆ R⁴;
R⁴ is independently selected from the group consisting of OH, NR⁵R⁶, O(CH₂)_{q}NR⁵R⁶, C₁₋₆ alkoxy, C₁₋₆ alkoxy-C₁₋₆ alkoxy, C₂₋₆ hydroxyalkoxy, cyclopropyl, oxetanyl, oxetanyloxy, oxetanylamino, oxolanyl, oxolanyloxy, oxolanylamino, oxanyl oxanyloxy, oxanylamino, oxepanyl, oxepanyloxy, oxepanylamino, azetidinyl, azetidinyloxy, azetidylamino, pyrrolidinyl, pyrolidinyloxy, pyrrolidinylamino, piperidinyl, piperidinyloxy, piperidinylamino, azepanyl, azepanyloxy, azepanylamino, dioxolanyl, dioxanyl, morpholino, thiomorpholino, thiomorpholino-S,S-dioxide, piperazino, dioxepanyl, dioxepanyloxy, dioxepanylamino, oxazepanyl, oxazepanyloxy, oxazepanylamino, diazepanyl, diazepanyloxy, and diazepanylamino, all of which may be optionally substituted with OH, OR⁷, oxo, halogen, R⁶, CH₂OR⁶, CH₂NR⁵R⁶ or CH₂CH₂CONR⁵R⁶;
R⁵ and R⁶ are each independently selected from the group consisting of H, —CD₃, C₁₋₆ alkyl, C₃₋₆ alkenyl, C₃₋₆ alkynyl, C₃-s cycloalkyl, —(C₁₋₃ alkyl)-(C₃-s cycloalkyl), C₃-s cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, and 5-11 membered heteroaryl; wherein R⁵ and R⁶ may be further independently substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo; or alternatively, R⁵ and R⁶, taken together with the N atom to which they are both attached, form a heterocycloalkyl ring of 4-7 members, containing up to one other heteroatom selected from O, S, or NR³, or a heterobicycloalkyl ring of 6-12 members which may be fused, bridged or spiro, and contain up to two other heteroatoms chosen from O, S(O)ₓ, or NR³;
R⁷ is independently selected from the group consisting of H, —CD₃, C₁₋₆ alkyl, C₃₋₆ cycloalkyl, phenyl, naphthyl, 5-10 membered mono- or bicyclo-heteroaryl, C₂₋₆ hydroxyalkyl, —SO₂-alkyl, NH—C₂₋₆ alkyl-NR⁵R⁶, C₁₋₆ alkoxy-C₁₋₆ alkyl, and C₂₋₆ alkyl-NR⁵R⁶;
alternatively, two R⁷ taken together with the same N atom to which they are both attached, form a heterocyclic ring of 4-7 members, containing up to one other heteroatom selected from O, S, or NR³;
p=0, 1, 2, 3, or 4;
x=0, 1, or 2;
L is a linker selected from a group consisting of —(CH₂)ₘ—, —NH(CH₂)ₘ—, —NH(CH₂CH₂O)ₙ—, —(CH₂CH₂O)ₙ—, —(CH₂)ₘCO—, —(CH₂CH₂O)ₙCO—, 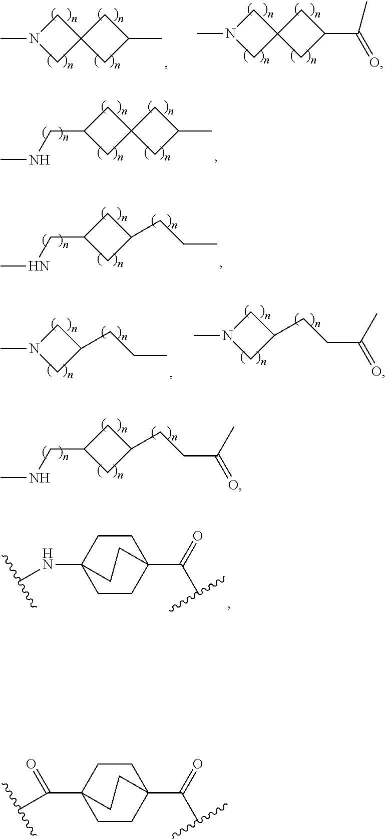
—NH(CH₂)ₘC≡C, and —NH(CH₂CH₂O)ₙC≡C;
m=0, 1, 2, 3, 4, 5, 6, 7, 8, 9, or 10;
n=0, 1, 2, 3, 4, 5, or 6;
Z is a radical of an E3 ligase ligand selected from the group consisting of:

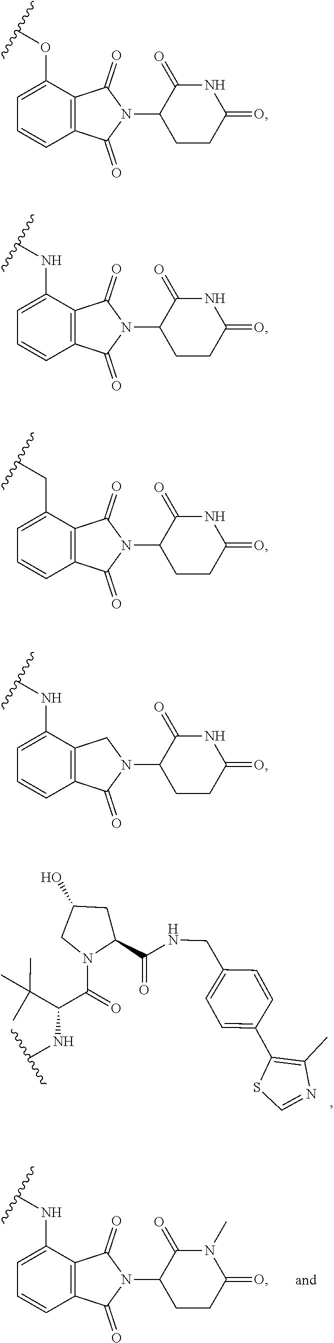

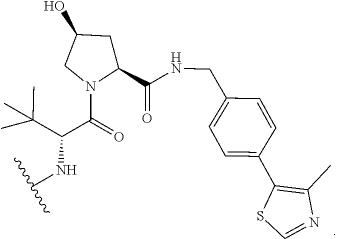

16. A compound described by Formula IV: 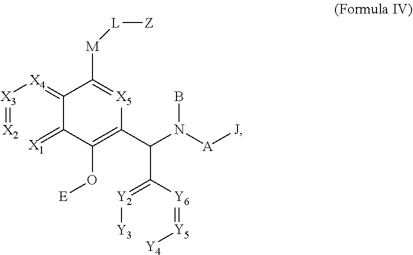
including pharmaceutically acceptable salts (e.g., 2,2,2-trifluoroacetate (TFA) salts and other salts) (e.g., physiologically tolerated acid addition salts), solvates, and/or prodrugs thereof;
wherein X₁, X₂, X₃, X₄, X₅, Y₂, Y₃, Y₄, Y₅, Y₆, A, B, E, M, J, L and Z independently include any chemical moiety that permits the resulting compound capable of inhibiting GRP78.

17. The compound of claim 16, wherein X₁, X₂, X₃, X₄, X₅, Y₂, Y₃, Y₄, Y₅, Y₆, A, B, E, M, J, L and Z independently include any chemical moiety that permits the resulting compound capable of one or more of:
serving as an effective therapeutic agent for treating, ameliorating, and preventing various forms of cancer, viral infections, and inflammatory diseases;
inducing ER stress-mediated apoptosis in the tumor cells implanted in mice without major toxicity to normal tissues; and
inducing ER stress and triggers UPR by inhibiting GRP78.

18. The compound of claim 16, wherein:
X₁ is either CH or N;
X₂, X₃, X₄, X₅ are independently selected from CR¹ and N, with the proviso that at least three of them must be CR¹;
A is selected from the group consisting of CO, SO, and SO₂;
Y₂, Y₃, Y₄, Y₅ are independently selected from the group consisting of CH, CR² or N;
M is selected from the group consisting of NH and CO;
B, E and J are each independently selected from the group consisting of H and R³;
R¹ is independently selected from the group consisting of H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano, CF₃, OCF₃, SOR⁷, SO₂R⁷, NO₂, COR⁴, C₁₋₆ alkyl-COR⁴, N(R⁷)C₂₋₆ alkyl-NR⁷R⁷, —N(R⁷)C₂₋₆ alkyl-R⁴, N(C₂₋₆ alkyl)₂-NR⁷, —O(CH₂)ₚR⁴, —S(CH₂)ₚR⁴, and —N(R⁷)C(═O)(CH₂)ₚR⁴, with a proviso that not more than three R¹ can be other than H;
R² is independently selected from the group consisting of H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano, 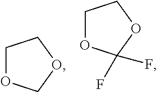
CF₃, OCF₃, SOR⁷, SO₂R⁷, NO₂, COR⁴, C₁₋₆ alkyl-COR⁴, N(R⁷)C₂₋₆ alkyl-NR⁷R⁷, N(C₂₋₆ alkyl)₂-NR⁷, CF₃, CO₂Et, CO₂H, —N(R⁷)C₂₋₆ alkyl-R⁴, —O(CH₂)ₚR⁴, —S(CH₂)ₚR⁴, and —N(R⁷)C(═O)(CH₂)ₚR⁴, with a proviso that not more than two R² can be other than H;
R³ is independently selected from the group consisting of C₁₋₆ alkyl, C₃₋₆ alkenyl, C₃₋₆ alkynyl, C₃₋₇ cycloalkyl, C₄₋₇ heterocycloalkyl, phenyl, naphthyl, 5-10 membered mono- or bicyclic heteroaryl, C₁₋₆ alkyl-C₃₋₇ cycloalkyl, C₁₋₆ alkyl-C₄₋₇ heterocycloalkyl, and C₁₋₆ R⁴;
R⁴ is independently selected from the group consisting of OH, NR⁵R⁶, O(CH₂)_{q}NR⁵R⁶, C₁₋₆ alkoxy, C₁₋₆ alkoxy-C₁₋₆ alkoxy, C₂₋₆ hydroxyalkoxy, cyclopropyl, oxetanyl, oxetanyloxy, oxetanylamino, oxolanyl, oxolanyloxy, oxolanylamino, oxanyl oxanyloxy, oxanylamino, oxepanyl, oxepanyloxy, oxepanylamino, azetidinyl, azetidinyloxy, azetidylamino, pyrrolidinyl, pyrolidinyloxy, pyrrolidinylamino, piperidinyl, piperidinyloxy, piperidinylamino, azepanyl, azepanyloxy, azepanylamino, dioxolanyl, dioxanyl, morpholino, thiomorpholino, thiomorpholino-S,S-dioxide, piperazino, dioxepanyl, dioxepanyloxy, dioxepanylamino, oxazepanyl, oxazepanyloxy, oxazepanylamino, diazepanyl, diazepanyloxy, and diazepanylamino, all of which may be optionally substituted with OH, OR⁷, oxo, halogen, R⁶, CH₂OR⁶, CH₂NR⁵R⁶ or CH₂CH₂CONR⁵R⁶;
R⁵ and R⁶ are each independently selected from the group consisting of H, —CD₃, C₁₋₆ alkyl, C₃₋₆ alkenyl, C₃₋₆ alkynyl, C₃-s cycloalkyl, —(C₁₋₃ alkyl)-(C₃-s cycloalkyl), C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, and 5-11 membered heteroaryl; wherein R⁵ and R⁶ may be further independently substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo; or alternatively, R⁵ and R⁶, taken together with the N atom to which they are both attached, form a heterocycloalkyl ring of 4-7 members, containing up to one other heteroatom selected from O, S, or NR³, or a heterobicycloalkyl ring of 6-12 members which may be fused, bridged or spiro, and contain up to two other heteroatoms chosen from O, S(O)ₓ, or NR³;
R⁷ is independently selected from the group consisting of H, —CD₃, C₁₋₆ alkyl, C₃₋₆ cycloalkyl, phenyl, naphthyl, 5-10 membered mono- or bicyclo-heteroaryl, C₂₋₆ hydroxyalkyl, —SO₂-alkyl, NH—C₂₋₆ alkyl-NR⁵R⁶, C₁₋₆ alkoxy-C₁₋₆ alkyl, and C₂₋₆ alkyl-NR⁵R⁶; alternatively, two R⁷ taken together with the same N atom to which they are both attached, form a heterocyclic ring of 4-7 members, containing up to one other heteroatom selected from O, S, or NR³;
p=0, 1, 2, 3, or 4;
x=0, 1, or 2;
L is a linker selected from a group consisting of —CO(CH₂)ₘ—, —NH(CH₂)ₘ—, —NH(CH₂CH₂O)ₙ—, —CO(CH₂CH₂O)ₙ—, —NH(CH₂)ₘCO—, —NH(CH₂CH₂O)ₙCO—, 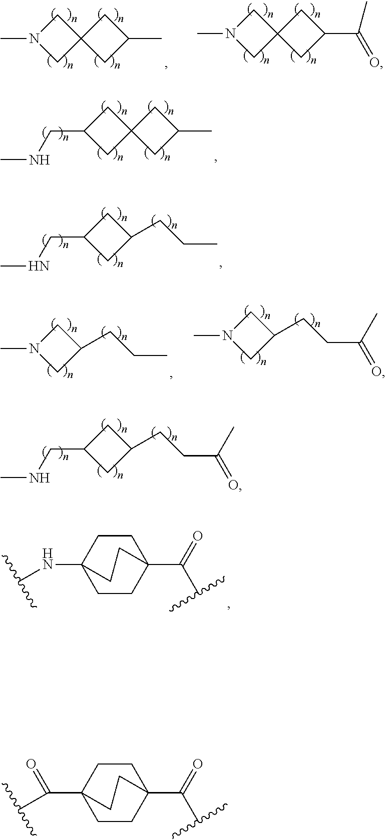
—NH(CH₂)ₘC≡C, —NH(CH₂CH₂O)ₙC≡C—CO(CH₂)ₘC≡C, and —CO(CH₂CH₂O)ₙC≡C;
m=0, 1, 2, 3, 4, 5, 6, 7, 8, 9, or 10;
n=0, 1, 2, 3, 4, 5, or 6;
Z is a radical of an E3 ligase ligand selected from the group consisting of:

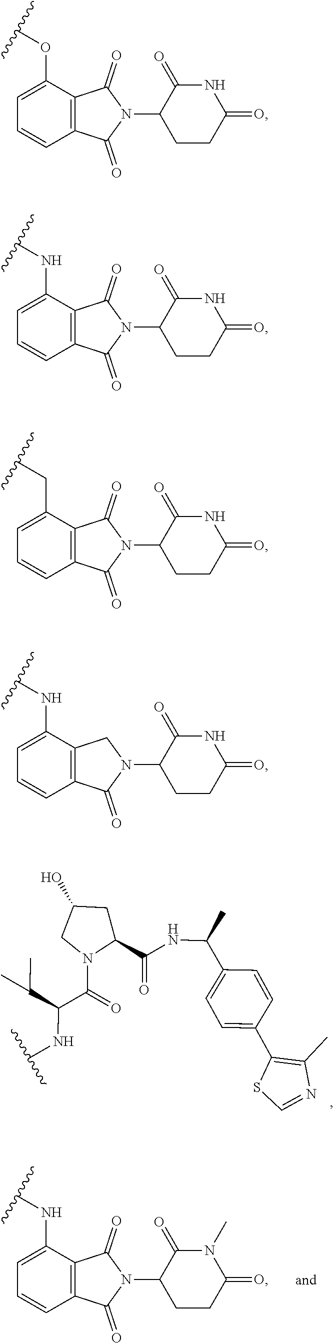

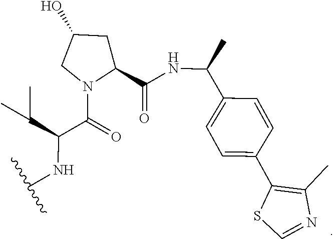

19. A compound selected from one of the compounds recited in Tables I or II.

20. A pharmaceutical composition comprising a compound recited claim 1, 4, 7, 10, 13, 16 or 19.

21. A method of treating, ameliorating, or preventing a disorder related to GRP78 activity in a patient comprising administering to said patient a therapeutically effective amount of the pharmaceutical composition of claim 20.

22. The method of claim 21, wherein said disorder related to GRP78 activity is a hyperproliferative condition and/or inflammatory condition.

23. The method of claim 22, wherein the inflammatory condition is a chronic auto immune disorder and/or a viral infection such as SARS CoV-2.

24. The method of claim 22, wherein the hyperproliferative condition is diabetes and/or cancer.

25. The method of claim 24, wherein the cancer is one or more of pancreatic cancer, leukemia, colon cancer, CNS cancers (e.g. glioblastoma), non-small lung cancer, melanoma, ovarian cancer, renal cancer, breast cancer, prostate cancer, esophageal cancer, cervical cancer and colorectal cancer.

26. The method of claim 21, wherein said patient is a human patient.

27. The method of claim 21, further comprising administering to said patient one or more agents for treating the disorder related to GRP78 activity.

28. The method of claim 27, wherein the agents comprise topoisomerase I inhibitors and HDAC inhibitors.

29. The method of claim 27, wherein the agents comprise anticancer agents, wherein said anticancer agent one or more of a chemotherapeutic agent, and radiation therapy.

30. The method of claim 21, wherein administration of the compound results in induced ER stress-mediated apoptosis in the tumor cells implanted in mice without major toxicity to normal tissues.

31. The method of claim 21, wherein administration of the compound induces ER stress and triggers UPR by inhibiting GRP78.
